SEQUENCE LISTING (69 sequences)

SEQ ID NO: 1 - DNA, 159047 bp, Homo sapiens
gtagatttgctataacaggctgaaagctcagtcagtggtgtatgcttcctggcacactatggtgttcatgtgccacctggctagctctggcccaaaagatgcaaatgagagtccattggatcagggttctgggaaagcttttgctttctgggtataaagggtcagactcagctgtcacatgcccttttttcttttctttcccctccttcctgcctgaaatgcagtcatgatgcctcgggtggagcaggaatcttgctagcgtgaggagcaggccacatgctgagagagtggtggagcagaaagcaggaggcagcctgatggtcctgggagctgttgtcccatccaggatttcctccctcttggcttcttgatatatgaggaaaagtaaaatctgatttggttgggcaactatggctgggtttctgttacatgtagccaaatgcaaccctaacatagacaatgccctacactcattgcatcccaagaaaagcctggaattttcagatcaagttttagcctatcagtggcccacacttagagacttgccacatttttcaggtgagggacctggatccctgtatttgtttcttgtcactactgcaagaaattaccatatacttagtggcttaaaacaacagatttattattatagttctggaggtaagaagtcctaaacttgaagtaccagcagggctgcattccttctggaggctctaggggagaatccaattctttgccttttttagcttccagaggccacctgcatttcttggctcatggcccctttgtattcaaggccagtagcacggtatcttcacacctctctctctgacctctgcttctgctgtcacacctccttctctgactctgactctctcctgcttccctctttcccttatgaggactcatgtcattatactgggcccacctggaccatccaggataatctccctacaggagcataggctctgcaagatctaagtcccttttgccatgtcaggtaatatatttagaggttgtggggattgggaggtggacatctcaagggggcactattctgtctactgtagtcgtagaaatgaaaagggacttgcccaagagtacccacagagctgactgaaaagaaacaactatgggtaggctcacaccactggcagaagcaccccttttcctcctccttggcatgaagctaaagcccagccattctccaaggtccatttcaggctgcccctgcccagaagccacagctgccctcctaggcctgcagaggtcatcctcttgactgtgtctgtgtcactcattgccacttggcacacatttggtcagcagtcgtgaaggtcttccattgttctctgcccagtcttgatcctggaaggcaaggatcacaccctagaacttttttgtgcccactgcacagagtagggtctcaatacgtgtacggttcatgatgttcctagcagattctccaaactgactcactcagcccctggaacactaaaaactgcatttcccagactcctttgcagcgtgggctaggttccaccaagccagacacagtgaaagtgagtgggtggccacaggtggagggcacagtggtggatatgttttgttcttttgggggcagctatgctgggggctgcactaagtgatggggcagtggtcccaatggagtagtaaaattaagtgtttctcgtggctgcatggctcctggctgtgtaggacttagacccgtcatctgtaagctccttcacctcatgtaaacaaaacccctctagctggagtggtgtctattgtctgcgactaagaacccttatataaggtatactactccccaaccccattagtggaaatcccaaagggtaggaactgtattttatttcacttgtaaacagctcccctagtaagcatgtcaacaaaatatacacaattcattgaaccccataacatttcaacgaattcctcatcctttctgtgaatcaagagcctgaaaagaaatggtgaaataatatgatcctctcttctttgaaagctcaaagctatgttggaccagaagtaaagtgttctcgtttctatttaataacttgaaaggttccgaggggccattgaggaaactcctcccttttaatatcaatgtgtatttattgcaaaaataatgtagcatcgagtggtattttatagcttatccaaaaacctcctgggtttaacgcattgtgatagtcccgttttcttctcagcccaggtcctatgcatcctcatctatgcagggctgttatctgcatataattttttttttttttaagacaaagtcttgctctgtcgccccggctggagtgcagtggtgcaatctcggctcactgcaacctccgcctcccaggttcaagcggttcttccgcctcagcctaccgagtagctgggactacaggcatgcgccaccacacctaggtgatttttgtatttttagtagagacaggggtttcaccatgttgaccaggctggtctcgaactcctgatctcaagcgatccacccgcctcagcctcccaaagtgctgggattacaggcataagccactacgcccggcctcaattttgtattgtactttttctttctttctttaatagagacagggtctcactatgttgactaggttggtctagaactcctgggcacaagctgtccgcccgcttctgcctcccaaagtgctgggattgcaggcgtgaaccaccgcccctggctacaggtgccttcttgtctcaatttgcctttgacctttcttagggacttgttttctgcttttcctgctctttgtccgctgatctcctgggaagaaagcttccgaaaaggacaccgtttcaggggcgagtgacgccggggtgcccaggccgcgccccagttccgggtttgcacccggtcttcttgccctgccccgcccgcgggactacagttcccaggcgcccctgcgcggccgcggcgccggcgccggcgtcggttgggacgcggaggcggagctgaggagcagggccgggcgccatggcaccgtggggcaagcggctggctggcgtgcgcggggtgctgcttgacatctcgggcgtgctgtacgacagcggcgcgggcggcggcacggccatcgccggctcggtggaggcggtggccaggtgagtgggccccgggacgccgctggggccgccgagctctaagctcagcccgctccctggctgccggcagggggcggggcggcagggggcggggccgcggcgcaggccccgcctcggtctcccccttcccaccccggtgcgcgcacagtgctgaccacggacgaccccactgttgcccccggcgagcaccaggactctgctggttagggctgcgcggtcagacagggcggccacctggtacgtgcgctgctgagcgcttgacctgcggccagtctgaattgagatgtgctgtaagcgtaaaatgcataccgattaacaagacttagccgggcgcggtggctcgcatctgtgattctagcacttgcagaggcagaggagggaggatcgcttgaggctagggggttggagaccaccctgggcaacatagggagaccccgtctctgccaaaaatttaaaaattagccgggtgtggtggtgcacgcctgtaggcccagccacttgggaggctgaggcaggaggatcgcttgagcccaggaggtcaaggctacagtgagctgtgatcaccccactgtactctagcctggactgcagagcgagacggtctcataaacaaaacccccaaaaacaccagacttcgtgagtaaaatgtaaaataaaaacatgaaaatgggttatgttgtttgcctgttgaaatgatatactttttgtttgttttgttttgttcttttcagatggagtcttgctctgtcgcccaggctggagtgcaatggcacgatctcggctcactgcaacctccgcctcccgggtgcaagtgattctcccccttcaggctcccgagtagctgggattacaggcacccgccatcatgcccagttactttttttatttgttttgtttgagacggagttttgctcttgttgcccaggctggaatgcagtggtgcaatctctgctgactgcaaccgccgcctcccaggttcaagcgattctcctgcctcagcctcctgaatagctggaattacaggcatgtgccaccacacccggctaattttttgtatttttagtagagacggggtttcaccatgttgggcaggctggtctctaactcctgacctcaggtgatccacctgcctcggcctcccaaagtgctgggattataggcgtgagccacatgcccagttgataatatttttaatatactgttttaggttaaaatatttgaaattaagtataccttttttttttaacttttgttaatgtgcttattagaaaactttaaattacctttgtggctctcattataattctgttggactgcattggtttaggtgagaggaggaggaagagaacatcgtgccagccaagacttgaccatgaggacacttttttttttttgagacagttttgctcttgttgcccaggccggagtgcagtggcgccatctgggctcactgcaacctctgcctcccaggttcaagtgattctcctgcctcagcctcccaagtatctgggattacaggcacccgccaccacgcccagctaatttttgtatttttggtagacatggggtttcaccatactggctaggctggtctcgaactcctgacctcaactgatccacctgcctgggccttctaaagtgctgggattacaggcgtgagccaccgcacctggctgacacttttctttgtataatttttttttttgagacaaggtctcattatgttgccagggtggtcttgaactccttggctcaggcagtcctcctgccttggtctcccaaagtgctaggattacaggcatgagccaccacacctggcctaatttttttttctttttttttttttttttttgagacaaggtcttactttgccacccaggctggagtgcagtggttcaatctcactgcaacctctacctcaagggctcaagagatcctcccacctcagcctcccaagtagctggaactacaggcacgtgccaccatgcccagctaatttttttgtgttttttgtagagatggggttttgccatgttgcccaggccggtcttgaactcctgggctgaagtgatctgcctgcctcagctcccagagttctgggattagaggtgtgagccactgtggccagccagaaggacactttcagtagcatggttggcaccactggacctgggctttgtaaggcctcctgttggcatcccaggtcagcactgccatgtgttgataggcagccgggaagggctaactgccaggacttggcctcccggagtcactagctgaggcaccctggcttggtagcctgatgtcactgggactcaatatacctatctgtaggtggcaattaactgagtctcttggtccttcttagagaccccattctaccaatctgcatgtctgatgtctgggagctgctgagataatagggacccaaacggtcccgcatagtgaagccggaggtgtttgggaggctgggaactgggaacagggagacttggcttccaggagcaagcacaaagggcagcccagacctaggacactccgctgggtcctgggtctgctctcagtgttggctgaagttcagggttaagtgcattcctgaccttattctccattactccaaagctttagactagctcagtcacagatgagagcattctgtgagtggcactggtgggaatccagcagagggctctgggcagtgttttggggtgaagtgggcctcatctggggagcaggaagccagagctcaatgctgtgactggggcaggcaaggcctcatgggagaccctgccactcattgcttgcggaagatacatttgacattatctaccagtggttccacttctgagaatttgtccttgtctcatgcgtgagaaatgacatgagtacaaggtcattcctggcagtaccttttaaatagcaaaagactggaaacagctcagatacccatcagtaggggagtattagggtctgatgtgtggaggggagagggacagtaaagctgcaggcacacaggtaaatcatcctcacaggtgggcacacgggtaaagcgcatcatcacaggtaggcctaaggggtgtgaagacgggagggtactgagagtgtgatggacgagggtggcctgtgctagagagggggtcgggacagctctgaggagaaggcactggacagacccggagggataagagggaactgtctgcaggaagaggcggaggaagcgcattccaggcagaaggaatggtagctgcaaagacctcaggaagagagcacttggcgagttcgagggcgtggggtgggggagtgggtacaccaggcaggaacttgaggggcacggagaggtgtccggactttatcctgagtggcaggaagtcagtgaagactctagataggcatatcatgtggtctgatttatatgttttaaagaaatacaaatgtaaaaagtgagacatacaagtcaaatgaaagcaagggagatttcctttgaaaccttggagagaaggcctttacatccaggccttgaaatctagaaagcaaaaacaacgaacaagcagcccaatagcaatatgagcaaaagatgcaaacaggtcatggaagaggaagtgagagaggctccgccttactcaaataagaggtgtgtgatctcggctcactgcagcctctgcctcctgggttcaagccactcttgtgcctcagcctcctgagcagctgggactacaggcatatgccaccgtgcccagctaatttttggtatttttagtagaggcaggattttaccatgatggccaggctggtcccgaactcctggcttcaagtgattcacccaccttggcctcccaaggtgctgggatcacaggcgtgagccactgcgcccagcctgagaaatgcaaattaaaactatatcaagctgccctgtttcatcaatttgacaaaattggccaaaatccaaaagtttgagaacactttctctgtgagagtttgtggaaacagaccctgccacacattgcttgcagaagatacatttgacattatctaccagtggttgcgcttctgagaatttgtcctatctcgtgtgagaaaggacacaagtacaaggtcattcctggcaatgccttttaaatagcaaaagactggaagcagctcagatatccatcagtaggggagttttagggtctgtctgtttatacagcagaataacatgcagtgtaagaaggaatgagtaccttgtctatagacatattactagtggagacagcaagcacacagcaggatgcacggtgcgctgtttctggtatagaaaatgcagaaatgcaagaatgtatttctcctgctgtgtttgcataaagacactggaaggacgcaggctgcttcagagtctgggaagagctggaggataaggaaagtgggggccactgtgtggttggagacagggtaggagaaagactcttcactgtataactttgtataccaacgtttttgtttttgtttttgaaacagagtgttgctctgtcacccagattggagggcagtggtgcaattttggcttactgtaacctctgcctcctgggttcaagtgattatcctgcctcagcctcccgagtagctgagattacaggcacctgccaccatgcctggctaatttttgtatttttagtagagatggggttttgccacattggccaggctggtttcgaactcctgacctcatgtgatctgcccaccttggactcccaaagtgctgggattacaggtgtgagccaccgcgcccagcctgcatatcatttttaagatttttaaactatgtgaatgtgttttctattcaaaatattacaattcaaatattaaattgaatattaaaattcaaaatattaacgtctgggcacagtggctcatacctgtaatcccagcactttgggaggctggaggtagaaggattgagaccagcctgggcaacacagcgagacccccatctctacaaaaaatacaaaaattagccgggcatggtggtgcacgcctgtagtcccaactacttgggaggctgaggcaggagaactgcctgagcccaggagtttgaggctgccgtgagctatgaatgtgctactgcactccagcctgggtgacagagtgagactctaaaaaaaaaaaccattgttttttaaataaaatatttcaaggagatctctctggctgcagtgtggagaacaggctggcagtagggcaggaggtgggggggcttggactgggtagaaggcaatggagatggggaggaaggcatgaattaaagaggtgttttggaagcaggacctatgggacctgcggacatacggggtattgtgggggtcagggagaacttgaaataaagggtggtttccggtgtctgactggaacagctggcagggtggagagggggcagggagggggtcatggggcgggaaccatgatggctcatgatgtatgtcaagttcctggcctggtgtttggcacacagtagacccacaacagtgtaacattaaagaccactttcttttctttttcttttttttttttttttgagacaggatcttgatctgtctcccaggctggagtgcagtgacacaatcacggctcactgcagccttgacctcctcctaggctcaaaaaaatctttctacccaagtttcccagatagctgggactacaggtatacctccacatgcaggtttttcttttgtgtgtgtgtgtgttttttgttgttgttgttgttgttgttttgagacagtctcactctgtctcccaggctggagtgcagtggtatgatctcggctcactgcagcctccgcctcctgggttcaagggattctcctgcctcagagtagctgggattacaggtgcctgccaccatgcctggttaatcatttgtatttttagtagagacggggtttcaccatgttgaccaggctggtctcaaactcctgacctcaagtgatccactcgcctgggcctcccaaagtgctgggattacaggcatgagccaccatgcccagcccccaagggacagtttgagccctggtctgcccaacccagacccctacttggaatccttcagggagaagggggtgttgggaaatgtcacaggcttctctaacagcttattttgagcagatgacccccacgtgatatagaactgtccaacaaacgtgcagattccaggttgtgacattggaaagggttctgttaacttctctgggttctgggttggtgtcttctgactgattgattgccaggagggttttgtttggatttgctttggctcctgcagatttatctagctgggggtgtctctggtagcagctatactgtatacatctaacagcaatgtaataacaatcccgaaatacaccacagcctgttatgtagctgggagaggctaaaatctcccttcccaaaagcatatgggattatttttggtgaggggacatcacacccgtgatatgggaggttttgttattgtgctgtctgtgggcctgcagaaccgatggcctggtctctcaaccccatgggcttcccatgccataggaccaagtccccatgtcctgccttgtccacgaggaccttctcaactgacctttgtggcctcttccactggcccccagttacacagacttcttggtggggttttcactgaaggggccgctgagctgtccagcacccacaaacctgtttccgcccacaccagcgtgcccttcggctctcggcagaccttggcagggcccccttcagcctttctgttggcttttaagggcaaggcccctccactctggcccagtgcctctggggccagatcatcaccctcagggcccgggaagctccgtacaccctccctcgcatgcctgtggggctcgtggcagatgcccagagtggctgcgaaggtggtgcaggaaacgccccctccaggaaagtgtgcctgcttttggaattttccctggggattttccagagatgcaggacacctgttttcctcgcctggtgattgagccgggaagccttcatggagcaggccctacttgcccaggtgaagtctgtgtggcctgcagccacgggggcgagtggccctgcgcctttcatgctgtggcctccttgatatgtacagcctgctttgaagtcctgcaggagctcaaaggaagttcctggggtgagaccagccgccgcagagggaaaagtgtgattggagtggggtggggggtcttagggttggcagggggaacacatgcctccgtggcagctcccggcaccatgggctccctggcagcatcgtccgttctctgagcctgcgaggggcgacacactgtcaccattcttcttcttggattttctgtgatgatgggtcctccgattttattgagggccacggagcacgggggaagtagaagctcaactttttaaaataatatccacagcaacagcagtcaccccgcatcgagggaggcctgtcgtgcgccaggcaggtgcttaaccacccaccttcccttcacaccagcccctgaggtggggggggcttcctcttttcaaagtgaggaccaaaaatcggagagattcaggaactggcagaagtagaaaatgttgccgagccctcattaaagatgcatgcacacacacacattcgttcatttattccttttttatttttttttcattcattcattcctttactcatacactcaacaagcaaacattgagtgcctctgctgtactaaagacaccatcctaggccctggggatgtgcagcagtgaacaggtggagccgatgctgcagtcatgggggaccagcaggcgacagcgtgcactcgggaaggctagagcatctgagacacgcagtgaggtggggagccctgggctgggtacctggcctcgcagatcccatggcgagggagctgggattggaggatgctgagcgagccagccaggcaggcatctgggctaagagcatcccagatgaaggagcaggaggcccaaacctctgaggccacactggtgctgctggccaggcagggcccacttgctgctctaggacttgtgggtgggcgtggtcacaggccggaggacatctgtaaccggccgtgtccccccaacaggccctttattgaagccctttcatgggcggattgggttccccgaggggagccattcagaggcagggcccagcacagcgaggggtgcagaggccggcctcggggccctgggcctatggctgggaggcatctgcatgtgaaagggggtgggcatttactgcatcctgtcttgggccactaaagcacatctgctgggcccagcccaggcccggctcggcgtttttggagccggcggaaggaagaagacttggcccagggcagcctcataaagcaattcccacccaggaccgccccgatcgatcggcctggtgctgtggagccaggcccacagcccttccgtccagtagcacctctcaatcccctgtgtgtctgggtgccttcgggatcctgataaaacgtaggtgcctcctagtaggtctggaacagcgaaggccagggccaggggatctttgaagtccagctggtcctggcaccccagtatcccctccttcatgcccggcttccctggtaccatccagccctagggacacaggtccctgggagtagcagcaatagtaacaacaacaacaacaacaacaacaacaacaacaataataataatgtaggctgtgggcctgggctggaacttcatctctattgcctcatccaacccccacacgcggtgtggcacctgccggaggggtctggcaggacattgtagggagagggccatttgtttgcatttgtttaagccaacaggagttttcctgatgcaacctggaccttggacaaggggtgtttgtcgagaacgtgctgtcgtcacctgtggtcccatttgctgtcaggaggtgaagcactttgtcttcagagatgggagccggtgctctttcaccctgggttctgtgatttcagctgtcatttcaacattttaagtttcataagaaataggtggcctccttatgtaaacattctcaccagcctgtgaatatacagagtgaaaagtccaagtcctccatcctcgaccaagtcccagtgacttccccagggtgacacaatacatccttttccttcgtattcctcaaaagcacagaggattttgagatgcagccttccctgactcttgtccagttctgcgggcacctgccccatcactcttgaatgccaccgccccaccattgcccaaggcatgtgtttggttagtcacaggtcactggaggctggagaccccttgcaggtccccgggccaggagttttgaattctttgtagcctcagagcacttatccaaccaaatgaaattttgagtagaactccaaaattatatatatataatatatatatttatatattatatatatatttatatattatatatatttatatatttatatatatttatatattatatatatttatatatttatatatatttatatattatatatatttatatatttatatatatttatatatattttatatatatttatatatatataaatttttttatatttatatattttttatatatttatatatattacatatatttttatattttatatatatttatatatgtatttatatatatgtatatttatatatatttgtatatatatttatgtatatttatatatatttgtatatatatttatgtatatatatatttgtatatatatttgtatatatatatatttatatataaatatttatgagaacagtgtttgaatagtatattatttttaatagggatgggatcttaatatattgcccaggctggtctcaaactcctggcctcaaggagtcctcctgcctcagcctacccagtagctgggattataggtgtgtgccaccgtgtctagctcagaatggaatatgttttctgtgttcaaatttataatgtttgttctgaggtcaactgatgagaatgatggaagtgtttttatgaataacaatgttgggctgggtgcagtggctcacgcctgtaatccctgcactttgggaggccaaagcgggcagatcacataaggtcaggagtttgagaccagcctggcccacatagtgaaacccccgtctttactaaatatacaaaaatcagctgggtgtggtgttgtgcgcctgtaatcctagctacttgggaggctgaggcgtgagaattgcttgaacccgagaggcagaggctggggtgagccaagattgtgctactgcaccccagcctgggagacacagcaagactctgtctcaaaaaaaatgttgaggtctgcagcagtgaaccacacaacacagtttatatgcttagaagtttatgtgagtgctggaagcgagcttattaggagggatgagtgaatgttttctggtttatccagacttcagactgttagacagctatgaaagaatgacttcgctctgtttggtgctgacattagtttatatgatatgagtgctgtatataccaagtgaaaaagctgtagaataatgtgtataaacttacttttttttttttttttgagacgcagtctcgctctgtcacccaggctggagtgcagtggtgcgatctcagctcactgcaaccttggcctcccgggttcaagcgattctcctcctcagcctcctgagtagctgggactacaggcgcctgccaccacgtctggctaacttttgtatttttagtagagacggagtttcactatgttgaccaggctggttttgaactcctgacctcaggtgatccgcccgcctcagcttcccaaagtgctgggattacaggcgtgagccactgcgcccagcctaaacttccatttttgttaagagaaaacaaaggaaacacaaccacccccgtatatctggggtagagtcataatagaatcacaaacttgaactgtatggaaagagactgaggaaaatctgatggctgcttgaaagctttcccgtggctctgctggcaggagtaaggtctgcttctcagtttctgatctcagccttggtgggcgggggtcaccctgtgaaaatcaagggcagccagggtgggtctggcacaggattattggtttaccctgcaggtgctgcctctggttggaagcccttggccaattacaaggggagtgtggcacaagttacaaagctcatgaatggagtcctgcccctacccccagtgccccacaacgtgcaagaagccatattggcaaagcagtgccacacctgcccactgtggtgtgcagggtgatttcccaggcccccggcaacaggcacgttgctgtccagcttgtagaggaggaacttaaggttcacagaagtgataggacttattcagacaccttttgagggggactgagggctgctgcttctgccagggcaggcaccacctcttacctgttgcaaagcttaatcctgtcacagaaagtgccaggtgtcagctctagtgtgagagcgtgggtttccaatcccagctctgccccatagttgccatgtggcctcaaggaagtcccttagcgttccgtcaccccattatggctgcagctcaagggccccagtttcctatgtgtaattcctgtacccaggctgcaagaattaagtgaaatattttctcttaaaccaatgtttgggtggggcgaggcgactcatgcctgtaatcccagcactttgggaggccgaggcaagtggatcacctgaggtcaggagttcaagaccagcctggccaacatggtgaaaccccgtctcactaaaaatataaaaaaattagctgggtgcagtggtttgcacctctaataccaactactcaggagactgaagaatcacttgaacctgggaggcggaggttgcagtgagctgagattgcaccactgcactccagcctgggcaataccagcgagactcggtctcaaaaaagcaaaaacaaacaaaagccaacgttttcctttttttttaagtttttgttttcttttcctttttaccttaaactatcaggagataaaagccaacgttttacttattccatatctaggagctaaaggagcattttgttgttgaagggtcaaataattttgaagagtgggttgctttgccctttttttttttttttttttttttttgagacagggtctcactttgtcacctaggctggagtgcagtggcacaatcttggctcactgcagcctcgacctcctgggctcaagcgatcctttcacctcagcccccaaagtagctgagactacaagtgcacaccactgcacctggccaatttttgtatttttagtacagacaaggttttgccacgttgcccaggctggtcttgaactccagagctcaacggatctgcccacctcagtctcccaaagtgttgggattacaagtatgaaccaccgtgcccggccatatgaagtcttgtagttgttgaactcggtgtacatgctaaaccaaagcacacagttctgctcaaggagctgtattttccacatctgtttgtattttcatgaagccaacataaaaggattctcatcccagttctgtccctagacaagtcactgaacccctccaaaccacaggtcccatctccaaacagcaggtcccatctctaaaccacaggtcccatctccaaaccacaggtcccatctgtaaatggaaacatgaacccacctctcagactgtcaagaggattcgatgtgaacatgccgttggggctcggtggaggaggagccagtcaccgggcgctgctgtagccactgctggggctcagttcaccctgggctccctgctggtgtaaactttcccctctttctaaaggtgcccctgggcgatacactctgggctcctcctgctggcctccttcccagggcacctggcttcctctcagaatgtacctgccccaacagtgggtttgtcagagacgtggttgtcattgtcaccaccacagcaattactgccatttatttagtatttgaaatgtgccaggtgcttctcatgtcttttttttttgaaatggagtctcgctctgtcgcccaggctggagtgcagtggtgccatctcagcgcactgcaacctctgccacccaggttctagtgattctcctgcctcagcctcctgaatagctgggattacaggtgtgcaccaccacacccagctaacttttgtattttaaatagagatggggtttcactgtgttgttcgccatgttggccaggctggtctcaaactcctgacctcaagtgacccacccgccttggcctcccaaagtgctggggattacaggcatgagccggtggtcaggagttcgagaccagcctggccaacatggtgaaaccccatctctactaaaaatacaaaaaataagctgggcatagtggcgggtgcctgtaatcccagctacttgggaggctgaggcaggaggatcacttgaacctgggaggcagaggttgcagtgagccaagatcatgccactgcactccagcctgggtgactgagtgagactccgtctcaaaaataaaataaaataaaaaataaataatatgatcttcatgacagccctgtccagtagctactgttggtgcctcattttacaggtgaggaaactgaggttgcagagggttaggtgacaagccacagtcatacagggaggaggtgtgtctgcttcccgagctgagctcccaccttacttacaggggcctggcctttgtcccctgcagccaccatggccctcagggacgtcgggctgctgctgaggctcctaggctgaggagtggaagaaaggagccaggatccccacccctcacccccacatgccacgaggcatttcctgtccctctgggccttagtatccccctctgtgaaatgagacactcgggctgtgtggtctccaaagtctctggctgccgattcccatgtacttcattctaaattgcgtttattgattcctgggacatcgatgcttgtgggatcaacatgtccctgtgatctgggctccgtggcacaggcacttcagagaacactctagatgggagttctgccggcaggtgaggaggcgcaggtacgggagggtgtgctgcgtgttttataagtgccctagctgggggccggccccagcttgacctcattgccgtcccgctaaggtgactgcccagctgacccagaccaggaggcccagctaggtcccagctcctgcgtcactgtgtctcttctctccattcaaactttggactctgatgactcaaggctctgtgcaggtgctcaacagcgtctagtggacctgttgccctggccggtcagctggaaggaggagcaagtgcaggaggctcagctggagccagtgtgaggccgtgccctgtgccaggggcccgtggcatctctgctgtgcccagcctttacctcgtctccttcatggcagccctggagagctggaaggctgggcaccaccatcacccatcactgtttatggctcgggttccaagactccaccagcctggtgcgcgcgccagggtgggaagccagaccccacgcccaggggacagatgccgatctgttgctgctcctacctgcaggtggtcccccagcccaagctggtgtgcttcatggtgcagggcacggggtgggctcatgcgcacatcagtgtgggcgtccccggggcatatgtgaataactgtatttttctattgaaaaaacaactctgctctgcttacggggctcctgcttgaccttctgttttggaaaattggatttttggaggaaatcagcaccttgtgggtttcaattctccaaacatgcgtgttcaatctccattgctttccaaaaggggagcgactgaagatctgaaaattagacagtggctagcactgcccagaggccccgttattaaaaacatttcaaggccgggtgccggtggcttacgcctgtaatcccagcactttgggaggctgagactggcggatcacgaggtcaggagttcaagaccagcctagccaacatagcgaaacccgtctactaaaaatacaaaaattagctgtgcatggtggcacgcgcctgtagtcccaactactcgggaggttgaggcaggagaatcgcttgaacctgggaggcagaggttgtggtaagttgagattgtgccactgcactccagcctgggcaacggagtgagactctgtctcaaaacaaaaacatttcatgccagtggcattgctgagggcctggggctggtgctgcacctgcatctcttttcactacaacccccggggaagggggtggtcaatgtcctttcccctctctgaggaaactgaggcattgagcttgtatttctctgcactggttcccatttcagtagtaatagggaccctccagcacaccaggtgatgtctgcaatgtggccccagaggcaggagcaggctgggagttaggaggctgaagcctgatgtgggggagcgctgccccccaaccccactccctgggaaccaaccctccacccctttgccctgctgcctccacacccctttcctctggctgtgaatttctccattaccaagtgggcccgtcagatctggaacttgggtcagttaggtgcttctcccagtaaagcacctactgtgctgggtactgggtactgtgcccggtagattttgggcaggattctagagagcatctggcaaagttgctgcaggaaagggccagtcactgcctgccagtctcggcccctgtgaatgcacgagttgacatttctcaaattccagcctcactttgtgggccaggcagcccttctgacctatgagctgccaaactgagccttttgatgacactgcccccgaggattccctgcagccctatgtcctcctgagcacctggtggcccctctgtggcctccgagaccctggcctgggtgctcactgcccctcatccctacacaggagatgctggggtgactcctctgttggtaaaccaaggctctggttgctgaggatggggctggtcctgcgccagggtgcccccaggtgctgatcttggctccactgtggttgggcaccaggcatggcagaggtgctgagcagacaaaaccctgccctcccaagccccgggcttgcagaggaggcaggaggagggaggtggtgatgtcaggaccagagcagcaccaagggctgtgtgggcgcagctcggaggatgggatctggctgggtgcatggctgtggggcctccaggaggaagaggtgtatgaaccctgctttgtaggatgatcagggtcaggccccaggggagcatgaaagcaaagactcagggtgctatgagggcctctgggtcacacgaaggtgaccagtaccggggctagagggcagggggcccagctgggaggggctcatgctgcccttttcctgaggccccctgctgcctgcccagtctcccctccgagggatcccattctccctccagcctcaggaccctcggcggtccccagagggctagctattgagctgtcccaaggtcactcaggatgtccaataattgtcctaacagtttacctgctgtggaacagtaatgagagggttttcattatttgcgggtgagcgctcctggcagatgccaacagccagcagatgtggagagtccgaggtgatttgtaagggccggttcatctgatgcacggtaattgccccgggcgatgttgtcactcagaggctcgtcctggcctgctgagatgaggtaaaggtcagttcaaagatccagtttgggccaggcgcggtggctcaggccgggtgccatggctcacgcctgtaattccagcactttgggaggccgaggcaggtggatcactggagattgggagttcaagaccagcctgggcaacatggtgagaccccgtctctactaaaaatataaaaattagccgggcatggtggtgggcacctgtaatcccagctactcgggagactgaggcaggagaattgcttgaacccaggaggcggaggttgcagtgagctgagattgtgccattgcactccagcctggctgacagagtgagactctgtctcaaaaaaaaaaaaaaaaagatccagtttggctacaggaagtgggagcaaatccccactcccatgggactcctggggaggaggcagcgtgctgaggtgggggccatggtctcagaggggctatctggcagccaggaccctgcagaaggcccctttccccaaggttcttgggtggtggggtgggaacagcccatcccagagctgggcttgtccttctttgcaggggctctttgtacagctctctgcaagccttgctggggtcctggagccgcacctgactagggctttcctgatattttggaattcttgtacttcatgcctcccaaggcttagtctgtgacatctggtggcaggccccacggagggatgacaagggtttgcttccttagctgtcctagtcggcctgggctgccataacaagtgccattgactggggacttcaacagcagacgtttattttttcacagttctggaggctggaaggctgatatcagggggtcggcatggccagtttctggtgaggccccttttctgggcttgtaagcaggtgccggcttgctgtgtgctcacatggcctctttgtgcattcatgagggtgctctctggtgtctcttcctcctaggacactgatcctgtcatgtgggagtcccacccttatgccttcatttcacctgaattactccacaaaggccctgtctccagatgcagtcacattgggggttaggactccccatctgggctcacaggccttgctgcttacaagctgtgtgaccttggtcaggtctctgtctcctggggcctccatcttctcctttctagagtggagataatggttcttgcctcataggctcgtttacccagtgggtacgacttactgagggccacctgacagccaggcacctgggccttggggatgtccattagggacagcaaataccttgggcacgaggacaaaatcagatcatggatcctgagtgacaccaccgtccttacccagatgcaaggctcagtataatgttgtattaatattttactctttaaacaatagtgcctactttctttattttcagtaatttaaaataaggaagaggcaacctggggctggtccctggtggtcagaatgggtctctggtgacggctcaaaggggggtgtggtccggggcgcagaaggacagaacccaatgggagcggacttaccccagcatttcacatccgatttttcagggatagatttcgtctctaaaactagcacttgatattataagagcatataggctgaatgaacttatattgctatttcagaggaggccagtggcttaagagtcccaaagtgagttctgtgacttactcatgtttcatccacatctgaagttgtgtttggaataacagatgtgaggcactgtcatctcactgtcacatcgattgctcactgttctgagaaggtcacatcatcattcattagcaataattagtcccaggggaccccctgcacttcttcactcaactgctccattaacttgggtgcaaatcacacgttccaggtgccaatcaatggagatctatccataatgtacaaggtgacgctattagttacaatatattaactgcctaatttaaaaataaaactatctttatgaagggcaattaaccactaagtgtaattgataattcataaacctctgattaggaaagacaaataataagaaaggaatgaatcacccatcctattgaagaagacgttgccagcctctggtctgcattcactatgtggtcagcagatctacagatttcttcctaaattgcacctgtcagctccagggctgggggtctggaggctcatttcagtgcagaaacatgtgttttcaccactgaagcctgtctgtttctgttgggggttataaggaggctgcaggctcaggacaccctcagggaagtagtgggtgaggtcatttagacttctcagtcagtcagtcattcagcaaatgttaatgggggattgtggagtgccaagccctgtgggaagccctggggatgccgtggagacccgaatagattcatccctgaactcctggagctcgtaggccagtgagaaagacaattgcgtaaatcagatgattggagacactggttagtgttacaaacaaaaactgacagagacagggcggaggcaatggcacactggatggtgggaggaagggacattggcctgagtcctgccacctttttgtagtagctgtcactaccactcatggctgtatttggggccctctctcactggtggggaatcgtgcctggcttatctgccccctttttttttttttttttttttgcgacagagtctcactgtgtgacccaggctggagggcagtggcttgatcttggctcactgtgacctccacctcctgggttcaagagattcttctacctcagcctcccgagtagctaggactacaggtgcccgccaccacacctggctaattttttttggtatttttagtacagacaggatttctccatgttgaccaggctgatctcaaactcctgccctcaagtgatccacccacctcgacctcccaaagtgctggggttacaggactgagccaccatgctctgccttatctgccctctcagtggtcctccactccagcctcatttcctggctccaccttccacataccccacgctgctcgagttctttctatccgagagggtggggccagtgcacacctgcctgtgcgcaaggtgggtggcccttcccaccacctggcctgccctttcctgccatctccttgtccgtccctcaggtctccagttgggcctgaatccccagtcttttaagaccccacactgggctgtgtcctttccccactatgtgggagagctccaggagtgctgatttcctgttcctgactccactgtgtgtagatatcagctccatctccaaagtctcaggctcaagagcggaacagaaagcccttgacatttctgggattcagtccctcacccataacatggataaattacattcacactgggttcagtttgagttgagtttttagttgggtctcacttgctcagatggaagttgatttcaagccgtagttctagtaactgggttgaatctgcgtgtattcatttcattgggccacatctgtaaacagaacttgagcacatgcctcctctctatgtgtgttttctataaattttaaattattcatttaacactattgcttaggttataaaaaatacacaacttgaaagaataggtgaagagccaagatgtctgcatctttggagatcagtggaatgtgtgtgtttcgggacctgtctgagaacagtgccagatgcaattagcatggctgattaattttactagggtttcttaaaagtgcctcccggcccttgggcttgcagcttgatgtcttttccacgagtccacactgcagctccatgcagatgtcaaatatgaccgctgccttgttggggccggtttgtccaggaagggatcttgtgatcttggcgagggagccaggtcctgcctgtcaccctccccccaggtctgagtgacccaccccgcctgcctctgtaggttcctctagcacgctcgggtcccttcctggtgggcgccaggtcctccagcctcctctccctgcacagaagcaccacaccctggccctctctcttgcttccccagggaggcctcccctgaccttcctaccccaccttgtcaccctctgtctgctcaccctgtttatattctgcatagcgtctctctcttctcagtcacaaattgcccttcctttgcagggtctcttgttcctattccaacctcctcctattccaacctggatggtggcctggagatcagggaacttttccatgctcacgactacagctctgtgcaccaggccagtgccaggcacgtagtaggcgctcagtgacagtttgcagaagggtggatgttacttcatttggtgcgttctgagaacttgcccaggtgcagcctggattcgcgggtctcctatgaagtcaggagggaagaagctcggtgccgccggccagtggcagcatcctgaagttcatgagacctccaggctgggtttgggcagggtccacgtcctgtctgtccatcccggaatgttggtgtccctgatgactttggcaaacctttctccattgacagtgatgagaagacaaacctgtctcgggccatctgcctttgtactttttgggaagtatggatccagagttagtaaggcgtggacgtgtgcagctcacttgcaagtggaaatgccaaatgtggtggaaagggacagagaaatagctgtggacattcagaggtggcccctagccaggctgagcaggattgtggagaggaggtattgcatggatctcaggggctgggtagaagtggccaggcaggtgggccaggcacggtggctcacacctgtaatcccagcactttgggacacctgaggtcaggagttcgagaccagccttgctaacatagtgaaaccccatctgtactaaaaatacaaaaaattagccaggtatgatggcaggcgcctgtaatcccagctgctctggaggctgaggcaggagaatcgcttgaatctgggaggcggaggttgcagtgagctgagatcatgccactgcactccagcctgggcgacaagagcgagactccatctcaaaaataaagaaataaaaaaaataaagaagtggcacaggtagagaggcggggggacagagttgcattccaggcaaggggagaggcaatgaaaaggggggtcaggtggggaacctcggggagcctcaggtgtatgaagtccagtgaggatggaaaagagttgtcggggcgtggtatgggggtcctctgagaagggatctgcttcagctcagaaatcccagcctgaacacatcctggatcagccaggaccccctcaggggctcgcagtggcctagtctgctctgggctttgctggatgcccttcgagaatcaccgcgcaacctccttcagaggcccaacactccacgtgctgacttcccgggcctgtgtctcccctgccgcagactgaagcgttcccggctgaaggtgaggttctgcaccaacgagtcgcagaagtcccgggcagagctggtggggcagcttcagaggctgggatttgacatctctgagcaggaggtgaccgccccggcaccagctgcctgccagatcctgaaggagcgaggcctgcgaccatacctgctcatccatgacggtaggcctgtcggacaccaggacctcacgggggtgaaagctcccctttcccagggtgggggctgtgcagagagcctctttcactgggccaaaccactgactgagctaggccaccaacactcatgggttgggggtaaaaacctcatgggacttcctgctgggggctgggggcaggttaggacccagctctgtccattccttggcctcacaccagagggtccttagagcaggttctggatggctcctgggaggaaaatcctaggctctctttcctgatctaggatggaccaagcggccagggaattgtcaaggtggcggagttctagctcctgttgagaaagagacagagagagagagagagagagagagagaaaatgagaatgagaatgaatgaatataaatgagaagctacattgaggtccatagcttctaacgtctggcaccatagccttcaactatgtgaaaaccagccctgctctttggaacagtttgcaaatggcgtaggaacaaggtttgcatcctgattttagtctgaatgcaagaacagacagcccctttgaccctcagtggggaggttttcttggaatgaagcagaaggaacaatattactggtgcctcctgaatatttaaaaagaagagacattagcaattccagacacttctccattggtccactgcaccttcttccttcctctgcacttgatatttttagttttacaaactgctaggcagaggcaaacatggttttttcctttccagggcaaggtgggggctctcaagaagtggcgggtgctccctggaaagcctagatgtcgacttcatagccctggtgccagttaagggtgacaggcctggcctttagaaatcatgtttctcaaaattccttctctgtaatctaggatccctgctagtgcctttagaacatggtcttcaaagaaagaagatttaagaaaaatatcttgccccaccctccagaaaagggttgctggacccgggagtggagctggaagaagccagcaggagagggaagtgggcatccgcttggagggtggccttggccagtgaatcagacaagcagacggcaaagctgggaggctgaagggaggatgaatgcccttcctcagctgtaatcctgcatccgtgttttcaataactagacccttgttttcagacttggaattttttttttcagtcattggtggattttctgtctaacattttatgaaaattttcaagcatccaacaaagttcaaataatttttacttggaatagttttggttttttttttggagacagagtcttgctctgtcacccaggctggagtacaacagtggtgtgatcttggcttcctgtgacttccgcttccctggttcaagcgattcttctgcctcagccccccaagtagctggggttacaggtgtgtgccaccacgcccgcctaatttttgtatttttagtagagatggggtttcaccatgttggccaggctcgtcttaaactcctgacctcaggtgatccacccaccttggcctcccaaagtgctgggattacaggcgtgagccaccgcctccggtcttacttggcatagttttatacccttcacctagattgtcaatgacattttttgatattttcttcactttatatccatccatccctccgtctgtctatccatgttattatttagatgcatttccgagtaaaatgcagacacgggtatactcccctatacacttcagcatatcattggagtttggtatttgtttacggtttttcctgtttgatgtaaaatttacatacaatgaaatgtacacacgtcaagtgtacttttgctgagttttgacatatgcatgcacccatggaacccaaccccgtgaagagctagaccatcaccatcactgacagctctgacagctacctcgcaccttgcccaggtggtcttcaccccttgtccccttagaggcaaccactgtttggattttttccgttataaataagttttcctattctagaattttatgtaaatagaatcatacagtatataattgtgctggggcctccagtgctacccccaggctgaatgattcattagcaggactcacagaacttagaaaaggtattacactcccagttagacttattactgtgaaagaataaagattaaattcagcgaagagaggagacacatggggcaaagtctaggagaagacaggcatgaacttccagttgtcccttcgaaatggagtcctactgggcacacttgattctcccagcgatgatgtgtgacaacacatgggaagcattggccaccagggaagctcactggagcctcagtgtccagggtttttgtgtagacattagcacctgtgtgactgactttagcgactcagtctccagccccacagagtccaaactgatacagctgggccagggccgcagggatacagaaacaggtgttcactgtaagccaagctgttagcgtaaatgatctggtcagactgagagagcagggcccgaggccttgagcatacaaaaccccttatcaggtagaacattgcagtggctcagaggttatctcccaggagccagtcctgaagacaggccttttttggaatgtacagggtttgagcaacctagtcctgctgagttaaccctttccgtcacagtagtcttttgtgtaaggcttcttttacccaacgtaatgtttttgagactcatccatattattgcatgtatcagcagtgttgttcatttttattgcagaatagtattccattgagtgaacagaccacagtttatccattcacatgttgttgatacctggattgtgaccagtttcagtctattatgagcaaatctgctatgaacattcttacacagttcttttgggtaaatacctagcagcgaaattactgggtcatagggtacatgtatgttcacctttgtaaaattctgccagacctttttttcagagggattgcaatattttacacgtcctccatctgctgcagctgttcagtatcctcaccagaccttggtgttatcagtctttttaattctcaccattctggtgggtatgaaatcgtatctcattgtgtatttcagtttctttctttcttttttttttttttttttgagacagagtctcgctctgttgcccaggctggaatacagtggtgcaatctcagcccactgcaacctctgcctctcgggttcaagtgattctactgcctcagactcctgagtagctgagattacaggcggccgccaccgtgccgggctgattgttgtatttttagtagagatgggattttgaccatgttggccaggctggtctcgaactcctgaactcaggcgatccgcccaccttgacctcccaaagtgctgggattacaggcatgagccaccgcacctggcctcagtttcttttcaatttagaaaggtcaacatctagtcctaagagagatgacagcatgtttcttctaggatctgaaaacacagatccaatgatttgatgtatccttgcaaattttcctattttaatttaaaatgggggaagacaaagaaggagaaggaggaagagagtttcctaagggagctggtgaactggacctgtggggatggaattctgctttttcttcaggacatatgtgtggggttgcctgaaatgtgtcaacggttcttacagtttgggaccagtagtgccaaccctccaaggcttgtgcagccccatttcctcctgctgcaggctctcagtgtcttctgagcagatttcccttctcagtagcaaagctgtgggtttcctgcctgcgatagtgacctcagggagctcctggggctgatgtggtattgggatggagggtcctgggtgtgtccacctttctcctcataagccctctctgaggagccgaattccctcctctgtcaaataggaataacccttctgtgtctacctcccatggcagctgtgatgatcagaaagaaatgcatctgagaaagggggaggacatgtgaaaggtgtccctggtaggagatggggaggtaggccatgtcctcccagggctccgtggcactctgtctctctctctctttccaggagtccgctcagaatttgatcagatcgacacatccaacccaaactgtgtggtaattgcagacgcaggagaaagcttttcttatcaaaacatgaataacgccttccaggtgctcatggagctggaaaaacctgtgctcatatcactgggaaaagggtaagttggctccagggagagtcatttctcggtgctttagatgatgctgtgccagtctccaacttgcggaatcaggcagagaaggatgcagaaggggaggtgggaggagcaccggtgatcttcttttccctccctttttcatgctttctctcttcatttctttattcctctcctttaatcatttctttaggaaataggaacactgtcttcaggagctggttgatcaagcttccatgtggagtctagactagaagagggcgtcattgcagagggagaccctctggagatctcagaggccaagtgttgattgtcccaagtttagagcaaagaaggagaaggtgcatttgcataggatgtagcagggtgttggagaatgctctcaagggttgtctaattagtaaaacataggcttgagggcctacccagtctgggcaccgacagtctctggccacagatcactgtctctgtgaagttgtgattttgtgatcagtgttttgtctgagcctttagcacatgccttgccatctctttcttaaacaaaacagtgttggttatttttaacatttttcttacactgagatagcacatgttcattgaagaaaaattagacaatacagatgagcaaaagaagaaaccagaaagtatttttagtcccactgcacgaggaaaacacctgtccctattttggtgtatgtccttgcaagtggcttcctgtgcgaatagactattatggatggagatgttcacagcatcgatgtcacacttgaccttgttttgtcatctgttttttattgttcaccaaacaatatttgtgactgacttagcatgcagcaaatggatatgtcttggggcatggttatctatggtgtggctatcttgtatatatatatattttttaagactgagtctcgctctgtcacccaggctggagtgcagcggcgtgatctcggctcgctgcaacctctccctcccgggttcaagcgattctcctgcctcagcctcctgagtagctgggactacaggcgcccgccaccatcccagctaatttttatatttttggtagaggcggggtttcaccatgttggccaggatggtctcaatctcctgaccttgtgatccgcccgccttggcctcccaaagtgctgggattacaggcatgagccaccgcccccaacctcttgtaatttttttctttttttttttttctgcttataagtttattcaatgcaaaataaccctcaccagttttactgaggtggctgaccatgtccacgaccaaatacgcctgtaaactgaaattcggttgctgacccattcccagcctcagctttctcactggcaccagggggacagcactccatctgtgggtgtctctttctctctatggctgtctgtctgtgggtgtctctctctgtctgtgggtgtctttcgccatctgtgggtatctctctctgtctgtgggtatctctcccatctgtgggtgtccatctctgtctttgggtgtctctctttgtgagtgtctctgtctgtggttgtctctgtctgtgggtgtctctctgtgagtgtccctgtgagtgtctctgtctgtgggtgtctctcctcgtctgtgggtatctctccctgtctgtgggtgtctctgttggcttccccacttgtgggtcttgcaggtcggtcacgctccagacctttaggccgcagcctgccagtctccagaccgctgtggcatggggtagcagacacgctctccaggggcagatggtggtaatcgcagagattctggatccccatgtgggtgaggtaccagtagaaatgtctccaggcaaactccttcctgcaacctcaggacctgagagactgcctggccttcatgacgtgaaggttgggcacattctcatctgccagctccgggtcttaggcaggtggacattcttcttggctaccgtgactccctccttaaaaaggagttcataaatagcaatctggttcttcttaggcatcaacatctctgcagctgtagggtccaggtccggggctggaaagcatgatttttttctaactgatctctgctgatggcatctagattgttcctggtttttcaccataccagggctgtgatgagcatcttggtgcatttcggatgacgtctccagatacagttacagaacgagtatttttgaggttcttgaggcatgttgccaagttgtttccagaaagctgcacagacttattctgcacagcctagaattctagaatcacagggttctgcacaacctagagttctggaatcacagggttctgcacagctagaattctagaatcacagggttctgcacagctagaattctagaatcacagggttctgcacaacctagagttctggaatcacagggttctgcacagcctagagttctggaatcacagggttctgcacagctagaattctagaatcacagggttctgcacagcctagagttctggaatcacagggttctgcacagcctagagttttggaatcacagggttctgcacagctagaattctagaatcacagggttctgcacagctagaattctagaatcacagggttctacacagctagaattctagaatcacaggctcccagggttgcaaggacactttggagtgtctacctcagcatctcatgaagtgtgggaattccgaggcggtggcggaggaagtgttttccatcttcggtgctttcgttgcttctggtgacagcgctcactgcctctgcttgctgtacgggaccagctgatggaaccgacagggagggactttttatctggccattggccactgccacacactttgtgtaccccgttttgtgtaattctgactacaaccttgtgggatctaggcaggtcattgctgttttgcaagtggggttgttgaagccacaggagatgaaataagctgctgtcccccagccattgagtgctgataggatcaggagtgccagttggtgtggctgaccccagaccctgtgcgtgttacctctaagctacattctagagcagactttttgcccacacaagccttaaatgtgggctggggacagtggctcacgccggtaatcccagcactttgggaggacaaggtgggcagatcacctgaggccaggggttcaagaccaggctggccaacatggtgaaaccctgtctctactaaaaatacaaaaattagccaggtgtggtggtgcgtgcctatagtcccagctactcgggaggctgacgcatgagaattgcttgaacctgggaggcagaggttgcagtaagtcaagactgcgccattgcactctagcctgggcgacagagcaagactccatctcgaaaaaaacaacaaaaccttaaatgtatttttgaggctgtgtttaaaaatggggatattttacacaaaatatccagatttctggattcttttgaagaatcagaagatctgacaatacggagcctcacattcctgcacacacagcagccatagctggagccactgcctccattagtttgaatttactgcagaccccactcctccctgtcgtccctgtctccagaccacagagttagttgtcattgatcgtgtgccatttgttgtttttttcaaagtagagaagtacttcttcacgctgtgtctctatcaaaaatggacaagtgaaagatgtttcaagaaatgaaaagatttttttagtgacaaaaaatttctagtatgtttctcatataaataaaatgtgtcctgtatgtagtcagggttcctcagagaagcccgaagctacaggatatagatatgtagagagattgtggaggcttggcgagtccaaaatctgcagggcagggctggcaggctagggactcaggaatgcgcgcagcagagtcgtaaggctgtgtgctggtgggattcttgctcggggaaggtcagtctttgttcttgtaaagcctgcaactggttggatgtggtccacccacattgcggaagggaatgtactctcctcctagttcaccgatttaaatgttaatctcatccaaaaacaccttcacagaaacatccagaataatgtttgaccacatatctgggcaccgtggcccagccaagttgacatattaaattaacccttgtagtccctttttaaacttacacccattgcaatttaggtcgctgctatggagcaagccacagaacctggcctcttaactcatttacccgggctgacccattaggcctttgagtcaccaacacctcactagagaacaagcataatgaagaagctctgctgtaattcgttaatgttaacacttttttctttaaagatgtctcatgctgagcttcgtggcacacgcctataatcccagcactttgggaggctgagatgagaggatggcttgagctcagaggttcgagaccagcctgggcagcatagtaagattccgtctctacaaaaaagaaaagaaaaaaagttgtctcataattattaaaaaccactattccagatcatggataataatagtcagaacaggtatattgttgaaaaagaaaaaaaaaaaccggaagcagtcgctttcagctttgtaataccgcagcaaatgtcatagtagacaagggtttttttaaagtaggagattagtggcgtctctgctgatgggggttctgggtgtttaaaaaccagagaaaggaaccggcggttttagggactaggtcccagcttctcaggacagcctgaggaccccccgagagctgccatcaccatgccgtcctcggctgtgggtcactgctggtgaggtccatcctgcactgcgggacacaggggcacaggctgagcggtgcccaccaaggaggcaggagcagcgggtggaggcgccggggcagactgtccctctcctggaaatggagtgatttacatttggcatctgtttggaccccagagtgggaattggctttgtaatttcttctgtcatgccgctctcagaaaattaagctgtttacccgacagactatggaaggttaacggctcttctgggaaaggtcaagtggtcatttgcagggaggctgtgctgaataatctgaaacggcagaagaataaagcataaaatcgcatctgactcctcctcccgggcttccccctttctttccagcatccacgggttcctctttgtggctagaacattcacgtcctaagtggggctgccgttggctgggttaaaccagtaaccagtaacaggagaaatacgtcgcatctctagtgtgtggagattaccgggcctttatattaaaaaaaaattttaagtgcttatatttgctctgtctctaaaaaatgtttccaggtaacagttggcctcaagatgcctttttgtttttctgaatgaataaggaataaagaaaaattctgggcactataaaatcaagcccagattcagttttttaaaaaataaacgttaaagtttctcattttgatttctggaagttcgtgttctccttggactctaaagattagcatgcaaagacaaggttttgattgtgtattttcagttgcacgttcagtttttttaaaatcccatccaggactaaatttaaacataagaaaatccaggtttctttcgctttcgaagaggctgcagggaacttgaggccagaggaagtggcgccgcgtgggtccccttaggaggttttgccagatgatgggaggtgcttgctcccgtccacccaacctctagctcctccagccggccccagggtagctgagggacaggggtctgggcccagaagcccccagaatcctcagctccaaacagagctgtacctgtcgcaatggagtagttttgttgatttcaggttttcttttgttgctgggtaagcatttcctgtgtggggtatattctttcctccgtcctggcttacttccacacatcacttcattagggaggccctccctacccacctcgatgccctccttctcctggcacaggtgggcaggaggggacgagggtcatggcctttgagggcctgcagagggccttgcctgtggtggcgtggccaccctggtcctcagcagggtgctcagtggattccaaggtgctccctggacccggacctccccttcacttccttttagctcaggtctcagctttccatgcctctgctgcctccctgaacccagcctgtcccccgccctgttcaagcagactgcccctcccctgggtcctgccctttaaagtttgtctgtgggcttctttgcttccccaccagactgtgagtccctgtggagcaggctggggctgggagtatccctgagtcctgcaaagggctgtggagcatctgggatcgttaatgccaagggggaccaaggtaacctgggccatttgcagcaccagccagtgcaggccccaggacccggtgtcccctcactgtttcctgagaactctggaccccactaaggaaaccatcagaccaaagtagctgattgtccaactgggaaaaagaaaaagcagtacttttttaggccatgtaaatgcaaaatatacatttgaagtctgcgtttaagacatgaacttagagatttctaagcctgtttttgggggtcactctaaaagggagggcattttcaggtcccccctctgtgagcattcacagcatcccagccagcctgtatccctgtgccgtgaccccagagcacagagcctcagaggctgtggaacaggcctggggttcacgtggagagtcccaagcccacgtcatgacaggagatgcccacaccttcaacaaacagactccaagctccaaagctgcctaacaggagtggcagggggaccagccctggcaaggcccaggtttggctgaagcccgtacctgcagcccagcttgtaagtttagccgggaagcatctgaggcaggcagtggaggacgcacaggggtgagcggttcatctcacaggtgtggctgggtgcctgctacagcttcaaggcatcaggtggggcagaaagggacaggcagggtgcccaggagtggcggagctggaggaagggcacagccccagggcagctcccgaggagctcagttctcacaccccaggtggtcacagaacacatttcagcccccaaattgaacagggctgtgttcattttttccttcccccagctcatcttattgcttgtgtcagcctgggtgaaaggttatttataataatattctcctgtctcatctgggcagcttctgcatggaaggtaccgagttaataactttgcatgcgttattttgttcaagcttcacagcaatccgtgaggtaggtattgtttgttgttcgttttgcccattttacagattaggaaactgaggcccacaggcgtaagcaacccaaagtcacaccgttagaaggcagtgagatctgaatgctcaagtcagcatgcgctcttcttccctctcgttttctgcatacgtgctttctccacgtctcccatatcgtaagggcatggcaggaggagagggaaactgatgtgatggtggatggcaaaacattttagtggttccaagaatcaagcccccttgtgtattcttggctttcaatgaactgcgtcttcagagcagcgcaggttaaaccactttctcgcctcattgtagtcaattggcttagttcagccagagccaggtctggcccaccatgtgtctggactgtgagtgccttgaggctcagtgtgacgcgatcgtagggtgagcctggccgggctgcagccagccaccaagccagccctgggctcaaagagcagtgaggccattttgagagaggaatgctccctcgtgcagagcctgtctccacctccatgagacgggaaggaacagttgtcttctcataaaagcatctggcgcccatgaaacctggcacctcgcttgacctccggctaacgggatgcggcagggtgctgtggaactaatggagttctcttcttcagccagcaattaggggttaattattagcaattaggggttcgccacataccccgtggacctgctgttccttcccaggacacaggtcacttgtcattctctgtgttagcaactgaaaccgtctgccttctgggtcccctgttaatcattgcagcagggtccccacgagtctgacagcagggttgcgttctgtgcgacctgtggcatccctggagctgctggctgctgcgctcgccccactgggtgtggcggcctgccgcccggctctgtggtgctggtcccctgcggtcattctcagcgtagcgcctggactgcccctcagccgcggcttggctctgcagccagcgggacaggcccggtgctcagctcccgatacttagcatcctgcggtcagctccccgtgctcagcatcccgtgctccgttctgctctctcctaggcgttactacaaggagacctctggcctgatgctggacgttggtccctacatgaaggcgcttgaggtaccagccctggttctgtcccaaactctcttcagacctcagggcacctgggactttttgggtctttgttgagaagaggctgtgcttaattaacgtacaaatggccttttgggcttccacaaaggctgccttcctattctgggctcccctggccgggccctcgggccctcaggccctcagctgtcctcctccctccccatctctcccttcccatccttcccatcctccccatcctccccatcctccccatcctccccatcctccccatcctccagtgggcgcagccccccctgcccgccgtggaccctgccgctctcccagctgctctctgccacactcctctcgggcttgtggcttctgtatcagtgctgaccaccgctctcattcctgaccctgggcatctgcagcacctcaagcttgacgggtcctcactggcctccttcctgacttccccttcagtttccaaggccatctgtggccaggcctccgggtggggctcccctgggtccactctggcccaggtcccattagctgccccaggtcagcctcacatcccgtcccatcctcacagctgcagcccctcctgcctgcctgccgtcaggtgggagggagaggcagacgagcagatggagccctgtgatgagctgcgagtgggcaggggagaagagggggagaagggatgagaggacggaattgcaggcgagtgagcagagcactgctctgctggcccttgggagccctgtagttttgccagctcttggatgccttctgctgtgggtaggcctttgagtggatggcagggtgcctggtgctaggggacctgagtgacccgggcagccctttctggagttgtgaggctcctgcctgtgagatgagttctcaggcccacagcatcctgcttcttcaaggcccttgactcttgggggcacatttgggacagtgtttccgtggctgggcatacacctgttccttgatcccaaacttatgccatgtccctctctcttttcccagtatgcctgtggcatcaaagccgaggtggtggggaagccttctcctgagtttttcaagtctgccctgcaagcgataggagtggaagcccaccaggtaggttggcgccttgtgaagtgggtcaggggaggcagccccgtcagggaggccctggagcttggaatggattacaggactcaggcagcctgtggggttggccaggcagccaagcgtgggctccctgtgaaagctgacctggctgggaaggagggggaagacagcaaacgaaatccactgaatagtttcaaccgtgaagttactttcagtatgaaagcaagaagcagaaatgcctgcggcttttcctgagtttttgctgcttctctgaaaggataagaattgacaagtcctatcagtgtgttaatatatctcactggcaagacagtgtaacagcaagattacaacaatatggaggaaataataaagtcactcattttgcgacctttatattttgactattttgggatagattgcctttcaaacttccaaattttagaaaggaaaaagaatcggttatgattttattgtctacacctaccccaaccctaagtgagtctggcttcgtcctccagtgggttttcttttctttttcttttttttttttttttttttttgagccaatgtctccatttgtcaccctggctagagtgcagtgtagtggcacagtcatggctcactgcagcctggacctcccaggctcaagcgatcctgccatctcagtgcccccttaacccccttactaaccaggccccccccccgccaaccccaactgagtagctgggactacaggtatatgccaccacgcctggcaaactttttttttttttttttgagacagagtctcactctgtcacccaggctggagtgcagtggcgctgtctcggctcactgcaacctccacctcctgggttcaagcaattcttctgcctcagcctcctgatgagctgggactacaggtgtgcaccaccacaccagctaattttttatttttagtagagacagggtttcaccatgttggccaggctggtctcaaattcctgacctcaggttatctgcccgccttggcctctcaaagtgctgggattacaggtgtgagccaccatgcccagccccggctaacttttgtattttccatagagatggggttttgccatgttgcccaggctggttttaaactcctagctcaagccgtcctcctgcctcggcctcccaaagtgctgggattacaggcatgagccaccaacacacagccccctccagcaggttttctgaaagattgtaaggaatagtgggcagccggcatggaggctgacgcctgtagtcccaacactttgggaggctgaggcaggtggatcacttgaggtcaggagtttgagaccagcctggccaatgtgatgaaaccccatctctactaaaaatacagaaaaacaaaaaaaattagctgggcgaggtggtgcatgcctggtaatcccagctacttgggaagctgagggatgagaattgcttgaacctgggaggcagaagttgcagcgagccgagatcatgccactgcactccacctgggtgatagagtgagactccttctttaaaaaaaagaaaaaagaaacagagtccacagctgccccaggctgtcctgctgcctcctcagaagtgggcagcagaagcatgggttgggggtccctcaggtgtcaagcctggtgcatgtttcccctgtgtgcatttcggtccagaacactggtgcctcatggtgctttgagaaagatggcccaaagctgccaaaatctagacactgcacctgcacctgccctgagatccatagagatgtcagaagatgcaggctgtctggagtccccagaaaacacctgggaaatgtcacattagtctgttgagaaggaaaggtttgtgttttgaatgagtgagaactgcagcaataaataacctggctttttactaccttgcttcatagggcagaatttctttttttattagagatttcaaacatacagaaaagtagagagaatgatacagacaccccattatatccatcatccagcttcagtggttaagattttgccacattcctttcttctatcctcttttttttccttttaaaaaatttactggctgggtgtggtggcccacgcctgtaatcccagcactttgggaggctgaggcaggtggatcacctgaggtcaggagttcaagaccagcctggccaacatggtgaaaccccgtctctactaaaatataaaaattagccgggtgtggtggtatgcacctgtaatcccagctactcaggaggctgaggcaggagaattgcttgagcccaggaggtggaggttgcagtgagccgaaattgtgccactgcattccagcctgggcgacagagtgagactctatttccaagtaatagtaatagtaatgataataataaatttacttatttttttgcttcatattttaaagcaaatctcaattgtatcctttcatgttgtagcatgacatttaaaaatcacgatttttggatgtaaatacaatacattaaaactcctaacatggccaacaggcatacaaaaagatactcaatatcatcattcatcaggaaatgcagatcaaaactccagtgagataccacttcacactaagatgactgtatagttaaaaaaaaaaatagaaaataacaagtgttggcaaggatgtagagaaattggaacccttctctattgctgctgggaaggtgaaatgacaatgctgccacggaaagccacttggtggttcctcaaagttaaacatagaatttgccgtatgactcagcaacccatttccaggtctatacccagaagaactgaaacacgtgttcaaacaaaaacttgtacacagatattcatagcagctccatacacaatagccaaaaggtaaaaaaagccaagatacctgcccatcaactgatgaaaggataaccaaatgtggtatgtccatacagtggaatattattcagccataaagaggaatgaagttctgacacatgctacagtgtagatgaaccttggtagcattatgctaagtgaaggaagccaaacacaaaaggacacatattgtattcttccatgtatagcaaatgtctagaatgggcaaatccatagagatggaaaggagattagtggttggcaggggtcgggtgcaggggaatggggagtgcagcttcagggtacgaggtctccttttgggatgatacaaatattctggaactgggccgggcacagcagctcacacctgtaaccacagcactttgggaggctgaggagggtgaatcacttgaggtcaggagttcgagatcagcctggccaacatggtgaaaccccgtctccactaaaaatacaaacattagccagacgtggtggtgtgcacctataatcctagccactcgggaggctgaggtgggagaattgcttgaacccaggaggtggagattgcagtgagccgagatcgtgcaactgtgctccagcctgggcgacagagccagactctgtcttaaaaaaaaaaaaaaaaaaaagaaaaatattctggaactggacagtgatgatggtggcacgacgttctgaatgtgctcgatgccagtgaattgaatactctggtcacggtcataaattttatgtaatgtgtattttaccacaataaacattttaaaaccacctaaaaaattaacagtgatttttgttagtattatttaatagcaacccatatttaaatttccttgactgtctcagaaatgctcttttaccgttgatctgttggaatccaatctgaagaagctccgtgacttgtgtgtcgtttttatgttttctgagcctctctttttagccctccatcatagaacatttgaaatatctatgtcagtagagagggcagctcagggaactcctaggtacccatcactgtgctttaataattatcaactaataaccactttaaaattgttttcaacttttcattttgaagtaacttgacactcacaaaaagttgcaaacatagtacaaaatgttcacgtgtaccgttcagcagcttccccatcatcatcgagcagtgataaaaccgggaaatcaacactggcacaaggcggttagctaacctagagagcttattcagacaccaccagtcatcccactcatggctttctggggacacgttgcatgtagcagtcatgaccctagtgtcctttatctgtgaactctgatcacctgttgaggaggtgtcttccagagttctctactgtcattgctatttgtccctttcagtaattatctttttttttttgagacggagtctcgctgtgtcacccaggctggagtgcagtggcgctgtctcagctcactgcaagctccgcctcctgggttcacgccattctcctgcctcagcctcccaagtagctgggactgcaggcgcccgccaccatgcctgagtaatttttgtttttgtatttttagtagagatggggcttcaccatgttagccaggatggtctcgatctcctgacctcgtgatctgcccgcttcggctcccaaagtgccgggattacagacgtgagccaccacgcccagccttttttttttttttttttttttttgagcaggaatctcatcctgtcacccaggctggagtacaatagcatgatctcagctcactgcaacctcctccacctcctgggttcaagtgattctcctgcctcagcctcctgagtagctgggattacaggcacctgccaccaagcccggctaatttttgtatttttagtagagacagggtttctccatgttggccaggcttgtcttgaactcctgacctcaggtgatctgcctgccttggcctcccaaagtgctgggattacaagcgtgagccactgcatccaaccctaataagtatcttgtgggaaagttctttgacactatgtaaatattctctttctgatcattagttctaattttggcatctcttgataattcttttatttttttattttttgagacagagtttcactcctgttgcccaggctggagtgcagtggtgcctgggctcaccacaaccccctcctcctgggttcaagcaattctcctgcctcagcctcttgagtagctgggattacaggcatgtgccaccacgcccatctaattttgtgtttttagtagagacggggtttctccatgttggtcaggctggtctcaaactctcaatctcaggtgatccgcccgcctcggcctcccaaagtgctgggattacaggcatgagccaccacatccggccttcattggtgtattttatgtgtggcccaagacatttcttcttccagtatggtgcagggaagccaaaagatcggacacccctgctctagaggtttatgaccaaaatactgtatttaaagctgctgggaataattcttttttgtgggtggtattcccccaagtggacgctctttgaattcattttcattttgatgtttagggaattggcttaacatttacatttgttttgtagttatgtaaaacatttatatggttctaaaatcaaatccacaaagcaagacatattcaaagaagtccaacttcttttccttctattttgtttttacctgtctctggtaggtagccatgtttataaatattaaagctataaatattttataaatattatagctttttttatttaaaaaataagcaactatgtatatatttgtattcccctcataggatgaatttaagagacaaattctcaaaataatgtgttttatatatagagagaaaggctaatgctttaagtttaaaaatcagtatttaaaagtttataagtaggctgggcatggtggctcacacctgtaatctcagcactttaggagtccaaggagggcggatcacctgaggtcagaagttccagaccagcctggccaacatggcaaaaccccatctctactacaaatacaaaaattagccgggcctggcagcaggtgcctgtaatcccagctactcaggaggctgaggtgggagaatcacttgaacccaggaggcggaggttacagtgagccgagatcctgccactgcactccagcctgggtgacagagtgagactctgtctcaaaaaataaaataaaataatataagtggtatgatcacaaccatctcaaacagcccccttcccccgacccaaaacccaaacaaaaacaaaaccctgccactgcctggaaaataagacctggccaggtgccatggctcacgcctgaaatcccagcactttgggaggccgaggtgggcaggtcattagagcccaaaagtttgagaccagcctgggcaacacggaaaaaccccgtctctacagaaaaaatgcaaaaaaaaaaaaaaggcagatgtggttgcatgtgcctatggttccagctacttggggggctgaggtgggcagattgcttgggcccaggaggttgaggttgcagcaagctgagattgcaccactgcactccagcctgggcaactgagcgagaccctgtctcaaaaaaaaaaaaataggaaagcaagaaggaaggaaggaaagaaggaaggaaggagggaaggggaagggaagagaggggaggagacgagactggctgtccagatggcattgctggtgatatttggatggtctgaccttgggtggtctccctgcctttttcttgtggcattttcagttattttcacagtcagcttccttcacatttcgaatgagatacagcccacctcaccgccgaaagaagacccctcatgggggtctggtgaatctgggtttgtcgttttcggatgctggggcagtgcgtctgggagggagcggggctgtggattcctggtgggtcaccgtggctgcttgaggctttggtccctgccgctgccatccaccctgagcatctgtggccctggagagcgaaagacttgtttcctctggtggctccgagcatatgtggcaaagctgtgtcttatttacctcccacatcatcatcctctcctaccctcatatgacatttctgcctgtccccttgacagtggagtagcagctgcatgttccgaacgtccccaccctgccattttcctaaaatgctcactaacgttatttgactctcaggatgtgtagacattttaaagctgtatttcaagataaggccatttcaaatgaactgtgtcttatgcacagggtaataattctctgttcctggcttttttatttttaatgaaaaatgcctgcaaggacctctcttgctgctgaatgccaggctgtggcaaaccttaaatttttccagctgctttaatcaataaagttatcaccagtcatttcccttgatcactgctgcttcccgagaccctgacctgcttcttgctgctgagttttcagtctgcactcagaatagatataactccccaaatccaattttcattttaacctttcctcagtggcctagaaatcacagattgctggtattatgctaatagattggaaattcaaacctcttagaggcaacctggccctttctaggattcagtaataaggcaaagaataacataccttaaaggttacttctagaaatatctaattctcacggtttaagcttctgaatcagtggatggttttatagcttcatatgagcacccaagtgatgtgaaggttttggcagttctccatcatcataagaagttctgtgggatgcaaatcctgcaggtttagtggagattgagactgttcaaaccagagcacttagttatatgtgacagccaagaaccctcgcctggaggggcccgtttcacaggtgaggagcctgaagcttagagatgaaacaccttgtcataggcgctttgcattcccaccatgaggttaagacaggatttctcaggctgggcgcagttgctcacacctgtaatcccagcactttgggaggccgaggtgggtggatcacgagcccaggagttcgagaccagcctgggtaacatggtgaaaccctgtctctacaaaaaaatacaaaaattaactgggcatggtggctggcacatgcctgtggtcccagctgctcaggaagctgacgcacgagattcgcttgaatctgggaggcaggggttgcagtgagccaagatggtaccactgtactctaggctggacaagagagtaagaccttgtctcaaaaaaaacaacaaaccccccccccaccccgcggtttctcaacctcgacacttggtgtttttggactgggtcattttgtggtgaggggatgtcctatgcattgtagggtgctaaacagcatcccactgtccgcccactacatgggaccacccccttcccaagctgtgaaaccaaaattgtctgtagacattgccagatgtgccctggggagaaccgcagcattaagcggtattaagagaacggttacggagaactctagtactgaacccaagattactttgccaatgaggagtgaagccaggcaccaaaccatcgtcttccagccccgtccagtgctctttgccatagcccaactctcagggaggaccagcggtgcagcagaatttcttagggaggtggggagggtagggaggatttcaggttggggggtagggaagatttcaggttggcgggtagggaagatttcaagtggggtgggtaaggaggatttcaggttggggggtagggaagatttcaagtggggtgggtaaggaggatttcaggttggggggggtagggaggatttcaggtgggggggtagggaggatttcaggtagggggtagggaggatttcaggtggagaggtagacaggatttcaggttggggggtagggaggatttcaggttggggggtagacaggatttcaggtgggggggtagggaggatttcaggtgggcgggtagacaggatttcaggtgaggggggtagggaggatttcaggtcggggggtagacaggatttcaggtgaggggggtagggaggatttcaggtgtaggggtagacaggatttcaggtgggggggtagggaggatttcaggtgcaggggtagacaggatttcaggtcggggggttagagaggatttcaggtgggagggtaggcaggatttcaggtcagggggatagacaggatttcaggtggggggatagggaagatttcaggtgtggagggtaggcaggatttcaggtcgggggggtagacaggatttcaggtgggggggtagggaggatttcaggtgtggagggtagacaggatttcaggtgggggggtagggaggatttcaggtgtggagggtagacaggatttcaggtggggggatagggagtatttcaggtggggagggtagggaggatttcaggtgtggagggtagacaggatttcaggtggggggatagggaggatttcaggtgtggagggtagacaggatttcaggtggggggatagggagtatttcaggtggggagggtagggaggatttcaggtgtggagggtagacaggatttcaggtggggggatagggagtatttcaggtgtggagggtagacaggatttcaggtgtggagggtagacaggatttcaggtggggggatagggagtatttcaggtggggagggtagacaggatttcaggtgtggagggtagacaggatttcaggtgggggggtagggaggatttcaggtgtggagggtagacaggatttcaggtggggggatagggagtatttcaggtggggggggtagggaggatttcaggtgtggagggtagacaggatttcaggtgggggggtagggaggatttcaggtgggggggtaggcaggatttcaggtcgggggggtagacaggatttcaggtggggagggtaggcatgagggctgcagtgatcttcatggctcgcaccctcagaactgcctgtgaaaaatgggggtgaggtttgcaggcttatccagaggcctggccatataaactcaaaggtgccctaccatttcgttccctgattctcagggcatcagtcctgactataggggtctggggacagtctgtacccctagggggaggctggtgagtggggagtacatgtctgtccacatgggggccactccggcccagttctttctgctgtggcctccctcccccatgggtgtttccactgaggcctccccacgaactctgttggctgagccccacctcccagccgtgggcattttctttggattgtcagtgtggggatgaccctgctcaactcctgactgggagcaggctggcctgctgggccgtgcccacttccagcactgaaagaatgggcccaggacgggggtcagccctcctgcagccctggtttgaattggaaactctgtccaccgatgcctggcacctggcacctagccctgcagctgtgggtctgaaactgacgggagttgcaggagcttccggccctggctttgtctttgtgatgtgtcctagtcataaggtgctctgagacctcatggcttagttttccttcagagttttttctcccttggaggcttttttttttttttaagtagattttattttcttctcaaaacacaagtatttaaaatatctgattatgaagcaactcatgctcattaaaggagctaagaaaaattcaggaaagtacaagaagaaaagaaaccactcatttcaccagcccagaataacacaaatcatgcttgatcttgccaattttttgtgtctatatatacatttttaacacaaaattgggataatcttgtatctgctgtttgaaatctgagttttcccccttaatatcctattggggacaatttttgttaacttatattttaggttcagggatacgtgtgcaggtttgtgatacaggtacattgtgtgtcttgggggtttggtgtgcagattattttgtcctccaggtcataagcctagtacctgttggggtagtttttcaatcctcaccctcctcccaccctccaccctcaggtaggccctggtatctgttgttgccctgtgtccatgtgtattcagtatttagcccccacttataagtgcaaacacacagtatttggttttctgttcctgcattagtttgcttaggataatggcctccagttccatccctgtagctgcaaacgacatgaccttgttcttttttaggggacagctgtctttgtgcttattgaagcactttaacgaagcagttttcacagctgcatactattccactgttcagactcccgtcatattcttgaccatttcctattgatggagatgaggctgcttccagtttcgctcttgtgcgtaaccctgatgaacacatcttgtatgtacatcctcttgtacatttctaattatttgctccaaatcctagtggtgtggctgggaggctgtgatgcatgttgcttatttctgtttcagaaaggtgtgtccgttcaccttcctgacctgctgcgtatacagatgcttgtttccccacatccttaccaacaccatgatttctgactttttttttttaaagtggtctttgagttttgctgttagtcttgttccttatgaaggtgtccgaccacagtggcaattactcagcattgcactgtctccaggtctctgtgggagcctgggttcctttggggcctggattcagggctcatcctagagtgagctgtcatctcagggcatctccctgggggatatgtgcaccccagagctgcagttacagacactgagctcactccaccttcaccacccagccctgctgcttcccgccctaatcctcctggcagagcccctggataaggaaaatatttgtgaaccacggaagctctttggtgctggaattttgcactccaaagcatcgccgcctgttagatgatgtttccctccagcacctaggacactgaagaggagaccagggcaggcaggcagatgcgtaagagggaggattgactgctgtagcacccgacccaggacacctgcctctgggactgtggcgcctccatccggctctctggatcctgggttctggggggtccgacctttcatttttttctccgcctcacaccccaccatgtcgtgtctgaaaggaagcgaagcccagccctggcaggtggagtatttttatttcccctgacagcgcacctcctgacctctatcttcttgccagtgattttttaaatcctaaatgatttggcaataattgctcctccttgcgtgattattgcccttcaccgatagtaaatctgggaatcccctggggtcctgagcgcactttggtatgcggagcccacaagctggcctggacaggcatcactgctccaggcagctgtgcacccagccggccctggcgtcccggcccccagcctccgcctccctcgccgacagcttcccggggctctgcctggaggcagcctcgctttatcccgcagaacctccacgctgttcctgcatgagaactcttcctccaagagaccacgttcctctggtcctgtctccgtgggccacatccacaaatgttccagcccaggaagaaccaagtctgtattctgggagggaaacctgtgtgctgtctggttttgtatgtgggaggagcatgcacacagtttcattttccccggaagcatggaggtgtaggatgacatgtaatatttggaagacccattgcatttccaaggaaccttctagcaggatggccctggagtctctggcagcagtggacatggtcctgaattggagtgctgtgaatgttcactgctgacctcacggtttgaagacaattccttgagaagggcagaggggacccgaatacgggggctctggccggccctgctgccagatgccagcagccgtgggggcttgcccggccagccctggcttttcccaaccctcagagtgccccttgtgcatgcagcagacactggtgctggtcccattcccgggagcacgcggtgccactggctgtgtcttgcctggccaggtgttggtttgatagactcgaaaacctcatttggatttggcaggattttccctaacatcagtcattctggaaaaaacactttgaatttttaccatatctgggtatttttaccatatctgggtacttgccactagctctatttacttcacgtttcttaaaattgattcattttattacttaaataaatgtattttaaaaggaacttttgatgactactataaatggaaaaccagaatctctcacttaccataatagattaatctaaaaataaagaccacatgattacattaaaatgtatgtccttgctctgcctggagtcaaatagtatgtcacccgtgtgcatgacacactctgggaagcacaggggtgggtggaggaaagatcagagaaaatccgtggaaagtatgaagtagagagcaccacaccaggaagacgtggttttatttcagtacctcatgtctcgcggtggcttgcggctaccagtaactggcagaattctcaacattgtggccctcagccacctcctccgagctctctctcagcaccaccatccgccgtggcttctctgagggaggcccctctggcctctgcttgactatgatacatgtcgagtccatgccgagtggcagaaaagctccgttttcagctgaaactacctccctggcctcagggggcctcattggctctgtctttgtactttctcaatggctcctgatgattccttggttatctgaaggcctgtttgggagacggttcccaggacagttgcttcaggcttatatgtgtgtgctttctggaattcacagcatggcgtgccctggtgagtggtggggagatgggtagtgaacacgtgtggtagcccctgctgggcgtgtcccacctgtcctccatgtcctacaacctggttggccccttttgcttgtcagagcaaagctcagaacctcacctcctctgggaggcctttcctgaccacccacaagcccagtaggtcaggaggtcaggcctcacagcctcctgtgtgttatcccaggccctgatgatgagactgtggaattacctgattaatgtctgtctttagcacttgtcgtgagctccgtaagtgctgggactgcatgtgtctgattcacactgtgtgcccactgcctggcctggcactggcctgcgaaggcctagggcaaaggtatcaagtaaccagggcaagggtgcctgttgcaggtcgggggcctctctgatgtggtgagatagggaatttcctcaacgaaacatttcctcatgtttctgtttcctttgtaacatgtttaggccgggcgcggtggctcacacctgtaattccagcactttgggaggctgaggcaggtggatgacttggtcaggagatcaagaccatcttggttaacacggtgaaaccccgtctctactaaaaatacaaaacaaacagcggggcatggtggcagccgcctgtaatcccagctactcaggaggctgaggcaggagaatggcgtgaacctgggaggcggagcttgcagtgagccgagatcgcgccactgcactccagcctgggtgacagagcgggactccgtctcaaaaaaaaaaaaaaaaaaaaaaagtttaggtaatgtattatgtcctggccctgtgctgggtgcttcactaagatgatttcctttaaacgccacacctgttatacccattttacagatggaggtggaggctggcccaaggtcacacagccagcaaacaggagagctgggatctccaccccactgctctagctcagcgtgagacaggagtgagtctgtgcatctgctggccacatcgggcccgccccctctttctgtggtccccttcccccagatggagagcaaattccacctggacccgtccttgcaggcagagtccaaatctatgggacacacactcagtaagactcccctggcttgggcatgcttcatagtcatcgtttcattttgtcttgattttggtttgtgtatttatcggttagcccctcttagcatctgaattccagtgagaagacatgtagtttgtttgtttgtttgtttgtttgtttgttttaagacagagttttgctcttctcgcccaggctggagtgcaatggttgcaatcttggctcattctcctgcctcagcctcccaagtagctgggattacaggcatccgccaccatgcctggctaattttttgtattttaatagagacgggtttcaccatgttgcccaggctggtctggaactcctgggctcaagtgatctgcctccctcggcctcccacagtgctggaattacaggcatgagtcactgcgcccagcttttaatcagattttggagtaggggagtgagaggagctggttggtggtggtttttttttctttaaaattattattactttttttttctttaaaattattattacttttttttttttttttttttgagaccgagtctcactccatcgcccatgctggagttcagtggcacaatctcagctcactgcaacctccacctcccaggttcaagcaattcgtgtgcctcagcctcccgagtatctgggattacaggtgcatgccgggctaatttgtatatttttagtgaagactggtttcactatgttggccaggctggtctcgaactcctgacctcaagtgatccacccgcctcagcctcccaaagtgctgggattataggtgtgtgctaccactcccagctgttttttctttaaattacatatatttatttatattaaaaaaaaaaaaagagcatgagtccatttaagcaataacacatgttaatgtggcaaaaggagacccacaaatgactgacgtttcagccacactggcgaggactgtatgactccctccactctatgtaggtgaccaatgggaagtcttgtttgcgcctcataagtgtgtccagacacagccagccgtgccgtaatgcagagcactcagctgctcaagacacatttgctgagcgtcaaccaagtgccagttgtcaacccagattccttgtcctgaaggagctcctacttctggaagggtgggatgagcagagtccattatgctgcactatgacacgggtggcaccagggtgtgatcccagagtgctttgggagagccacacaacccagcctgggggcagaggaaaggtggtggtcaccagagctgagtcctgaaggcctggggggaattagccagcagaggagggaggtgagcaggcagaaggtcagcacgtgcccacaaggtaattttctggtgttgatgtgtatttccagctattgtcattttcccttcgcccaaaggacttcttctacatttcttgtagggtagatctcttggtgatgaattatttccgcttcggatgtctgaaatatctttatttcatcttcattttggaaagatatttttgctgggtacagaattctagatgggcagttttttcttttactgcttaaaagacggcactccacagtcttctcacttgcattgtttccaacaagaaatctaacgtcttaatcttcgtttctctgtacataacatgtctcttttgtctgactgttttataagattttctccttgtcactggctttgagcaatttgattatgatgtgccttaatacagttttctttgtgtttcttgtgtttggtgttcattgggatcttggatctgtggatttattatttttatcaagtttggaaaagtttcagtcactatttcttcaaatatatttatgtcttctctcatctcactttttttttttttaatctaatttagagacagggtcttgctctgtcgctcaggctggagtgcagtggcacaaacacagctcactgcagtctcaaactcccgggctcaagtgatcctcccacctcagtctcccaagtagctgggactacaggcgtgcgccaccacatctggctaattttttaaatttttatagagacagggtcttgcggtgttgcctaggctggccttggactcctgggctcaagtgatcctcccacctcagccttccacagtgctaggatgataggtgtgagccactgtgccccagctcatctcactttgaggaactctaattgcgtgtacatcaggccgcttgaagttgtgccatagctcactgatgctctgttcatttttgaaaataatttttctgcctctgtttcattttgggcagtttctattgccatgtctccaggttcattcatcttttcctctgcggtatctcttctggcattaatcccacccagtgtattttgatccctgacattgtagttttcatctctagaagcttggtttggattttttcttttatcttccatttctctacttttggaacacatgaaataccattacaagggctgactgaatgctctcgtctgctcattctcacctctctgtcagttctggctcagtttggataaactcatctttctcctccttctgcatgatactttccttctttgcacgtctgatcattttttattgggtgccagacgtgatcaattttaccttctttttgggtgctggatatttttgtgtgcccataaagaatttcgaactttgttgtgggatgcagttaaattacttgaaaacagtttgatccttccagatcttgctgttaagatttgggggcagcaccagagcagggttgccctgggtgaatcattcctcatagtgaagccagcggcttctgagatcgcactcaatgccctgtgcatcgtgagtgagctttcccagcctggctggtggggacataacgcagtcctgcgtgagacacaggcaccgttttccctagttatttggtgtgattctttcccaggctcagggagtttcctccctcacgtgtgctggcagtggtctgctgagtacttgaggggaccctccacgatctccacagctctttctccatgcagccttctcctctctgattccctgtcctgtggactctgctcctgggtctccccagaatcctagctttgtctccccagttcagggatcctactgggctctacctgggtttcccttggtattagtcagcccagtctgtcatacaaaacaccacagactgtgtggattcaacagcagaaactttttttctcactgttctggaggcttgaagcctgagatcagggtgccagcaagtccggtttctggtgagggctctcttcccagcttgtagatggccgccttcttgcagtgtcctcccatggcctctcctctgtgcttgcatggagagggcactccagtgcctcttcctctggtaaggacagcagtcccatcagatcagggccccctctgacctcattcgcccttaaagaattacctccttacaggccctgtctccaaatacagtcacgctgcgggctagtgaatttgtgggggacacacttgagttcacagcacccctcctgtgtccacagtctggaagcatcgaaggcagtaggctgggggcagtcacaggagccacctcatttgtttcctgcgtctcagaagtcattgtccttcgttgcttgatgtccggtgtcctgaaagctgttggttcataatttggttttctggttttttcaggtgagagagtttatcccagaggctagtttttgaaatgatacccaggctgtgagcatttcctctgtgtgtgtgtgaattttggtttgtatatattattttgtttttaaggcaataccttcaccattgaaagcaaaacctatataaagcaaaacttacaaagatttgccagagatttgcttcactttcatggaatcaagggcaaataaccaggttacagagtctttagggtcaggtgaaatttagcctgtgttccagcagaggccagacagcgttcagcatccccgcacgtccttggggcgccaaatcactttgacgtctgctgattgatttcaaattgcacagatatatactgttcaaagctgtggcctacgggggcagtgtgagatatattcaaggtgggttgacttttaatcaatacgatgacaatattatcttgtttaatttgtatgatggtggttgtaaacatcaaatcaagccatttattatgtcaaaaaaagatgaaggagcccgggaataaaactctcctgacatcacttctgagcgtatttcactgccgtgacaggccgtcatgattggggacgatatcgtgggcgacgtcggcggtgcccagcggtgtggaatgagagcgctgcaggtgcgcaccgggaagttcaggtcagtgccagctggagtcatttattcaccttccttccaggggatgaccacattctcattctgttttgttcttcaaaataaaggggatattctttccaaatcaaagagcagtatgtgggcattcactatcttgtatgtaatggacctctaagaacagggctgagtggtcttttatagcagacagtgataaagtagagtggcttttgatacagggtctgggttttggaatggcgtgcagtgcagagagaaataagcaaaggcactatccatcctggcggcggcccaccagggagttgggccgaatttgtgctgctgtccctttggcaatttgatcagaccctgcactacggcagttacagctgtgggcactcagccccttggacagcctccctctccttttgttcactttctccactctgtaagacacagagagcctggagacgacagagggttgctcaggcatgccctggagcagaacctccatctcaagaagaaggttgtgttttatttgctggcaaaagtctgcctggtctggagcagctgtcaggggttgggagctcccaggcatgtggagggccctgtggtacccaagctccagctcgcctggtcatcgagaggatcttggacatcagagtgttgccttgccttatgctagatggtttcgattgtggccattcagagaaagtacatctttgcagatgctggggagacttggttgaaacatggttacctggaggtacttggatttggggtgggggcagttacatttgtcctgagagatctgcttaggcaggaccagagaagacagatcagagggcccgaccgtcgctctgaagtctctcctgcgggttgggagctgtggctgagtgcgaggtgagtgaggaaggctcagcgcacctgggtccagggtcaggaagttggtcccttccagctacctcccagaccctcccaggcttcccccgacgtgtatgacatatgggccttctttctttcttgaagttgggttttgtggctccccttggttaggcctggcctaagggagtgagggagaccccactcacaagccacaggaacgtttttttccaagagaccagccaggggtcccccagtccccaatcctgcctttcctgcaaaggtcgcagagcctggtcttgctgctgctcccaggcgaggggctctccgctgacagcggacctcggggccctatagttgatgccgggtgctgcctggcgtcagccgtcaggagcccagcccggcgccccatgcgtctcagggcacacctgtgacccgtgagcacaagtgacacctccctgcttgggagcacagcggtgtggctcggcaggaggtggaaagggagaaccgtactgtgttgtgtactgggcttcttccagttccttggaggagaaacatgtctgtcctgaatgccacctcagttggggtctttctggccagcccaggccaggtgcccctataaactgagaatgggcctctgaagaaggccccccacagcccacgaatgcaggtgggcacgagggaggctgtgcccaccctacgccagcaagggttggtgcttttcagattccggttacctgcatgctatttaaagaagagtacgaggtgggcatcccccatgacactgtctcccggagagagagaaactctgtttttttgtttttgtttttgtttttttgagacagagtctccctctgtcgcccaggctggagtacagaggtgcgatcgatctcagctcactgcaacctccgcctcccaggctcaagtgattctcctgcctcagcctcccgagtagctgggactacaggtgtgcaccaccacgcctggctagtttttgtatttttagtggagacggggtttcaccatgttggccaggctggtctcgaactcctgacctcaggcgaaccacccgcctcttcctccggaagtgctgggattatacgcgtgagccaccgcgcccggccccagagagagaaactccatcaacagtttgccgaatccttccacactgccttctctgcatagataaacagactggtaatattttggttttttctttttataaaaatgggatgatattgttttgcagcctcctttttccttgaacactgttttatggcctctttctacaagtgtcttcataatgatttttttggtaatttttaaaatttcagtagtttttggagaacaggtggtgtttggttacatgcataagttctttagtggtgatttctgagattttggtgcacccatcacccgagcagtatacactgtacccagtgtgtagtcttttatttctcacctacctcccacccttcccggcaagcccccaaagtccattgtattatttttatgcctttgcgtcctcataacttagctcccacttataagtgagaacatacagtgtttggttttccagtcctgagttacctccattccttgaggtctccaactccatctaggtttgctgttaatgccattatttcgttcctttttatggctaagtagtattccatggtgtatatgtaccacattttttttctttgtttctttctttttttttctttgagatagagtcttgctctgtcacccaggctggagtgcagtggcgcagtctcggctcactgcaagctccgcctcccgggttcacgccattctcctgcctcagcctcccaagtagctgggactacaggcgcccaccaccacgcccggctaattttttggtatttttagtagagacggcgtttcaccatgttagccaggatggtctcgatctcctgaccccgtgatctgcccacctcggcctcccaaagtgctaggattacaggtgtgagccaccgcgcccggcctctttcttttttttttaacttttaagttcaggggtacatgtgcaggttcattacataggtgaacatgtatcatgggggtttattgtacagattatttcatcactcagatattaaacctagtacccattagttatttttcctgatcctctccctcctcccctcttcaccctccaacaggccccagtgtgtgtgttatcctgtgtatccatgtgttcagatgctgtaggaaaggaatcagtgtagtgccgggcacacagcaagccctcagcaaatggccagggtggggagcacgtgcggccactactgtccatatccatccgtatccatgcatatccatccatatccaccaagctccagacatggagtcacctgcctccattgaagaggatgggacttagcccatttgttcctaacgagatttaaaatgtcagaccttttgatttgtttgtaaggatgcaaatctgcctttgttaacatgcaggacaaagggagaagagtgttagcttggctaaggtagggacccagtcgctgggggaggccacatgggcatcaaaggaggtggggctctgcgtgtgtaggtgtgccccagttaaagggatgggaggtctcccagccattggtgactctctggagctgcactgtcagacacagtagcggcgagccgcagggggtgacttgtagctcgcccacagtgagacgtgcaggaagtgtaaaatacacactggatttcagagactgggtggggagaaaggattgtgaaagagctcatgagtatttttttatatcggctgcatgttgaaatgctgatatttcgtgttaatttcacccatttatttaaatttttgttaacgtggctactagaaaaggttcgctggcttctgcaggagtgttggtggccccaggtggcagctgccctcctcctgcactgtccccttctccctccagatggtactcttggtacacagatacactgaaatgctggaggggcgtggggtcctggatgagtggctgatggggcctcgctctctggagggctgttttgggactggaattgcctgcgtggtgctccctatggatgtgtggccagcctggggccagcacacagcctctggagtcagccgggcacctgccgctgggccggcatcccttccttccaggaaatacccccagccaggctctatggagcagcacctcgcccttccagggccacccccagccctctggcacctcagcacggccccattctgcacagcggtgagccccccaggggcagcagctgtgtctcctctgcagctgaggcatgtcgtcgcaaccctgtgctgagcctctcctctcctgcaagtggcccaaagaggcctcaacaggggccaatgaccactggatgggtcagctgggcatcagaaggccttccaggctctgtcctctgttggtggccttggcggaaatgggagaatgaagggggaggaggggtggccttgtcctgggcccaggtggcagagagccctgcagtgagtaactgcgctcacacttggtttgaggccagggcagcatggggaggccgtccaggctggggcagctggcgggtgggggcagcggtcagtcctgccagtcactggcaccaggaagggggagaagccagagggatgactgaggcctcccactggtcctgctgtgggacgtgccctcagccaaggtcttctagtacggagcacgcacacgtgcacgcacttacacgtacacacacacacacacacaattgtttctgaggcagctgtcaggagtctgagaggccgagaggtggctgaggagtgggattcggccgctgactggcagtgagatctccttgtcaccttagatgtatccatttctcttgttttcattataaaatggcccaaagagagtgaagcctcggggttgggagagattgcaggtggcttagctctggagggaaagttttgcagggcctgctccccaggaaagcatcatccccgggcaggggtgtctggagcttggcccaacagggctgcccaagagccctggacagggaagcacaaagctttcttttcatacctgctccctgtggaaatcctgcccagccagggggcggccctcacctggctggcaggtaaaggccttctgggtgttccctgcctgccctgttcccaggactcctggcgcattggacatggctgctcccccagactcctcgaggtcccatccagccacagcgttcctgctgcccaggccctggacaacagtgtctcctccgtggggggtcctgtgccatgggtggcaaatcctcatgatgccatgagtcctgcagccccagagggtgctgctgccctgccgtgagaggctttttcagatgggacagctaggaccttggccaaggccacctggcaaggttttacagaagctggactggaacctggccgcccccagtcccctgggcgggtgcttttcctgcacttttccctggtagaatgtcctgcccttggggccacattttgcagcagtcacagggcctggcctcactccagggctccatgtttggatgcgaggcccagtgggtaagtgcactgctgcccacgcccccccccaagcactgtctgctcctccctgcttgcctgcctgtcaaggcagccctcatcttccttggaggatggggctgggctgggagcctgcgccctcccattctgacaaatgagtagagggaagggacgttttgcccagttccaaggtgccctctctgtcctcagtggagaaggaagagccgctggcacatctgagccacagacccttcctgcctcttatatccccaccaacctgtgttcctgtggacatgccagaaaccaggccgtgtggcctctcaccaccccatcactctgttcgctgttccacgccacaccctgcagccaccacccagtcaaaggcgccactgcctgggaagcccctgcgctagtcctggtgctgctgtgtaaataaagccgcgatgcggagctcgcctctggtcttctcttattgtttacccaaggctggcagatgttttataagccccgaacgttcttgacaaacagtttgcccttcggagagggaaagtttcctggaccgtgattcccgacatcctgcaaggtcccttctagctgtggcctgggtgctgaaggggtttccccccagtggggtggccagccgatctaggattccagccgccttgcacaaagctggaagacgaggggctgcagggtgcatggctgtggagctggctgcgtcgggagggagagtgcccccacgcctggcctttgaccttggaagcggctgccgtcaaagcagcagcttgtcctgggtcttcccagacttcgcagtgacggatttcagagtgtttcctgttacgagaaggctgtggattcgctttggaacatgacagatttttcttctgtgctacgtggagtgtgagggaaggaagtgaaactggtgttaatttttcagcagtcacatgtcaaagtgagtggctagcaagcaggggggtccaggctgtggtggccctggtcagccttccaggagtcaggaccctcctgctgccctcctgtgtaaacagaccagccagggcaatccgagccagccccggtggagagaaagggccagtgctgggcttgggagctcaccccaagttcctcgggtactcatcctggcagtgcagggaagggcaccgggaacaggcctgtggaaaaatccactgtgcaatgagtccgtgaatcccagacatcctctcagcagccctctggtcctgcccgggatagaggctgtggcttctggtgtaggtgggcacttctgggctcagaggggtcatttagctcacctgaggtcacacggagtgggacccaccccaatttaagcctgtacacttttttcttttgtctcttttggtaacagctttattgagaaatcactcccacgctgtataactcagtggttttcatacattcacagagctgtgcagtcgtcaccacaattttataacatttcatcaccccaaaaagaagcctcaaatcgtttagctattattagcaaagaccccccacattccacccagccccagacaatcactaatctactttatgtcatactttctttcttttgttttcatttttttttttttttttttgagacagagtctcactctgacagccaggctggagtgcagtagcatgatcttggctcactacaacctccgcctcccaggttcaagcgattctcctgcctcagcctcccaagtagctgggattatagccacgcaccaccatgcccaactaatttatgtatttttagtagagatggggtttcaccacgttggccaaactggtctcaaactcctgacctcaaatgatccccccgccttgacctcccaaagtgctgggattacaggcatgagccaccgtgtctggccttatgtcatactttcatttgaaaaatttagaaaaattaaattggcccaggagtttgagaccagcctgggcaacatagcgagacctcatttctattaaaaatcaaaattagccaggtgtggtgacacccacctgtggtcccagctactcgggaggctgaggcaggaggatctcttaaacccaggaggtccacgaggctgcagtgagctgtgatcataccactgcacttcagcctgggtgacagagcaagaccttgtctcaaaaagaaatttttttaaattgtggtaaaatatatataacataaaatatgccattaaacaattgtgatgtgaagtgaataaaatttgaatacacagcataatgaataacaaagcaaacactctaaggtcaagaattaggaggctatcagctccccagaagacctagtccctgattgacatgcccagtcctgcagaggcggccccgccttgccgtgggcacagtggccgcggtgactgggcctgctttcttctcccacatggctggcttccctccaacatggcgctggctcttccttctctgacctttatccaagtggaatcgcacactgtaccgttgagctgtggcttgttcatcgtgtcctctgtggctccaacacccctctctgcccgtccctcagagggaacatctgggtgggtcccagtttggcccttctgcactggctgccaggaatgttctggtttttgtctcctggacacatgggcgtgagtccttctggggcggatctgctgatagatggcagtgctgggtcatggaggagcatatgttcaacttgaccagatgaggccagacgggtttatgcccccaccacggggggaccctgttgctccccgccccgaccatcacgagctgttctgggagggaggcccatgttcttcccagtttacggtcctgggaggtttctggacattgtcagcatgtggcagtcccggacgctgggcctcctgagagagtggggaggagacccgaggcggaggcgagtgtgtgaaaggaggctggtggggtccacatggctcagcaggtgaacatggggtttccaggggcagacaccccatcgtggcccccatggctttggttggggcagcatcagcttcctcctctgctgggtttcctgccttctgttggctgctggggcaggggagacaggattgggaacagtcaccctctctggtactgaccctcattagcattcttggttcctgagcaaggctgcttcatgtccagggcaagaggggaaagatgttaaaacctgaacccccctgctgggcttggtgccgcctgtgggaaaaccggtgggctgagggctggggggctctgagctgaggaagggggtcccaggtggaggccgggaggtggctgcagagtgaggccacaaggcctgggggttggcccagaaggtaggacccctgtaggcagcctcccaaccccaggaatttgcctcttgggaacgtgcaggtccctcgagggtcctggaaggcgctggagtggtgggacagggattgagggagcgtcagcagggccctctcttctccctagagccgctcaacacgtgttgcaaggcacgtgctctggacctggtgctcacacgtgctcttctcccaagacactccctgaagcgcgttccccaggagctggaagggggataacgcaggtatgcctcatggcggggccagtgctcatgcttacacacactcagcctcagctcgtctacaccctgcaacagcccagagaagccacccgccttcttacccctaccatttagatttgcaaatggaagctcagagaggtgaagtaacttgcccaaggccacacagcctaccagtgtcagaatcgggattcgaacccacctctgcctgactccaaggcctgtgcccctgaacattctcaagccaccccccaattagccaagtgttgctggagctctgagcaggggccgggcctcacaaaacaatagtagccagtgtgggggctcacaggactggtcagctcccgccctcccccgctttggccccctccacacacacacgacctgctgctgagctggacataagcaggacctggctctgggggcttctgacctagggcaggccctgaagttggtcattagaccccacccacccacctgagagcctggcggagatgtgacaggtgacagccccagaagtcccatcgttgctgcctttgtggttaaagtccgctgaacccaagcgctctcttcccctccagtgtgtggtggggaggtggggaggggacccaggcagatctgggaagccgtggccaggccgcagcaagcaggggaagaaggggcccgcagtgaacttgcagcaggagctgtggccaccgtcctagccaggccggccacctgcccaaaatgttctctaaccttaacatgaacctgggccacttgggctctgctggatgcccccagggctgccagagcctgcccgccacagagcccctctgagacctggagtctcacctgagccctctcgctgcccacagggctggcctctgcctttccgctgctgctctgcggctcggggacacggccagggctggtggcgcttacagcccaggggcaggggagggcacatcccccacagcattcaccattgctgctgtgttctgatgcatcagggaatctgcatttttgaccctttgattgctacatcttttccagcctggaaaagtgaagaaggaaggaaagggggctcagaggcccctgactcctgtccttgcaggcacagctggacgcagttcttattttcaaccatcttgcagcagtggttttatttgtgcacaggttcctctgtgtcatgggtgggtcccaggatgtgaacccggtgtgctcctctagctccactgtcctcaggttggggacagccagggacaaggccccctactccagctccaaacgtagttccaggctggggtgccctttgcccaggcccagcctggtagccccgggtgctctccctgacttttcatgagcccgggggctgaaaccagagcagcagggagacagaaaagcccagccccaccctccagagggggagtccagctgcccggagaccccaagagcactgccagaatctgggtatgagacccagtcccctgccgcgtgctaaccatgtaaccttggacgtgatctctttgtgcttttttttttttgtttttttctttttgagatgcggtcttgttctgtttcctaggtgggagtgcagtggcaccatcatggcccactgcagcctggaactcctgggcacaagtgattctcccgcctcagcctcccaaatagctgggaaacaggtttatgccaccacgcccaactaatttttaaattttttataaagatggagtctccctgtgttgcccagcctcctgggctcaagtgatctccccgccttggcctcccaaagtgctgggattacccggcttgagcatgatctttttgccttgcttggctctgggtttcctcatgtgtcaagtggggctgagccccagctgtgtgggctgaacaggggagggttaagctcggcccctagagtgagggcccgccatggtggtggctcccgtatggcatatttccctaacggttcccatttggtctgtcctgctagaccaggctggtgggacgtacactgactctggggttctgtccaccccgtgggtgtggtccaccacatggtcatggtggctttctagggtagacttgaccttccccactgcctttatttatttatttattattttatttttttttgaaacggagtcttgctctgttgcccaggctggagtgcaatggtgcgatcttggctcattgcaacctccgcctcccgggttcaagggattctcctgcctcagccttccaagtagctgggaatacagatgtctgccaccacacccggctaatttttgtatttccactagagacggggtttcgccatgttggccaggctggtcttgaactcctgacctcaggtgatctgcccacctcagcctcctaaagtgctgggattagaggcgtgagccacagtgcctggccttccccactgcctttaaaatctctatggtccccactgcccacgggaggaggcccaaccctgtcctctgttagtcccctggtccccgtagatatttaccaagcacctactatggccaggcgctgttgaggatgtgggggatcgagtgaatgtggcaggtgtcccctgccctctagggctgttcttccttctagggagggaggttcaagcccctctcctgatgcccagctggcctcccagcgtcagggatctctgctctgttggtcccctggagtggccctggggccaccacataacacttttgtaagaataagagttattttttatggcacaatcgcccctgccctgggtttgtgaatttgggggattctttttttttttttttttttttttgagacagagtcttgctctgtcgcccaggctggagtgcagtggtgtgatctcggctcactgtaagctccgcctcccgggttcatgccattctcctgcctcagcctcctgaatagctgggactacaggcacccaccactaccatgcccagctaatttttttgtatttttagtagagacggggtttcactgtgttagccaggatggtctcaatctcctgacctcgtgatctgcccacctcggcctcccaaagtgctgggattacaggtgttgggggattcttaaggaaaaccacctgaaagtgcggggttattaaaaacagaaaccaacccatgcacccaccactcacgagcagcccacacagctcccacctgaccagggctcccccttctcatgggtgagtgaaagttgctgaaaataggcctcctggtgaggaacctcccagccagccctgtcagtggcactgaggctgctctccgactgtggcatcccccatgtctgtcctcattactgagcaaaaatttgtgcagaggagtgctctacactttccaagggtttaatccaggagctgagggccaggggctccatgactgtagactcaacatcctgggccacagccatccgtgagggggaggacgaggggccacgccctgcactctgtcccagcatcccctctccctggagccatgcagagatggcctcatttcatcccacactcacatgcgggtcctagacaccagctcatgcacatacacacacacacacacgcacacacacacacacacacatgcgcacatgcacccacgcactctctttgcctgtgggagggcaggatcccagctgccccagcggggaaggggtgtggggaatcagccctgcagatgcggtgtgaggggcagacatggcatgctggtctttggcagtgtggtcccaagtgggggcggctggagaggtgccttgccacagggcccaggccacccgcatgcatcccatgaggccgtccaagcccgtctgccctgcatcagagaggacaccccaatgcggggagggaggaacctggtgaagccaggagcggtgggcaggggccagcaaggatccccaggcccccagggcagtgtgcatccggccttcttgcatgcccacctggcatcctgcctgccgcctcctgcctcccgccccacccctcctgcgtcccagcctggtggacctctcagggttcactctcatttcctcctccccacgccacctgctcgggccacaagagggcgccttgctgccagggatgggacaggccgggcctgctggagccagggccgatgcacgcacagctacatacactcacccgggcccacgtgtgctcacgaggggcccccatacccacacgatcccgcatccacatgtgcgcacgccaggccccacaggttctcaggcccaggccacacatccacaggtgcccactctcatgctggcctcatagtgcatgcacacacacacacacacacacatatacatacactcacgcccagacccccccacgccccaggccacacatgtgggcctgctcacaccccactcattccctcccagatgccgcaccacccccatgccctgtgtgccctctgcctctcggggccctggggccccagctccaagaaccacgggcttgctttagccaggcttccacaagcctgcccattcctcaggacactgaccctctcctctgccacccttcacgggtgcagacgatgagcatggagaacaccacagcgcacagggccgagggccaccatcagagtctcctgcagccctggattccctgggcctgaagcaggcagagcggccaaggtagcctcatcagcctcaccagcccgtccttcttgtctccctggcaccttgtcagagagaggcctaagaacacgctctgggcattgggggcctcccagtgttccctgcgagtttggagaggccaggtccctgggatgaggacacgcccacccgccaagctcccctcctgccagtcccacctggagcctttatgcatgctgtctgcgcccttcacctgtcccaccacctcttctttggcctgcctggatcctgccatatcctcaaggccttccctggtgatggaattccagagtcctcccaattgcaagggcctgagggatccgctgccctccaaccccacaccccgtctcaaggtcatgtgctaatcccgtgcaagctggggtgtgggctggctgccatgtcaagctcgtttctgttgtacccaaaacacagtctcacctccagaagaattacctggcactccctggagttcccgcagccttctcattttcaaataatttttcagatttccagaatagaaagaaattccatacacccttctcccagagacagaccccattcaagctttgccaatcattccatgaactcctctagagaggggaagacccagtccggggccaggcagctctctctggtctctctggactccgtcctggagcacttccctgtcctcgcctcagttctcacagattgccagttttgaagattacgagacagtcattttgtgtggtgttttctcatgatacaacccaagttacgcgtttggggctagcattgcttagaagtggtgctgtgcttgttctcataggcatgtaatgttggtttgcccaaatattgctgatgttaaccttgacctcttgtttaatggcaaccacataatgtgtttttcccctttgtgattgatacgtatttgtggggagatgctttgaggtgatgcgatgtcccactttcatctgccagttttagtgtcccttagtatttcttgtttgaatgaatgatttctatgatggtcgccaaataactccatcattcttccttgtatttactcctggtgttttactttaaggaaagatgttctcttctttccactgattgatgtatgccagtgtgggctcgtggcttcctatgtaattcagtgggttatcgtctgttaaccgtcatcactgggatattcagatggtccccgacgcaggcactggcagccccttcaggctggccctgtgacctttcggcacatcctcaccgttctttgagcccttccttgcattttggcccaaaaagatgtttcaggttcatttgttcagtctttgcttcagccctggaatcggccatttctccaaggagccccagttccttccagtggagggcactgtttagaatctcagcccagggccccaggtgtgcttgtgttgtctctgggcattgctgctcaggccctggcggtggacagaggtaggaaagatgcgcatggtatgcaggtgtgacacacacatgcgtccatgcttaccctacccacctggacctgcccaaaagcccgagctcacagcagtgcctccgattccagtccagcagcacagggttccttctcacctcatgcctttacacgttcatgccttccttccacagcagggagaagcctggcttcctcattctctctccatctgcttatcgctctatcccatgtagagccaagtccctgaccttgcctgtggtggacactctgcgtttgttaaactgaataacaacaatcctccctattgggagaaaccttttcagcttcccaggccatttctgcatttgtggttctcttccaagcctcttgggtaggcatagaaagtcttctctgttttccagggaagctgcagactcacccagcatcacccagctggtaagggtgtagcaggacagtgccagcatgtctgtcccaactgtccctatggcccctatggaagattagctatgtggtggcaaagtgtcccgaggaaggggcattctgggatgttcaggtgctcgactgggctgggacttgtactttggaaatgagaagaagcccaggctcaaatctttgccccactgcttgctggcaggaagaccactgagcctcagttttctcatctgtaaaatggacttattttggacttacttcccaggtatcctcagcacctttattacaggagctccattaatacatggctgcaggaggtagttcagggaagcaatgggagtggaatgctgggtgcatcacggcctggggttgtcagcctggcctctgcaggcatctgagatattcctgagggcagattcagccaggccaacacttctggatagaaccacagggccataaactgagaccattgagggagactgcttggcttggaaaactcgcccagggaccatgtgctctgatttgcaaaatgcaccccccagagcggcttcctgccttcactttgtgcagagttgggggcggcaggggcgtggggggttggatggaccatgtcacccaaaagtttgtgtcctcggatggggaggggagagcgtctgcccaagcacagcctggaggcttcccttacttccgatgaccttgtggcagtcagtgtcacccggacagagtgtggcacgccttggggaccagcccctcccagaaggataggcgcttgattggaaacgttgacttttcatttaaataagtaaaggtcaaggcctactgcatggcgaggaggagatggctcttccctgcctctgtcctcccttacccccgtgtgagtggacaaaccccccgtcctcacccaggtggcccgcttctgccacccgcactatatccaagacacagcccagctttgcaggcacttgccgtgtgctccgcagtgtgcagggtgatgagatgaacaagaccatgtggcctgaagggtcccacgctgcagagggccccagcccagggccagaccccaagtgtgtgcctggagtgactcaggggtgcgcgaggaggactcagtgggatatggaatgcaggggctgaggcatagggagtgacacaggggctcagggctttcccatgagtcgaggctcattgggttggcagggaccacatctgccctggtcgatgccctgcccatgcaggctctaacggcagagtttggggattggggcgggggatgcagggggcctggggtggggagcttgggggcagtgggccaggcaaagcagagccctttgctgtgttggaaacccctcagaggccctaggcgtgataagtgagtgtgacgcaggagagtctctctgcatgggccacccacaattgcagagaagacgccctgcatgtcataggcttggcggcccatgccgggaatttggcatggccgtctttcctccggtgctggggagctcagcagctggaggcagatgctggattgtctttctctaccgcagcagggcaggtagcttgtcttttagtttgttttgttagtctcctgtatctgtgactctgcagagagccaacggcaggaagagggcagcaccggcacgtcggaggccgaggacggcttagacgggaggtccggaggctctcagcctgaccagccctggcctagggccggggaaaggcctgggctttggggtcaagggcaggggatgagatctcagacccacgacctaaccattgacctgcctgggccacttcacccctgcatgcttccttccctgtctgtaaaagcggatcacgtgaggggccttgcaggaagctctgaggcccagtgaggcccccgggagacaggcgagaccgctgtgagcgcccgtccttcccgcccggtgcctggcaggttcctatgcgctctcggggcttcgggctggggccctgctgcggcgaggggggtgctccccaggctggcgctgtcggtggtgtggctttggctgacacgaggtggagaggatcttgttgacctgaccgtataaagtagcagtgaggccttacaagtagttctaggcctggaaaggaaagggaaagaaagagcaatggaatctggtattttctcaggtgtcctggactccatctgaaagatttcgccatgggaaacattccttcctgggtcctcggcgcctattgttacagcacgcaggcacccgggctgtggcctaggggtgaaacctctctcgattgtgtggccataggtggttacttaacttctctgtgcctcagcgttgccactgtgtgtggcgatagcaacatgtcttcttcctaaggttgttgggaaaacactgatcgcaacagtgacagatgcaggacccactccaccagaaattgttccatgagcttgtttacccaccagacgcctgtatgagagagaactgacatcgtcctcactttgcagagaggttaagtaacttgcctaaggtcacacagcctttgaaccctgggagtcgaactccgtcatctatgctctgaaccactctgccacatgtggtggttactgtcagttccatttgtttccacacagctttaagttagtcctgtggtttgcttttattttatttttaagacatggtcttgctctgtcacccaggctggagtgcagtggtgcaatcatagctcactgcagcctcgacttctgatgagattctcgcatctcagcctcccaagtagccgggactataggcacatgccacaatgcctggctaattaaaaaaacattttttttttttttgtagagacaaagctgttgctatgttgcccaagctggtcttaaactcctggcctcaagtgattctcccacatcgggatcccaaagtgctggaattagcttggcgtgagccgccatgcctggcctagtctggtgggtgtttaaaccttgctggcctttaggggtgcatgctgtgaagacctgtgggccagaggccaggtatcaggaggagacactcaggcctgttgaatcagctcagtgccacagggctgcaagagcagctgcttcaggctcctcaggccgtgggcccctgagcaccagttctgctctgctccctcaggggaactggggagatcaaggtggggcccctggacctcccctaattcagccagagaagctctcatctcttccggggtcccaggcaggattcccctgggctggggtgggaggttctgctgctttaaaaccactggaaaggcttgagaaccagtgatcttctgccctgttacagacgcggaagccggggtggagcaggcctgtaaggtgggggctggggccggcactgggtccccgggcttttccacccctgccgcgtgtgcgtgcagactggccctgcggtccatgggcttgggcctgccttttggcttgctcccgagtattttgcccagaggccttgtggctttcagggatgctgtctggggctggaagtcagtccttgttttgtcctggcagtagtcagcaggagggagaagaaggggttaacctgtctggaaccaggaaagaggaagcggtagtgttcccgcatgcagtcccaaatagactcctgctcctgctccgaggccaccccagccaggctggatggaagctccccggctgggccattgttccggcccctctccctccaccctgagaaagcccccagcccggtgggtcagggcaggacatggacaggccaaggctttgggctccagggaggccaagctgctggggcccagagaccctgctgtgtatcccagagccagagaagaacctccctgagggctctgtgagccagagggctgggggatgtcaagagacaggcagggcagggctgcaaggttagagtgaggggaggggtacctggtgccattttggaagggacccctgaaggcaggaagcaggacagaccccccgccattggcagaagacctctggaatcctgagatacttaaattccctgcggcagctccaagcctagggaaccagacagatcggggaggattggctagaacagtgcatcctggtgggtcgtgttggaagctctctgaggtccctctgctgctgagagatgaaggaatggagccctggctctgctgtgggatgccaggctgcccaggctccccgacctctccagcaacaaaaatcataaacacagcatatgtaatatataaataataaaagtaacagccatcttttcagaataatctagcacttagaatgtgctagggaacatacaagcacttttctccttatttacagtaagccttaaggtaaatttcatcatctccagattttaagtaaagaatttgagaatcagagacattcacagcaacctggtgcacccggagcctggctgaccccagcaccgctgctggcggccccacctcactcgcccagtctccctgcttccccagccaccgtccagctacctcgagagatcctgggagcatggtgattgtggggtgcatgggggcccgtgagctgttgggggtgtcagggttctgcccgtgagatcctccttgttgccagacatgaggacctggactggcagctgtgggtggcctcatgaatcagggatcagagatagcgggcagcaggcgggcccagcccggagcaagctgtgccattggcgatgcggggaggctggccccatcgaaggctggtgggactggtggagactcctgtccactgctcagcactaggcctgcagcagacaccatgagccccaaacttcccaaagcccttccccagtcccacaagatggtgtctgcggaccgtgctcgtgagagatggcagccaggcagtccccacagggcacccattttcagctgcccccgcttctcagacaaggaaactgaggccagaaagccaggtggcccaggagctggcttccccatttcctgctcctgtgggccccactgcagtgcccatgggccgggctgatattacccgagacttcggagctctcacgggtgcgagtaatttaggctgcatggacacaagctgctggcttgagtcgccccgttatgaatgtgtgtgggtctgtgcccctttcatgtgctgccacagggcccacgagtgtgctgaaagggaaggacacggccaaggggccatggtggacaggagaccttcttgggggttcggtggtgtccttgaccccactctgactgagcactgccccaaggcactgccattccaggcccccttccctgagcctcccaccccaggcccacccacctgctgggtcctcccacctgcggggcccgccatgcggggtcaccatgcgagtctcaccatgcagggtcaccacacgagtctcaccatgcagggtcaccacgcgagtctcaccatgcagggtcaccatgcggggtcaccatgtggggtcaccatgcggggctcaccatgtggggcttcaggagcttgctgagcaccctccccacccacggtcactctccctggggtctgtaagcctccctgggcctgagcagctcccagccttgctgctgcctttccacttcctggcagtgaggtctcctgggtgccttctctcagccctttgggatgttttttgtgaggaagggaggctttgatgctgtggagcatctgtagtgcccactccagtggcttcacaggagcagcaggctgtttgttctgagctgttccaccttgtgcctgccagaggggagatagtggacaggcctccctccccccaagtggtggggtggaccccctgcccgctgtggccccatacctgggggccacacaccactgccctgggccgtgcagctgctatgaagagtgtgctgctgagaccctggaagagacggaggatgaaattgtgttgccagatagtccatttgttgttctgagactcgcatgcctgggagaatcctgggaattaactagctccttctctcccatcccattttacagaaaagtgagacccaaggtggtttctgacttgcccagaggtcataactgcttggacagtcatggtcctcagagcccacgtttgctgaccagtgcaggctctcacagccactcagctcctgcagccgtggcgtggcagaggagggaagcacttcctgggatttatgctgcctccctgacatttcaaggcccttcatttctctaaatattggaggagttgaattatttttagttgagcctcaagggatcagagaataagcttgcagcaacgttggcagatgggcttcttctagcagagagtggttattcggggcctcttattgagagaatcgggtgatttgaggaaatctggggtgtcctgaggcataccagaggacccccaagtttttcctgtggctcgtctgccatcaggaaaccaaaatgactcccctcgtcctgagctctccagggtgtggacctggaatgcttaaggggaggcaatggcatatctttaagatgagcacagctccggagccactcgagcacccaaggccacgtcctgctcagggcacttcgggcctcagtttccttatctttaaaatggacagagttggccgggtgaggtggccctgcctgtaatcccagcactttgggaggccaaggctggcagattgcttgagcccaggagtttgaagccagcctgggcaacatggcgaaaccccatctctactacaagtacaaaaatttggccgggcatggtggctcatgcttgtaatcccagcactttgggaggccaaggagagcggatcacttgaggccagaagctcgagaccgcctctactaaaaatacaaaaattagccaggcgtggtggctcacgcctgtaatctcagctactcgggtggctgaggcaggagaatcacttgaacctgggaagtagaggttgcagtgagctgagatcgtgccactgcactctagcctgggcgacagagcaaaaccctgtctcaaaaaaaaaaaaaaaaattagtcaggcatggtggcacacacctgtagtcccagctactcaggaggctgaggtgggaggattgtttgagcccagaaggttgaggctgcattgagctgagattgcaccactgcactccagcctgggcgacagagcgagaccctgtctcaaaaaaataaaatagacataataagagtacctaccacctacggctggggagaccagaatgagatatcctgccaaaagcactcaccgcacttcctggcacacagcaagggttcagcagttacccgcttctgctcattttcttggtgttctcatcagtatgattatttagggaaacttgggtccccagataaccgtggggaggggagggtttacctgcaggtgccctggcccagccgttcatgcagcgccgtgctgtttctgtgtctgtgcgcttcggtggagatgctgtgggtgggtgggcaggtgtgccttgtgctgtggcctcccgagacaaggagggctcccacctaagcagggtcctgcagcccaggcacatagccctgccctggccctccaggtccacacggctgtgggttcccaccagggggcggcagggacttgcggccggggacccagcctggtttctcccgctttgcttcgtggccaggctagggccaggggggctgcgcaggatggggccttttcaccactgcctggagccgctcgcccaccaaccccactgacggctctcagtttccctttccccgagcgcccttcaccctgaggcacagcacagcctcgtgctcctggccccactggcctgctcccttaagctgagttttgcctttgcagtttgagtgacatttttgctggggatccggggctgctcggggtggcctcgcacacccctgcactttggctgcctggaacgtggcttggggttcacggtgctccatggtttgactcacaggcagggttccagcagcagcagagagaaagagcatcttctccagccggccctacagaggccccgggaccccaaaggctgcactgaggtggccaccaaaaccagggggcagagtcctcctaagttctggccctagatctaggctccagatctcccgggagttggatccagaggaacctgggctcacttctttagcaggttctggacacacgatgcctttaggggctcaagaaaatgtttaatttattttaaaattagaaggaaaatatctctggaacatttagggccaaagaaaatgttttaatgttttttctcactttagaaataataattcaaaaatccattaaattatttaacagcttttttaatggaggaaggggccccccaaatcacaaagtggccctgggccctgggatggccggaggagagcctgccatacccacgcagtgtgcctgagtcctggagtgcgatgtattctgagccacccagaatggtgagtcaccaagaggcagcttggaggtgggccgctgcccctgcacactgtgcttgggggacacagccgacacgaccagcctgcagtaccaggatagacaatttgggctggagccaaacttgatcagagggaggctgactttatcgagggctgctttgtggcgggcgttcattgtggaaagagcccaggaaggagctctctgccttgcaagaacattgcctgtggctcaaaatggccacatgggatgcagttcctgaggtggcagaagagccagcctctgctagccccagacctaacggagttgcggctcatggccaccctgaggaattccacctgggcgtttggtgtccctagcatatctcgaatggcaggctgaggctcgaaagtctcatgcctgtctgcaggacccccgcgtgagcctgggaccacccgtggggacagtgtgtgtctaggcaaatgccttgggcactcgagggggcactgaggccctggagggcttctgtgggtgggagttcccatgacgtcatccctcagtacaagctacgagggcttcttagaacacgctggacactggggagggctctaatggtatttgcacaaatttgcaatgttggagaccagagtggagtgggctgggcgggccaggccccggcccagatggggtgacctttccttcacccctacttccccactgcaagggtgctgcctccccagctcctacctggggagatgctgacatctggtcattcgcagagtgccctgtgcccacatgctgtccctgtccagacatgaaacggacagcaggagggggctttgaggggtctctcctgagacacctctccagctgaggctcctggagttggaagcccctgcagactcggtggctgcctcaccaaaggctccatctgcatttgcccccatgaccttcccactgtgcctgggaccgcagttagagctgaccagcacctggggcagtctggggtctggggctggcgttcgggccagcatggaacacagatgccttcagtggctctggtgggcacggggcttgcacagatcggactgtgaccccaggctgtgggactcagccaccaagcgccttcacttatttactgaccttgaagttatccagcacttatgtgtttacagatacctgcacaacagccagagaggtacgaatacatctccacctacaggtgagggcacagagaggttaggacccacctgaggggatgcagccagcaggtgggacttgggccctggcagtctgtttccagcctgtgtgctcaggtccagggtctccctgggactggagagggcgctgggggaatggagccgcctcctgcatcagagcttctcctgctccagttcagcctgcagctctcctgcatccccaggtggggaccagagagaggggcaggctggtaccagcctgggggagggggcagttaccagaagaccttggtccccatcccagccctgtgcaccctggggcccagccaggctggaggcccatttttcccagaagcccacctctctgcctccttccaggggctcaccccgccctgctgcctcttcttgatcgcctccttgcccctactctcccctctgccctctcgcctcccctcacgttcatggggttttgtggacgcgcctgcctgcctttcatcagtggtgtccagccgtctccctgaccgaggctgagggtcctgcacatctaggccccgtcacggtcgttctccccggtccctgcctgcccatggctcttgccacagtggggatgcagcagctctgggttgagtgtctgtcgttgcccctgcactgcgtgtgccctgtgagtgggggctgctgtgccacacctcactgccacccatgcccatctcagggtgtgggcccagcgggtcctcaggctgtacctgtcagggggctggatacctggacagggccttgtccgagggttgactgggagagaaaatgcccccgttccagctcgggcagtacaagccttcatcctcccagactccacctccctggctgctccttcctgtatggcccccacaggggttggcagggcgcactccccagagggacaggagtttgtggccaacctgagggtagacactgcctgtcctccagacccctagacacccccggtcggcccaggtgcggggtctgcccgcatcccacagccatctttccagcgaggacagtgcaggcgcaggtggctgtaccgtggctgcggtgatgtgctgactgggaacggcagaggtgccctgggaaggcggaggtctgggagccccacaagggctgagccttggggcctcaaaacagggagtcttctctgggctcagggcctttctggagatgggacttctaggttgccagggccactagccgtgaggagttcagctgcccatggtggcggcggctgggctgtatcttgcacttctcagctgtgtggaggggtccgtggggcctgtctgggcccagcacagcagcgcacagaaaagccactttatatatttgggattctattaatccttctagtttgtacattttttatggcaccttcaatttttttcttttaaaaattaatatatttttttttctgataatataagaaatgcatgccttctttggaaaatacagaaaatcacaaagaaaataatccacaatcccaccttccaaaaggaaccacagttagcattttgttcagcctgttttttaaaaaacaaacaaacaaaaaatgcatactaaaagcaacgatttataaactgtggttgtagagttaagctcatacccagagttgccggcatctcaccagttcctctcctgggacagtgattgtgccttcccagagctgccgcaccctggcctcgcagccaagcctggtgacagcagtggggtgaccctgcgaggcccctcggatggctgatgctgagagcccagcagagctggagggggtaggggcagcctccccaggactgatctgacagacgggtatgtgcctcacccggcatcacccaccggggaggcctccgtccgcccccatgcagtcatgctgggtccccccatctcgtgtcttctggggaatggagcggaagacctcccctctctggaaccaggggtgggagggactgtgctccccagaatgaatattccttggctgtgggtgagcctgccagcccagagtccccaggggtcaggagcagactggcaggggccatggcaacgactgggacacctggggagccaggtgtccaggcaggctccagtgtccactcccaacaagtatggggcattgagatcctgggtggatcacaggggagcaggcgtgctcaagctgacacagcccaagctgtctttattcaccaccagacccgaatccacctctgggccaacagtctcaccctgcaggaggcctggcacctgtgtttgtattttatcaccatgaggttgtgtgggctgctgagagcgtgtgcagagctctgggcacaagcctcggctctgtggccggctgagtggccgggcctcggtgttctcgtctctgaagtggggagaaggctgcccccgtcacgggacctcacagtgttagatgagattgcagatgcgatgaggtgggggcccagcaggcctgctcagggcgtggctgtccctcctggtgcagacaccatcacgtggaaggcgatcacatcgagtgttctggccactgtggcgcgagtgcatatgtgcgcagggctgtgcatgggcggcctgggttgggcttcctttctcggcttgtctgggaggacggtggctcccagtgggtgtctccctgtctctgccccactctgaggatgtgggtgctttgagctgagtgaaggaggaacttctctaggagagagctctgcctgccacctccctggtgcccccgcccccagagcagagctgtccttacagggccacagcctctgctggccccgaagcccctttgcagtgaaggggggctcctgtgtgcggctccctccagatggacaaaaaccaggtttggatgccggggcctcggctgagtccctcgcttctctaagcattcctttcctcgtctggaaagtgggggtgtagtattgtgcgaacctcccaggccatcccaggatgaactgagatcatgcccataacatagacttccgcctggtccctgctcagagctccccgaatggtcattgccacatctaaacaaatttttctttcagctgggggtgctggcggcccctgcagcggtgcccacggcagcccatgcacccaggcaggaagctgctttgtgcaggagggtctttgtgcaggggcagtgtgggcgagcctggcagcctcctgagcggcaggactatgcaggcgccttattgagctgtcagcggctggcagctgctcccaggtagcaggaagaagggccgagctccacgtgggcatgaaaggcccagcgggtgtgtcagggggcggggtggggtggaagtgttagtgcgaatgtgagtgtgtgtgcacgcttgcaggataaagatgagtttttaaaatgtgtctcctgtggtttttgatttttttaatctttcccaaaatcaatacaaacaaaccacaggcataatttgggtctgtaaaggccgacgctgtcattctgctgcaagagggagggaagttcagttggggggaaaatgcagcgcttttgctgtcattaagtccaatggtggctggcgacagtcacggcgctggtggagctcagagcagaggtgggaggggtggtattgaccaggcgtgtgtgtttccggtgtgtgatgtgagaacccagggctcgctgggatgctgtcctccctccctccctccctctgccaagctggtgggaaggcccttgccaaagcgcacaagaccccttcacacagcaggggcacaagctcttcgaggcagagtcgcctgcaggtggggtggaaggggtgcgccccagacccaagaatcgcccgcctctccaagaccatccctggctgctggtgagcagggtaggagggttgttggcacctgctgtggccggggccgtgccaggtctgtacacattgtgctctttgctgggtggcagccaccacgtattctcaagggcctgtgctaggcctctggcgggccctgggttgttcccttccttatgagctcccattattaactaaagaagacactgaatgatctcatgtggttgtatgtttttttgtttgtttgtttgtttttttgagacggagtctcactctgtcgcccaggctggagtgcagtggcgcgatctcggctcactgcaagctctgcctcctgggttcacaccattctcctgcctcaccctcccgagtagctgggactacaggtgcctgccaccacgcccggctaattttttgtatttttgtatttttagtagagatggggtttcaccatgttagccaggatggtcttgatctcctgacctcatgatccgcccgcctcagcctcccaaagtgctggaattacaggcgtgagccaccgcgcccgcctcatgcggttgtatcttggtgggtaaggcaggtctttctatgacccacctgccagcctcatcatccgtgctccccacctcaccatgcatgcttcggttgctgtttgtccacaggcgtacttactttgttttatatcagtttttagatacaaaaatagtacaggtagaattcggtattctacattttcagggaatatcacatcacacacacgctgctctttcactatggttttcatgttttctgcacctctcccgctcttaacccgcctcccacccagatttagctctgttaacccttcaggcttctttttctgcacacacacacgcacacgcgcgcacacacacaggaggttgtcctgtttggtttttagaaatggaattgtcctgttcctctttgcaccttgctcatttcacttcatggtgcgtcccagataccactttctaagtcaccggcagaagcaggaccttgtgcttcctgatggctgtgagctgtcgtggcggcacacctgtcctcggtgtggagttccagttcattcctgcagtcccctgttaacagatgtttagactgtttcccttttttccccttttcattacaaacaaaaaaaattgctttcttaaacttattttcatgtttttaaactttctaatttctcataaatatttcaactttagattctgtgagtgagtctattagtgtcaggctttgcagaggaaaggaaagttctagaagagtctgcagctcgttctcccaccccctgcccccagtgtccacccacagaggtgtgagcatgcaaatagggtgagcttctgtctcccgtctcttgggtgtcttttccacctggggcctccctggggccatgctgggggagtgaaaggcccctccctggaagcccgaggcccctggcctggatgctgtagcttgcctgaaagcggaggaattctggctgcctggtggaggtcggggctggccctgcaccgccaggagaagcagtctgagggagggctgctcaggctggacccagccctgaccagcggggccatcgggcgtcctgggccctggcaggatgtcgttccagtgatacagcacttccttgcttctaaactgctgagtggtcagtgtcggttatcagctgtccacatatattgcataatctcagatgctggcttgtaacagtgagtggaggtgggtggccacactgtccctttagaataatccacatggccctgtccaggcagggcagcctctgggctcccgcccaccgcctgatgcccctgtgcctggtgagcctgggccggggtgtgtttgctgtggatgggtgtgttccgagttctccggtcagcaagctccctttgtctctaggggagttcctgggaggtaaaggggagccttttcaagccacaagatcacccagaaaacctacgtaagcactaaagttcagaaaaatccagccagccttggagctggggagactgcttcccttggggcccggccactccagagccatttccgtgatggtaaaatgagtgggggccccatgctgcctctgcctcctgtgaacgccatcctctggttcttcaccttcaggctcccgcatgagctgggtggttggtgttaactcctctcttctctctttctacccggaaccccctgcccagagctccagggagctccaccatagccagtagagggagggctctgtttacctccttccaagttcccaattttgctctactgatttttgtctgcggtgcataatcaatcagcctggaccccagagaggccttctgtcgaggtgggtgggtctcagaacggcaggctgctccccctcctacctcggggcccccactgtgcacctgcagctgaggcatgttttctggggttcctggctttggcctgcacggctgggtgggaggagggcatgggaggtgatgctgctcactgagcagggcctgctttgaccacctcgggatcaagagtcctgttagatgcccaggtagagacccttgctgggcgggcaggtggatcttcgagaatggagctccgagggacaggttggctgatctcagtgaaccctgaggcttccccaaggccatacagcttgtctggcagagcctcagcaggagcccgggtccccggacaccgaggctagctctttctgctgtggccttctcacagaccaaggtgggatcgcagccttggtagccttgaagtctccctaacctggcaagacagtggagaagggggacagccagcgggacattccactgtagaataaatacatgcatggcaaaacacttgacatctattaacttctaagtggcaagagtgattcttgtggttagacctgaatgggaacttgtaagtctaaagtgaaaatctaattagatttttcccacaaaacaattttccgcaacgcattgtggacataaaggtttgatgaatgtatagccaggcacagtggctcacatctgtattcccaacactttgggaggccaaggcaggaggattgcttgagcacaggatttgagaccagcctgggcaacatagtgagactctgtctctataaaaaaataaattagcagggcatgacggtgtacacctgtagccccagctactcgggaggctgaggcaggaggatcacttgagctcaggagttcgaggttgcaccatgattacacctgtgagtagccatgcactccagcctggacaacacagcaagaccctgtctcaaaaaaaaaaaaaagatttggccaatgaattttatcagctaaactgggatatttaagagaaaggaaggaactcctaatttctgagccctggttctgaaaaagctttcttttttctttcttcactttagtcttgttagcaaaaccttaacagttcatagaagaaatcattttttacttttttccccttaatctgagcacaacatctggggttgccatggtggtccattggtggtgatttgcaggacgggaccttacatctggggaaggggctgccaggctgggcctgcctctggccctgtgggcatggccggggtgggcatggccgggaaaggcatggcctccagaatcagaggcctgggttgaatcctggctgagtgcctagtggggcactttgggacaaatgaccacctttctgagccttgcttttctcatctgtaaatgggggtggcaatgcctacttagttgaatgttaaataaaatgaagtcgtgtctgtgaagcacctggcatggtaatcggtggccgtgaggatgacgctggcctcgcagggactgggtggcacaggtgtcctgaccttggcatgcagggtggatgggccaggcttccagccagtgctggatctcagaggcccaggggttagagaactaagggcctccgagccaggctcctcgttgcaaatggacttctctcctttgcgtgctgtgaactgggcgtgaacacctcctgtgatggggagctcactccttgccgagggagcctggtaagctgccggcacagtcatcctcataccctctctcctgacctgtgtcttatggcctctgcccccaggcagctgcggggcagggggctggggtgacccccgtcttggttgttgagcccttccactagctggagggtgactgtccccccttctcccccgacacaaggcacagcctttcttgtggctgtcaggagagagggcctgtcctcctttgcgcccacgctgcccctgcgccttgtggcactgcattcaaagtgagtgcgcctgcttttgtttcttcctaggacttctgaaaacgtgtgggtgggggagggtgtggagggttcgtgcaccgggtggtgtggctgcagtggcgtgggaggcggccccagaggggcagaggcgcagtgtgtttatgtgcacagggccgttcccttcctgtccctcatgtgaacaccagtgtacaaatgtgtgcacacaggcgtgtcccccgtgccttttccctaccttggctgatcgtggggaagatagactcacttcccctctccaggcggcgattccggaaaggaggctctggaatgccagctgggggcagggaagccagggcagccaggagggacctagcaggcccacagaggtctgggacagctgagccctcctgcccacggccctgccatccctgtgtgtccccaaggtaacgtccaagctctgcagcagggaccagccctgggtcccacagaggaggctggtccaggtggtggactagagctccgcttgtgccagcctaggtgcagaggcccggcccagcccttcctaaccttgccgtcctgggccagctgttcggtctctgcgtgcctgtttctacacaacccatctgtaaaatggggccatgggagccaccacctcccccaggcctgtcgccatcactggcacctccaccagcattagcaccagtgtctccatgaagttggggttcctggggagatggctccccccagcctcgcctcctccctgcctcagagactgcgcagaaggagggcagctgggctgatgagcaactcgcctggggccccagagaggcccgatcccatcccgctggggcacgccccaggtccaagcctgtctgggcacccccagggctctggactcacctccacccctccctctgcatccttggtggtgacaccatcagcaagtttggcacccccggccctcacactgaccccagcaccccttccccaggctctggtggactccttgctcacgctgcctgctctgcagggtgggggcgggggggcagagcctgccagcccctcccacacttccctcaccagatcacaggaccctcggggtggaggctggtgcgtggggaggtaagggtcaggtggtgggaggccatcctcagcaggggtcccagagtgggagacccagagctttaggaccccaccatgcggaggggccagggtccctgggatggggagaggcctcagccagggtgagcagaagagctgcagcccctgagacccgcgggcttggccggccccccgggaagaagacccacagctgggagggcagttcatgcagggcaccctcccctccacctgccggccttggggagcaccaggcaaggctccctggggagcagggctgccacctctgacctcgctccagcctgacctcaccacggaggcgcacgcaggatgtccgcaggcccccgggtggcagcttcctacagtgcttttccaccagggaccgtgctgaggcccagggccctgagaccctcaggcccatcacagcctccccactgagcacaggcacgtccatcccctccccttctccacctgtcacctgtctgtcttccaggcttcgtgggacttcccagtgccccattcctgcttgtcagaagaggggctgggagaatggcagggcctggcactccaggctgcccctgccctcctgcgctcttgccctccagcctttccccaccctggaagtgagttcatgatgttaatggcagcagcagccatggtgcaccaggcactctgctaagcactttatgtacattcttaaattaccggatccccattacggaaacggagtcttaggttgaaaaactacttgaccaaggtcatcccagctgtaagtggcagagcaggccctgaatccaggcaacccaggctgatcaccaccctgccaccacccacccctctctcacatacaggctctaggcctcctgtctatactggaatcccccacagcagatccacatggagctgtctaaagaagcacctgcccctcccccagacaaggcaagaaagccatctctacctggagggagagcagtgaggggcatcattaatgggctgttgggtgtcctgatttctttgagagaagacgagctgagcaggcacagctcttaatcctccagccactgccccccaggctggggcctgccagcccccgcccctgtagtcatttctaaatcgggtctagatggactggacgcgtggctgccagagaccatttgaaccccctacctccaaattccccaaggtccccgcctgcaaaggaccagccccagcatgaggccctgaaagtggcaacatttcaatagcctatcctggacaatcaggacgatccctttactaatttatatgctatacgtttggggtttaattggagcgtaattgtaatccatatgctgttaaggtaattacacttataaacctaatataatccatagatcaatgggtttggagctaattaaggcaaccgtaattaacatttaccaaaaaccttgcaattactggaatatcctttaagctggccttccgagctgataatgagcgccgctcagcgctccgcggcctcctagcgtgtctttattctgcacaagggggccttctggggacctcgatgttgggagggcagtcagtgcctgcttttagcacccagggactctgatcagggacccagctgatgagtggcagcaccatgggaagccaggtccctgtgacttggccatccccagaacacggctgattttctggctcaatagactcagcctgacaaaggggaacttggagaaagagaacaagaaatccatatgctgagctgggacagaacccaagtggggttctcatgtggggagcatatggggctttcctcctgcccaccccctgcacctgcagccaggatgcacctgagctcggactgccctgaaaccccaggaaaatcccaacagcggcaccatagggccaggattacagccattctttttttttttttttttttttttttttgagacggagtctcgctctgtcgcccaggctggagtgccgtggcacaatctcagctcactgcaagctccgcctcccgggttcacgccattctcctgcctcagcctcccgagtagctgggactacaggtgcccgccaccacgcctggctaattttttgtattttttagtagagacagggtttcaccgtgttagccaggatggtctcgatctcctgaccttgtgatccgcccgcctcggcctcccaaagtgccgggattacaggcgtgagccaccacgcccggcctacagccattcttgaatgttgtatgactcccggtgatattgtcaccttgggagctgcacagagacctcccccgggccaggcccctcctctgtctgcagaaggaggtaaggggcccaccacatctgtcttaccacaggctaagctgttggcatctgttcttttcctgctccaggagggacagagcccccctgttgtccttgcggcaccatgctctgggaactgggcgctcctgtggacagctccatgtgagtcttcttcgagagactccactgcagatagaagaaggcctcgagcctgtagttgcggggcggtggtcagcctgggctgcctggattcagctcctgctctgacatttacagctgggacgacctgggccaggtagaggtttttcaacctaagactcagtttattatgtatttattcatttattcattttatttatttattgacagggtcttgctctgtcacctaggctggagtgcagtggtgcaatcatggctcactgcagccttgaccttctggactcaagtgatcctcccacctccgcctcctgggtaggatgcatgcctgaggtgggactactggcatgcaccaccacaccggctagtttttaaaattcttgcagagacagggtctccctgtgtggcccaggctggtcttgaactgggctcaagtgatcctcctgccttggccttccaaaatactgggattacaggcatgagccactacacccggcctggactcagtttctataatgggatatggcagtttaataatgtgcatgcagtgcttagcctagtgcccggtgcagagccactgcttcattaatgctgctgctgctgtccttgtcatctctatcattgacagccctagacgagctgaaggggtgggcccaggtgggaggggccaggtgggcggggccaggtgggctgtctgcacctcgcttactcctgggtgggctgttagcttggcgggtcccagggctcacttttgccccatcatcatttctgtacactttttccatctctgggccttagggcctttccaaagactttgcaactctggcagatatttctagacagaattaggttctttaacgaaggaatgaggcaaatgtggacaggagattgttcataattgcggctgtcatttattgagcacctgctgtgtgctcttagcctctgggctaaacactgcacagacataattcactgattgccaacagcagctttatgcggctggcattttagtctcactttagggtgaggaaactgaggctcagagaggctgaatcactggtttgagtacgtccagcgagagggcagctgggccaggatttgaagccagccattctcaacaccactgtgctctctgcctcccgagtggaggccctgacccctttgctctcttcctgggtgggccagtggggatgggtgaggggtgagtgtgtcaatgtttccctcagcccagtagtggagaacaggcagggtgggcgaactcctggctggaccacagccacaggcctgcaggaggcacccttgccagggcgagtgagcactgtagtctgggagaccctagagtgagatgggggctctacaaggcacagctcctccctggaggaagggaggaagggagggtccctgccttacagggcctggccctccccacttgtccaaagcccggctgggtcctggtgtgtcagcgctttgcagtacaccctgtgctacccacccaacctgtgtccccgtctgatctccctggaccctcccacagcctgggaagggtcccccagctgtgaggtcagcatgggggaccttgctctgcagagggcacctgtagggctggccttattggagtccctcctttctaggcccacagtcctgcgctgcctggtgtgcggggcctgaatgtcattgtttcatatattttatctgattttctagttgctttaaggtactttgtctatcttgactgttaggctctttatttttcaaaaaaaaattcaatataatagtttgcattttttcttattgataataaaaccactgacattattgagctcttgttctgggctcagttctgccccgagctgtttgtacatgttaccttgctgaacctgcacccaagtcctcaggccatattcccccttacagatgaatgaagtcactggcccgaggtcacacagctgggacgtagtgcaaccaggctgggccctagctggttgaccccagtgcccactatttatttggtgcttacatgcactggctcattttgtctctacagccttgcaaaggccacagttatgagatggcagaggtggtattcaaattcaggcccctaaacttgtgtctgtgttccctcttgaaaacttcagtaaactgaagaagtgaaaggcagagggtaaaccaccgaccagcccaccatccagaggtgacggtgacctctgatgccgctgggtgtccaccttccaaactgtgtgcacaaatgtgtcctctgtgttctagtccacaggtgaggtcaggagcacaggctgttttgggacatctcttcccagtgcccagttccagaacacccttccaaaccaccgtgcattctccggggccatcgttttaatggctgcaccctgctcccgcgtgtggacgcatcctaaacagtcccttagtattatggttagatgctccatgtgtttccaattcttcattattgtaaacccaactgcattgctcatccttgtagcatagctttgcacttattttggatttttgctttaagataacttctcacacatggaattgctgggtcaaagggtatgcaaaattctaaggtgtttggtacttctgcagaattctccctccagacatcaagttgctctcccatctgcactgtggataattctgcaactctcgacaccctcaccaacaaataacaccgaggactagcagtgtcatttttaaaacgcgcctccatcaatgggaaaagaatgggatcttctttgattgataatgagactaaaggctttttctgtcttcctcagtcgctgatatttgtttcttctataaattgcatattcatatcctttctcagtcttttttgtatgttttttaaacgaaggctcttctttaatgggaaatctgcatttcatcaaagatggagcgcagagtctgtggttcatttattctctggagaagatgcttcttttttgttctctaaccaatcagagaccagcctgaagatgctagcaaattaatttcaggcgcacgagacaggcggtcggtgctcatgaggatcctggctgaggaaatcacattttagtcccaactcctcgaagtgttgaggccgcctggagtccacgcggaccagggttcaaatccccactcggccacttcctagcagcatgactttgggatgtccctcagccatcagagacttcactttctgggctgtaccatgaggtgttggtgtgcagtttaaaggaagtttggacggctgtggtgtggatagttctgagtgctcagtaggcagacctggttttgagctgatagagaaggcccacgcgaacgggcagagctgggcctcgccagggcctggcactcagtagtgctcaccaaatgcctgttgcacaagggatggtgcccaggtaacctgggtgagctacaaaccagagccgctggacgagttccaaagggaactcgggcgcactagccccctttgtttgtgtttggagtgggagtcaccgtatagctttatttgattactttttacccaaatccccatatcctcaggaattcaaggacaaggatacataacaaaatgatttttgtctcagagtaaatcacagtccacttggaaaatctccatcctagaggagggtgagggtgcaggtcttggggtgaggctggggatgaagcctcccatgactttgtggttaccctgctggtgggctcactccctgcagggccattggtggagtcacatgatgccaggcaaggctggccaagcagggtacagagcacactgggggagggatggctgcacaggcgtggaccgtgggggatccagggtagggatcccacctgggggtcttgggctgagtggtggagcgtgttgcctgaacacatgagcccgtggacatctagtgagcttagggcatggcaggggtggggtgcgttgcctgcagcctggggcggcctcggctccttcatgtcagcgcgtcctgacaccgtgacactgcgcatggtcacatggagcctgtcctcttgtatctgcttcatctctggctttcctgtccatctgcctggtgctttggagggtgtaccaggctgtgagggagaagcagccacgtggctccgttttcttgtgctgcagctgggccagcagatctaggggaaaggccacttcctagtggcctttctggtgggagggagtcatagtgatgagtctggaagctgagccctggcttgggagcagctggaaaggctgcctgtggcctccctgagtggacgtcctctagcagttgttcagcctccctgtccccagccagccaccacctagttggcactcccagtgggccccacgagccgcgtgttgctatgcacagcccttacgagaatcccactttccagatgagggcgacaggaggaggaagcatcactcacttgcccaaggtcccaccggcaaggggctctgagtctggacgtggccctctgagctgctctgtccccgcttggcgttggagcgtctcagcctctcccaagagtgtgtgcccttgtgtcgctggttcagaagccagaaggcttccaggggcagacctaggcagaagccctgagtgggcatcctggtcagccctgtgggggttgggggccagtgggcaggggcactcaccatgctacccaaccccccatggcacccccaatgtggaacgcacgggctcaggactcacttttattttggtagagcttggagtccccatttccatgttttgtttttctttacagtcccagtgtcaagcttagtaggacacagatgtctcagcaaacaaaactaggagccatccgagcaggcccctgcggccatatgcattcccatgcgtagcgaaaccaggccctcttgccaagcactctttgggaggatatttggaaaaaatgtcaaggcattcatttttctctcgaccttttctgttggaatcgtggggccttaacactggaagtaaaaggagttataagacacagctggcttcctgcacccgggacagccccagtggccaggcctggccctattctcccatctggccctcggtggtttctgcagaggttaccatgcacaagggccaggccgcggcccccacctatgtgcaaagaagttggcaatgatgtccaggtagcaggtgcagcgggcaaggtttctacccccaggctagttaaggacacaggagactcgaggagggagaggaccagcagaggacactctatgggggtggcttctggcaaaacagtgctatctgaagatgagtttgaagttaggaatgtttgctgagttggcctggatattggaagtgccctttcttttgagccctttggctctcttggcgttgaccatctgctggtggaacatgggtgggctcccagagcacctgcccctgtggtgtataccagctggatctcagctacacctacggacctgcactggtatagatttgggtcccaggttccccaccaggggcctggctggcttccccaggtccacacccttccagcgaagtcacatcacaagttgtgtggaaatggaggtcaccaccctggccacagaggtgaggctggtgaggcccagagtgtacctgttggggatgctgcaacacttggaaatccggctgcccgaggctggaagccgtcttgctgcgtatgtcacgtaggataatgagtggactcttttctcagtcctggaggacttcctcggccagtctggagtaagccatggctggggagatcagttcttggaagtgctgggttttcctgcatctttggtcctttggtggcacaggccttcccttgcaggcctttggcacctgtctggggtgtgtgccattggctcagtagcccatgggcagtaagttccgtggaatcaggcacattttcagccagcgctgccaagtgctccctgcaatggtgttgccacctgaggtgccacccaaagcagaaaaccctggggctgggcaagacagcagctttcaggagctgggaggaggcatcatttccactttccaggccctgcgatctggtttgtagcctgtaccagggcactggagtcctgccttggcacagctcatggtctagaggaataacctcaggtccaggtgagacactcagcctgtgatggcttcatatggtcactacgttgttgtatatggagagaggactctttcttggttttaaattttgtttttaattttataaatgtaatatattcacatggaaaatttggaaattataaacaatcaaaaagaagcagctaaaaaaagtcctttcataatccaccggcaacatcagttaacatgtggcatatttccttcccgtctcctccactcccattttttttggacttcttaaacataattgagattgtgttttatgtacaattttatagcatgtacttgttagctttgaaacagacccatctgcgtttgcaatttggcctgcagggaaaagaccagattgctgagtcttagctaaagctgtgggagaaggaaatctaaaaataccctcctcgttggcctgctcagggagcgccgtggcctcttgttctcctccctgtcagtacagcagaattagccatgactcagagtgggtctccgtggtgattctgcctctagagaacatgccgtgctcccgacgtcaggctcacaggtgagcgggggcactagccggggacgaggggctggagcctggccggcccaactacagaggggcctggggtttggctcctgccggaggagcccctctagggctctccggggtcttctgcagagcgaacactttgaggggagcatctgtcagagacacatgatgcttcttcacccaaagccccaccctggctgcaagaggagaaaatcaaaaccgtgaagagcattttagagacttccacttccagcaagatggagcagaagtgcctttccctattcctcctgccaagtgcaatgaaaaaccctggacgtcagttatgaaacaaacagaagaggctgaaaggaagagaggagaaggcagactgaccagggacctcaggacctggggatcagtggaggggtgaattccctgggtttcctttgtgccttgcacatctcagacgtggagctgaagaagccagcaagagcctctgcagaagcctgctctccctggctgaaggaccaagccaggggcacctggcgagacagaaaacatttaggcaataactgtggtactccagccaaacaccacagaaaaccctgtggccccctcagctactcgtgccagcaagggctcagcagggatcctggaccaaaacactgaccaggctgaaatgagcccctaccactgggggtgtcagagaaggccaaggagggagccaggagcagggtgggaatgaggtcccccagctggagcatcagtggagaccacatggtaagcctgtgcttcccctagtcagtggtgacaagacatcccttcccctcccactggacgggcagcatcatactgaagagtcaggacttcatcgccactcagaggttacaaggctatccgccccccccacaacagtgacaatggagcccgcatgggacacagtaatgagggattcctgcacctcccaaccaggggaaaacttcctccttgaagagcagtaatgagagcacctcccccacctcagtgtcagcagaggccaagtggggaacctggacgtccaccccacccaagcagaataaggcagcactaccctcttcccctgcctgagttgtgtccagagaaggccactgaaagggaaggtttaaataagactcagagtctcacaccacacacccaaaatgtccagcttgtggtacaaaaccacctgtcatgctaagaaccaaggaaaccccaacatgaataagaagagaagaccaacagacaccagcactgagatgacacagatgtcagggcaaagactaaaacagctactggaaaaatgcttcaaccagcaattgcaaatatgctcaaaacaaatgacaaagtagaaagtctcagcaaagaaatagaagatacagggccaggtacggtggctcatgcctgtgatcccagcactttgggaggctgaggtcggtggatcacttgagcccaggagtttgagaccagacctgggcaacacagggagaccttgtctctacaaaaattaaaaaattagctgagcatggtggcacacacctgtagtctcagctacttaggaggctgaggtgggaggatcacctgagcctgaaaggtcaaggttgtagtgagctgagatcacgccactgcactccagtctgggcaacagggtgagaccctgtctcaaacaaacaaacaaacaaacaaataaatagaagatacaatgaagagccaaatgaaaattttagaactgaaaaatacagtagccaaaatagaaaactccatggatgggtttgacagcagaatcaaggggacagaggaaagagtctgtgaacttgaaggcaggaaaatagagatgacccaatcagaacagagaaaaaatagactgaaaagaatgaacagacatttcaccaaagatgatatccaaatagcaaataagcacataaaaagatgtcatgagtcactagggtaacgcaatttaaagccacaatgagacatcactacacacctatcagaatggctaaaataggccaggcgtggtggctcacagctgtaatcccagcattttggaaggctaaggtgggtggatcacttgaggccaggagctcgagaacagcctggccaacatggcaaaaccctgtctctattaaaaatataaaaattacctaggcatgatggtgctcgcctgtaattccagctacttgggaggttgaggtgggaagatcacttgaactggggaggcagaggttgtagtgagccgagatcatgccactgcacctcagcctgggcgacaaaacaagaaagataccatctaaaaaaaaaaagaaaaaaaaaaagaatgactaaaataaaaaatgccgatgagaacagtgagaatctgaatcactcagacattgctggtgagaaggtaaaatgatacaaatgctctggaaaatagcttggcagtgtcttaaaactctaaatgtaaaactgtcacatgactcagccatggtactcctgggcagttatcagagaaatgaaaaattataatttacacaaaaacctatacacaaatgttcgttgcagttttattgataatagccccaaactagaagcaatccaggtgtccttcagtgggtgacgagaggaactacatccatgtcatgcaatagtactcagcaataaaaagaaacaatctctctctctctttttctttttttttttttttgcataaaatccagatcctgcagaaaaaagaaacaaatttgatactcacaatgacttggatggactcccagtgaaaaaagcctatcccaaaaggttatgtactttctgattccatgtatagaacatcctcaaaatgacaaaattctatcatgcaaaacagatactgttggatggggtggggcggggcaggagggcagtgggcgtggctataaagagcagcctgaggggtcctggtggtgatggaaagcttctttatttcactgtatcaatgtcaattgttgactttgatattgttctatagttttgcaatatattactgttaagagaaactgagtcagtggcgttcttacaactgcatgtgaatctccaattagccccaaattaaaagtttactaaagcaacaattgaacaagaaaaagaaaagcctttgtacctagcaacccagaatgcctatgataagtggcaggagctgatgacaggcgttcttggttcagaacaatgacaactctgggacaagcatgccgctgggagaaagagaccaggagctgcacagccagacagatgcttttgcagtgggatggcagtggggagttggaatggttcatcatttactttttatatttaacacttttttttttaagagatggaatgctctgtcacccaggctggagtgcagtggtatgatcgtggctcactgcaacttcgaactcctgggctcaagcgatcctcccacctcagcctcctgagtggctggacctacaggtgcacattgaagcacccagctgatttaaaaaaaaattttttttttttttttgagacggagtctcgctctgtcacccaggctggagtgcagtggcgcgatcttggctcattgcaacctccacctcccaggttcaagtgattctcctgcttcagcctcccgagtagctgggactacaggcacgcagcaccacacccagctaattttttgtatttttagtagagacagggtttcaccatgttacccaagatggtctcaatctcctgatctcgtgatctgcccaccttggcctcccaaagtgctgggattgcaggcgtgagccaccgcgcccggccaatttttttttttatagagacgagtctcactgtggtgcccaggctggtcttgaactcctgggctcaagtgatcctcccacttcagcctctcaaagtgctgggattacaggtgtgccccaccgtgcccagcctcggattgtcatttagcggtctgtctgaacatcagtctcctgacgtccttcctacagatatgaacagagcccctgctaggcatgagtattcggagcataagctcctggttccccagtgagagacggatgtgtaaggaggcagtctcagggtaggatgttgagtctgggctggggacagtgcactgtgccgtggaattatggagagtttttcctcctatctctaagtccaatgtgtggcatggttatattacatttttaggatggaaaagttataccatatatttaaatgaattaacagcatttgcaatgatctggatgagattggagactattattctaagtgaagtaactcaggaatggaaaaccaaacatcttatgttctcactgatatgtgggagctaagctatgaagacgcaaaggcatgagaatgatacagtggactttggggacttggaggaagagtgggagggaacgaggtgtccttgtccagcaagcgctctgtgtgaggctccatgcatctttatgcattctgctctgatgtttgcaaacacgacgcccggcaataagtgaggcccccctgaggtactcaaggggcttcactcgccgaagctcccctccccacagcaacctgagcttctgcaaaagctgagaagcacgcatgagaagggcacctcccctgcttggaaataattttttggttgcagtagggggttttgtttgttgtttgttgcttgctgctcgctagcttgttcttgcagaaggaagagctggtctcctttgccatcccttactgagggtcgagaaatacagacaggtgtcttcaggctctccgtgcaggccatagtccccaagaattcacttgtgagcctctgtggcagtttatgttgaggattaggtttagtttggggagaggaggatccatggagctggggttccagatttgatgtctaattttagaaaggacactatttttaaatcaatagctgtgcagcctagcatatcactctgaccctgtgcccttaagggacgcactggggaggacattgttcaggacggagcctgacaagaaaggaatgctaccccacgccgtgcgccccaggcggccactgtcgtccaagaatttccaggtcttttctggtatcacttcttggcacaggcggaagaagccatgttcccatcgtcccaacatgcagctggggacctggagaccgctttggcctttctcaaggtcacaggaaggcagggggaaggctggcgagggccctgtggatccgccctgctccagcccggcgtcgggttacagcagtggggacaggagggggaaggggtgtcactgcaggagctaaggagaaatccagggttgacaccctcagaaactggagaagaccaagaaagcagagcggagagtgccccctgcagtccgcagagggcctttgccgaggtgggcctggagccgtcgccgtacccctgggtggcccgcaaggccaggcttggctgcctggcaatgtttgttgtgggcagttgccatgccactccggcggcagcaagagcagggacaggtgtggcagagcccggtggttccagcccagcctggcctcccagcatcaccttctccagttacttgaagtgggccaggtcctggagaccgggcgtgtggttatgggctctccagattcttggcgccttcctgattgcaggagtctggttggcatcatgggacccctgggagactcactttcaggaggggttgtcctttcccccctgggggacacactctgcttacttgtcccctgggagcgctgggcagggagactgcaggtgccagccactcctgcagcttctgcccagcctcctctggtgtcctctgcactgtcaccaagccatggcctcaagcaacttgtgggtgcctgtgatttaggccttggggtgttggagcctgcttggacccactggcaggagtcagttgtgatgtcttttgccagctctcttctgtagcttgaaattggccacggtggaagtatttacaccatggaagttggcaaatgcacggacccagcacccctaccccatcccggagagccagttaccagcacacgtctgccttttggggtcttttgtgcaggactgatggatggagtcgcagcagacacttggcctggccactgtgaggctgtgtggtgggaggtcgggggcacctgagacagagggaggaggctgagagtctgagggggcagccagggctggagggccatttgggattctgttggatttccctgcactttgtccaggggcagaagcctccctcccaaggtcaggccccatttctagccgtgctggtcgcacatccctgtgagcaggggtgcagaagagaaatggtccctcaggagccccacacccgctcccatccagctctcctccagatgacatctccaggcactgggaccccacagaggcaggccctgctcagcctgcagagctcccggtggagagggagggcgggggtccaggtggcctgaggagaggggtgcaggccagctgagggggctcccagaactccacgagctcagagacgggctcttgcccttgcagccccatccttgggatgccccagcccttcatccagacctctgctgtgattgtgtcaggacctgtccctgcagtggactggaagctccaggtgggcaggccttgccgtggtcacctgtgtccacaggcctgggcagagcaaactgatgagtgtctactaaatcagagagaagacacaaccagggagcgagtgcacgcccgggccgaccagggctgcgggcatcatatcccgctcagccgctgcctgccctccacgcgcccctggctgcctctctcagcctcggccccctccactcatgagtgggaataaataagagagtagaaaacgtaagtgaggttcttgtctggccgcccggctcatagcaatgtttaatagatagtaactggtgttatgactgaagacgtcctttgtgggctattggagttcttaatatccccgagtaaccacagggcaacgctctggcttgacaaggggcggcactgctggtcagcagcccagccagccgcagcggccacttgtgttcccagctgtggcttgtgatctatggtcgactgagctgttactctcctcggttagacacgcatatgcacacacatgcacactcatgtacttacacatgtgcacgcgcatgcaacgcacaaacacatggacacacatgcgcatgcatgcacacactgtacccacgtgcacacacgagtgcacagacacgagggctcactgtgatctgctctaatgcatggtcttaatagctatataatattccacggttcttttttctttctttcctttttaagatggagtctcgctctgtcgccaggctggagtgcagtggcgcgatctcggctcactgcaacctccgcctcccgggttcaagcgattctcctgcctcagcctccttagtagggggggactacaggcgcgtgccaccacgcccggctaattttttgtatttttcagtagagacggggtttcaccgtgttggctaggatggtctccatctctcgacctcgtgatccgcccgcctcagcctcccaaagtgctgggataacaggtgtgagccaccatgcccggctggttcttatttctttaattttattgaagaataacatacctacggagccgtgtgcatatcataagggtacaatttgatatactttcatggagtgaaatacatccctgaaaccagctccggatcgagaagcaggacatttcccaccctcttcttgccctttgcctgcaacccccatccccggggtgaccattccccggcctctgcccccgggacagttttgttcgctcctttgctttatggaggtgaaaccgcgcgatgaggcccctttcgagtctggctgcttttgcttagccctgcgcttgtgagatccctccgcggtggcgtggagaggtgtggccacttcccgcttcgtggcaagtcgtgttctgccatttctccgaataccgatggaggcttaccgtgctggaccatggaatggggtctggaatgttctcatacaggtcctgtggtgcaatgggcgtgtgctggcactgggattctgtgtctaatgctcttcatcctagacctgtgagaggagactgttgtccccagggcagtgaggaggaggccagctttggagtggtcaccctcacctcgccgttacacacgtcacttcatttaatttttaaacaacagtcctgctgccggtggccttttctgtgtgctatagaagtgagagcaggtgcctggggacacttggaggcccccctaggtttgtctgtaatgagcttgtgctggcgactggcatgttctgtcccccccacggctcccagcgtgctcccagcgccctcccacagctttaggcaattaagacacgggcttggagtggccattgcaggacctgctggtaatgggggtgaggggtaagctttgtttccaaggcaggtctgtgccctggggtcttgtccccctccacatgcacactgcctgggtggggctcattcctgcgggtaaggcccgggatcaggaatggctgtctttcttctgcccagagggagctgcttgtggcttgaggtggccctgagactgtgagacaggccactgggaggcccaggctctagcaaaagtggggatttttttctctccctagttttggatcaggtttttgaggttgtgcaaacgtaggaatgaggcctttcaccccgtcagttgtctgtcgggctgtgtcgcacagtggcccctcgggacagcctacagagctgctgtctcttccaggcattgctgcagggcctgctgtctctgctgtcctgggccaggcgctgagttctgtcgcggaaagtctgagggcagagctgggagggcagcgtttccaaagcaaatggaaaaggcaaacgggaacgtggcgggcagggtgggcagggcagcctggaaacaccctttgtcccatgcagctcattcactcggccaggatttgccgagtggcttactgcactctgtggccctgaaccagggctgggggatctcagggacatgtgtgtccacagagatggtcacctgccacctccaccatcacagatgccatgaaggctgtgaagggaaacagacctgtaaggcagcggagccgagatggggttgatgagtgtcaccagtcacaagcaaggtgtctctgttctctcttctacatgttgatcgaggtataacacccaggaaaggacatcaagtgtacagtttgaatttttgcacacacgtacacccgtgtaacccccacacagatcaagatgtagaacattccagcacccagaagtttccccgtgccccttcccagtcagaccccccgaccccaggtcacccctgccctgacttgcagtgtagagctcacccggccacttcctcttttgcccacagcaggtgaggagggccttctactgggaaaaactgtgactattaatttaggtttaaaatgtattggtaacaggcagatctggccagtccctccctggcttcaatctccttggtagcttccaggattactcttaggagggactggaggaaaagcccctctctgtccccatggcacccacagtgaccgtcacagccctcctctgggccctcactgccaccagtggactcactgtctccctctgccctgtgcacagcttcttaatttgggtccctctggtttagctcagtgcagccagaattcacccagtgcctatgtgagtcgggcactgggctggcccaggggcctttggaatgaaggggacagtcggtgagctgaggttgtccccaaatcagaggtgtgaccctatgagcagccaggtttttaattttgtaattgagatattcacatgccatacaattcctcttaaaatacacaattcagtggtctttaggtttttaatatattcacaatgctgtgcaactatcacctctaattccagaatattctgtcactccaaagagaaaccctgtacccattagcagtgacttcccactccccacacccccagcccctagcagcctccagcctctttcaatctctgtggattcgcccgctctgggcatttcatgtaaatggaatcagacgatacatggtcttttgtgtctgactccattcactttgcttaatgtcttgaccgttcatctgtgttacggtgtgtatcagagtttcacttctttttgtggctgagtaatattccactatgtggataggccacattttgttgatctgttatcagctgatggacatttggattgcttcctgatatggtttggctgcatcccaacccaaatctcgtctcgaattcccatgtgttgtgggaggtaattgaatcacgggggcaggtctttcccatgttgttctgttctggtgatagtgaattagtctcacaagatcagatggttttaaaaagcggagttgccctgcacacgctctcttctcttgtctgccgccatgtgagacatgcctttcaccttctgccgtgacggtgaggcctccccagtcacgtggaactgtaagtgcattaaacctctttcttttgtaaactgcccagtctctctggtatgtctttatcagcagtgtgaaaacggactgatacacttccactttttggctattgggaatagtggtgctgtgaacactaatgtataggtttttgtgtggacacgtttccagttgtcttggatatatacctagttgtggaattcctggctcatgtggtaattcatgtttaactttttgagtaacgagcggccatttttaaagactggataggaatatgtaaggaagaaagtaaatttattcacttccgttcaggaaatttacctaccaaacaacatgctgaatgggaacctttaggggcttggctgaatggccctgcacaaggtgtccaggatgcactgcctaaaagctgtggaccctgtgcctgggaggaggaccagcaaagggcagagccccccgtgtttcagatggaaaggttaagtaatgtgactctggtaatgcagtccagttcatttatgagatcatggacctcttatactacagactttttgtgtttttttgctgagtcttttgctccctaaccgtgttccctggctggccaccctgccagcaccacgctggtcctcaggaagcatcatcagtcagagacacaggcctttgctgtggcctcacatgctagcagaggaagatagacaacatggcgatggatgaggaagttgggttgtctgtaagaggatgagaagggctgtcattaaacaggtgggttcaggtgggtctcattgaaccatgagatttgaaccaagacctaggggggtgagtgaggggttggggaagggagattaatgcgagttctggggaaggaacattccaggtgaggaaacagccactgcagagccccttgggtgttggaggaagacactcaggtggctggaggggtaggggaagcattggcagggagaggccagggctggcaggcctgccgaaggctttgcttctcttctgccaggctggccatcacgggccttgcacagaagagagcaatgacctgccttcaggtttcaaaaggtccctctggccgctgtttgagagaaggtcacagtgggatgagggcagagcagggggactgctcaggaggcttttgcagtaaccgacggagtggtggcggtcactggcaccgagaagtgtacatgctgcaaagaaaatactctgttcaggagaccaggtgcggtggctcacgcctgtaatcccagcactttgggaggccgaggtgggtggaccacttgaggtcaggaattcgagaccagcctggccaatatgctgaaaccctgtcaatactaaagatacaaacattagctgggcgtggtggtgggtgcctgtaatcccagctgctcgggaggctgaggcaggagaatcgcttgaacccaggaggtggaggttgcagtaaaccgagatcacgccattgcactccagcctgggcgacagagcgagactccatctcaaaaaaagaaagaaagtaagaaaggaaaggaaagaaaagaaaaaaaagaaggaagggagggaggaaggaagggagggagggagaaaggaggaagggagggagggaggaagaggggaggtggggagggctctgttcacataataaagaaatccagaggtttgaccatgggcgtcatttcatcactgcacttggcagaggtcatcgctgctgctcccatccagctcagtcctgtggtccctgtcgtgtgtagaaacacatactctgtacgttgctgggatcatagcagaatatcagctccaccaatcctggttcctcaatggcgacacccacgcactcagggcatgtatcagtgtgcatggagttgcctcgcgttcaactgcgtggtgtgggtaacgcagttgtgccattgtgtgtctcacaattcccagactggtaaataatgttgcttctagttttttgctacttaagcagtgtcacagtgaacattcttttataaacgtgtttgtgttttaaaaataagcttattttggaataatttcatatgtacagaaaagttatggagttaggatgttttcatccactcttcatccagtttcctctgtgaacacctcacacatctgtggtacatttgttacaaccaagaaactgacattggtacccacactattcatgagacaccagactttatttggatttttttctttttgagatggggtcttactctgtcattcaggctggagtgcagtggcacagatcatagctcactatagtctccacctcaagtgatcctcccgcctcagcctcccaagtaggtgggagtacaggtgcacgcccccacacccagctaattttttattttttgtagagacagggtctcactgtgttgcccaggctggtcttgaactcctgacctcaaaggatcctcctgccttggcctcccaaagtgctgggattacaggcatgaaccactatatccagtctttatttggattttactacaaacatatgcttctgtatacttgtgtgactatttgtttaaggtacagtccctgaagtgggattgccgtgcctgatccttgctgccacatcatcatcccataggggacagggccccactgtcccccatcactgaacagtgtgcccaggtgagatcatggacttgggccccctaggccagcccagtctctttgcagccaaggaaagtgaggcttagctgtcgggggctgtggggggatgcagcttgccacacttgacacccaaacgctgattttgtttacatgagactcaggtccgagtttatgtctgagacctgggaatcaaaaagccacccccacagcctccagggacctggctcccctggagccaccgtgctgccccacaggtgtcacccatggaggctgcaggagcacctggcgtggtccaggggacacgaagccctggtgctggctcctcccaacgcccctgtgagttctgcctcccatagaccacatgtgtcatctgcgtgcacatgtgtgaaacccgaaagacaagcagatgagacccgcctgccaaaatatttgctgccgcgcagaacagtaagtcgtttttgacatatggatctcagcgcgcagccacagcaaacaggcagcgtggcgctgaggggcacaggtttgctggggggcgtgagccgggagcatcgccagcagcaggctcgcccccctgcgagggcgcgcacggattcgctcgccaccaagagcagggccccaagaggggccctcggcatagtcccctcgagtccccagaacttcacaggcagagagcctggctcgaggatgggcgggagctcagtctggctttgatctgcgggaggtgttgtgtgcagaagctgtgggcaccgggggctgcagagcttcagacctggagcctgctcccagggccgatctgaacccggcacctgtgctgacccaagggtcaaccaaagtcctgagtttggtggcgccgagggaggggagtggtcgcatcccaccgggtgcctggaatggtgctggaggaggcagcatctgatttgggccccgtagggccagaggggggatttgtagggacagagcaggagggagtggagctgaggctgggggagggcatggtcggaggtccagaggtgggagaggggccgccatcctgaagcacacccctcctcgtcttcctgaggcctccatcccttcctgatcttccaggtcatgggctccaggcttgggcccactgctgtccttctccactctctccccagggaacccacctgggttgtggccttaaacccagccagatgatgcttctccagcctctgcctcctgccgggccctcccctgagctcagaagctgccatcagtagccatgcaacctcccccttgccatcatgaagtatctcaaacttggcagcctcgaaacagagcccttggggagtttaagcagaaacagaatatttttgcatagtcttaaagtaattccccccacccccaaatattgagcaattacaaagggcaaaataggaagtttacggtggagaatcctggtggacacagccttggccaggtgatcaaggttcatgtcaccagtcatgaagcggcgtgcacccctggtgtgctgagaagggcagcttacctcctgcaggtgcccttgccccaacgcaccactcagcctaaccatggggagcatccgacaaaaccaagctgtccgcatctccaaggacaggcacactggggcttagggcttcagtgggggcggaggtacagttcactccctaatacgccatttacttggagaagccctctccagctgcctgaggcagaagcacccggttctctctctgcaggacccctgctttgttcttcctaggatgcgtcagcccctgattttccatgacagtcccctgtgcccatgtcctgcagcctctcctgggggcaggagctacagggtttcttgactcctacctcagcactttggtccaagctctgagcagagacctctgtaggcgtctgcaggactggctggcccttgtgtctgtctttcccctcgtcctggccccctttgtccactcctgcaccagcttccctggacccactgccactgcgtcacgtggtcggatctgtgtcccctgcactctcaccacctccaacttgcatcccggtcccccacaccctgacccccaaaggcattgtgcatgtggatgagtgagtgaaggaaaggctggcgtgttgggtgtgcgaggtcgcaggggcatgaggtgcctggacggcagagagccagggctgggtgaggacctgggtgccaggtcgggggtctgggcagcctggtggccccctgaaaacgttcacatcccttggggcctgctgtgatacatactaatcagtgcttcatgagaaaggtatgagccgaaagaaatgggggttcccactggattggtcgtttctgcattgtccggagctctcagacctggtttcttaaccactgtgttcactgaacaccccgggcttgatcagctgctgagggtgacttcacatgagattataattgtactgcctaatagagtgctcacacatgttaaaactacctggcaggcaggagtgagaatacagctttcccagaaacgtgttcaaagaaaaacccattaccaacccggcagaacataaagtactagtgtggggtatgaggcagcatgtgggggcgctggggtggcagcagggccagaccccacttcaggggttgcccccaggaccccccttgcggtacccgtatatcccacccccagactcctttccatgagctgctcccagaccccctcatatccatcctgcctccagaacccctatatcttgctcccactccctaccatatgctgttcccagacccccctccatggcctgcccccagatccccctttgctgccacccctatatcttaccccagactcccccatatctcgcccccagaccctcctccatggtctgcccccaggcctgctggtaatatgggatacagaggtctcctcactgcacacacaaaactccttccctgagcgagggataggcagagctgagggcgggggtactggggggctgaggggggttctttagggcaggagttacctgccctgtagacggggagaccacattagtgtttgtgcaaagccctgccctcgtgcacctgtccttccgtagcccacgatacctcatccagcccgggcccgtgggtgggctgctgagcacccgaccacagccaggcctccatgtgccactgtcaaggggacttggcccgccatgatcctgagtgcacaggtacattgctccgccctctcaacagccacgacccagaaataaagtaggcactgtaatttgcattttgaggataaggaaaccgaataatagagtctactgagcgcatactcagtctttccagaccctagaaagtaggtaccattgtcatcctcatttcagagctaggagagcagaggccagacaggttccatagcctgctcaggtgggtccgtgctggaccaggagaagggccaggtaggctgatgggtcctgagcttgtgctcccgaccgccaggctgtgtgtctgatggccgaggccagggtggctcagccaagcccagccaacccaagactagaccatccccagcgtccaccccagcctcctgggcacaggggtcacctcccacccctggcccacccatacagccttttcccatctaaccttttctttaaaggaaggcatcccttggaatgagtccaccctgagattttatggatccaaagaaaaaacttaattagctgcataaactgtcagttgagactctccagaggacacacctccggctttcaggttccccagggaatctgaggctctgaggcaggtggacagccaggtccctctttacccagcaccaggtaaccttagaggcctggccctgccttcctcattttgaaatgtcaccctgtggtcatctctccttccctgatgtgaagcagtgaccagttctgccccatcctacacatgtgtgcacacatacatgcacacatgtgtaaatacacatgcatgcacatgcatatgtgtgcacacatatgcacataccttcaccttcctatccctccccaggggaaggccttccttggggctgcccctcctccactctggcagcattgggacttgaatcttgtggccccgtcacaactgtactcagctactccagctccttgtgtgacctctcagcccccaggctctgagctacctgaggtgaggtggggccacactggtccccacctcggtgcagcacacacagccccatcctgggagtgccacccaggtttcctctaactgtgctttccaagggctggaaggacctcttcctctatgccaggtgtgctagagtggccccagccgagaacctgactgggaccaagccccaggacagtggtcaggcacacacctgagtcgctcttcctcccatagacacatttggagttcccactgccttcctaggccccaccacatgctgtcacctccaggaagccttcacaggttgctccctgctgatagccttgacctgcttcccagggcatttgcaagggagtagaacgaagaccaagtccgtgcaggggctgctggctctgtgctcagcacacgccccgtgaccacctcaggggcggctctgcgagtccccacttgacaggtgagccacagggccacaggcatggcttggggtcctctgcggagcccgatgtgtcactggccctcacgtgtccttctccgatctgctcactcattcatccattcatttattcagcaggggttcttgagtgtctctgaggtggagcgtctgctgtgaaggactggcgggacgcaaacggttaccactggtgtgacgggctctgatcaggaggcacagggtgttggggagctcagagggtgacatctcaccctgccttggccagtgtgggagggagagggttcagggaggatgtaactgtgtgtgtggcaggtgctgtggtggcacagagctgaggggacataggcacagttgctggatgcagggcttacaccgcgccacgcgtcagaagctagcaggggctgagcagcatctctgccctccccgcttgcacccccaccatccagtgcagcccagcacctagggggcatggggccagtgcctggcacattttgtcctgactgtggaaaaaagctcttcacagccctgagtgctgctttagctttggctccacccatcttaggctgctcagaggcctcaggcctccctggagggccagaagggattcccaggggcaggatagcacggacgggggggtccgctgtgtaagcctgggaaattctggctgcttgaagctggcacactcccctgaggctgttccaggcacagtcactgggtctccccgtcgaagtggctctaaggcaggtcttcccggttaccagcaagcagatacatgttgtaacgggctgagagtgggtgtgtatctcatattccccagaggagcttacggacagcccctctttaaggaacatctcccgggccgagcgttctgagcgccctctgagcaggcagcccaccaccggggcgctggccagtctgagtcagtcctgcctgggaggcagtcaactctggggaaggcagaagaatcacagcactgagtgccgtccagtttcccctcaactatattcgcggacacggcgccaacaggggaaaacttacatagcacctattcattctgaggcctggccggcctctggaaatcagcatttcagtgttggccaggaaggaggccagaactgtgctgtagcatttcaatgtatggctgtcccacaatttatattccagtcccctgatgatggacatgtaggcggtttccagtttttttcatgatcacagatcacacttctgtgaacagctctggatgtgtctccttgtgcaaatatggtgctaggtgttcttcagagcatttttctccaaacttgccgggtaatatggtcaccttggggagtttgttaaatatacatatttccaggtctgctctggtgggtctgatatttggagccaggaatcttttctttcttttaacagatacttaagtgattcctgtggtccaggaagttttgcaagctctgatgcagaagagctcgctggagcaaacatgtatgttctgttttactaattagatatacgctgttgatgactccaaagtggacgtgccatttctgtcccctccagcagtaggaaagaatcccattatcccacatcctggccagttcttggtattgtgagatttttttaaacttcttctcttaagactatgatggtattttgttgtaatataatccacattttctatattattactgtgcttgagcacttcttcaaatgtttgatgaccttgaccattgaaacttcctttttgtgaattacctattcatatcctttgtagcttcactgtggaatgcttgttactgatttgtagccattctttatacatttcactcccactcgtttgtcagttatatgtggtgcacacatctgctcccagtctgtggtttgtctttccactttgtttatggcctcttattgggacacagttttaaattaaaatataatcagatgtattaatcatgatgttgtagtttgtgcattttctatcttgtctaagaaattcatccctctccttgttatacttagaaagaaggatattctacattctccttcaggagttctaaagatttgctttgcacattcaggttccaaatctacttgaatttgacttgtgtatatggtgtgaggtagggatttgattttattttctatgtggataacagttgttccattatcacttttgagtagtccaccttttctctcactgacttataatgccacctctgtcatgtcagcttttcatatttatcagttcctgttaaatgtatctttaaagctttattgagacataattcacatgatacaatttacccatctaaagtgaataattaatgtttttattatattcatacaccatcgctatcagtcaattttagaacattttctttttttttttcttttttttttttttgagatggagtcttgctgttgtcacccaggctagagtgcaatggcacaatcttggctcactgcaacctctgcctcctgggtttaagcacttctcctgcctcagcctcctgagtagctgggattacaggcacccgccaccacacccagctaatttttgtatttttagtagagacggggtttcgccatgttggccaggctggttttgaactcctgatcttgggtcttgggatccacccgccttggcctcccaaagtgctgggattacagacgtaagccaccatgcccggctattttcattacccccaaataaaccttgtaccctttagttacctcctcatctctgtaaccctaagcaaatactcatctgctttctgcctttgtgggtttccctgttctaaatattttttatgaatggagtcatatagtatgtggccttttctgtcttcttttactctgcgtgttttcaaggttcatccatgttgtagcatgtatcaggacttcatttctttttatggctgaataatgctccactgtatggatagaacacagttatttattcattcctgtgttagtgtatatttgggttgtttctgcctttggctattgtgagtaatgctgctataaggattcatgggcaagtttttgtgtggacataggtcttcatttctcttgaatatatgcctaaggagtggaattgctgggtcacgtggtatctatttttaatcacttgaggaattgccagactattcaaaagcagctgcaccattttatgccattttacattcccaccagtagtgtatgagggctgatttatctatgtccttaccaacgcttcttatctgactgtttaattctagccattctagtggctggtgaagtactatctcattcagattttggtttgcaattccctacaatgatgactagtgatgtcaggcatcttttcttgtacttattggccacctgtatgtcttccttggagaaatgtctattcaaattttttgcacaattttaatacatacatatatgtatatacacacacacacacacacacattttttctttttaaacagattctgctttgtcggccaggctggagtgcagtggcacaatcttggcttactgcaagctccgcctcctgggttcatgccattctcctgcctcagcctcccaagtagctgggactacaggtgcccaccaccacacccagctaattttttgtattttttagtagagatggggtttcaccatgttagccaggatggtctcgatctcctgacctcgtgatccacccaccttggcctcccaaagtgctgggattacaggtgtgagccaccgcacctggcctatttatatatatattttttaaagtcaggatctctgtcacctaggctggagtgcagtggcacaatcacagctcactgcaaccttgaactcctgggctcaaaccatccttctgcctcagtctctcaagtagctaggactacaggtatgcaccaccattcttggtgaatttttatttttatttttatagagatgggatcttgctatgttgctcaagctggtctcaaactcttagcctcaagcaatcctcctggctaggcccctcaaagtgttgggattataggtgtgagccaccacatctagcctcctttgcacattttaaaagtggattagttgcctttttatcattgagttgtaagagttgtttatatattctggatataaggcctttatcaaatatatgatttacaggtacagttgacccttgaataacatgggagttaagggtgcttgctgcctatgcagtagaaaatctgtatatacctttgacttccccaaaatttaactacagataccctgctgttgactggaagacttactgaaaacataaacaattggttaacacatattttgtgtatgtattacaccctatattcttacaataaaggaagccagagaaaagaaaatttaagaaaagcataaggggctgggtgtggtggctcacacctgtaatcccagcactttaggaggccgaggcaggtggattgcttgagctcaggagttcaagaccagcctgggcaacatggtgaaaccttgtctctattcaaaaaaaaaaaagaagaagaaaatatatttactgttcatgaagcagaagtgaatcatcatgaaggtctttatcttcattgtcttcacattgagtaggatgtgagtaggctgaggaggtggaagaagaggagggttggtcttgctgtctcaaggtggcagaggtagaagaaaaactgcatataagtgggcccatgcagttcaaacccagttgttaagggtcaactgtattttctcatattttgtgggttgtcttttcattttcttgatgttgttttttaaagcataaaagtttttaaagttttgatgaagtccatttgatcttttattttctcttgttgcttatagttttggtggcatatctaagaatcctttgataaatctgaagccatgtagatttacccctgtgttttctcctaagaattatatattcttagctgtaacagataggtttttgatacattgttaattcacttttgcatatggtgtgaggtaagggtacaacttctgttcttttgcatggtgctatccagttgtcccagcagcatttgttgaaggctattctttcccctttgaatggtcatggcacacttgtcaaaaatcagttgaccattgtagtaggctgttcttgcactgctataaagaaatacctgagcctgggtaatttataagaaaagaaacttaaatggcttacagttctgtaggttgcacagaaagtaaagcagcatctgcttctgggaagcctcaggaagcttccaatcacagtggaaggcaaagcaggagtaggcatctcacatggcgagaatgggagcaagagtggtaggggtgccgtatacttctaaatgaccagatctcatgagaacttactcactatcgcaaatatggcaccaagccctgagagatctgccccatgacccaaacacctcccaccatgccccacctctagcatggaggattacaattcaacgtgagatttgggtggggacaaatattcaaactaaagcaaccatacacacaaagttttatttccagatttacagttctattgcattgatcactgacatgattactgttcctttgtagtaagttttgaaattaggaagtatgaatcttcctactttgtacttctttttctagatcattttggctattctgggtcctttataattccatatgaattttagattcagcttgtcaatttcaacaaataagtcagctgggattctgaagggggttgtgtcacatccgtagcccaatttggggagtgtcttagtccatttgtgctgctataacaaaatacctgaaagtgggtaatctataaataagagaaatttagcttgggcaacatagggagaccccatctctgaaaaaaaaaaaaattaggcatggtggcgtgcacctgtagtctcagctactcaggaggctgaggtgggagaatcacttgagcccaggaggtcaaggcagcagtgaaccatgatccactattgcattccagcctgggcaacagagcgagactctgtctcaaaaaaaaaaaaaaaaagaaaaagaacagcaatttatttcctcacggttctggagctgggaattccaatatcaaggcactggtaggttggtgtctggtgagtgctactctgtgctttcaagatggtgcctcttggtttgtcctcacgtggcgaaggagggaaaggagaaacagggacgaacagtgttcatagcagctcttttatttataaattgctaatcctgttcatgagagcagagccctcatgactgaatcacctcctaaaggccccacctcgaaatactatcacgttgctggtttagtttcaatacatgaattctggggggacattcagaccgtagcagggagcgttgccatcttaacaatattaagtattttgctccatgaacatgggatgcttttctgtttatttagatcttctttaatttcttttaacaagtatcattaatccaaaaatctgaaatctggtatgctccaaaacctgaaactttctgagcactaacatgacagcaacagtggaaaattccacccctgtcctcacgcgatgggtcgcagtcaaaacccaatcaaaaccttgtttcatgcacaaaattatttaaaattttgtataattaccctcaggctatgtgtctaagcatatatgaaacacaaattttatgtttagatttgggtcccatccccaagatatctcatgtatatgcaaatattccagaatcaaaaaatatatatagagagaaatttgaaacacttttggtcccatgcattttggataagggattctttacttgtattttgtagtgttcacaatataaactataatataatacaaacaatataagctaaaatataaaatttgcacttccgttgtcaaatttactactgagtattttattctttttgagctaccataaatgggactgcattcctgttagattttttctttctttcttttttaagaaatggagacttaggccaggagcggtggctcgtgcctataatcccagcagtttgggaggccgaggcgggcagattgcctgagttcaggagtttgagaccagcctgggcaacacagtgaaaccccgactctactaaaatacaaaaaattagccaggcatggtggctggcgcctgtagtcccagctactcaggaggctgaggcaggagaattgcttgaacccaggagatggaggttgcagtgagctgagatcgcgccatcacactccagtctgggcgacagaacgagactctatctctaaaaaaaaaaaagaaaaagaaaaaaaagaaaagataaggagccttgctgtgttgctcaggctgacctcaaactcctgggctcaagtaatccttctgcctcagcctcccgagcagcaagcagggaccgagcagcagggaccataggagtatgtcactgtgcctggctagatgtgtgtgttttctttttttggagatagagtcttgctctgtccaggctggagtgcagtggcacaatcttggctcactgcaacctctgccgcctgggtttaagtgattctcctgcctcagcctccggagtagctgggactatagctgtgtgccaccacacccagctaatttttatatttttagtagaggcggggttttccatgttggccaggctggtctcaaactcctgaactcaagtggtctgcctgccttgacctcccaaagtgctgggattacaggcgtgagccacactacacccggcctagatatgtttctaaaacaacattgtgtaggaaatttttcttaggctggttcaaggctaacatgctgaaggtcccaggtagtgaagcaggtgaaccctgaaggaccgtgcccctcccacacccaccctagcagctgttccacattgtagatggatccctgcgtccctacattgtcacctttctgtctaaatgagaggtggggagaaggaggggtgaagaccaatgaggttagtcacctcaaggggtggggccggtgcagagaaagatgggaactgttccctagtcctcttccttctggagttgggagggggaaatagccctgccccaacatgtgatccagcatggactatggtcagttacaaatatgagtgagccaggcccctgccctgaaggacctcccagatttgtggggaagacacaaactcagcacagaatggaaaggggcctggggacacagtgctgtgccgagcttggaatttgtttcatgaagggacagccagcagtgtctgtgcaggtccagggaggctccacaggggaagtgagatttgagctgaggcttgaaagaaaggtggaccgagaggagacgactttccagggcaggaagtggcctgcacagaggccccgaggcgagaaggaccacaactctcctgtggacctcctctttgtacgatcagttttaattacgcaaaaaaattatgcagttaaagctttgctcatttaaacagaccttccctggattaagagtaattaggttttattgtcattgtcttgaaaattgccttgccatggacctgtgaacacagaattatttatatatgtgtgcatagaaatcatctcatggatttttttttaactgagatgaacttgggggattttaaaggtaacaaacaaccccttctggctggtgtgggggaagccctaatttgtagtgcttgctacttttttttttttttttttttttgagacagtgttttgctctcgtcacccaggctagagtgcaacggcacaatcttcactcattgtaaaacctccgtctcccgggttccagcgattctcctgcctcagcctcccgagtagctgggattacaggcacctgccaccacacccggctaattatttttgtattttcagtagagatggggtttggccatgttggccaggctggtctcgaactcctggcctcaagtgatccgcccgcctcagcctcccaaagtgctgggattgcaagcataagccaccgcgcccggcctccactttctgtggtgtaaatattcctaccatggctgatttcctctggggaagccatgtcccctccacttctaccttcatggtctttgcttgtttattttcaaatgtggcccaaatccaccctgaggcaggcgcagtggctcacgtctgtaatcccagcactttgggaggccgagacgggcggatcacctgaggtcgagagtttgagccagcctgactaacacggggaaaccccgtctctactaaaaacacaaaaattagccaggcatggtggcacactcctgtaatcccagccactcgggaggctgaggcaggagaatcgctggaacctaggaggtggagattgcagtgagccaagatcatgccactgcactccagcctgggagacacagccagactccatctaaaaaaaaaaaaaaaaaaaaaaaaaaaccaaaaaaacccaccctgagtttaaaagaaaaaggctaagaaatgcttgctgggctgtctcaggggtggcagtggagctcccgtgcctcccacagatgaggacgtggaggccctggccgagtgctgaatgcaggttgggggacagggctgccggcctctgtaaggatggggaccccagacccacccggtggagctggggctgaaggccgtgataaacacagcctcaccctggcattgcccacctccactggccaccagctttgacggacagccccactgccactgcccctctccaggcaggccacattcacaggtgttctgtacagagagcagaagtttgagaaaacgtgacccactgaacagacacttgctgaggcccgggggcttcatgtgtcaggcccagaggtggccacaggcacaacacgcgtccctcagccttgccacctctgctgccctccaaggtgccctgggtccagctggccagaagcactgctttctggagcagccacttatgcttttacttttttctcttctttttttttttaagactgagtcttgctctgttgcccaggctggagtgcagtggcacaatcttggctcactgcattctccagttcaagtgattctcctgcctcagcctcctgagtagctgagattacaggcatctgccaccatgcccggctaatttttgtatttttagtagagatggaatttcgccatgttgaccagactagtcttgaactcctgaccccaagtgatccgcccgcccaaagtgctgggattacaggcgtgagccacagtgcccggcccagctttttcattttgaaatcaatacaagtttttacagatgtctgcaaaaaatatccatattgatggatcctggctcctcagaatctgaggtttttacaaatgtctgcaaaaaatatccatattgatggatcctggctcctcagaatctgaggtgacagcagcttgcagaggacactgctcccccaacactgccttccaagggagacatgccctaccccaaggcctcccttgtcccctccctcctctgacagtcaggtccagggggtcctggagagggcactggcccaccctccacacatgttgcacagtgccctgggaatgaggaaggaagggcaaaggttagggaaaaccagacgtcagtttcttgaccagccacagccggtgcagcgcggttatttatagccctggtggagcgcccataaatcaaggctttccccagcagcgcggctgtgagggctccgagatgatctcatggtgcccctcccttgaaccatcccagagaaaggaggcctgtcaccttcaaggaccaatgcttctgccgggactcaagcagaacctgttcctgctgctgcaaggtttacaggcagccttggggacagtccgtgcagaaatgtcaggagccttttcctgcataaggcatctcagagcttatagcacctccttggcggtggccgtgctcgcagcctgatacatgggacctctgtctcacttgcagtgtgcaccgcaggtcctaattccttatctgttccaaccacaaaagcttccactgctggatggagccctgttatcattcaggcttctgagccacctggaaggcaaaggcctctctgtgaaaatgaaaacttggctttaaaaagctggcatgggctcagccatggagttgcttagatgtagttcacaatctcgcagacccacggcaggattccacccaggagaaggtcctggaggtggccatggggctcagcagaatgagccgggaggagcaggactgttgacacgagagcaggggtcaggctcagtgcacaggtcccagtgtgcagagctcacccggccccagctttcatcacccatctgagatcggcctccttcactcctgttctctgggacttccttgtgtgttagagactcgagcaagaccctttcttgtctatccccggacggacccgcctttcccgcatctctgggggtgaggtcggctctcgttcaggcctgcagggtccctggtgccgcagcccctctggttggaaggccccctgcgtgcagccccgtcctccagccccgctcggctctgaatgagttcattatgtctcagcgcgccctggccgccaggctggcagttcttcgggctggcggggctcgggagctgtcagcctgccaaatccagttgtttgacttcatgtttcaaggccaaatcctttcaggaaagttctctccatcccaccactccaccgccccccgcccttagaaaaataaatgcgtgattgactggctttgcaggttttttatccatcgttctttccaaagaatagtgtgaacagctcattcgattcgttcatgtgacgtcctccctctcccaatccctgtctctctctctccttccacagccacatgaaattgaagtgggagaacatggtgtgtgagcattattgggggtggggtgcggagcaggcactggctttggttgaggtctgcaaaggaaagcacccccactgccaggctgctcaggagtggctcccaccctcgactgcggggaagtgctggaaccctccgcacgagggcaacctttcttgggctctgaaggcgcctctcatcctctgagccaagaagacttctgacccagaattctgagttgagttccgacgcaggcgtgggcgatggtgagcaactccaggctaccccgagaaagccgctgtgtgaccccattaggggacttggctctcctgccaagcccccacccagcacctgtcccttgtgtcatgactggacttgttacttctagcccaggagctccaggtgaaggggactgtgttggtcctgtctgcccaggactctccccacccgcacagtgtacctgtcccacagtggcaccaacaaggctcagctgagcccttgccagaggagacacggtgggcaccagccccagcgctgccacctgcctgcctcctctgccaccgagggaagccatgtcgccttcctgagcctgcttctctgtccacaaaacaaagactagataattagagtgcacacttggagggacaggatggcttcgtatccatctggctgtgccctgtaactcaagggatgggactgtgtcattggtaaacactgcaacaggcctcctttccaggaaacaggatccctgtctcagacagaagcagtctcgcccatcctggggcctgagagcatcattcctttgtgagactcaaatgtgaggaattcctgtttcctcaagtctgtgtggggagtgcagagcagccctctgagggggtagcgtgtgcaggaaataagccggggggaagggggcaccgcgggtgggtgttaactgctgcttcggggtccagtcctgttaatgaaagcaggggtgaggcaagcggggaagactctctgtacctgatcagtggcatgtgtgtggctccagccggcatggcctgagctgccggtgagggagaggcctcagggagaaaagacaaaagctggggtgcccctgaaggtggggcagagctggggcagtcaggggctcttccacaggtgcaggcctggtgctggaggacacagtccataccacctccaacccccaccacctcccctgccatggacagtgtggggcaggctcctgaggatgaggcagcctgacccctacccctgggctgtccccccgactcctggtttctctggaagcctggggttccaggccatgcaggtagagaacaacaggtttaggacaaagtcattgtggacactgaggcccagaaaagggacttgcccaaggccacacagcaggtggccagcactgtgtccgtgtctctgttattccttcctctgcttgctttgggtttattttgtgctccttttctacttccttaagatgggagcttcagtgatcaatttcagatctttcctctcttctaacaagcatttggtactatgaatttccctctgaatcctgcttcagctgcatcccaacttctgataccaggtaggagtaattgttctaagctccgcagctgactacagagagcggccacatttgagaaacattctcatctctctgatcttgggtctctgacaatggagctagcaacacttcctggttttaagggctctgaggagcaaattagactgtctggtggtgaagaactttgtttaggaacaatttaagatgccaacaggttcatctgctctcctgcctccttctccccaccttgtaacacaccctagacaagtcagcactcaagtttatatggacaaataagcaagtaaaattagttaggaaatcaaatattggggttggggaacaataagagaaggcaagccctatgggatggtaaactgcagtataaagccccatttgctggcacagtgtggtctggcctgtgactaggtgagggacagtgcggggtggcctgcctgcatatgccaggttctgtgtatcacatctctcaccaggaaggcaaaacctggcagatggcacccgggcctgatggctcctcaaccctctcagcaccccaaagagggaagtcccatcaccctcactcacccagccccgggctggccctgtcagctctcgaactgcagcatacccgagctcttaaagcacactggacctagggctcaggtttgagctttcatgatgagaccctgaggtcactggcggggaaaatgagtcactggggcattccccgaacttgggaaaagccctgaccccagaatcctgagcctaaattgctgccaagttcccgatttcccttccagtcctcagtttccccttctctccggcaccctcctgaggaccactgagccccaaccaccaccatgcccatggctggtgcccaggaggtgggagctgtggagctgcctccaggccttcccggaggccccatggctgtcttacttggtttgtggcttcccagtaaacggtgggacaggaccagggtctgaggaaaagcaaagcagtgttgacagagtgtcctggagggcagcttgtctccctggcctggagatgaaaactgaagaaaacaagattccgtctaggaatcgtccagggcgcggcagcccgccaggagcccgggcagcttggatggggtccctgtgggcacaatcggcccagtgtggggcgcaccgccccaggcaggctggcgtcccgggggccagaatgaatggaccctcactgggccgctttgacagtttatgaggtgatgacgttgcagctatgattgatgaggtggcctcagtcggcgtttcggggaaccgggtacaagttgctagggaaatacagggggagggtgctgccctccccactggcccgctctgggtccctgtgctatgcctcagtctccccatctgtgtggaggaactgatgaagacagcacatgccccctgccctggtccctggattgcaagtccagacagaagagagcgtcctgggaagccagctgagagcacctctttctctttgggtcctgcagcccgtgggtggcagggcggtaaccccatctgcccagaaccaggctgcgctctcgggagggaaacagactcttatcctcttaggtacagcctccctggccgcctcggatgagcatctgcccctcccgggcctcagtttccacacctgagatgggagaggttgcatcctaaaggccctccatctctcaggttgggggatcactcttgtttccctgcagttgtttccagcattagagtcacactggggtccttgttctagcccccgtgtgttggctgcatgacccgaactttactcccatgtcctcctcttgggggtgtgacatgaggtcttccccaagtggccccgagtgaggcaccaccatggacctgatgggcagatcccccagggcactctatccagagggtggtggacccaaacgcgacgtccccagtggcggtggctgaggagaggggttggggcgaggaccagctctgggcagtctccaggtcatgcgcgtttgacgcaggaggttttgccggcgccaggggctccctcggctgaccgagggtgtcccttactccataaggtactgatatggtctgcggagcaggtggcatttgccatgcctgccctgcttgggtatttggccccagggccagggctgtccctcccacagctattgaacaccaggtccttgctgcgtctggctgggctgcagaatacccttgacctttcagcatcctcagtacctctgtggagtgctgccatgaccaaactagagactgttattattacgcacattttaagacagcatcggccggacgtggggggctcccaccagcactttgggaggctgaggcaggcggatcacttgaggtcaggagttcaacaccagcctggccaacatggggaaaccctgtctctactaaaaatacaaaaaaactagctgggcatgatggtgcatgcctataatcccagctactcaggaggcggaggcaagagaatcacttgaacctaggaggcagaggttgtagtgagccaagatcatgccattgcactccagcctgggcggcagagtgagactccatctcaaaaaaaaaaaaaaaaaaaaaagacagcatcactgtagccaagggaacttaaaagaaaaaagtgcatgtttagacagttgttcatttcctccagtttcctgccagcccccttctgtagctgtgggcacgtgcacatataagtcttctctttctcaccctgctcattgttcatagctgcataatattttacagagcagataaactctgccggccttaactactcccagggtcgaacgtctccttttctatcgtaaatatctctgcaattcctatagcttttttccttcaattcagtaaactttttttttttttttaatttgagatggagtctcactctgtcacccaggctggaatgcagtggtgtgatctcagctcactgcaacctctgcctcctgggtccaagcaattctcctgcctcagcctcctgagtagcttggaaaacaggcatgcaccaccacacccggctaatttttgtattttttagtagagaggggtttcaccatgttggccaggctggtcttgaatgcctgatctcgagtgatccacctgcctcagcttcccaaagtgctgggattacaggcatgagccaccgcacccagcccctttgctcttcctttgtcttctgccatgattgtgaggcctccccagccatgtggagttgtgagtccattaaacttctttcctttaaacattacccagtctcaggtatgtcttcattagcagcatgaaaacagactaatacaccatgtcatttaatgaggctgggcctgcctctttctgatggaatgaactctccccaggtgacagcctctggtcccctctgcctttggagtgacttctcctgcatgggggttctgggcagggttggggggtgaacactggagagggatcagtcccagctctgttcttccccacccgctgcaacctggttggggacgcgaggagggcacccagcaggctgccagggggaggaggagggactgcagtctgggcccagagcccggttgtcccccatggagccacacagcaggcacctcagagtttgcatccattggagatggccaggagtctgctcgtgtgggaagaatggcttctgagcgccaaggtagcctgtgctggagccttggctgccatagaaggccattcacgtccctcacaccctggcctgtgctgctgggaaggctgtcactcctaaacagttatctgcttctgtgttaacctacagggattccctctttgctttggctgccagggaactcaaagctaagttttttcgctcacattcccaggcttctggtgtagatatctcctttcttccttactgtggtttctgagccatctggtgtagatatctcccttcttccttactgtggtttctgagccaccctgcaaacttcctctgcatctgtcccagtacaaatatcactgtgtgcccttctaggctatctgggacacttaaaaataaataaataaagacatctgtctgaggcttaacttgcatatagcaaagtgcatgaatctgaagtgtgtggctgatgaatctgtacacaccgctctccctgcacagccacccagatgaagacgaggagcacctccaatacccaagcctccctccacccagtcaacactcttctccagagggagctagcccgtgccttcacccccgtcttctgcctgacggtaaacttgaggtgaatgggctcctacccatgaacacccaccgttcatttcatccgtgttgttctgtgcagccagactccatcctacggcatccgtgggtgatgtttcatcacgaggctggaccacgatgtgtttatcatgtcactgttgctgtcacatgggctgtttctggcttggggtccttatgaacagagctgatcagaacatatctgtccatggctcttggtggacatagcctcatttcatgtgtgtgactcaggagtgaggttgctgggtcccgcggcattcatgtgtttagtgtaggagactcttgccaaacatttctccagagtgtctgatgtaccatgttgcgctcccgccagcctcgctgagttcccatagcaccatgtcttctgcagcacacgatgtggccggacctctccacgctggccattcaggtggacgtggagcggtgtcatttccttcctgaggggcctgcatgaatcccttttaggtggaacacacacagagtgggccggcgctcataagcagtcccgtgtgaatccttggagagctgtggaggtagcacagctgcatctctaggttggggccactcttttcttggaacaagagccctaaacgtgggaccctaaacaacacccagggaggagcagcctggcctatgagcagggggacaccatgaagctgggtggacgggtggggaggcccgactttaagggacctgcttcctaagcaaacaggccaggacagaggctgcaggtataagccctgccctgccaccatgatggtgaatatgaggtgtcaacctgattggattgaaggatgcctagatggctggtaaagttttgtttctggctggttaagtgtgtctgtgagggtgttgccagaggagactgacttttgagtcggtggaccgggtgacggagacccaccctccgtgtgggtgggcaccatctcatcggctgtccttgtggctggaacaaagcagatggaggaaggtgggaagactttgtttgctgggtcttctggctttcatccgtctcccgtgctggatgcttcctgctctgggacatcagactccaggttctttgacctttggactctggtttagctggagctctcgggaggtgtcggcttccctacttttgaggattttggactcggacttggccattaccagcttctctcttccccagcttgcgtacggtctgtcgtgggacccgcctagtgatcgtgtgagccagttctccctaataaactccctttcatgtatagacatggatcctatgagtcttttccctctggggagccctgcctaacacagtggcatagagctccaggcaggcccctcacctggccttcagcttcagtttctttgcctactgggctgaatattggtgtcaccacccaggatagcaagtccccatcagtgtcactgccacctgtcgaagagatcccccagcccagcactcgggtctgtgagtggcagtggagatgacactaggctctaagtagtggggctgcgtgactccagcgcccctaggaccagcaagggcacccagagcactgtgggaggactggtgccgcctccctgcccccctgctttctcctgggccactgggctagacatggggcctccctgcccccatgtggtttggcctgaaaaagagaggaggtggggtggcacttctggggcagcccagagtgagtgccctgccctgagcccagcgtgtgccctgcctgtgcccctgggtggggggtgcatacagacctcggggggatctggccgttggagtgcgcactgggagggacggagtcagctctttttaatctaaacacacaccaggtgcttaacgtgtttttatcaaaatatcaagcacagctgcaagaggctgctctgctccaaggccctacgcccaggcccgtccctctgccaaagcgagccttttaggtgagccctgccggccacctgctagcccaggccccggctcctggtgggccctggaccaggctgcaagcatatccaagtgcatggcagtgagacgtccaggcctcccagccccaactctgagcctccgttgggtcagtggtaagatggggcagtggcacctacctccccggctgctgtgaaaaccaagtgagcggaagcatggcagcccctcaccaggtccccagtgggaggactctcaatgattggcactgccaggagtaatgttatgagaggagcgtcccatcagcctggaggggacacaggctgggtcagcacccacaggggcttccgcttctctgtctcagggggtccagggtggccccttgccactggtccccaggcacagccgctacacaggtgcctctgcagggtctgggagtcagctgtccagggcctgcttggccaccgcagggcaggagtgtggcactgtgagcctggtggcctggctgggcgtggccttgaggttctattgataaccccatggggaaagcccccctgaggtgcttgggcatcggacctgcccaccatctcctgaggtggggcctgctctccagacagtcaggtcagcatgagttcccatgaggccagggccagcacgaggaattgctgggagcggtccatccacccatccgtccgtccatccatccatccatccatccatccctatccacctgtccaccctgatgcagttcctgccatccacgtgtccatctgtctacccgtacacctgttcatccccatccatccatccccctgtccatccctgcccattcatccaccctgatgcagcgcctgctacccacccttctctgtccatccccatccatccatctctgtccatccatctgtccacctgtccatccccctatccatccctgtccattcgtccacccatccaccctgatgcagcacctgccatccacccttttctgtccatccccgtccatccatctccatctatccatctctgtccttccatctccatctatccatctctgcccatccatccatccatccatccatccacctgtccatccccatccatccatccctgtccatccatccactcatccaccctgatgcagtgcctgccgtccacccttctctgtccattcccatccatccatccatgtccatccatccattcattgcccatctgtccactcacccatccactcatcccacccacccatccatccacccatccgtccccagccatccatcctcctgtccatccctgtgcatgcatcatccttccaactgtctgtttgtccattcccatccatctatccacccatccacccaggcgagcacctgccatgcccacgacctgcaccatctcggtgaatctccatcgtagccccaggaggggctcttttcagctgcactttcaggatgagagaagggaggctctgagtggccatttgcccaaggtcacacagagctcagggccagcgcttcccatggccaaagcccaggccccctctgccagtcccgcctccaggcttgcaggtggagcagccaggggttccgtactccctgccaggcagctgccagccctccccgctcccctctcagttcctcagcctggctatgggaggcccaagtcccacgtgtcactggaattcgggaggcctggctctcaaagctttctgtacaaaacaaggagaaaaagagaagccacttttgctgtctggacatgctgtgggtgtcctggggaatgtgctggcccactgcttggggcctgacttggagtgcccaggagctgcgtctgcactgtggcttgaaccgagtgtcgtgctctgtggacagagccatccactctgggggtgctcgtcccatggcactgtcagcccccattctggaaggtcccaactgggtcccccaggcctgtgtcccagagaccttccaggtcttcattcactgatcatgcatttattttgggggtaggataggtcctgaagaccacagggcagggatctctgagttggggcttttgcggagtgagggagtgagagggtgttccaggcagaggaggtggcagagaaaggcaccaagtgaggcaggggcagttggggacatgagtgctcgggagcggggagagggggcagctggtgggtaagcagaggccagctccaaggcccccctgtgggccacattagggagacagggtctggccttgttggtagcgcggcccctcgcagtgatgggtcccctgtcagacctccaggatcctgggttgctggtttggcgcagccccggcttctgtgagcttttgtttctgttactggcacctgcagctcttcctaggcccctgccctggggcactggtgcctgcttcgccaaaggcacagagggctgcaggcgactggggcagggtggggggctcgcctcttagggctcagcttcactggccagaaacagggctggctcgatctgggtgttggcctgggtggggtggccagcatgcccacacctctcagtctggatgtggggagaggttgttccatccagggcagttggtctccagaaggaaattctctgtatctgggtgctgcaacctaggtgcccggggtctcacgcagtggggcctccccggaggaggtgaagtgcatgtggcccctcgtgctggatggagctggactctagggaggaccaggagggtgagctcaggccagggcgccaaggccctgagccccaggggcctgcctgtctgcccatcattcctccactcatggggagggactgccccccagcccagatttctaggagggccgaggctcacaggaagccaatgccaggcgataacccacagtccccagtggtcagtaggaaggaggctgcctgcttgtgtgctcattaggagccaaggccatgaggccactaacggaatgtccctgatctcagggatctcagtgtggaggtggcagcacacaggtaaccaaacaattgtggcagattgatgggtgctttgatggggtccacacagttaccgtggagtgcagagtgggcacctaactgaggaggcaagggttggagggaggaaggtttcagggaagacttcctggaggaggtgacttgttcttagggaatgaggcccagggagctaaagctttagggcagggcagcagattcagagacacccccaggggtagggggaagtgcactagagtgacgtgtgcctggcgtgggaacagggagacattagagaatagtgggagtgtggcaggctgggcccgccttgcccctttcaaggaagcagctgccgcgtcacacgctgcagcgcagtgctgcccatggtttgtcggcccatattgtgggatctttcagattttctcaagaagccaggtttccagctcctaggtttgaaaagttctatgtgcgcttgaccggggggccttacgtacgtgaattgggtgagggcctgagacaggcgggtgagcacttgccccacactccagccaggattgggggttcaaggtaccgcatttgccacgtgagggagtctctggagaccaccaacccgctggtgggcggccaagcgtcaccaccagtccctctcagtgcgtcccacctctgttctcagcccctgcggtgcccgcagtcagtggggcagtgagaaccgcgcaggaaatagctggcgccatggtgtcggttctggttgacagagtgccacctggctaaacccagccgtaaataagccccgtttcctccctgtggacagagactggtgggtgccaaggttcacggcaaaaacccttccccgccaggccccactgctctctgcctggctggcacacgcccttgcctcaggcctggctccccgggtcctgttgaccaccaggctgcagatcccaaggaatgcaggcatctgggtggccccacgtggagcaccagccatggggaggccgtgggctaccaggccctgcgtgcactcgcagcatgcaccactccttctccaggtcccgttgacctccatgtgtgctgtgccccgtcctccgctgcactgcctcttcgtagtgccccagctccatccgggggtgggtgggagtggagacccctgcctacaggcctccgggtctagccagagtcaaccagcccaccagagacacccagatgcaggctatgccccacgtgcccagggaggatctgctgacgtgagcccagatttcgattcttgtgcgagggagacgaaccaggtttaaaccccagctctgccacttctggctgtgactttgagcatggctgttcatctctgggggtctctctcctcatctcagagaagcagatcatggtgcgcaccccatggcgcgctgtgcagagcacatagagacgccacccacagctggcttactgcagaggtgtgggcccattgtggtatcgctgatgtcctggaggccacaagtctgctagctagaggagcctcacctgcatcccggggcctgcccactggctggtggagagctctgggttgtgggcagcaggcaggcctatttccagtgagtctgtggctcctgggggtgtgtgagaccttgggcagatcccttcctgtgcctcggtttacccactagggctcagagccctgggggactggctcagtgagtgcctccttctgcgaggagagacttttgtccttactagccagaggacaggtcagccggggcaggctctgggccctgggcaccacccactggaaggaaagggggttcagttagcccagccctgccccccaatggaagaaaattggggttcagttagcccagccctgccccaccccagctcttcacacatccagatccagatgggggccagaccaccaggagactgtaaggatccaaggccgaggggcaggagacgcagtgcgcccctctcctatttctgggttgcaaccgcccacacatgtgcacacagggaagatgtctcttttggttggggggcacaggaccccacctcactcttagctctgagacacccttgtcaggcctcctggacaggtgggagggacagggcagggtggggtgtggccagctgcagtccctgggaggggtactggggctgggccaggccagggcaggacagagttggggaatccaggcccctcaccattgccctggctctttgtatgtagtgctgcctgcgggcggcagcctagctggacacctgcagtgcgctcatgcccagcctctgtgggtgtctggctcaccccacgtgggaggaaggcgggcacgggtggtttgggctgggcaggcccagcccgggtgcacagctttctcatccacacacacattccatgtgtaggggctcagggggaggggtcacctcctccctccccacccctccttgttacagaggagcagatggccaaacggatgggggacaagcacctgacagggccactgctcctgctctcagctgggccagtgggaaagggagcttggcacctccaagggaggtcccagccaggggaagtgtgtccccatccctgctctggtccccaggccagagaggtgagggacccagagaggcccaggatgaggactccttcagtcctgggtttttgtttggattttttttttctgccaagtgacttcaaagccgcccgagaaggtcctgtatgtgtgagcaggttgggcaaggggagggcgggtctccaagctgggagcctcctcgcggccctcgcctcggcactgatgtggccctggaccctccggtccctccaggccacccttcgtgccaggacccagctggcttcaatggcaggaggtgccctgggctggagggtccccacgctgggctccggggctccaggttgactcagcagtccccaggcctgtgggcagagctggcctgggaggacacgggcctgagggctgagcaatccctgagtgagaacaggtagctgaggctggagcctgtgtcccccaggcagctgctaaggcccgggcctggggctggagtgctgggctcaggtgccatgtgagctctttgcaggcagggaccaggtcagagtcctttgtgtgcctcttgacccccaggtcctgggagaagaggggcttgggaggccttggctggatgcacaggctccatgggaaggagctcctgctcgggtttctttgtaagcagcacgacatggcaccttcccagaggtacatgggtctgggcatgtcattccccgacctgcttccagaacccaggggacggaggtcggagcagcctgcctgagatctgtggccatctccactccgccacccagctaccccgtaaggaggccaccgaccctcgggcacctgccagggacccagctcaggccaggtcacgggctggatgtgggatgttctgggccttctgcccacctcactgggccccagcaagctgctgtcctgagggtggccgtgctgggccagcacgtggacagcagggaaccccatgggcacaggcctagtgtgtgtgatgtaccggtgcagaccatctgataagcagccttcagagaagctgcgggtgtgcaggtggaggcagacctcccagctgcatcctccaggcacttgccagggtcaggctctggtgaacacccttctagaaataaccccccaggcccccgggggtcttgggctcctggggttctgatggaaggatgccccttgcccggcctcactacctccacctcatgaatcaccctctgtccttgtcctgcaagtctgtcggcacagccgagctggcgtcaccctctgatgccagtgcccagatccctgaattcactgggcagccgccccctgcacctggctcccagccactgcctttccaaagcaaatggggacagggatacctgcccctccctgggtaggcagctgttgcggcctccagttgtgagagtctctgccccaccctgccccttcccctgcccctgcagggtagagaggaggcgggtgggccaccgactgggagtcccgggcagctcaggtccctcaagagcccctcggggagaggccgcacccgtctcccttggtcagtgttccccaggaggcggtgctggcctgcgtgtgccctggaggaggcggcagggagactggcctcatggggctcttttgctggggcctggccaccattttccttcgattggtacaaagactgtccctgtgaagctggccaggccccaggcacagaaccgccttgttttctgcctctcactcacccgttccctgcattcggctgacagagcatgagggggaggaatcactgatgacaggcactggcctgcccagctgggggcctttgtttattcatttggtgggcacttcctgggtgcctgctctgggtcaggcctgtgggggggaccactgagggcaggaaacctggcctgtccctccaggaagcgaagtcaacactggcacctgcagatgaagtggcagagcagcccccagctttgatggcatggggtggttggggggcacattctgcatgctcagaagagagagcaactcgccctgtggaaggagcatacagtgggagatggggacaggtgagtggggtcttgaaacgtgaacaggagtttgcaggtggagcaggtttcaagagcattccagggcacagcatgggcaatggcagggagagctgggatgtctgaagagcagcagcccagacactgagtggctggcgaggcgagggaggtccgcaggtggggcccagtcaggatggactcatctgccatgctgaggagccaggctatgatgtgggcaatagggagccacagcagtttacacaggggaggcgggagcagctgagcatttagggaagccactctggctgcaaaagaggagagggggccaggcaggagggagagccagtggagggggagggggaaggggaagggaacaggcttcagcagaagacctcctgccttcaaggggcccgggtcaccctgtgggttccggtggttaaggccagcctcctggtcctgcagccacatgcctcccagctgtgtgcctccgcttctacctccgaaacccgggcagcaggacccaccctgcaggctcctgcgcaaggatgggggaggtacctggagggcccaccccacatcagaagcaggtcctcagccagagacgcccaaatgcagcccgagggaggctgcccccaggagaggagaggagaacccgcagccgccccgcaggcgcctggggaaatcactcactgctcccacctcccagccaggccccgagggccaggcactctggcttagtgaagaagccacccagcaaaggcctggtgtctccacaccattctcggggggacagcagcaaggacgatggacgggagcagtgctcagccttaacgtccctgtttccggaagcgtccctgccccgctcagtcctgccccactccctctcccatcagtgccctcagactccaggcccttttctttctttcccagcagtgctgacaactgtgagtgtggaactggactgtgaaccctggagggcagatggtggaggtgggagggggcagggccagcgccagggtgagctgcagggtccccaggtgcaggggaaacgccaacttcgtgtgcagtcttgcctcttttgtttcgtttcagagtggcccccagagccaagccctatgctaccttacagatgaggaaactgaggctcgggcaggtgaagagaccggccctgggccatgcacttttttggggacagagcagggtgctcagctgtctgggggcctgggctgggcctggggcagggggaggagcagcgctgtgacccccacggggagccgtcagccaggcccgtagccagggaagggcggaggggcttaacgcaggcccacatggccaggacacagaaccgtgggggcagccccagcccttccctgcctgttacaggcccctgcccctgttcctgtgtcccatgcctgtcccctccctccatgtgtctgtctctactcgcctgccctctctctgccctcttttctctgcttcatcctctttttccgtttctgcctgtctcatggttcttcctgtccctgccctccctccctccgtgtgtggccgtgtctctctgtctgtctcttcctcctcctccagctctgtcccagtagggccactccgggcaggatggtctgcggcccctcctgcttcaccctctccgctctgtacccactgccgaggacacacctttgctgggtttcttggttcagcttttgtaccgtgacatctctgaggtccacaggtcacagggcactggccagtgtggggcagggactcctcctgcccaggccattggacaaagcagagctgcttcaggggctttggggccaccaggccaggaacaggtcccctcctgcctaccccagctccctgggagacacagcccacagcagcgtcacgtaattaagacataaaagtccacgatttccgctgcgtgtgggcaaacaccggccgggtgctaatgcaatcctttcttttagtggattcttgaatttaaatgatatgtaattaaacataattagccacggacgccattcggctaggtacacgtgcggagtggggctggccggattctcattacaaagcgtcatcttctatggggagacctcttgttccctctcggggctggttgcttttgcctggacaataagcctggccccagggcacctggggaaggtgtccaaggagggggtcctgggggccagtcccccgggggctcggatgatgaatcatgggcggtgtttgctggaagaatttttgttccgcttcgggcaacaaggaacccaaacctctcactgttgtggggagaaaagttggaaatcagatgggaactggctgcggtaccagagaggacgtcagacccgggccgcggcctcagcccccgactccttgtaagcgtgggctgggtgcacacgtggcggctgcgagcagtgctttggagctcatctccccagaccctagtgggagctgagggcagcctggcagggctgatgcaggcccatgcacacaggccaggcagaggtgagaccagggaatagaagcgttgcaggggcagagggcaggagggggcagagcctcggcgtaggagtgagcctactggtccttcccgcaggcaccctccctagtgccaggtctgctgggactccaggctgttggtatgccccctagggtcctgcttggagcctttctgaccctcctgatccagtgcctgtgccgggcttcctccccactcaccctgtcctccctgtgccccatgtagcccacacatcagtgtgtccgtccgtctgcctgtctgccccccaccagcctgtgtgcccaggggaaggctcaaggccggtcatgctctacctccctggccagcccagccccagcccacggtgcattccctggacaggattgctgagttttccattttagccacaacagcgcccacacccgcgccaaccaacctgtccttgccacgaatattggctcctgccctcctcatgggagcccatggtgcatgctattatgacctcggtctagaggcagggacaccaaggcacaggggcttagggaagttgcctgaggtcacgtggctggtagggggcggagccagctttatcctcatggcacagatgaggaaactgaggcagagaggtgttttgccagggggctgatttactgagctagccagccgttctggttttgtctctgtctcgctcagagggacccccaccccacccacctgcctacccagcttgccgtgtcctcggccagggccagcagtctcagaggtagagctgctgagagaagcctgtcatctgtcctccaagagggatctgagtaagccggagagcatcagcagcctcaaggggaccttggacccctgcccacctttcattctgtaccagggcagggtgtgtctgcacagcctggagccaggtgtggctggcagccccaggaagacagggccaaccctgcccagcccagagcagccgcatctctgcagcagcggtgcacttgagtggaagcactggcttcccaagagtaagagcttgagaaggggcctgtccagagccctgggggacggtggacaggccagcccaggggtccaggcctttcctgtaggggccgaatcgttaccacttcaggctctgccagcttccatttggtctctcttgcatattactctcttctttttacacccctttaaaaatataaagacaggccgcaggtaggatttggccctctggcctacacaatagcaactgtgagcattatatgtggcttctctttctttttgagactctcatctgtcacccaggttggaatgcagtggtgtgatcacggctcaccacagcctcaacctcccaggctcaggtgatcctcccacctcagcctcccaagtagctgggactacaggcgcctgccactacacctggctgatttttgtacttttttgtagagatagggtttcaccatgttgcccaggctggtcttgacctcctgggctcaagcaatcctcctgccttagcctcccaaagtgctaggatgacaggcgtgagctaccacgcccggcccatatgtggctttttaattaaatttttgaggtataacattagtaaagtgctctaaaagtgaagaataggatgttagccccacccaggtcaagatctagaacattcctggcacccctattcacctttccgttaggaagctgccgtcccaccctctgtcactagtctgcctttgccagctctggagctctctgtaagtcagtcctgctgtgtgtattcctcgagtctggcctcagttgctcttcaggtctggggctcacccccattgccgcggcaggaactcaccccagattgagccgttgtggagtattccagtgtgcaattctgccgtggtctgttttcccatcctccccctggcgggcatttgtccagtgtttgacttttgctattgcaactaaagctgtgtggagcagtgtgcctttggtgaacgtgcctttggtgaacgtgaggatgcatttctgctgtgtacatacttaggagtgggactgcagggttgcaagtggatatcaacacgcagttttccaatgtggcccgtaccatagaaggcctcgtggtgatgccgagtccatattccagcactgacgcaggattgcgtggtgccaagtccctatttcagcactgacgcaggatcacgcatgtcttcaatgcccctctcctagatcgtagacaccctgtgtggcatcacagaatgacacacacaagacaagaggcctctaaagccctgacactgacacgggttgttcttgctggggttgctaatggagcgggtgcgggtgtgggtgcgggtgagggtgctgatggagcgggtgagggtgcgggtgagggtgctggtggagcgggtgagggtgcgggtgagggtgctggtggagcgggtgagggtgagggtgagggtgctgatgcagcgggtgagggtgagggtgagggtgagggtgctgatggagcgggtgagggtgagggtgttgacggagcgggtgagggtgcgggtgagggtgctgatggagcgggtgagggtgcgggtgagggtgctggtggagcgggtgcgggtgcgggtgcaggtgagggtgctgatggagcgggcgagggtgagggtgagggtgctgatggagcgggtgagggtgcgggtgagggtgctggtggagcgggtgcgggtgcgggtgcaggtgagggtgctgatggagcgggcgagggtgagggtgagggtgctgatggagcgggtgagggtgcgggtgagggtgctgatggagcgggtgagggtgcgggtgagggtgctgatggagcgggtgagggtgagggtgagggtgagggtgttgacggagcgggtgagggtgagggtgagggtgctgatggagcgggtgagggtgagggtgagggtgctgatggagcgggcgagggtgagggtgagggtgctgatggagcgggtgagggtgcgggtgagggtgctggtggagcgggtgagggtgttaatgaagcgggtgcgggtgagggtgctgatggagcgggtgcgggtgagggggccgcatactcatccccttgcctggtcccccaggctcctactttggtgtgtgctcattcaggagcagcagagtggccgtgcttgggacggccagccaagcgatccccactggtactggcatctgtaggtgcccctgcttggagcgggtgctggcaactgccctttgctggaaggggcagcaatgtccagcagtggtggaagctctcgaaagaacacagggctgggagtagaactcagtcagatcccagctctgctctccctgctgtgtgacctatagcaagtctctgaacctttctgagctgtttcctcacctgttcaagatgggggtgaccacttcttccttggaggatggaagggagagggatgtgaggctcgtgagtgctgtgctcggagactgtgcaggaggggaagcggccgtagccccatacaagcaggtgggcaaggaagtgggggcgctggaggggcttaggggacaaggggttctgtccttgggacctggacaagagccaaaaggtataccctcacagtcacaggggagcccacactgccctcccctcccagggactgcccctgtggcccccgtccaggtccacagcctcagcagtccctgagggccaggaggtagcctccaggccactgggctgagaacccttaaaagaaaaagcccccctgcccatgacagtcattttagggtcaagaaagccctttgcagaaaccaaaccctttgaaagggccaacacggcttttagatgacgtgagaaccaacttcaagcacttcctctcagaccagggtggcagcttccaaagtgggggcgctgctgccgggagactgtggtttccctgcttctcagggagcttgggccaggccgtggggagatccaagcaacatgtccctcggtagtggtaggtttgcagacgggccgctccccacttcctgccaagctccagcggcacctctgtgggtgcaaggctgtggggaggggccttctggctcacagccctccaggctgcccacagcccctaggatgaggtgccacttcctcaaatgactgtcaagactgggatgggggccgggcacggtggctcactcctgtaatcccagcactttgggaggctaaggcgggcaaatcacttgaggtcaggagtcggagaccggcctggccaacatggcgaaaccctatctctacactaaaaaatacaaaaaaagccgggcgtggtggcgggtgcctgtagtcccagctactcaagagccgaggcaggagaatcgcttgaaactgggaggcagaggttgcagtgagccaagatcgcaccattgcactccagcctgggcaacagagtgacacttggtctcaaaaaaataaaaaataaaaaaggctctggatgtctccacgaacccacctgaccccttcctgctcctggggctctcctctcccacccacgtgtctacccgtagctcctgcctgccttcaggactcagcccacagccccttccacaaagtcccctggcagggcccggaggcctctgcactgtctcatggcttcattcctgtgtcctcagcatccagtgagacctcaggagatgcctggggaatggctgaaggctttggagaggttgctgcccccagaatcccagccagagggcagtttatccaggagcccggtgcctcctctgttgggtgggcgtggcctcagagggcaccaaccagaagacacatgctgtcactgacatgcgggcccccagcgttagggcaggcaggagctccgagtctctaccccagctgtccccacagtgcacatggactaggctcctcccacggggcactaggccaggccaggggtgtggggtgagcccctggggagcccagagcagggtacactcatgtccccaccatccaaggtgagatgtctgtgctggaggcccagagggagctgggccggctgtcactccaccacaaggctgagcaggctccccagtgggagacaggtttggggaggtgtccaggttgagggcacagtcaggaggggccatgtgtgcctgagtagggcagggggaggagggggatggccagtggccacgggccaggctgcctggcaggacctggaggtttcctcaagctaaggccaggcccatgttagatgctgggagggtgggtgtggctgcagaggggtcagcgtgggggcactgactaacctccggccatcctctccaggcccagtgacgagcaccatccggaagtgaaggctgatgggtacgtggacaacctcgcagaggcagtggacctgctgctgcagcacgccgacaagtgatggcctcctgggagagccccgcctcctccacccctgcctctcctccacccctgcctcccctccacccctgcctctcctccacccgcccaggagagccccacctcctccacccctgcctctcctccacccctgcctcccctccacctgccccagtgcccagaccaaccaaggccctgacagccctgccttctgccctctgccctgcatgggcaggcatttgttccctacctgggtggcctgctcccctgcctgggccctgacttcagctccctgtagtgaagtccaggagggtgggacaggcctgtcaggcctctgggaatctcccaaatcccagaactcaccactcaccatgggcctttaaatgcagtaaactccacctaaccagattcaggggcactatgcccactgcctcctcttcagactctttgcatttcagtgaagagcctggaagaaacccaggggcctcctatgcacagatcttgcagcccagaaccaagtcagcctccctgcgactgcccaggcacactgcccaccaccccacccccgaaacaatgccagcccgctgctttttctatcctcccagtcacctttgcagacaaagaccaggggcagctcccgagggcactgtgaaggctcccatgccacacagtgagaactgtagcctctgcgtccaaggcacacagggtactttctggacccactgctggacagacttgaaggtgtcatgcccggtgtgtgcaggaggaaactaacagttcagtaaactctgccttgaccagcagcctttgactcaggctttgactgctaggaagaaccgtatgggggaggccaccagcagggtctgggccactgtctctagtcctgccttatgcttgagccactgaatatcagaggtgcgagggacaagggccctgaaacacctcacctgctccagccccttcacttagcagatggggaaactgaggcccagaggggccagtgagctgctgttggccctatctggaacgaggcagtccagggcaaactttggcactgccttccctaacggaacagcctgtggcctgggggtggtgagctttgctttcccgaccaagggcgcggcggccttcccacaggggccctgggaacaaatcactcataactgaagtttcaggtttcagaatcagtcagctgaacagataacttgattgctccatttctccccaaatcagttcagaagctactgataacccttgagagactgcttctttattttattttattattattatttttgaaatggagttttgctttcgttgcccaggctggagggcactggcgtgatctcagctcactgcaacctccgcctcctgggttcaagctattctcccgcctcagcctcccgagtagctgggattataggtgcccgccaccacgcctggctaattttttgtatttttagtagagacagggtttcaccatgttggtcaggctggtctcgaactcccaacctcaggtgatccgcccacctcggccttccaaagtgctggtattacaggcatgagccactgcgcccggcctggagagcgcttcttatgagagaagccatgggtctccggagcaggatttccaccacatgaaggtggacgataagaaagtgtgggacccacgccggggaggggcaagtacagcccccgcaaggccgcccattaaatcaggattgggggaaacggtgacagtcttttcagcaagatgagaggccaccagccgggccagtgtcactggatttgagggagggccatttggccccttagttggaaaatatgcagatggctggctccttgatggagtcccctgatggcagctccttaggagacaatacaggacgttcaggcggcggctgcagttcagtggcagcctggaggggctgtgcgtgatgccgggctgtgcgtgatgccgggctgtgggtgatgccatgctgtgcgtgatgccgggtttgtgggtgatgccgagtttgtgggtgatgccgggctgcgggtgatgccgggctgtgcgtgatgccgggtttgtgggtgatgccgggctgtgcgtgatgccgggtttgtgggtgatgccgggctgcgggtgatgccgggctgtgcgtgatgccgggctgcgggtgatgccgggctgcgggtgatgccggggctgtgtgtgatgccgggtttgtgtgtgatgccgggctgtgcgtgatgccgggctgggggtgatgccgggctgtgcgtgatgccgggctgtgcgtgatgccaggctgtgcgtgatgccggtttgtttgtaggtctttagttttttctttcttttgtgtttaatggggaagcttcccgccaagacagcaacccctctgggggaaggagttcaagctgagacctctttttttttttttttgagacggagtctcgctctgtcgcccaggctggggtgcagtggccaatctcggctcactgcaaactccgcctcccgggttcacaccatttccctgcctcagcctcccgagtggctgggactacaggtacctgccaccacgcccagctaattttttgtattttttagtagagacagggtttcaccgtgctagccaagatggtctcgatctcctgaccttgtgatccgcctgcctcggcctcccaaagtgctgagattacaggcgtgagccactgcgcccagccgagacctcctctttcaagttgctacccaggagtggggtgcccagagggacagcccctggaggtgcagccccctgctttgttggggcgggagcatgccaccctctgagccgcggtgggctcagtgaagtgggtgttgtcatccttcccactgcacgggtctagagtctggggtgcggtgagctcgcggcttgctgagggccaacagttagtgaggggtggagctgcgacttgcaggaaggtccagccccgcctcccaggccctttctagccccttctgttgccgtgctgcttcccacccttgcctggaatcctcctgtgggggacgcatgtggtggcccctgtggctgactttccggctgagccagggcaccagtgtcctcactctagagatgagctcttctgaggggtgaggtccagccacacctgtggtggggtggtgtagcccgagcctgttaccccgtctataaaacagagggattcacttcttttacaggcacttcctggccctgcggggtgccctgtctctggctggagctgatgagccgtggtgtgatgcttgcctgggggtcggtgttgcctagaagtgaggaatgacctccttcacccctggctccttccggcagcctggccacttccagcatgtggagggggcccgggccctggcagagacggtgtggaaccgccggccgcatgcggggctggagttcggggctgccaatgggctaaggccctgtgttcaacacatgtcgcggaactgtcgtgaaaaaaagactcaggaccacaggtccggggtgctcccaggggtctttcccaccgaggctgggagtgagcagcagcctaggcccagccacacctcccaggctcgggagctgccaggccaggcaaccgtgaagggtagcagggtggggggtggaggggaggacagggagagcaggcctctcgggcctcacccacccttgcaccttccagctctagggcccacacagtaccaagccctgggctcaatgtaccacttatgaaaactcaattcaaattggcttagtgcaacaaggtatttggtggtctcacatgacttgaacatccaggcctggctgtcacgggaaccgcatctcttcccattgcagctacttggcaggtggcgggatgtcccccagccaccgacgtccccctgcctgctccgcaaccccagggcctgcagaaaaggcccacgagactcagactggcagagacttaggcggaccaggaacaggggcgcagtctccgtcccacccaaaccctaaccagagagaacacggcacgttgtgccagacggaggacggatgccagcgagggtccatgtcctcactgccgacaaggctgggaactgggccaagtgaagcagaggcctccacgtcagatgtgagcgccaccggcccaggtgactgcagttcttccctccttccgttcggcttgagccctccagaggatcggaaaggctgaggcctgacctggtgccgctgtcctgggtgggtctgtcctgctggtcggttcctgcccctctcggggaggttggctggcagctggcaggtggaaagcctcctgtgttcacctcagggcagaggtggggacacagggcgggacgggcgagtgtggtgcccctctggggtgggtgctcttgggtccgcctcccgtgccagagtgcgtgtcaacagttccagctgcccctcagaactgtcctggtttaggaggtgaacacacggggcagcctacattctacgtggtttttttaacattataaaagcagcatgtgttattacaggaaatttaacagaagtatataaaaagaaaccagaagtca

SEQ ID NO: 2 - DNA, 1193 bp, Homo sapiens
  CDS (351)...(770)
  CDS (811)...(1161)
ttcggctgacagagcatgagggggaggaatcactgatgacaggcactggcctgcccagctgggggcctttgtttattcatttggtgggcacttcctgggtgcctgctctgggtcaggcctgtgggggggaccactgagggcaggaaacctggcctgtccctccaggaagcgaagtcaacactggcacctgcagatgaagtggcagagcagcccccagctttgatggcatggggtggttggggggcacattctgcatgctcagaagagagagcaactcgccctgtggaaggagcatacagtgggagatggggacaggcccagtgacgagcaccatccggaagtgaaggctgatgggtacgtggacaacctcgcagaggcagtggacctgctgctgcagcacgccgacaagtgatggcctcctgggagagccccgcctcctccacccctgcctctcctccacccctgcctcccctccacccctgcctctcctccacccgcccaggagagccccacctcctccacccctgcctctcctccacccctgcctcccctccacctgccccagtgcccagaccaaccaaggccctgacagccctgccttctgccctctgccctgcatgggcaggcatttgttccctacctgggtggcctgctcccctgcctgggccctgacttcagctccctgtagtgaagtccaggagggtgggacaggcctgtcaggcctctgggaatctcccaaatcccagaactcaccactcaccatgggcctttaaatgcagtaaactccacctaaccagattcaggggcactatgcccactgcctcctcttcagactctttgcatttcagtgaagagcctggaagaaacccaggggcctcctatgcacagatcttgcagcccagaaccaagtcagcctccctgcgactgcccaggcacactgcccaccaccccacccccgaaacaatgccagcccgctgctttttctatcctcccagtcacctttgcagacaaagaccaggggcagctcccgagggcactgtgaaggctcccatgccacacagtgagaactgtagcctctgcgtccaaggcacacagggtactttctggacccactgctggacagacttgaaggtgtcatgcccggtgtgtgcaggaggaaactaacagttcagtaaactctgccttgaccagca

SEQ ID NO: 3 - protein, 140 aa, Homo sapiens
MGTWTTSQRQWTCCCSTPTSDGLLGEPRLLHPCLSSTPASPPPLPLLHPPRRAPPPPPLPLLHPCLPSTCPSAQTNQGPDSPAFCPLPCMGRHLFPTWVACSPAWALTSAPCSEVQEGGTGLSGLWESPKSQNSPLTMGL

SEQ ID NO: 4 - protein, 117 aa, Homo sapiens
MPTASSSDSLHFSEEPGRNPGASYAQILQPRTKSASLRLPRHTAHHPTPETMPARCFFYPPSHLCRQRPGAAPEGTVKAPMPHSENCSLCVQGTQGTFWTHCWTDLKVSCPVCAGGN

SEQ ID NO: 5 - DNA, 1092 bp, Homo sapiens
tgagaacccagggctcgctgggatgctgtcctccctccctccctccctctgccaagctggtgggaaggcccttgccaaagcgcacaagaccccttcacacagcaggggcacaagctcttcgaggcagagtcgcctgcaggtggggtggaaggggtgcgccccagacccaagaatcgcccgcctctccaagaccatccctggctgctggcccagtgacgagcaccatccggaagtgaaggctgatgggtacgtggacaacctcgcagaggcagtggacctgctgctgcagcacgccgacaagtgatggcctcctgggagagccccgcctcctccacccctgcctctcctccacccctgcctcccctccacccctgcctctcctccacccgcccaggagagccccacctcctccacccctgcctctcctccacccctgcctcccctccacctgccccagtgcccagaccaaccaaggccctgacagccctgccttctgccctctgccctgcatgggcaggcatttgttccctacctgggtggcctgctcccctgcctgggccctgacttcagctccctgtagtgaagtccaggagggtgggacaggcctgtcaggcctctgggaatctcccaaatcccagaactcaccactcaccatgggcctttaaatgcagtaaactccacctaaccagattcaggggcactaatgcccactgcctcctcttcagactctttgcatttcagtgaagagcctggaagaaacccaggggcctcctatgcacagatcttgcagcccagaaccaagtcagcctccctgcgactgcccaggcacactgcccaccaccccacccccgaaacaatgccagcccgctgctttttctatcctcccagtcacctttgcagacaaagaccaggggcagctcccgagggcactgtgaaggctcccatgccacacagtgagaactgtagcctctgcgtccaaggcacacagggtactttctggacccactgctggacagacttgaaggtgtcatgcccggtgtgtgcaggaggaaactaacagttcagtaaactctgccttgaccagcagccttt

SEQ ID NO: 6 - DNA, 1022 bp, Homo sapiens
ctccgagtctctaccccagctgtccccacagtgcacatggactaggctcctcccacggggcactaggccaggccaggggtgtggggtgagcccctggggagcccagagcagggtacactcatgtccccaccatccaaggcccagtgacgagcaccatccggaagtgaaggctgatgggtacgtggacaacctcgcagaggcagtggacctgctgctgcagcacgccgacaagtgatggcctcctgggagagccccgcctcctccacccctgcctctcctccacccctgcctcccctccacccctgcctctcctccacccgcccaggagagccccacctcctccacccctgcctctcctccacccctgcctcccctccacctgccccagtgcccagaccaaccaaggccctgacagccctgccttctgccctctgccctgcatgggcaggcatttgttccctacctgggtggcctgctcccctgcctgggccctgacttcagctccctgtagtgaagtccaggagggtgggacaggcctgtcaggcctctgggaatctcccaaatcccagaactcaccactcaccatgggcctttaaatgcagtaaactccacctaaccagattcaggggcactatgcccactgcctcctcttcagactctttgcatttcagtgaagagcctggaagaaacccaggggcctcctatgcacagatcttgcagcccagaaccaagtcagcctccctgcgactgcccaggcacactgcccaccaccccacccccgaaacaatgccagcccgctgctttttctatcctcccagtcacctttgcagacaaagaccaggggcagctcccgagggcactgtgaaggctcccatgccacacagtgagaactgtagcctctgcgtccaaggcacacagggtactttctggacccactgctggacagacttgaaggtgtcatgcccggtgtgtgcaggaggaaactaacagttcagtaaactctgccttgaccagcagccttt

SEQ ID NO: 7 - DNA, 1186 bp, Homo sapiens
tgcattcggctgacagagcatgagggggaggaatcactgatgacaggcactggcctgcccagctgggggcctttgtttattcatttggtgggcacttcctgggtgcctgctctgggtcaggcctgtgggggggaccactgagggcaggaaacctggcctgtccctccaggaagcgaagtcaacactggcacctgcagatgaagtggcagagcagcccccagctttgatggcatggggtggttggggggcacattctgcatgctcagaagagagagcaactcgccctgtggaaggagcatacagtgggagatggggacaggcccagtgacgagcaccatccggaagtgaaggctgatgggtacgtggacaacctcgcagaggcagtggacctgctgctgcagcacgccgacaagtgatggcctcctgggagagccccgcctcctccacccctgcctcccctccacccctgcctctcctccacccgcccaggagagccccacctcctccacccctgcctctcctccacccctgcctcccctccacctgccccagtgcccagaccaaccaaggccctgacagccctgccttctgccctctgccctgcatgggcaggcatttgttccctacctgggtggcctgctcccctgcctgggccctgacttcagctccctgtagtgaagtccaggagggtgggacaggcctgtcaggcctctgggaatctcccaaatcccagaactcaccactcaccatgggcctttaaatgcagtaaactccacctaaccagattcaggggcactatgcccactgcctcctcttcagactctttgcatttcagtgaagagcctggaagaaacccaggggcctcctatgcacagatcttgcagcccagaaccaagtcagcctccctgcgactgcccaggcacactgcccaccaccccacccccgaaacaatgccagcccgctgctttttctatcctcccagtcacctttgcagacaaagaccaggggcagctcccgagggcactgtgaaggctcccatgccacacagtgagaactgtagcctctgcgtccaaggcacacagggtactttctggacccactgctggacagacttgaaggtgtcatgcccggtgtgtgcaggaggaaactaacagttcagtaaactctgccttgaccagcagccttt

SEQ ID NO: 8 - DNA, 1005 bp, Homo sapiens
ctccgagtctctaccccagctgtccccacagtgcacatggactaggctcctcccacggggcactaggccaggccaggggtgtggggtgagcccctggggagcccagagcagggtacactcatgtccccaccatccaaggcccagtgacgagcaccatccggaagtgaaggctgatgggtacgtggacaacctcgcagaggcagtggacctgctgctgcagcacgccgacaagtgatggcctcctgggagagccccgcctcctccacccctgcctcccctccacccctgcctctcctccacccgcccaggagagccccacctcctccacccctgcctctcctccacccctgcctcccctccacctgccccagtgcccagaccaaccaaggccctgacagccctgccttctgccctctgccctgcatgggcaggcatttgttccctacctgggtggcctgctcccctgcctgggccctgacttcagctccctgtagtgaagtccaggagggtgggacaggcctgtcaggcctctgggaatctcccaaatcccagaactcaccactcaccatgggcctttaaatgcagtaaactccacctaaccagattcaggggcactatgcccactgcctcctcttcagactctttgcatttcagtgaagagcctggaagaaacccaggggcctcctatgcacagatcttgcagcccagaaccaagtcagcctccctgcgactgcccaggcacactgcccaccaccccacccccgaaacaatgccagcccgctgctttttctatcctcccagtcacctttgcagacaaagaccaggggcagctcccgagggcactgtgaaggctcccatgccacacagtgagaactgtagcctctgcgtccaaggcacacagggtactttctggacccactgctggacagacttgaaggtgtcatgcccggtgtgtgcaggaggaaactaacagttcagtaaactctgccttgaccagcagccttt

SEQ ID NO: 9 - DNA, 1765 bp, Homo sapiens
  CDS (166)...(975)
aagaactttaaaaatcacctaggtgtgggccgggcacggtggctaacgcctgtaatcccagcactttgagatgctgaggcaggtggatcacgaggtcaggagatcgagaccatcctggataacacggagaaaccccggcggagctgaggagcagggccgggcgccatggcaccgtggggcaagcggctggctggcgtgcgcggggtgctgcttgacatctcgggcgtgctgtacgacagcggcgcgggcggcggcacggccatcgccggctcggtggaggcggtggccagactgaagcgttcccggctgaaggtgaggttctgcaccaacgagtcgcagaagtcccgggcagagctggtggggcagcttcagaggctgggatttgacatctctgagcaggaggtgaccgccccggcaccagctgcctgccagatcctgaaggagcgaggcctgcgaccatacctgctcatccatgacggagtccgctcagaatttgatcagatcgacacatccaacccaaactgtgtggtaattgcagacgcaggagaaagcttttcttatcaaaacatgaataacgccttccaggtgctcatggagctggaaaaacctgtgctcatatcactgggaaaagggcgttactacaaggagacctctggcctgatgctggacgttggtccctacatgaaggcgcttgagtatgcctgtggcatcaaagccgaggtggtggggaagccttctcctgagtttttcaagtctgccctgcaagcgataggagtggaagcccaccaggccgtcatgattggggacgatatcgtgggcgacgtcggcggtgcccagcggtgtggaatgagagcgctgcaggtgcgcaccgggaagttcaggcccagtgacgagcaccatccggaagtgaaggctgatgggtacgtggacaacctcgcagaggcagtggacctgctgctgcagcacgccgacaagtgatggcctcctgggagagccccgcctcctccacccctgcctctcctccacccctgcctcccctccacccctgcctctcctccacccgcccaggagagccccacctcctccacccctgcctctcctccacccctgcctcccctccacctgccccagtgcccagaccaaccaaggccctgacagccctgccttctgccctctgccctgcatgggcaggcatttgttccctacctgggtggcctgctcccctgcctgggccctgacttcagctccctgtagtgaagtccaggagggtgggacaggcctgtcaggcctctgggaatctcccaaatcccagaactcaccactcaccatgggcctttaaatgcagtaaactccacctaaccagattcaggggcactatgcccactgcctcctcttcagactctttgcatttcagtgaagagcctggaagaaacccaggggcctcctatgcacagatcttgcagcccagaaccaagtcagcctccctgcgactgcccaggcacactgcccaccaccccacccccgaaacaatgccagcccgctgctttttctatcctcccagtcacctttgcagacaaagaccaggggcagctcccgagggcactgtgaaggctcccatgccacacagtgagaactgtagcctctgcgtccaaggcacacagggtactttctggacccactgctggacagacttgaaggtgtcatgcccggtgtgtgcaggaggaaactaacagttcagtaaactctgccttgaccagcagccttt

SEQ ID NO: 10 - protein, 270 aa, Homo sapiens
MAPWGKRLAGVRGVLLDISGVLYDSGAGGGTAIAGSVEAVARLKRSRLKVRFCTNESQKSRAELVGQLQRLGFDISEQEVTAPAPAACQILKERGLRPYLLIHDGVRSEFDQIDTSNPNCVVIADAGESFSYQNMNNAFQVLMELEKPVLISLGKGRYYKETSGLMLDVGPYMKALEYACGIKAEVVGKPSPEFFKSALQAIGVEAHQAVMIGDDIVGDVGGAQRCGMRALQVRTGKFRPSDEHHPEVKADGYVDNLAEAVDLLLQHADK

SEQ ID NO: 11 - DNA, 1669 bp, Homo sapiens
aagaactttaaaaatcacctaggtgtgggccgggcacggtggctaacgcctgtaatcccagcactttgagatgctgaggcaggtggatcacgaggtcaggagatcgagaccatcctggataacacggagaaaccccggcggagctgaggagcagggccgggcgccatggcaccgtggggcaagcggctggctggcgtgcgcggggtgctgcttgacatctcgggcgtgctgtacgacagcggcgcgggcggcggcacggccatcgccggctcggtggaggcggtggccagactgaagcgttcccggctgaaggtgaggttctgcaccaacgagtcgcagaagtcccgggcagagctggtggggcagcttcagaggctgggatttgacatctctgagcaggaggtgaccgccccggcaccagctgcctgccagatcctgaaggagcgaggcctgcgaccatacctgctcatccatgacggagtccgctcagaatttgatcagatcgacacatccaacccaaactgtgtggtaattgcagacgcaggagaaagcttttcttatcaaaacatgaataacgccttccaggtgctcatggagctggaaaaacctgtgctcatatcactgggaaaagggcgttactacaaggagacctctggcctgatgctggacgttggtccctacatgaaggcgcttgagtatgcctgtggcatcaaagccgaggtggtggggaagccttctcctgagtttttcaagtctgccctgcaagcgataggagtggaagcccaccaggccgtcatgattggggacgatatcgtgggcgacgtcggcggtgcccagcggtgtggaatgagagcgctgcaggtgcgcaccgggaagttcaggcccagtgacgagcaccatccggaagtgaaggctgatgggtacgtggacaacctcgcagaggcagtggacctgctgctgcagcacgccgacaagtgatggcctcctgggagagccccgcctcctccacccctgcctcccctccacctgccccagtgcccagaccaaccaaggccctgacagccctgccttctgccctctgccctgcatgggcaggcatttgttccctacctgggtggcctgctcccctgcctgggccctgacttcagctccctgtagtgaagtccaggagggtgggacaggcctgtcaggcctctgggaatctcccaaatcccagaactcaccactcaccatgggcctttaaatgcagtaaactccacctaaccagattcaggggcactatgcccactgcctcctcttcagactctttgcatttcagtgaagagcctggaagaaacccaggggcctcctatgcacagatcttgcagcccagaaccaagtcagcctccctgcgactgcccaggcacactgcccaccaccccacccccgaaacaatgccagcccgctgctttttctatcctcccagtcacctttgcagacaaagaccaggggcagctcccgagggcactgtgaaggctcccatgccacacagtgagaactgtagcctctgcgtccaaggcacacagggtactttctggacccactgctggacagacttgaaggtgtcatgcccggtgtgtgcaggaggaaactaacagttcagtaaactctgccttgaccagcagccttt

SEQ ID NO: 12 - DNA, 1673 bp, Homo sapiens
  CDS (166)...(798)
aagaactttaaaaatcacctaggtgtgggccgggcacggtggctaacgcctgtaatcccagcactttgagatgctgaggcaggtggatcacgaggtcaggagatcgagaccatcctggataacacggagaaaccccggcggagctgaggagcagggccgggcgccatggcaccgtggggcaagcggctggctggcgtgcgcggggtgctgcttgacatctcgggcgtgctgtacgacagcggcgcgggcggcggcacggccatcgccggctcggtggaggcggtggccagactgaagcgttcccggctgaaggtgaggttctgcaccaacgagtcgcagaagtcccgggcagagctggtggggcagcttcagaggctgggatttgacatctctgagcaggaggtgaccgccccggcaccagctgcctgccagatcctgaaggagcgaggcctgcgaccatacctgctcatccatgacggagtccgctcagaatttgatcagatcgacacatccaacccaaactgtgtggtaattgcagacgcaggagaaagcttttcttatcaaaacatgaataacgccttccaggtgctcatggagctggaaaaacctgtgctcatatcactgggaaaagggcgttactacaaggagacctctggcctgatgctggacgttggtccctacatgaaggcgcttgagtatgcctgtggcatcaaagccgaggtggtggggaagccttctcctgagtttttcaagtctgccctgcaagcgataggagtggaagcccaccaggcccagtgacgagcaccatccggaagtgaaggctgatgggtacgtggacaacctcgcagaggcagtggacctgctgctgcagcacgccgacaagtgatggcctcctgggagagccccgcctcctccacccctgcctctcctccacccctgcctcccctccacccctgcctctcctccacccgcccaggagagccccacctcctccacccctgcctctcctccacccctgcctcccctccacctgccccagtgcccagaccaaccaaggccctgacagccctgccttctgccctctgccctgcatgggcaggcatttgttccctacctgggtggcctgctcccctgcctgggccctgacttcagctccctgtagtgaagtccaggagggtgggacaggcctgtcaggcctctgggaatctcccaaatcccagaactcaccactcaccatgggcctttaaatgcagtaaactccacctaaccagattcaggggcactatgcccactgcctcctcttcagactctttgcatttcagtgaagagcctggaagaaacccaggggcctcctatgcacagatcttgcagcccagaaccaagtcagcctccctgcgactgcccaggcacactgcccaccaccccacccccgaaacaatgccagcccgctgctttttctatcctcccagtcacctttgcagacaaagaccaggggcagctcccgagggcactgtgaaggctcccatgccacacagtgagaactgtagcctctgcgtccaaggcacacagggtactttctggacccactgctggacagacttgaaggtgtcatgcccggtgtgtgcaggaggaaactaacagttcagtaaactctgccttgaccagcagccttt

SEQ ID NO: 13 - protein, 210 aa, Homo sapiens [sequence omitted]

SEQ ID NO: 14 - DNA, 1516 bp, Homo sapiens
  CDS (166)...(729)
aagaactttaaaaatcacctaggtgtgggccgggcacggtggctaacgcctgtaatcccagcactttgagatgctgaggcaggtggatcacgaggtcaggagatcgagaccatcctggataacacggagaaaccccggcggagctgaggagcagggccgggcgccatggcaccgtggggcaagcggctggctggcgtgcgcggggtgctgcttgacatctcgggcgtgctgtacgacagcggcgcgggcggcggcacggccatcgccggctcggtggaggcggtggccagactgaagcgttcccggctgaaggtgaggttctgcaccaacgagtcgcagaagtcccgggcagagctggtggggcagcttcagaggctgggatttgacatctctgagcaggaggtgaccgccccggcaccagctgcctgccagatcctgaaggagcgaggcctgcgaccatacctgctcatccatgacggagtccgctcagaatttgatcagatcgacacatccaacccaaactgtgtggtaattgcagacgcaggagaaagcttttcttatcaaaacatgaataacgccttccaggtgctcatggagctggaaaaacctgtgctcatatcactgggaaaagggcccagtgacgagcaccatccggaagtgaaggctgatgggtacgtggacaacctcgcagaggcagtggacctgctgctgcagcacgccgacaagtgatggcctcctgggagagccccgcctcctccacccctgcctctcctccacccctgcctcccctccacccctgcctctcctccacccgcccaggagagccccacctcctccacccctgcctctcctccacccctgcctcccctccacctgccccagtgcccagaccaaccaaggccctgacagccctgccttctgccctctgccctgcatgggcaggcatttgttccctacctgggtggcctgctcccctgcctgggccctgacttcagctccctgtagtgaagtccaggagggtgggacaggcctgtcaggcctctgggaatctcccaaatcccagaactcaccactcaccatgggcctttaaatgcagtaaactccacctaaccagattcaggggcactatgcccactgcctcctcttcagactctttgcatttcagtgaagagcctggaagaaacccaggggcctcctatgcacagatcttgcagcccagaaccaagtcagcctccctgcgactgcccaggcacactgcccaccaccccacccccgaaacaatgccagcccgctgctttttctatcctcccagtcacctttgcagacaaagaccaggggcagctcccgagggcactgtgaaggctcccatgccacacagtgagaactgtagcctctgcgtccaaggcacacagggtactttctggacccactgctggacagacttgaaggtgtcatgcccggtgtgtgcaggaggaaactaacagttcagtaaactctgccttgaccagcagccttt

SEQ ID NO: 15 - protein, 187 aa, Homo sapiens
MAPWGKRLAGVRGVLLDISGVLYDSGAGGGTAIAGSVEAVARLKRSRLKVRFCTNESQKSRAELVGQLQRLGFDISEQEVTAPAPAACQILKERGLRPYLLIHDGVRSEFDQIDTSNPNCVVIADAGESFSYQNMNNAFQVLMELEKPVLISLGKGPSDEHHPEVKADGYVDNLAEAVDLLLQHADK

SEQ ID NO: 16 - DNA, 1287 bp, Homo sapiens
  CDS (166)...(948)
aagaactttaaaaatcacctaggtgtgggccgggcacggtggctaacgcctgtaatcccagcactttgagatgctgaggcaggtggatcacgaggtcaggagatcgagaccatcctggataacacggagaaaccccggcggagctgaggagcagggccgggcgccatggcaccgtggggcaagcggctggctggcgtgcgcggggtgctgcttgacatctcgggcgtgctgtacgacagcggcgcgggcggcggcacggccatcgccggctcggtggaggcggtggccagactgaagcgttcccggctgaaggtgaggttctgcaccaacgagtcgcagaagtcccgggcagagctggtggggcagcttcagaggctgggatttgacatctctgagcaggaggtgaccgccccggcaccagctgcctgccagatcctgaaggagcgaggcctgcgaccatacctgctcatccatgacggagtccgctcagaatttgatcagatcgacacatccaacccaaactgtgtggtaattgcagacgcaggagaaagcttttcttatcaaaacatgaataacgccttccaggtgctcatggagctggaaaaacctgtgctcatatcactgggaaaagggcgttactacaaggagacctctggcctgatgctggacgttggtccctacatgaaggcgcttgagtatgcctgtggcatcaaagccgaggtggtggggaagccttctcctgagtttttcaagtctgccctgcaagcgataggagtggaagcccaccaggccgtcatgattggggacgatatcgtgggcgacgtcggcggtgcccagcggtgtggaatgagagcgctgcaggtgcgcaccgggaagttcaggcccagtgacgagcaccatccggaagtgaaggctgatggactctttgcatttcagtgaagagcctggaagaaacccaggggcctcctatgcacagatcttgcagcccagaaccaagtcagcctccctgcgactgcccaggcacactgcccaccaccccacccccgaaacaatgccagcccgctgctttttctatcctcccagtcacctttgcagacaaagaccaggggcagctcccgagggcactgtgaaggctcccatgccacacagtgagaactgtagcctctgcgtccaaggcacacagggtactttctggacccactgctggacagacttgaaggtgtcatgcccggtgtgtgcaggaggaaactaacagttcagtaaactctgccttgaccagcagccttt

SEQ ID NO: 17 - protein, 257 aa, Homo sapiens [sequence omitted]

SEQ ID NO: 18 - DNA, 2264 bp, Homo sapiens
  CDS (166)...(1008)
aagaactttaaaaatcacctaggtgtgggccgggcacggtggctaacgcctgtaatcccagcactttgagatgctgaggcaggtggatcacgaggtcaggagatcgagaccatcctggataacacggagaaaccccggcggagctgaggagcagggccgggcgccatggcaccgtggggcaagcggctggctggcgtgcgcggggtgctgcttgacatctcgggcgtgctgtacgacagcggcgcgggcggcggcacggccatcgccggctcggtggaggcggtggccagactgaagcgttcccggctgaaggtgaggttctgcaccaacgagtcgcagaagtcccgggcagagctggtggggcagcttcagaggctgggatttgacatctctgagcaggaggtgaccgccccggcaccagctgcctgccagatcctgaaggagcgaggcctgcgaccatacctgctcatccatgacggagtccgctcagaatttgatcagatcgacacatccaacccaaactgtgtggtaattgcagacgcaggagaaagcttttcttatcaaaacatgaataacgccttccaggtgctcatggagctggaaaaacctgtgctcatatcactgggaaaagggcgttactacaaggagacctctggcctgatgctggacgttggtccctacatgaaggcgcttgagtatgcctgtggcatcaaagccgaggtggtggggaagccttctcctgagtttttcaagtctgccctgcaagcgataggagtggaagcccaccaggccgtcatgattggggacgatatcgtgggcgacgtcggcggtgcccagcggtgtggaatgagagcgctgcaggtgcgcaccgggaagttcaggcccagtgacgagcaccatccggaagtgaaggctgatgggcacttcctggccctgcggggtgccctgtctctggctggagctgatgagccgtggtgtgatgcttgcctgggggtcggtgttgcctagaagtgaggaatgacctccttcacccctggctccttccggcagcctggccacttccagcatgtggagggggcccgggccctggcagagacggtgtggaaccgccggccgcatgcggggctggagttcggggctgccaatgggctaaggccctgtgttcaacacatgtcgcggaactgtcgtgaaaaaaagactcaggaccacaggtccggggtgctcccaggggtctttcccaccgaggctgggagtgagcagcagcctaggcccagccacacctcccaggctcgggagctgccaggccaggcaaccgtgaagggtagcagggtggggggtggaggggaggacagggagagcaggcctctcgggcctcacccacccttgcaccttccagctctagggcccacacagtaccaagccctgggctcaatgtaccacttatgaaaactcaattcaaattggcttagtgcaacaaggtatttggtggtctcacatgacttgaacatccaggcctggctgtcacgggaaccgcatctcttcccattgcagctacttggcaggtggcgggatgtcccccagccaccgacgtccccctgcctgctccgcaaccccagggcctgcagaaaaggcccacgagactcagactggcagagacttaggcggaccaggaacaggggcgcagtctccgtcccacccaaaccctaaccagagagaacacggcacgttgtgccagacggaggacggatgccagcgagggtccatgtcctcactgccgacaaggctgggaactgggccaagtgaagcagaggcctccacgtcagatgtgagcgccaccggcccaggtgactgcagttcttccctccttccgttcggcttgagccctccagaggatcggaaaggctgaggcctgacctggtgccgctgtcctgggtgggtctgtcctgctggtcggttcctgcccctctcggggaggttggctggcagctggcaggtggaaagcctcctgtgttcacctcagggcagaggtggggacacagggcgggacgggcgagtgtggtgcccctctggggtgggtgctcttgggtccgcctcccgtgccagagtgcgtgtcaacagttccagctgcccctcagaactgtcctggtttaggaggtgaacacacggggcagcctacattctacgtggtttttttaacattataaaagcagcatgtgttattacaggaaatttaacagaagtatataaaaagaaaccagaagtca

SEQ ID NO: 19 - protein, 280 aa, Homo sapiens
MAPWGKRLAGVRGVLLDISGVLYDSGAGGGTAIAGSVEAVARLKRSRLKVRFCTNESQKSRAELVGQLQRLGFDISEQEVTAPAPAACQILKERGLRPYLLIHDGVRSEFDQIDTSNPNCVVIADAGESFSYQNMNNAFQVLMELEKPVLISLGKGRYYKETSGLMLDVGPYMKALEYACGIKAEVVGKPSPEFFKSALQAIGVEAHQAVMIGDDIVGDVGGAQRCGMRALQVRTGKFRPSDEHHPEVKADGHFLALRGALSLAGADEPWCDACLGVGVA

SEQ ID NO: 20 - DNA, 992 bp, Homo sapiens
  CDS (166)...(960)
aagaactttaaaaatcacctaggtgtgggccgggcacggtggctaacgcctgtaatcccagcactttgagatgctgaggcaggtggatcacgaggtcaggagatcgagaccatcctggataacacggagaaaccccggcggagctgaggagcagggccgggcgccatggcaccgtggggcaagcggctggctggcgtgcgcggggtgctgcttgacatctcgggcgtgctgtacgacagcggcgcgggcggcggcacggccatcgccggctcggtggaggcggtggccagactgaagcgttcccggctgaaggtgaggttctgcaccaacgagtcgcagaagtcccgggcagagctggtggggcagcttcagaggctgggatttgacatctctgagcaggaggtgaccgccccggcaccagctgcctgccagatcctgaaggagcgaggcctgcgaccatacctgctcatccatgacggagtccgctcagaatttgatcagatcgacacatccaacccaaactgtgtggtaattgcagacgcaggagaaagcttttcttatcaaaacatgaataacgccttccaggtgctcatggagctggaaaaacctgtgctcatatcactgggaaaagggcgttactacaaggagacctctggcctgatgctggacgttggtccctacatgaaggcgcttgagtatgcctgtggcatcaaagccgaggtggtggggaagccttctcctgagtttttcaagtctgccctgcaagcgataggagtggaagcccaccaggccgtcatgattggggacgatatcgtgggcgacgtcggcggtgcccagcggtgtggaatgagagcgctgcaggtgcgcaccgggaagttcaggtcagtgccagctggagtcatttattcaccttccttccaggggatgaccacattctcattctgttttgttcttcaaaataaaggggatattctttccaaatcaaagagcag

SEQ ID NO: 21 - protein, 265 aa, Homo sapiens
MAPWGKRLAGVRGVLLDISGVLYDSGAGGGTAIAGSVEAVARLKRSRLKVRFCTNESQKSRAELVGQLQRLGFDISEQEVTAPAPAACQILKERGLRPYLLIHDGVRSEFDQIDTSNPNCVVIADAGESFSYQNMNNAFQVLMELEKPVLISLGKGRYYKETSGLMLDVGPYMKALEYACGIKAEVVGKPSPEFFKSALQAIGVEAHQAVMIGDDIVGDVGGAQRCGMRALQVRTGKFRSVPAGVIYSPSFQGMTTFSFCFVLQN

SEQ ID NO: 22 - DNA, 952 bp, Homo sapiens
  CDS (166)...(939)
aagaactttaaaaatcacctaggtgtgggccgggcacggtggctaacgcctgtaatcccagcactttgagatgctgaggcaggtggatcacgaggtcaggagatcgagaccatcctggataacacggagaaaccccggcggagctgaggagcagggccgggcgccatggcaccgtggggcaagcggctggctggcgtgcgcggggtgctgcttgacatctcgggcgtgctgtacgacagcggcgcgggcggcggcacggccatcgccggctcggtggaggcggtggccagactgaagcgttcccggctgaaggtgaggttctgcaccaacgagtcgcagaagtcccgggcagagctggtggggcagcttcagaggctgggatttgacatctctgagcaggaggtgaccgccccggcaccagctgcctgccagatcctgaaggagcgaggcctgcgaccatacctgctcatccatgacggagtccgctcagaatttgatcagatcgacacatccaacccaaactgtgtggtaattgcagacgcaggagaaagcttttcttatcaaaacatgaataacgccttccaggtgctcatggagctggaaaaacctgtgctcatatcactgggaaaagggcgttactacaaggagacctctggcctgatgctggacgttggtccctacatgaaggcgcttgagtatgcctgtggcatcaaagccgaggtggtggggaagccttctcctgagtttttcaagtctgccctgcaagcgataggagtggaagcccaccaggccgtcatgattggggacgatatcgtgggcgacgtcggcggtgcccagcggtgtggaatgagagcgctgcagagccgctcaacacgtgttgcaaggcacgtgctctggacctggtgctcacacgtgctcttctcccaagacactccctgaagcgcgttcccca

SEQ ID NO: 23 - protein, 257 aa, Homo sapiens
MAPWGKRLAGVRGVLLDISGVLYDSGAGGGTAIAGSVEAVARLKRSRLKVRFCTNESQKSRAELVGQLQRLGFDISEQEVTAPAPAACQILKERGLRPYLLIHDGVRSEFDQIDTSNPNCVVIADAGESFSYQNMNNAFQVLMELEKPVLISLGKGRYYKETSGLMLDVGPYMKALEYACGIKAEVVGKPSPEFFKSALQAIGVEAHQAVMIGDDIVGDVGGAQRCGMRALQSRSTRVARHVLWTWCSHVLFSQDTP

SEQ ID NO: 24 - DNA, 979 bp, Homo sapiens
  CDS (166)...(804)
aagaactttaaaaatcacctaggtgtgggccgggcacggtggctaacgcctgtaatcccagcactttgagatgctgaggcaggtggatcacgaggtcaggagatcgagaccatcctggataacacggagaaaccccggcggagctgaggagcagggccgggcgccatggcaccgtggggcaagcggctggctggcgtgcgcggggtgctgcttgacatctcgggcgtgctgtacgacagcggcgcgggcggcggcacggccatcgccggctcggtggaggcggtggccagactgaagcgttcccggctgaaggtgaggttctgcaccaacgagtcgcagaagtcccgggcagagctggtggggcagcttcagaggctgggatttgacatctctgagcaggaggtgaccgccccggcaccagctgcctgccagatcctgaaggagcgaggcctgcgaccatacctgctcatccatgacggagtccgctcagaatttgatcagatcgacacatccaacccaaactgtgtggtaattgcagacgcaggagaaagcttttcttatcaaaacatgaataacgccttccaggtgctcatggagctggaaaaacctgtgctcatatcactgggaaaagggcgttactacaaggagacctctggcctgatgctggacgttggtccctacatgaaggcgcttgagtatgcctgtggcatcaaagccgaggtggtggggaagccttctcctgagtttttcaagtctgccctgcaagcgataggagtggaagcccaccagttactttcagtatgaaagcaagaagcagaaatgcctgcggcttttcctgagtttttgctgcttctctgaaaggataagaattgacaagtcctatcagtgtgttaatatatctcactggcaagacagtgtaacagcaagattacaacaatatggaggaaataataaagtcactcattttgcgacctttata

SEQ ID NO: 25 - protein, 212 aa, Homo sapiens [sequence omitted]

SEQ ID NO: 26 - DNA, 1590 bp, Homo sapiens
  CDS (344)...(772)
tgagaacccagggctcgctgggatgctgtcctccctccctccctccctctgccaagctggtgggaaggcccttgccaaagcgcacaagaccccttcacacagcaggggcacaagctcttcgaggcagagtcgcctgcaggtggggtggaaggggtgcgccccagacccaagaatcgcccgcctctccaagaccatccctggctgctggcccagtgacgagcaccatccggaagtgaaggctgatgggcacttcctggccctgcggggtgccctgtctctggctggagctgatgagccgtggtgtgatgcttgcctgggggtcggtgttgcctagaagtgaggaatgacctccttcacccctggctccttccggcagcctggccacttccagcatgtggagggggcccgggccctggcagagacggtgtggaaccgccggccgcatgcggggctggagttcggggctgccaatgggctaaggccctgtgttcaacacatgtcgcggaactgtcgtgaaaaaaagactcaggaccacaggtccggggtgctcccaggggtctttcccaccgaggctgggagtgagcagcagcctaggcccagccacacctcccaggctcgggagctgccaggccaggcaaccgtgaagggtagcagggtggggggtggaggggaggacagggagagcaggcctctcgggcctcacccacccttgcaccttccagctctagggcccacacagtaccaagccctgggctcaatgtaccacttatgaaaactcaattcaaattggcttagtgcaacaaggtatttggtggtctcacatgacttgaacatccaggcctggctgtcacgggaaccgcatctcttcccattgcagctacttggcaggtggcgggatgtcccccagccaccgacgtccccctgcctgctccgcaaccccagggcctgcagaaaaggcccacgagactcagactggcagagacttaggcggaccaggaacaggggcgcagtctccgtcccacccaaaccctaaccagagagaacacggcacgttgtgccagacggaggacggatgccagcgagggtccatgtcctcactgccgacaaggctgggaactgggccaagtgaagcagaggcctccacgtcagatgtgagcgccaccggcccaggtgactgcagttcttccctccttccgttcggcttgagccctccagaggatcggaaaggctgaggcctgacctggtgccgctgtcctgggtgggtctgtcctgctggtcggttcctgcccctctcggggaggttggctggcagctggcaggtggaaagcctcctgtgttcacctcagggcagaggtggggacacagggcgggacgggcgagtgtggtgcccctctggggtgggtgctcttgggtccgcctcccgtgccagagtgcgtgtcaacagttccagctgcccctcagaactgtcctggtttaggaggtgaacacacggggcagcctacattctacgtggtttttttaacattataaaagcagcatgtgttattacaggaaatttaacagaagtatataaaaagaaaccagaagtca

SEQ ID NO: 27 - protein, 142 aa, Homo sapiens [sequence omitted]

SEQ ID NO: 28 - DNA, 791 bp, Homo sapiens
  CDS (166)...(675)
aagaactttaaaaatcacctaggtgtgggccgggcacggtggctaacgcctgtaatcccagcactttgagatgctgaggcaggtggatcacgaggtcaggagatcgagaccatcctggataacacggagaaaccccggcggagctgaggagcagggccgggcgccatggcaccgtggggcaagcggctggctggcgtgcgcggggtgctgcttgacatctcgggcgtgctgtacgacagcggcgcgggcggcggcacggccatcgccggctcggtggaggcggtggccagactgaagcgttcccggctgaaggtgaggttctgcaccaacgagtcgcagaagtcccgggcagagctggtggggcagcttcagaggctgggatttgacatctctgagcaggaggtgaccgccccggcaccagctgcctgccagatcctgaaggagcgaggcctgcgaccatacctgctcatccatgacggagtccgctcagaatttgatcagatcgacacatccaacccaaactgtgtggtaattgcagacgcaggagaaagcttttcttatcaaaacatgaataacgccttccaggtgctcatggagctggaaaaacctgtgctcatatcactgggaaaaggcatccacgggttcctctttgtggctagaacattcacgtcctaagtggggctgccgttggctgggttaaaccagtaaccagtaacaggagaaatacgtcgcatctctagtgtgtggagattaccgggcctttatattaaaaaaaaattttaagtgcttat

SEQ ID NO: 29 - protein, 169 aa, Homo sapiens
MAPWGKRLAGVRGVLLDISGVLYDSGAGGGTAIAGSVEAVARLKRSRLKVRFCTNESQKSRAELVGQLQRLGFDISEQEVTAPAPAACQILKERGLRPYLLIHDGVRSEFDQIDTSNPNCVVIADAGESFSYQNMNNAFQVLMELEKPVLISLGKGIHGFLFVARTFTS

SEQ ID NO: 30 - DNA, 659 bp, Homo sapiens
catgacggagtccgctcagaatttgatcagatcgacacatccaacccaaactgtgtggtaattgcagacgcaggagaaagcttttcttatcaaaacatgaataacgccttccaggtgctcatggagctggaaaaacctgtgctcatatcactgggaaaaggagccgctcaacacgtgttgcaaggcacgtgctctggacctggtgctcacacgtgctcttctcccaagacactccctgaagcgcgttccccaggagctggaagggggataacgcaggcccagtgacgagcaccatccggaagtgaaggctgatggtgaagagcctggaagaaacccaggggcctcctatgcacagatcttgcagcccagaaccaagtcagcttccctgcgactgcccaggcacactgcccatcaccccacccccgaaacaatgccagcccgctgctttttctatcctcccagtcacctttgcagacaaagaccaggggcagctcccgagggcactgtgaaggctcccatgccacacagtgagaactgtagcctctgcgtccaaggcacacagggtactttctggacccactgctggacagacttgaaggtgtcatgcccggtgtgtgcaggaggaaactaacagttcagtaaactctgccttgaccagc

SEQ ID NO: 31 - DNA, 2294 bp, Homo sapiens
  CDS (311)...(808)
tgtgccattggcgatgcggggaggctggccccatcgaaggctggtgggactggtggagactcctgtccactgctcagcactaggcctgcagcagacaccatgagccccaaacttcccaaagcccttccccagtcccacaagatggtgtctgcggaccgtgctcgtgagagatggcagccaggcagtccccacagggcacccattttcagctgcccccgcttctcagacaaggaaactgaggccagaaagccaggtggcccaggagctggcttccccatttcctgctcctgtgggccccactgcagtgcccatgggccgggctgatattacccgagacttcggagctctcacgggtgcgagtaatttaggctgcatggacacaagctgctggcttgagtcgccccgttatgaatgtgtgtgggtctgtgcccctttcatgtgctgccacagggcccacgagtgtgctgaaagggaaggacacggccaaggggccatggtggacaggagaccttcttgggggttcggtggtgtccttgaccccactctgactgagcactgccccaaggcactgccattccaggcccccttccctgagcctcccaccccaggcccacccacctgctgggtcctcccacctgcggggcccgccatgcggggtcaccatgcgagtctcaccatgcagggtcaccacacgagtctcaccatgcagggtcaccacgcgagtctcaccatgcagggtcaccatgcggggtcaccatgtggggtcaccatgcggggctcaccatgtggggcttcaggagcttgctgagcaccctccccacccacggtcactctccctggggtctgtaagcctccctgggcctgagcagctcccagccttgctgctgcctttccacttcctggcagtgaggtctcctgggtgccttctctcagccctttgggatgttttttgtgaggaagggaggctttgatgctgtggagcatctgtagtgcccactccagtggcttcacaggagcagcaggctgtttgttctgagctgttccaccttgtgcctgccagaggggagatagtggacaggcctccctccccccaagtggtggggtggaccccctgcccgctgtggccccatacctgggggccacacaccactgccctgggccgtgcagctgctatgaagagtgtgctgctgagaccctggaagagacggaggatgaaattgtgttgccagatagtccatttgttgttctgagactcgcatgcctgggagaatcctgggaattaactagctccttctctcccatcccattttacagaaaagtgagacccaaggtggtttctgacttgcccagaggtcataactgcttggacagtcatggtcctcagagcccacgtttgctgaccagtgcaggctctcacagccactcagctcctgcagccgtggcgtggcagaggagggaagcacttcctgggatttatgctgcctccctgacatttcaaggcccttcatttctctaaatattggaggagttgaattatttttagttgagcctcaagggatcagagaataagcttgcagcaacgttggcagatgggcttcttctagcagagagtggttattcggggcctcttattgagagaatcgggtgatttgaggaaatctggggtgtcctgaggcataccagaggacccccaagtttttcctgtggctcgtctgccatcaggaaaccaaaatgactcccctcgtcctgagctctccagggtgtggacctggaatgcttaaggggaggcaatggcatatctttaagatgagcacagctccggagccactcgagcacccaaggccacgtcctgctcagggcacttcgggcctcagtttccttatctttaaaatggacagagttggccgggtgaggtggccctgcctgtaatcccagcactttgggaggccaaggctggcagattgcttgagcccaggagtttgaagccagcctgggcaacatggcgaaaccccatctctactacaagtacaaaaatttggccgggcatggtggctcatgcttgtaatcccagcactttgggaggccaaggagagcggatcacttgaggccagaagctcgagaccgcctctactaaaaatacaaaaattagccaggcgtggtggctcacgcctgtaatctcagctactcgggtggctgaggcaggagaatcacttgaacctgggaagtagaggttgcagtgagctgagatcgtgccactgcactctagcctgggcgacagagcaaaaccctgtctc

SEQ ID NO: 32 - protein, 165 aa, Homo sapiens
MGRADITRDFGALTGASNLGCMDTSCWLESPRYECVWVCAPFMCCHRAHECAEREGHGQGAMVDRRPSWGFGGVLDPTLTEHCPKALPFQAPFPEPPTPGPPTCWVLPPAGPAMRGHHASLTMQGHHTSLTMQGHHASLTMQGHHAGSPCGVTMRGSPCGASGAC

SEQ ID NO: 33 - DNA, 3350 bp, Homo sapiens
  CDS (192)...(779)
tttttttttttttctgcttataagtttattcaatgcaaaataaccctcaccagttttactgaggtggctgaccatgtccacgaccaaatacgcctgtaaactgaaattcggttgctgacccattcccagcctcagctttctcactggcaccagggggacagcactccatctgtgggtgtctctttctctctatggctgtctgtctgtgggtgtctctctctgtctgtgggtgtctttcgccatctgtgggtatctctctctgtctgtgggtatctctcccatctgtgggtgtccatctctgtctttgggtgtctctctttgtgagtgtctctgtctgtggttgtctctgtctgtgggtgtctctctgtgagtgtccctgtgagtgtctctgtctgtgggtgtctctcctcgtctgtgggtatctctccctgtctgtgggtgtctctgttggcttccccacttgtgggtcttgcaggtcggtcacgctccagacctttaggccgcagcctgccagtctccagaccgctgtggcatggggtagcagacacgctctccaggggcagatggtggtaatcgcagagattctggatccccatgtgggtgaggtaccagtagaaatgtctccaggcaaactccttcctgcaacctcaggacctgagagactgcctggccttcatgacgtgaaggttgggcacattctcatctgccagctccgggtcttaggcaggtggacattcttcttggctaccgtgactccctccttaaaaaggagttcataaatagcaatctggttcttcttaggcatcaacatctctgcagctgtagggtccaggtccggggctggaaagcatgatttttttctaactgatctctgctgatggcatctagattgttcctggtttttcaccataccagggctgtgatgagcatcttggtgcatttcggatgacgtctccagatacagttacagaacgagtatttttgaggttcttgaggcatgttgccaagttgtttccagaaagctgcacagacttattctgcacagcctagaattctagaatcacagggttctgcacaacctagagttctggaatcacagggttctgcacagctagaattctagaatcacagggttctgcacagctagaattctagaatcacagggttctgcacaacctagagttctggaatcacagggttctgcacagcctagagttctggaatcacagggttctgcacagctagaattctagaatcacagggttctgcacagcctagagttctggaatcacagggttctgcacagcctagagttttggaatcacagggttctgcacagctagaattctagaatcacagggttctgcacagctagaattctagaatcacagggttctacacagctagaattctagaatcacaggctcccagggttgcaaggacactttggagtgtctacctcagcatctcatgaagtgtgggaattccgaggcggtggcggaggaagtgttttccatcttcggtgctttcgttgcttctggtgacagcgctcactgcctctgcttgctgtacgggaccagctgatggaaccgacagggagggactttttatctggccattggccactgccacacactttgtgtaccccgttttgtgtaattctgactacaaccttgtgggatctaggcaggtcattgctgttttgcaagtggggttgttgaagccacaggagatgaaataagctgctgtcccccagccattgagtgctgataggatcaggagtgccagttggtgtggctgaccccagaccctgtgcgtgttacctctaagctacattctagagcagactttttgcccacacaagccttaaatgtgggctggggacagtggctcacgccggtaatcccagcactttgggaggacaaggtgggcagatcacctgaggccaggggttcaagaccaggctggccaacatggtgaaaccctgtctctactaaaaatacaaaaattagccaggtgtggtggtgcgtgcctatagtcccagctactcgggaggctgacgcatgagaattgcttgaacctgggaggcagaggttgcagtaagtcaagactgcgccattgcactctagcctgggcgacagagcaagactccatctcgaaaaaaacaacaaaaccttaaatgtatttttgaggctgtgtttaaaaatggggatattttacacaaaatatccagatttctggattcttttgaagaatcagaagatctgacaatacggagcctcacattcctgcacacacagcagccatcgctggagccactgcctccattagtttgaatttactgcagaccccactcctccctgtcgtccctgtctccagaccacagagttagttgtcattgatcgtgtgccatttgttgtttttttcaaagtagagaagtacttcttcacgctgtgtctctatcaaaaatggacaagtgaaagatgtttcaagaaatgaaaagattttctttttagtgacaaaaaatttctagtatgtttctcatataaataaaatgtgtcctgtatgtagtcagggttcctcagagaagcccgaagctacaggatatagatatgtagagagattgtggaggcttggcgagtccaaaatctgcagggcagggctggcaggctggggactcaggaatgcgcgcagcagagtcgtaaggctgtgtgctggtgggattcttgctcggggaaggtcagtctttgttcttgtaaagcctgcaactggttggatgtggtccacccacattgcggaagggaatgtactctcctcctagttcaccgatttaaatgttaatctcatccaaaaacaccttcacagaaacatccagaataatgtttgaccacatatctgggcaccgtggcccagccaagttgacatattaaattaacccttgtagtccctttttaaacttacacccattgcaatttaggtcgctgctatggagcaagccacagaacctggcctcttaactcatttacccgggctgacccattaggcctttgagtcaccaacacctcactagagaacaagcataatgaagaagctctgctgtaattcgttaatgttaacacttttttctttaaagatgtctcatgctgagcttcgtggcacacgcctataatcccagcactttgggaggctgagatgagaggatggcttgagctcagaggttcgagaccagcctgggcagcatagtaagattccgtctctacaaaaaagaaaagaaaaaaagttgtctcataattattaaaaaccactattccagatcatggataataatagtcagaacaggtatattgttg

SEQ ID NO: 34 - protein, 195 aa, Homo sapiens
MAVCLWVSLSVCGCLSPSVGISLCLWVSLPSVGVHLCLWVSLFVSVSVCGCLCLWVSLCECPCECLCLWVSLLVCGYLSLSVGVSVGFPTCGSCRSVTLQTFRPQPASLQTAVAWGSRHALQGQMVVIAEILDPHVGEVPVEMSPGKLLPATSGPERLPGLHDVKVGHILICQLRVLGRWTFFLATVTPSLKRSS

SEQ ID NO: 35 - DNA, 21 bp, Homo sapiens
cacgtacccatcagccttcac

SEQ ID NO: 36 - DNA, 21 bp, Homo sapiens
cctgtggaaggagcatacagt

SEQ ID NO: 37 - DNA, 22 bp, Homo sapiens
cccagtgacgagcaccatccgg

SEQ ID NO: 38 - DNA, 20 bp, Homo sapiens
caacactggcacctgcagat

SEQ ID NO: 39 - DNA, 17 bp, Homo sapiens
ccaccccatgccatcaa

SEQ ID NO: 40 - DNA, 22 bp, Homo sapiens
aagtggcagagcagcccccagc

SEQ ID NO: 41 - DNA, 19 bp, Homo sapiens
cccgcctctccaagaccat

SEQ ID NO: 42 - DNA, 22 bp, Homo sapiens
ggtacactcatgtccccaccat

SEQ ID NO: 43 - DNA, 18 bp, Homo sapiens
gcgcaccgggaagttcag

SEQ ID NO: 44 - DNA, 20 bp, Homo sapiens
tgcaagcgataggagtggaa

SEQ ID NO: 45 - DNA, 21 bp, Homo sapiens
ggttgtccacgtacccatcag

SEQ ID NO: 46 - DNA, 22 bp, Homo sapiens
cccaccaggcccagtgacgagc

SEQ ID NO: 47 - DNA, 18 bp, Homo sapiens
gcgcaccgggaagttcag

SEQ ID NO: 48 - DNA, 27 bp, Homo sapiens
tgaagaacaaaacagaatgagaatgtg

SEQ ID NO: 49 - DNA, 31 bp, Homo sapiens
ccagctggagtcatttattcaccttccttcc

SEQ ID NO: 50 - DNA, 27 bp, Homo sapiens
ccaccagttactttcagtatgaaagca

SEQ ID NO: 51 - DNA, 26 bp, Homo sapiens
tatcctttcagagaagcagcaaaaac

SEQ ID NO: 52 - DNA, 24 bp, Homo sapiens
cagaaatgcctgcggcttttcctg

SEQ ID NO: 53 - DNA, 19 bp, Homo sapiens
cgccccagacccaagaatc

SEQ ID NO: 54 - DNA, 20 bp, Homo sapiens
caggaagtgcccatcagcct

SEQ ID NO: 55 - DNA, 23 bp, Homo sapiens
cccgcctctccaagaccatccct

SEQ ID NO: 56 - DNA, 20 bp, Homo sapiens
agcaccatccggaagtgaag

SEQ ID NO: 57 - DNA, 22 bp, Homo sapiens
gctgcaagatctgtgcatagga

SEQ ID NO: 58 - DNA, 29 bp, Homo sapiens
ctgatggtgaagagcctggaagaaaccca

SEQ ID NO: 59 - DNA, 22 bp, Homo sapiens
caatttaggtcgctgctatgga

SEQ ID NO: 60 - DNA, 22 bp, Homo sapiens
tggtgactcaaaggcctaatgg

SEQ ID NO: 61 - DNA, 27 bp, Homo sapiens
cctggcctcttaactcatttacccggg

SEQ ID NO: 62 - DNA, 19 bp, Homo sapiens
tggaggcagcctcgcttta

SEQ ID NO: 63 - DNA, 22 bp, Homo sapiens
ttggaggaagagttctcatgca

SEQ ID NO: 64 - DNA, 22 bp, Homo sapiens
cccgcagaacctccacgctgtt

SEQ ID NO: 65 - DNA, 23 bp, Homo sapiens
tctcccactgtatgctccttcca

SEQ ID NO: 66 - DNA, 22 bp, Homo sapiens
ctctgccacttcatctgcaggt

SEQ ID NO: 67 - DNA, 21 bp, Homo sapiens
cacgtacccatcagccttcac

SEQ ID NO: 68 - DNA, 24 bp, Homo sapiens
gaatctcccaaatcccagaactca

SEQ ID NO: 69 - DNA, 24 bp, Homo sapiens
acaccgggcatgacaccttcaagt

(19) United States
(12) Patent Application Publication
(10) Pub. No.: US 2007/0256148 A1
(43) Pub. Date: Nov. 1, 2007

(54) DEP2 and its uses in major depressive disorder and other related disorders

(76) Inventors: David A. Katz, Chicago, IL (US); Jeremy C. Packer, Wadsworth, IL (US); Anahita Bhathena, Evanston, IL (US); Christopher Neff, Salt Lake City, UT (US); Victor Abkevich, Salt Lake City, UT (US); Donna Shattuck, Salt Lake City, UT (US); Srikanth Jammulapati, Salt Lake City, UT (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/412,184

(22) Filed: Apr. 26, 2006

(51) Int. Cl. A01K 67/027; C12Q 1/68; C12P 21/06; C07H 21/04; C07K 14/705

(52) U.S. Cl. 800/14; 435/6; 435/69.1; 435/320.1; 435/325; 530/350; 536/23.5

(57) ABSTRACT

The present invention relates to DEP2 and its uses in connection with major depression or related disorders.

### BACKGROUND INFORMATION

Mood disorders, of which major depressive disorder is the most common, affect one person in five during their lifetime. The World Health Organization estimates that depression is currently the fourth most important worldwide cause of disability-adjusted life year loss, and that it will become the second most important cause by 2020 (See, Science, 288:39-40 (2000)). Pharmaceutical treatment of depression is frequently inadequate. In randomized clinical trials of the current best treatments, one-third of patients or more do not achieve remission, even after several months of treatment (See, Journal of the American Medical Association, 286:2947-55 (2001); Biological Psychiatry, 48:894-901 (2000)). Even when today's drugs do help patients achieve remission from their depression, the onset of action is over a period of weeks and there appears to be an increased risk of suicide during initial antidepressant therapy, although this risk may be less than that just prior to therapy initiation (See, Neuropsychopharmacology, 31:473-492 (2006)). Further, there are high recurrence rates approximately 85% of patients who achieve remission will suffer another episode of major depression (See, American Journal of Psychiatry, 156:1000-6 (1999)). Finally, currently available antidepressants are associated with side effects that lead some patients to stop taking their medications at risk of sinking back (further) into depression, and to morbidity in others (See, New England Journal of Medicine, 353:1819-34 (2005)).

The currently available antidepressants work primarily by increasing the activity of certain neurotransmitters, serotonin and norepinephrine, in synapses. Some medications (such as monoamine oxidase inhibitors) inhibit the degradation of these molecules, others (such as selective serotonin reuptake inhibitors and dual serotonin/norepinephrine reuptake inhibitors) decrease removal of neurotransmitters from the synaptic space, and some medications (such as receptor antagonists) stimulate norepinephrine release or inhibit negative feedback of serotonin signaling. Because these medications are all based on a single principle, the strength and range of their efficacy is similar. The improvements of the last half century have involved the development of safer and more tolerable drugs. However, despite this, today's drugs are neither completely safe nor completely tolerable for many patients.

Thus, there is considerable need for new drugs that are effective in a broader range of patients (particularly for patients whose depression is resistant to available pharmaceuticals), that have a faster onset of action, that are safer and more tolerable, or that complement the efficacy of existing drugs. It is possible, but unlikely, that further improvement in any of these dimensions will be achieved through development of additional serotonergic or noradrenergic agents. Therefore, alternative pharmacological approaches must be developed and pursued.

Part of the challenge in developing new drugs lies in the complexity of demonstrating efficacy of a major depression treatment. For example, the development of novel antidepressants is constrained by the limited understanding of depression's etiology. Because of this, there are relatively few pharmacological targets that can be considered for antidepressant development. Thereupon, there is a need for the identification of drug targets for depression. Genetic linkage can open new windows for the development of novel depression drug targets. Specifically, if a genetic variant is identified as being linked to depression in families, the gene in which that variant occurs is likely to be involved in the etiology of disease. Such a gene can be a target for the development of novel antidepressants. Additionally, such a gene can lead to the identification of previously unknown physiological pathways that may be modulated for effective therapy of depression.

Several genes have been identified or proposed as factors for depression or related phenotypes. Among these, most have been associated with disease in population studies of candidate genes selected on the basis of existing hypotheses about the etiology of depression. Many of these genes relate to serotonin or norepinephrine. Examples include: (1) associations of a HTR1A (serotonin receptor 1A) promoter variant with depression, suicide, bipolar disorder, panic disorder with agoraphobia, neuroticism and anti-depressant response; (2) associations of the HTT (serotonin transporter) promoter short allele with depression, suicide, depressive behavior response to tryptophan depletion, bipolar disorder antidepressant-induced mania and lesser anti-depressant response; and (3) association of a variant in HTR2c (serotonin receptor 2C) with both recurrent major depression and bipolar disorder and with major depression.

Thereupon, as evidenced by the above, there is a need in the art to identify proteins and genes associated with the pathophysiology of depression that are proteins and genes that relate to other than serotonin or norepinephrine. Such proteins and genes would be useful in the diagnosis of depression or a related disorder, and in the development of new drugs that could be used to treat patients suffering from depression or a related disorder.

### SUMMARY OF THE INVENTION

In one embodiment, the present invention relates to an isolated nucleic acid or fragment thereof comprising (a) a nucleotide sequence having at least 90% identity to SEQ ID NO:2; or (b) a complement comprising a nucleotide sequence having at least 90% identity to SEQ ID NO:2.

In another embodiment, the present invention relates to a purified polypeptide or fragment thereof comprising an amino acid sequence having at least 90% identity to: SEQ ID NO:3 or SEQ ID NO:4.

In yet another embodiment, the present invention relates to a vector comprising:

a) an isolated nucleic acid sequence comprising a nucleotide sequence having at least 90% identity to: (i) SEQ ID NO:2, (ii) nucleotides 351 to 770 of SEQ ID NO:2; or (iii) nucleotides 811 to 1161 of SEQ ID NO:2 or a complement comprising a nucleotide sequence having at least 90% identity to: (i) SEQ ID NO:2, (ii) nucleotides 351 to 770 of SEQ ID NO:2; or (iii) nucleotides 811 to 1161 of SEQ ID NO:2; operably linked to

b) a regulatory sequence.

Additionally, in yet another embodiment, the present invention relates to a host cell comprising the above-described vector.

In still yet another embodiment, the present invention relates to a non-human transgenic animal. In one aspect, said non-human transgenic animal comprises:

a) an exogenous and stably transmitted nucleic acid comprising a nucleotide sequence of SEQ ID NO:2; or

b) a knock-out of a nucleic acid comprising a nucleotide sequence of SEQ ID NO:2.

In another aspect, said non-human transgenic animal comprises:

a) an exogenous and stably transmitted nucleic acid having a nucleotide sequence selected from the group consisting of: SEQ ID NO:9, SEQ ID NO:11, SEQ ID NO:12, SEQ ID NO:14, SEQ ID NO:16, SEQ ID NO:18, SEQ ID NO:20, SEQ ID NO: 22, SEQ ID NO:24, SEQ ID NO:26, SEQ ID NO:28, SEQ ID NO:30, SEQ ID NO:31 and SEQ ID NO:33, with the proviso that said animal does not comprise an exogenous and stably transmitted nucleic acid having a nucleotide sequence of SEQ ID NO:2; or

b) a knock-out of a nucleic acid comprising a nucleotide sequence selected from the group consisting of: SEQ ID NO:9, SEQ ID NO: 1, SEQ ID NO: 12, SEQ ID NO: 14, SEQ ID NO:16, SEQ ID NO:18, SEQ ID NO:20, SEQ ID NO: 22, SEQ ID NO:24, SEQ ID NO:26, SEQ ID NO:28, SEQ ID NO:30, SEQ ID NO:31 and SEQ ID NO:33, with the proviso that a nucleic acid having a nucleotide sequence of SEQ ID NO:2 is not knocked out.

In yet another embodiment, the present invention relates to a method of modifying or altering the expression of SEQ ID NO:2 in a cell or animal. The method involves the steps of:

a) exposing said cell or administering to said subject a nucleic acid molecule, wherein said nucleic acid molecule modifies or alters the expression of SEQ ID NO: 2; and

b) modifying or altering the expression of SEQ ID NO:2.

In the above-described method, the nucleic acid molecule can be an antisense molecule, a small interfering RNA, a co-suppression RNA, an aptamer, a ribozyme or a triplexing agent.

In yet another embodiment, the present invention relates to a method of modifying or altering the expression of a nucleic acid sequence having a nucleotide sequence selected from the group consisting of: SEQ ID NO:9, SEQ ID NO:11, SEQ ID NO:12, SEQ ID NO:14, SEQ ID NO:16, SEQ ID NO:18, SEQ ID NO:20, SEQ ID NO: 22, SEQ ID NO:24, SEQ ID NO:26, SEQ ID NO:28, SEQ ID NO:30, SEQ ID NO:31 and SEQ ID NO:33 in a cell or animal, with the proviso that the expression of a nucleic acid having the sequence of SEQ ID NO: 2 is not modified or altered. The method involves the steps of:

a) exposing said cell or administering to said subject a nucleic acid molecule, wherein said nucleic acid molecule modifies or alters the expression of a nucleotide sequence selected from the group consisting of: SEQ ID NO:9, SEQ ID NO: 11, SEQ ID NO: 12, SEQ ID NO: 14, SEQ ID NO: 16, SEQ ID NO: 18, SEQ ID NO:20, SEQ ID NO: 22, SEQ ID NO:24, SEQ ID NO:26, SEQ ID NO:28, SEQ ID NO:30, SEQ ID NO:31 and SEQ ID NO:33; and

b) modifying or altering the expression of SEQ ID NO:9, SEQ ID NO: 11, SEQ ID NO:12, SEQ ID NO: 14, SEQ ID NO:16, SEQ ID NO:18, SEQ ID NO:20, SEQ ID NO: 22, SEQ ID NO:24, SEQ ID NO:26, SEQ ID NO:28, SEQ ID NO:30, SEQ ID NO:31 or SEQ ID NO:33.

In the above-described method, the nucleic acid molecule can be an antisense molecule, a small interfering RNA, a co-suppression RNA, an aptamer, a ribozyme or a triplexing agent.

In yet another embodiment, the present invention relates to a method of determining a genotype of a subject at a polymorphic site in nucleotides 1 to 315 of SEQ ID NO: 2 in a test sample. The method involves the steps of:

a) obtaining a test sample comprising DNA of a subject;

b) analyzing the test sample for at least one polymorphic site in nucleotides 1 to 315 of SEQ ID NO:2;

c) identifying the allele(s) present at said polymorphic site in said test sample; and

d) determining the genotype of a subject based on the identification of the allele(s) at said polymorphic site in said test sample.

The above-described method can further involve the step of analyzing the test sample(s) for a C-G polymorphism at position −1019 in a human serotonin receptor 1A (HTR1A) gene.

The analyzing performed in the above-described method can be accomplished using direct sequencing, polymerase chain reaction, ligase chain reaction, a fragment length polymorphism assay, a single strand conformation polymorphism analysis, a heteroduplex assay, hybridization, Taqman®, Molecular Beacon, Pyrosequencing, a microarray, Southern blotting, an Invader assay, a single base extension assay, or mass spectrometry.

In still yet another embodiment, the present invention relates to a method of determining a genotype of a subject at nucleotides 77402 or 79906_of SEQ ID NO: 1 in a test sample. The method involves the steps of:

a) obtaining a test sample comprising DNA of a subject;

b) analyzing the test sample for at least one polymorphic site selected from the group consisting of nucleotides 77402 and 79906 of SEQ ID NO: 1;

c) determining the allele(s) present at said polymorphic site in said test sample; and

d) determining the genotype of a subject based on the identification of the allele(s) at said polymorphic site in said test sample.

The above-described method can further involve the step of analyzing the test sample(s) for a C-G polymorphism at position −1019 in a human serotonin receptor 1A (HTR1A) gene.

The analyzing performed in the above-described method can be accomplished using direct sequencing, polymerase chain reaction, ligase chain reaction, a fragment length polymorphism assay, a single strand conformation polymorphism analysis, a heteroduplex assay, hybridization, Taqman®, Molecular Beacon, Pyrosequencing, a microarray, Southern blotting, an Invader assay, a single base extension assay, or mass spectrometry.

In still yet another embodiment, the present invention relates to a method of identifying a subject having major depression or a related disorder, or at risk of developing major depression or a related disorder. The method involves the steps of:

a) obtaining a test sample subject comprising DNA of a subject;

b) analyzing the test sample for at least one polymorphic site in SEQ ID NO: 1;

c) identifying at least one allele at said polymorphic site; and

d) identifying whether said subject has major depression or a related disorder or is at risk of developing major depression or a related disorder based on the allele(s) identified at said polymorphic site(s) in said test sample.

The above-described method can further involve the step of analyzing the test sample(s) for a C-G polymorphism at position −1019 in a human serotonin receptor 1A (HTR1A) gene.

The analyzing performed in the above-described method can be accomplished using direct sequencing, polymerase chain reaction, ligase chain reaction, a fragment length polymorphism assay, a single strand conformation polymorphism analysis, a heteroduplex assay, hybridization, Taqman®, Molecular Beacon, Pyrosequencing, a microarray, Southern blotting, an Invader assay, a single base extension assay, or mass spectrometry.

In still yet another embodiment, the present invention relates to a method of providing a prognosis for or predicting a response to treatment for a subject having major depression or a related disorder. The method involves the steps of:

a) obtaining a test sample comprising DNA of a subject;

b) analyzing the test sample for at least one polymorphic site in SEQ ID NO: 1;

c) identifying at least one allele(s) at said polymorphic site; and

d) providing a prognosis for and predicting the response to treatment for a subject having major depression or a related disorder based on the allele(s) identified at said polymorphic site(s) in said test sample.

The above-described method can further involve the step of analyzing the test sample(s) for a C-G polymorphism at position −1019 in a human serotonin receptor 1A (HTR1A) gene.

The analyzing performed in the above-described method can be accomplished using direct sequencing, polymerase chain reaction, ligase chain reaction, a fragment length polymorphism assay, a single strand conformation polymorphism analysis, a heteroduplex assay, hybridization, Taqman®, Molecular Beacon, Pyrosequencing, a microarray, Southern blotting, an Invader assay, a single base extension assay, or mass spectrometry.

In still yet another embodiment, the present invention relates to a method of detecting or quantifying an mRNA which comprises nucleotides 1 to 315 of SEQ ID NO:2 in a test sample. The method involves the steps of:

a) obtaining a test sample subject comprising mRNA of a subject;

b) analyzing the test sample for a mRNA comprising at least 15 contiguous nucleotides of nucleotides 1 to 315 of SEQ ID NO:2; and

b) detecting or quantifying said mRNA in said test sample.

The above-described method can further involve the step of analyzing the test sample(s) for an mRNA transcribed from a human serotonin receptor 1A (HTR1A) gene.

The analyzing performed in the above-described method can be accomplished using reverse transcription, quantitative polymerase chain reaction, cDNA microarrays, or Northern blotting.

In still yet another embodiment, the present invention relates to a method of identifying a subject having major depression or a related disorder, or at risk of developing major depression or a related disorder. The method involves the steps of:

a) obtaining a test sample subject comprising subject mRNA;

b) analyzing the test sample for at least one mRNA transcribed from SEQ ID NO: 1; and

c) identifying whether said subject has major depression or a related disorder or is at risk of developing major depression or a related disorder based on the presence, absence or amount of at least one of the mRNAs recited in step b) in said test sample.

The above-described method can further involve the step of analyzing the test sample(s) for an mRNA transcribed from a human serotonin receptor 1A (HTR1A) gene.

The analyzing performed in the above-described method can be accomplished using reverse transcription, quantitative polymerase chain reaction, cDNA microarrays, or Northern blotting.

In the above-described method, the mRNA transcribed from SEQ ID NO: 1 can have the nucleotide sequence of: SEQ ID NO:2, SEQ ID NO:5, SEQ ID NO:6, SEQ ID NO:7, SEQ ID NO:8, SEQ ID NO:9, SEQ ID NO:11, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:31 or SEQ ID NO:33.

In still yet another embodiment, the present invention relates to a method of providing a prognosis for or predicting a response to treatment for a subject having major depression or a related disorder. The method involves the steps of:

a) obtaining a test sample comprising mRNA of a subject;

b) analyzing the test sample for at least one mRNA transcribed from SEQ ID NO: 1; and

c) providing a prognosis for and predicting the response to treatment for a subject having major depression or a related disorder based on the presence, absence or amount of at least one of the mRNAs recited in step b) in said test sample.

The above-described method can further involve the step of analyzing the test sample(s) for an mRNA transcribed from a human serotonin receptor 1A (HTR1A) gene.

The analyzing performed in the above-described method can be accomplished using reverse transcription, quantitative polymerase chain reaction, cDNA microarrays, or Northern blotting.

In the above-described method, the mRNA transcribed from SEQ ID NO: 1 can have the nucleotide sequence of: SEQ ID NO:2, SEQ ID NO:5, SEQ ID NO:6, SEQ ID NO:7, SEQ ID NO:8, SEQ ID NO:9, SEQ ID NO:11, SEQ ID NO:12, SEQ ID NO:14, SEQ ID NO: 16, SEQ ID NO: 18, SEQ ID NO:20, SEQ ID NO:22, SEQ ID NO:24, SEQ ID NO:26, SEQ ID NO:28, SEQ ID NO:30, SEQ ID NO:31 or SEQ ID NO:33.

In still yet a further embodiment, the present invention relates to a method of detecting or quantifying the amount of a protein having an amino acid sequence selected from the group consisting of: SEQ ID NO:3 and SEQ ID NO:4 in a test sample. The method involves the steps of:

a) obtaining a test sample subject comprising at least one polypeptide of a subject; and

b) detecting or quantifying the amount of a protein having an amino acid sequence selected from the group consisting of SEQ ID NO: 3 and SEQ ID NO:4 in said test sample.

The above-described method can further involve the step of detecting or quantifying the amount of a polypeptide encoded by a human serotonin receptor 1A (HTR1A) gene.

The analyzing performed in the above-described method can be accomplished using ELISA, RIA, Western blotting, fluorescence activated cell sorting or immunohistochemical analysis.

In still yet a further embodiment, the present invention relates to a method of identifying a subject having major depression or a related disorder, or at risk of developing major depression or a related disorder. The method involves the steps of:

a) obtaining a test sample comprising at least one polypeptide of a subject;

b) analyzing the test sample for at least one polypeptide translated from SEQ ID NO:1; and

c) identifying whether said subject has major depression or a related disorder or is at risk of developing major depression or a related disorder based on the presence, absence or amount of at least of the polypeptides recited in step b) in said test sample.

The above-described method can further involve the step of analyzing the test sample(s) for a polypeptide encoded by a human serotonin receptor 1A (HTR1A) gene.

The analyzing performed in the above-described method can be accomplished using ELISA, RIA, Western blotting, fluorescence activated cell sorting or immunohistochemical analysis.

In the above-described method, the polypeptide translated from SEQ ID NO:1 can have an amino acid sequence selected from the group consisting of SEQ ID NO:3, SEQ ID NO:4, SEQ ID NO:10, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:32 and SEQ ID NO:34.

In still yet a further embodiment, the present invention relates to a method of providing a prognosis for or predicting a response to treatment for a subject having major depression or a related disorder. The method involves the steps of:

a) obtaining a test sample comprising at least one polypeptide of a subject;

b) analyzing the test sample for at least one polypeptide translated from SEQ ID NO:1; and

c) providing a prognosis for and predicting the response to treatment for a subject having major depression or a related disorder based on the presence, absence or amount of at least one of the polypeptides recited in step b) in said test sample.

The above-described method can further involve the step of analyzing the test sample(s) for a polypeptide encoded by a human serotonin receptor 1A (HTR1A) gene.

The analyzing performed in the above-described method can be accomplished using ELISA, RIA, Western blotting, fluorescence activated cell sorting or immunohistochemical analysis.

In the above-described method, the polypeptide translated from SEQ ID NO:1 can have an amino acid sequence selected from the group consisting of SEQ ID NO:3, SEQ ID NO:4, SEQ ID NO:10, SEQ ID NO:13, SEQ ID NO: 15, SEQ ID NO: 17, SEQ ID NO:19, SEQ ID NO:21, SEQ ID NO:23, SEQ ID NO:25, SEQ ID NO:27, SEQ ID NO:29, SEQ ID NO:32 and SEQ ID NO:34.

In still yet another embodiment, the present invention relates to a kit. In one aspect, the kit can comprise:

a) at least one reagent for determining a genotype of a subject at a polymorphic site in SEQ ID NO: 1 in a test sample; and

b) instructions for determining the genotype of the subject.

In another aspect, the kit can comprise:

a) at least one reagent for determining a genotype of a subject for a C-G polymorphism at position −1019 in a human serotonin receptor 1A (HTR1A) gene;

b) at least one reagent for determining a genotype of a subject for a polymorphic site in SEQ ID NO: 1, in a test sample; and

c) instructions for determining the genotype of the subject.

In another aspect, the kit can comprise:

a) at least one reagent for determining a genotype of a subject at nucleotide 77402 of SEQ ID NO:1 in a test sample; and

b) instructions for determining the genotype of the subject.

In another aspect, the kit can comprise:

a) at least one reagent for determining a genotype of a subject at nucleotide 79906 of SEQ ID NO:1 in a test sample; and

b) instructions for determining the genotype of the subject.

In another aspect, the kit can comprise:

a) at least one reagent for determining a genotype of a subject for a C-G polymorphism at position −1019 in a human serotonin receptor 1A (HTR1A) gene;

b) at least one reagent for determining a genotype of a subject for a polymorphic site at least one of nucleotides 77402 or 79906 in SEQ ID NO: 1, in a test sample; and

c) instructions for determining the genotype of the subject.

In still yet another aspect, the kit comprises:

a) at least one reagent for detecting or quantifying an mRNA transcribed from SEQ ID NO:1 in a test sample; and

b) instructions for detecting or quantifying the mRNA transcribed from SEQ ID NO:1 in the test sample.

In still yet another aspect, the kit comprises:

a) at least one reagent for detecting or quantifying an mRNA from a serotonin receptor 1A (HTR1A) gene in a test sample;

b) at least one reagent for detecting or quantifying an mRNA transcribed from SEQ ID NO:1, in a test sample; and

c) instructions for detecting or quantifying the mRNA from a serotonin receptor 1A (HTR1A) gene and the mRNA transcribed from SEQ ID NO:1 in the test sample.

The mRNA transcribed from SEQ ID NO:1 in the above kits in can have the nucleotide sequence of: SEQ ID NO:2, SEQ ID NO:5, SEQ ID NO:6, SEQ ID NO:7, SEQ ID NO:8, SEQ ID NO:9, SEQ ID NO:11, SEQ ID NO: 12, SEQ ID NO: 14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:31 or SEQ ID NO:33.

It still yet another aspect, the kit comprises:

a) at least one reagent for detecting or quantifying a polypeptide translated from SEQ ID NO: 1 in a test sample; and

b) instructions for detecting or quantifying the polypeptide translated from SEQ ID NO: 1 in the test sample.

In still yet another aspect, the kit comprises:

a) at least one reagent for detecting or quantifying a polypeptide encoded by a serotonin receptor 1A (HTR1A) gene in a test sample;

b) at least one reagent for detecting or quantifying a polypeptide translated from SEQ ID NO:1, in a test sample; and

c) instructions for detecting or quantifying the polypeptide encoded by a serotonin receptor 1A (HTR1A) gene and the polypeptide translated from SEQ ID NO:1 in the test sample.

The polypeptide translated from SEQ ID NO:1 in the above-described kits can have an amino acid sequence selected from the group consisting of SEQ ID NO:3, SEQ ID NO:4, SEQ ID NO:10, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:32 and SEQ ID NO:34.

In still yet another embodiment, the present invention relates to a method of screening a composition for the ability to bind to a protein translated from SEQ ID NO: 1. The method involves the steps of:

a) exposing said protein to a composition for a time and under conditions sufficient for said test composition to bind to said protein to form protein/composition complexes; and

b) detecting presence or absence of said complexes, wherein the presence of said complexes indicates a composition having the ability to bind to said protein.

The presence or absences of the complexes in the above-described method can be detected using mass spectrometry. Additionally, a composition identified pursuant to the above-described method as having the ability to bind to a protein translated from SEQ ID NO:1 can be used to treat major depression or a related disorder in a subject.

In the above-described method, a protein translated from SEQ ID NO:1 can have an amino acid sequence selected from the group consisting of SEQ ID NO:3, SEQ ID NO:4, SEQ ID NO: 10, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:32 and SEQ ID NO:34.

In still yet another embodiment, the present invention relates to a method of detecting binding of a composition to a protein translated from SEQ ID NO:1. The method involves the steps of:

a) subjecting said protein to nuclear magnetic resonance and recording the resulting spectrum;

b) subjecting said protein to nuclear magnetic resonance in the presence of said composition and recording the resulting spectrum; and

c) detecting the difference between said spectrum of step a) and said spectrum of step b) and comparing said difference to a control, said comparison indicating whether said composition binds to said protein.

A composition identified pursuant to the above-described method as having the ability to bind to a protein translated from SEQ ID NO:1 can be used to treat major depression or a related disorder in a subject.

In the above-described method, a protein translated from SEQ ID NO:1 can have an amino acid sequence selected from the group consisting of SEQ ID NO:3, SEQ ID NO:4, SEQ ID NO:10, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:32 and SEQ ID NO:34.

In still yet another embodiment, the present invention relates to a method of identifying the structure of a composition bound to a protein translated from SEQ ID NO: 1. The method involves the steps of:

a) exposing said protein to a composition known to bind to said protein; and

b) observing the resulting X-ray diffraction pattern of said resulting bound composition of step a), said diffraction pattern indicating the structure of said composition.

Additionally, a composition identified pursuant to the above-described method as having the ability to bind to a protein translated from SEQ ID NO:1 can be used to treat major depression or a related disorder in a subject.

In the above-described method, a protein translated from SEQ ID NO:1 can have an amino acid sequence selected from the group consisting of SEQ ID NO:3, SEQ ID NO:4, SEQ ID NO:10, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:32 and SEQ ID NO:34.

In still yet another embodiment, the present invention relates to a method of screening a composition for the ability to modulate the activity of a protein translated from SEQ ID NO:1. The method involves the steps of:

a) providing a composition;

b) exposing the protein to a substrate for sufficient time and conditions to allow the substrate to react with the protein in order to produce a reaction product or complex;

c) exposing the protein to the composition; and

d) measuring said reaction product or complex, wherein a decreased or increased amount of said reaction product or complex, as compared to the amount of reaction product or complex produced in the absence of said composition, indicates a composition having the ability to modulate the activity of said protein.

In the above-described method, a protein translated from SEQ ID NO:1 having an amino acid sequence selected from the group consisting of SEQ ID NO:10, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:32 and SEQ ID NO:34 may be capable of modifying the phosphorylation of the substrate. Additionally, the substrate can be selected from the group consisting of: phosphohistidine, phospholysine, phosphodiimide, pyrophosphate and a peptide or protein phosphorylated on histidine or lysine.

Furthermore, a composition identified in the above-identified method as having an ability to modulate the activity of a protein can be used to treat major depression or a related disorder in a subject.

Moreover, in the above-described method, a protein translated from SEQ ID NO:1 can have an amino acid sequence selected from the group consisting of SEQ ID NO:3, SEQ ID NO:4, SEQ ID NO:10, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:32 and SEQ ID NO:34.

In still yet another embodiment, the present invention relates to a method of screening a composition for the ability to modulate the activity of a protein translated from SEQ ID NO: 1. The method involves the steps of:

a) providing a composition;

b) simultaneously exposing the protein to the composition and to a substrate, wherein the protein is exposed to the substrate for sufficient time and conditions to allow the substrate to react with the protein in order to produce a reaction product or complex; and

c) measuring presence or absence of said reaction product or complex, wherein a lack of said reaction product or complex indicating a composition having the ability to modulate the activity of said protein.

In the above-described method, a protein translated from SEQ ID NO:1 having an amino acid sequence selected from the group consisting of SEQ ID NO:10, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:32 and SEQ ID NO:34 may be capable of modifying the phosphorylation of the substrate. Additionally, the substrate can be selected from the group consisting of: phosphohistidine, phospholysine, phosphodiimide, pyrophosphate and a peptide or protein phosphorylated on histidine or lysine.

Furthermore, a composition identified in the above-identified method as having an ability to modulate the activity of a protein can be used to treat major depression or a related disorder in a subject.

Moreover, in the above-described method, a protein translated from SEQ ID NO:1 can have an amino acid sequence selected from the group consisting of SEQ ID NO:3, SEQ ID NO:4, SEQ ID NO:10, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:32 and SEQ ID NO:34.

In still yet another embodiment, the present invention relates to a method of screening a composition for the ability to modulate activity of a protein translated from SEQ ID NO:1, in a cell. The method involves the steps of:

a) exposing said cell to said composition; and

b) measuring the amount of activity of said protein in said cell, wherein a decreased or increased amount of activity of said protein, as compared to a cell which has not been exposed to said composition, indicates a composition having the ability to modulate the activity of said protein.

A composition identified in the above-identified method as having an ability to modulate the activity of a protein can be used to treat major depression or a related disorder in a subject.

In the above-described method, a protein translated from SEQ ID NO:1 can have an amino acid sequence selected from the group consisting of SEQ ID NO:3, SEQ ID NO:4, SEQ ID NO:10, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:32 and SEQ ID NO:34.

In still yet another embodiment, the present invention relates to a method of screening a composition for the ability to modulate expression of a protein translated from SEQ ID NO:1, in a cell. The method involves the steps of:

a) exposing said cell to said composition; and

b) measuring the amount of said protein in said cell, wherein a decreased or increased amount of said protein, as compared to a cell which has not been exposed to said composition, indicates a composition having the ability to modulate the expression of said protein.

A composition identified pursuant to the above-described method as having an ability to modulate the expression of the protein can be used to treat major depression or a related disorder in a subject.

In the above-described method, a protein translated from SEQ ID NO:1 can have an amino acid sequence selected from the group consisting of SEQ ID NO:3, SEQ ID NO:4, SEQ ID NO:10, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:32 and SEQ ID NO:34.

In still yet another embodiment, the present invention relates to a method of screening a composition for the ability to modulate the level of expression of a protein translated from SEQ ID NO:1. The method comprises the steps of:

a) exposing an in vitro transcription and translation system comprising a regulatory sequence from SEQ ID NO:1 functionally connected to the open reading frame for a detectable protein, to a composition for a time and under conditions sufficient for said test whether said composition modulates the level of expression of the detectable protein; and [nb: the detectable protein may be a DEP2 product or may be a marker protein such as GFP or beta-galactosidase]

b) detecting the level of expression of the detectable protein, wherein a reduction or an increase in the level of expression of the detectable protein indicates that said composition has the ability to modulate the level of expression of a protein translated from SEQ ID NO:1.

A composition identified pursuant to the above-described method as having an ability to modulate the level of expression of the protein can be used to treat major depression or a related disorder in a subject.

In the above-described method, a protein translated from SEQ ID NO:1 can have an amino acid sequence selected from the group consisting of SEQ ID NO:3, SEQ ID NO:4, SEQ ID NO:10, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:32 and SEQ ID NO:34.

In still yet another embodiment, the present invention relates to a method of treating major depression or a related disorder in a subject in need of said treatment comprising the step of administering a composition identified as modulating the activity of a protein translated from SEQ ID NO:1, to said subject, in an amount sufficient to effect said treatment.

The composition administered to a subject pursuant to the above-described method can: (a) inhibit or reduce the activity of the protein; (b) increase the activity of the protein; or (c) decrease the activity of the protein.

A protein translated from SEQ ID NO:1 and that can be used in the above-described method can have an amino acid sequence selected from the group consisting of SEQ ID NO:3, SEQ ID NO:4, SEQ ID NO:10, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:32 and SEQ ID NO:34.

In still yet another embodiment, the present invention relates to a method of treating major depression or a related disorder in a subject in need of said treatment comprising reducing the amount of a protein translated from SEQ ID NO:1 in said subject, to a level sufficient to effect said treatment.

In the above-described method, the reduction can result from complete binding or partial binding of a composition to said protein. A protein translated from SEQ ID NO:1 and that can be used in the above-described method can have an amino acid sequence selected from the group consisting of SEQ ID NO:3, SEQ ID NO:4, SEQ ID NO:10, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:32 and SEQ ID NO:34.

In still yet another embodiment, the present invention relates to a method of treating major depression or a related disorder in a subject in need of said treatment comprising increasing the amount of a protein translated from SEQ ID NO:1, to a level sufficient to effect said treatment.

In addition, the above-described method can involve administering to said subject a therapeutically effective amount of a protein translated from SEQ ID NO:1. A protein translated from SEQ ID NO:1 and that can be used in the above-described method can have an amino acid sequence selected from the group consisting of SEQ ID NO:3, SEQ ID NO:4, SEQ ID NO:10, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:32 and SEQ ID NO:34.

In still yet another embodiment, the present invention relates to a method of treating major depression or a related disorder in a subject in need of said treatment comprising the step of administering a composition identified as modulating the level of expression of an mRNA molecule transcribed from SEQ ID NO:1 to said subject, in an amount sufficient to effect said treatment.

In the above-described method, an mRNA molecule transcribed from SEQ ID NO:1 can have the nucleotide sequence of SEQ ID NO:2, SEQ ID NO:5, SEQ ID NO:6, SEQ ID NO:7, SEQ ID NO:8, SEQ ID NO:9, SEQ ID NO:11, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:31 or SEQ ID NO:33.

In still yet another embodiment, the present invention relates to a method of determining the therapeutic activity of a composition used to treat major depression or a related disorder. The method involves the steps of:

a) determining the amount of a protein translated from SEQ ID NO:1, in a test sample from a subject treated with said composition; and

b) comparing the amount of said protein in said test sample with the amount of protein present in a test sample from said subject prior to treatment, an equal amount of said protein in said test sample of said treated subject indicating lack of therapeutic activity of said composition and a changed amount of said protein in said test sample of said treated subject indicating therapeutic activity of said composition.

In the above-described method, a protein translated from SEQ ID NO:1 can have an amino acid sequence selected from the group consisting of SEQ ID NO:3, SEQ ID NO:4, SEQ ID NO:10, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:32 and SEQ ID NO:34.

In still yet another embodiment, the present invention relates to a method of determining the level of therapeutic activity of a composition used to treat major depression or a related disorder. The method involves the steps of:

a) determining the activity of a protein translated from SEQ ID NO:1, in a test sample from a subject treated with said composition; and

b) comparing the amount of activity of said protein in said test sample with the amount of activity of protein present in a test sample from said subject prior to treatment, an equal amount of activity of said protein in said test sample of said treated subject indicating lack of therapeutic activity of said composition and a changed amount of activity of said protein in said test sample of said treated subject indicating therapeutic activity of said composition.

In the above-described method, a protein translated from SEQ ID NO:1 can have an amino acid sequence selected from the group consisting of SEQ ID NO:3, SEQ ID NO:4, SEQ ID NO:10, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:32 and SEQ ID NO:34.

In still yet another embodiment, the present invention relates to a method of determining the level of in vivo activity of a composition used to treat major depression or a related disorder comprising the steps of:

a) determining the amount of a mRNA molecule transcribed from SEQ ID NO:1, in a test sample from a subject treated with said composition; and

b) comparing the amount of said mRNA molecule in said test sample with the amount of mRNA molecule present in a test sample from said subject prior to treatment, an equal amount of said mRNA molecule in said test sample of said treated subject indicating lack of therapeutic activity of said composition and a changed amount of said mRNA molecule in said test sample of said treated subject indicating therapeutic activity of said composition.

In the above-described method, an mRNA molecule transcribed from SEQ ID NO:1 can have the nucleotide sequence of SEQ ID NO:2, SEQ ID NO:5, SEQ ID NO:6, SEQ ID NO:7, SEQ ID NO:8, SEQ ID NO:9, SEQ ID NO:11, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:31 or SEQ ID NO:33.

In still yet another embodiment, the present invention relates to a method of determining presence or absence of activity of a composition used to treat major depression or a related disorder. The method involves the steps of:

a) observing phenotype of a subject according to a method validated as a measure of major depression or a related disorder;

b) administering said composition to said subject for a time and under conditions sufficient for said composition to bind to, inhibit, increase or reduce the activity of, or increase or reduce the amount of a protein translated from SEQ ID NO:1;

c) repeating step a) with said subject of step b); and

d) comparing said phenotype of step a) and said phenotype of step c), a difference in step c) as compared to step a) indicating presence of activity of said composition and the lack of a difference indicating absence of activity of said composition.

In the above-described method, a protein translated from SEQ ID NO:1 can have an amino acid sequence selected from the group consisting of SEQ ID NO:3, SEQ ID NO:4, SEQ ID NO:10, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:32 and SEQ ID NO:34.

In still yet another embodiment, the present invention relates to a method of determining presence or absence of activity of a composition used to treat major depression or a related disorder comprising the steps of:

a) observing phenotype of a subject according to a method validated as a measure of major depression or a related disorder;

b) administering said composition to said subject for a time and under conditions sufficient for said composition to increase or reduce the amount of a mRNA molecule transcribed from SEQ ID NO:1;

c) repeating step a) with said subject of step b); and

d) comparing said phenotype of step a) and said phenotype of step c), a difference in step c) as compared to step a) indicating presence of activity of said composition and the lack of a difference indicating absence of activity of said composition.

In the above-described method, an mRNA molecule transcribed from SEQ ID NO:1 can have the nucleotide sequence of SEQ ID NO:2, SEQ ID NO:5, SEQ ID NO:6, SEQ ID NO:7, SEQ ID NO:8, SEQ ID NO:9, SEQ ID NO:11, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:31 or SEQ ID NO:33.

### BRIEF DESCRIPTION OF THE FIGURES

FIG. 1 shows the genomic DNA sequence comprising DEP2 (SEQ ID NO:1). The capital letters show the open reading frame for human phospholysine phosphohistidine inorganic pyrophosphate phosphatase (Lhpp) protein. Red text indicates single nucleotide polymorphisms. The bold underlined portions are forward primers. The bold italic underlined portions are reverse primers. The italic portions are repeat regions.

FIG. 2 shows the nucleic acid sequence of DEP2-1 (SEQ ID NO:2). The nucleic acid sequence contains two (2) coding regions, each of which are shown in capital letters.

FIG. 3A shows the amino acid sequence of the Dep2-1a protein encoded by one of the coding regions of the nucleic acid shown in FIG. 2 (SEQ ID NO:3).

FIG. 3B shows the amino acid sequence of the Dep2-1b protein encoded by the second coding region of the nucleic acid shown in FIG. 2 (SEQ ID NO:4).

FIG. 4 shows the nucleic acid sequence of a naturally occurring splice variant of DEP2-1 (SEQ ID NO:5). The coding regions are shown in capital letters. The amino acid sequences of the proteins encoded by each of the coding regions is the same as shown in SEQ ID NOS: 3 and 4.

FIG. 5 shows the nucleic acid sequence of a naturally occurring splice variant of DEP2-1 (SEQ ID NO:6). The coding regions are shown in capital letters. The amino acid sequences of the proteins encoded by each of the coding regions is the same as shown in SEQ ID NOS: 3 and 4.

FIG. 6 shows the nucleic acid sequence of a naturally occurring splice variant of DEP2-1 (SEQ ID NO:7). The coding region is shown in capital letters. The amino acid sequence of the protein encoded by the coding region is the same as shown in SEQ ID NO:4.

FIG. 7 shows the nucleic acid sequence of a naturally occurring splice variant of DEP2-1 (SEQ ID NO:8). The coding region is shown in capital letters. The amino acid sequence of the protein encoded by the coding region is the same as shown in SEQ ID NO:4.

FIG. 8 shows the reference nucleic acid sequence of LHPP (SEQ ID NO:9). The coding region is shown in capital letters.

FIG. 9 shows the amino acid sequence of the Lhpp protein encoded by the nucleic acid shown in FIG. 2 (SEQ ID NO:10). A polymorphic amino acid has been found to exist at amino acid 94 where arginine is replaced by glutamine (see the underline).

FIG. 10 shows a naturally occurring splice variant of LHPP (SEQ ID NO:11). The coding region is shown in capital letters. The coding region encodes a protein that is identical to SEQ ID NO:10.

FIG. 11 shows a naturally occurring splice variant of LHPP (SEQ ID NO:12). The coding region is shown in capital letters.

FIG. 12 shows the amino acid sequence of the variant Lhpp protein encoded by the nucleic acid shown in FIG. 5 (SEQ ID NO:13).

FIG. 13 shows a naturally occurring splice variant of LHPP (SEQ ID NO:14). The coding region is shown in capital letters. The amino acid sequence of the variant Lhpp protein encoded by the nucleic acid is shown in SEQ ID NO:15.

FIG. 14 shows a nucleic acid sequence of a naturally occurring splice variant of LHPP (SEQ ID NO:16). The coding region is shown in capital letters. The amino acid sequence of the variant Lhpp protein encoded by the nucleic acid is shown in SEQ ID NO:17.

FIG. 15 shows a nucleic acid sequence of a naturally occurring splice variant of LHPP (SEQ ID NO:18). The coding region is shown in capital letters. The amino acid sequence of the variant Lhpp protein encoded by the nucleic acid is shown in SEQ ID NO:19.

FIG. 16 shows a nucleic acid of a naturally occurring splice variant of LHPP (SEQ ID NO:20). The coding region is shown in capital letters. The amino acid sequence of the variant Lhpp protein encoded by the nucleic acid is shown in SEQ ID NO:21.

FIG. 17 shows a nucleic acid of a naturally occurring splice variant of LHPP (SEQ ID NO:22). The coding region is shown in capital letters. The amino acid sequence of the variant Lhpp protein encoded by the nucleic acid is shown in SEQ ID NO:23.

FIG. 18 shows a nucleic acid of a naturally occurring splice variant of LHPP (SEQ ID NO:24). The coding region is shown in capital letters. The amino acid sequence of the variant Lhpp protein encoded by the nucleic acid is shown in SEQ ID NO:25.

FIG. 19 shows a nucleic acid of a naturally occurring splice variant of LHPP (SEQ ID NO:26). The coding region is shown in capital letters. The amino acid sequence of the protein encoded by the nucleic acid is shown in SEQ ID NO:27.

FIG. 20 shows a nucleic acid sequence of DEP2-2 (SEQ ID NO:28). The coding region is shown in capital letters.

FIG. 21 shows the amino acid sequence of the Dep2-2 protein encoded by the nucleic acid shown in FIG. 20 (SEQ ID NO:29).

FIG. 22 shows a nucleic acid sequence of DEP2-3 (SEQ ID NO:30).

FIG. 23 shows a nucleic acid of AK127935 (GenBank: AK127935) (SEQ ID NO:31). The coding region is shown in capital letters.

FIG. 24 shows the amino acid sequence of the Dep2-4 protein encoded by the nucleic acid shown in FIG. 23 (SEQ ID NO:32).

FIG. 25 shows a nucleic acid of AW867792 (GenBank: AW867792) (SEQ ID NO:33). The coding region is shown in capital letters.

FIG. 26 shows the amino acid sequence of the Dep2-5 protein encoded by the nucleic acid shown in FIG. 25 (SEQ ID NO:34).

FIG. 27 shows the genetic evidence on chromosome 10 for linkage of a gene to major depressive disorder. The lower, curved line and lower, two horizontal lines show evidence from standard linkage analysis. The green lines show evidence from serotonin receptor 1A-conditional linkage analysis. The dotted horizontal lines indicate the extent of the linkage region as defined by a drop of one unit in the heterogeneity LOD score. The dashed horizontal lines indicate the extent of the linkage region as defined by a drop of two units in the heterogeneity LOD score. The right side of the figure represents the telomere of chromosome 10. The approximate location of DEP2 is represented by an arrowhead.

FIG. 28 shows a Northern blot probed with a polynucleotide complementary to SEQ ID NO:2, SEQ ID NO:5, SEQ ID NO:6, SEQ ID NO:7, SEQ ID NO:8, SEQ ID NO:9, SEQ ID NO:11 and SEQ ID NO:12.

FIG. 29 is a schematic representation of certain naturally occurring DEP2 transcripts and proteins. This figure was created using Genecarta software (Compugen, Tel Aviv, Israel). Dark lines represent transcripts and light lines represent coding regions. Transcripts shown include LHPP and naturally occurring variants thereof, DEP2-1 and naturally occurring variants thereof, and DEP2-2. DEP2-3, for which all supportive evidence has been generated, is not shown. AK127935 and AW867792, which share no exons in common with LHPP or DEP2-1, or naturally occurring variants thereof, are not shown. Although SEQ ID NO:2, SEQ ID NO:5 and SEQ ID NO:6 each contain the coding regions for both Dep2-1a and Dep2-1b, only the 5′-most (Dep2-1a) is associated with those transcripts in this figure. The location of Dep2-1b is associated with SEQ ID NO:7 and SEQ ID NO:8.

FIG. 30 shows a sequence alignment of DEP2-1 sequences either predicted by a bioinformatic algorithm (Genecarta) or determined experimentally by direct sequencing of cloned cDNAs h5173309, h 5194531, h5197955 and h4565014. Arrowheads indicate major transcription start sites determined by RLM-RACE. A single nucleotide polymorphism is indicated by an underlined base in the h4565014 sequence. The last line of sequence was found, downstream of a polyadenylate tract, in h4565014 and does not match DEP2.

FIG. 31 shows RLM-RACE results. MWM=molecular weight markers.

FIG. 32 shows the results of exon bridging PCR experiment #1 in Example 7. The lower band between 50 and 100 nucleotide markers are primer dimers.

FIG. 33 shows the results of exon bridging PCR experiment #2 in Example 7. Negative controls are reactions in which no reverse transcriptase was added.

### DETAILED DESCRIPTION OF THE INVENTION

Definitions

As used herein, the singular forms “a,” “an” and “the” include plural reference unless the context clearly dictates otherwise. Unless defined otherwise, all technical and scientific terms used herein have the same meaning as commonly understood to one those of skill in the art to which this invention belongs.

As used herein, the term “allele” refers to a particular form of a nucleic acid, either DNA or RNA, wherein different alleles of a nucleic acid differ in sequence, by either change or insertion/deletion, at one or more nucleotides at a polymorphic site.

“cDNA” refers to a DNA that is complementary to and synthesized from a mRNA template using the enzyme reverse transcriptase. The cDNA can be single-stranded or converted into the double-stranded form.

As used herein, the term “coding sequence” or “coding region” refers to a nucleic acid sequence that codes for a specific amino acid sequence.

As used herein, the term “complementarity” refers to as the degree of relatedness between two nucleic acid segments. It is determined by measuring the ability of the sense strand of one nucleic acid segment to hybridize with the antisense strand of the other nucleic acid segment, under appropriate conditions, to form a double helix. A “complement” is defined as a sequence which pairs to a given sequence based upon the canonic base-pairing rules. For example, a sequence A-G-T in one nucleotide strand is “complementary” to T-C-A in the other strand.

In the DNA double helix, wherever adenine appears in one strand, thymine (uridine in RNA) appears in the other strand. Similarly, wherever guanine is found in one strand, cytosine is found in the other. The greater the relatedness between the nucleotide sequences of two nucleic acid segments, the greater the ability to form hybrid duplexes between the strands of the two nucleic acid segments.

“Similarity” between two amino acid sequences is defined as the presence of a series of identical as well as conserved amino acid residues in both sequences. The higher the degree of similarity between two amino acid sequences, the higher the correspondence, sameness or equivalence of the two sequences. (“Identity” between two amino acid sequences is defined as the presence of a series of exactly alike or invariant amino acid residues in both sequences.) The definitions of “complementarity”, “identity” and “similarity” are well known to those of ordinary skill in the art.

As used herein, the term “DEP2” refers to a gene on human chromosome 10q26.2 that has been statistically linked and associated with major depression, and that is believed to be within the 159 kb sequence comprising SEQ ID NO:1. Transcripts that arise from DEP2 include: (a) LHPP (SEQ ID NO:9) (See, Yokoi et al., J Biochem 133:607-14 (2003)); (b) naturally occurring splice variants of LHPP (SEQ ID NO:1, SEQ ID NO:12, SEQ ID NO:14, SEQ ID NO:16, SEQ ID NO:18, SEQ ID NO:20, SEQ ID NO:22, SEQ ID NO:24 and SEQ ID NO:26; (c) DEP2-1 (SEQ ID NO:2); (d) naturally occurring splice variants of DEP2-1 (SEQ ID NO:5, SEQ ID NO:6, SEQ ID NO:7 or SEQ ID NO:8); (e) DEP2-2 (SEQ ID NO:28); (f) DEP2-3 (SEQ ID NO:30); (g) GenBank sequence AK127935 (SEQ ID NO:31); and (h) GenBank sequence AW867792 (SEQ ID NO:33). Proteins that are encoded within DEP2 include: (a) Lhpp (SEQ ID NO:10) (See, Yokoi et al., J Biochem 133:607-14 (2003)); (b) naturally occurring protein variants of Lhpp (SEQ ID NO:13, SEQ ID NO:15, SEQ ID NO:17, SEQ ID NO:19, SEQ ID NO:21, SEQ ID NO:23, SEQ ID NO:25 and SEQ ID NO:27); (c) Dep2-1a and Dep2-1b (SEQ ID NO:3 and SEQ ID NO:4, respectively); (d) Dep2-2 (SEQ ID NO:29); (e) Dep2-4 (SEQ ID NO:32); and (f) Dep2-5 (SEQ ID NO:34).

As used herein, the term “DEP2 transcripts” refers to the group of transcripts arising in whole or in part from SEQ ID NO:1, including but not limited to: LHPP and naturally occurring splice variants thereof; DEP2-1 and naturally occurring splice variants thereof; DEP2-2; DEP2-3; GenBank sequence AK127935 and GenBank sequence AW867792. As used herein, the term “DEP2 proteins” refers to the group of proteins encoded in whole or in part from one or more DEP2 transcripts, including but not limited to: Lhpp and naturally occurring protein variants thereof; Dep2-1a; Dep2-1b; Dep2-2; Dep2-4 and Dep2-5. As used herein, the terms “DEP2 polymorphic sites” or “DEP2 polymorphisms”, used interchangeably, refer to polymorphic sites found within SEQ ID NO:1, or if outside of SEQ ID NO:1, within a DEP2 transcript.

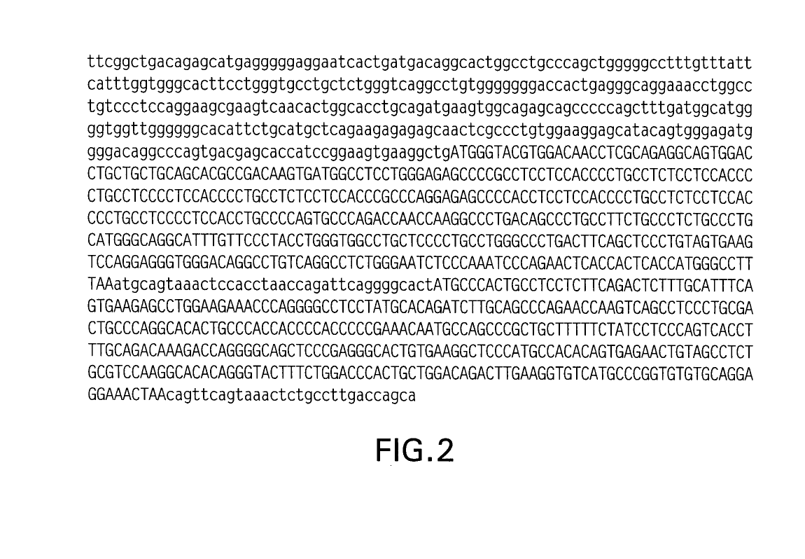

As used herein, the term “DEP2-1” refers to a messenger RNA shown in SEQ ID NO:2 and in FIG. 2, and DNA sequences that functionally regulate expression thereof. Experimental evidence that DEP2-1 messenger RNA is a naturally occurring transcript is disclosed herein. As shown in FIG. 2, DEP2-1 messenger RNA has 2 exons. Of these, an exon comprising nucleotides 1-315 of SEQ ID NO:2 was not previously known to be in any naturally occurring transcript. In addition, three naturally occurring polymorphic sites in nucleotides 1-315 of DEP2-1 messenger RNA are disclosed herein: (a) 135T>C, (b) 209A>G and (c) 241G>A.

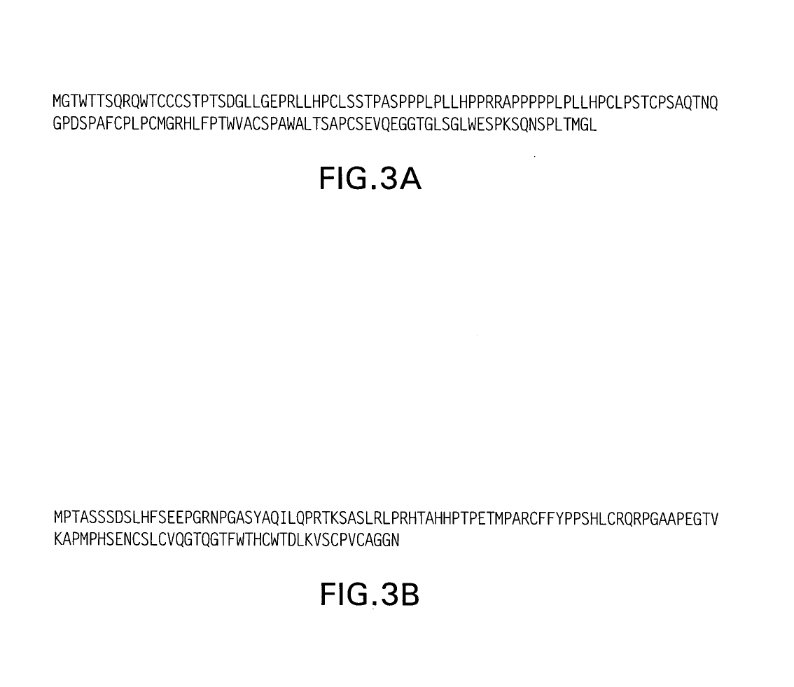

As used herein, the terms “Dep2-1a” and “Dep2-1b” refer to proteins shown in SEQ ID NO:3 and in FIG. 3A, and in SEQ ID NO:4 and in FIG. 3B, respectively. These proteins may be encoded from DEP2-1 as well as naturally occurring splice variants thereof.

As used herein, the term “DEP2-2” refers to a messenger RNA shown in SEQ ID NO:28, and DNA sequences that functionally regulate expression thereof.

As used herein, the term “Dep2-2” refers to a protein shown in SEQ ID NO:29. This protein may be encoded from DEP2-2.

As used herein, the term “DEP2-3” refers to a messenger RNA shown in SEQ ID NO:30, and DNA sequences that functionally regulate expression thereof.

As used herein, the term “Dep2-4” refers to a protein shown in SEQ ID NO:32. This protein may be encoded from SEQ ID NO:31.

As used herein, the term “Dep2-5” refers to a protein shown in SEQ ID NO:34. This protein may be encoded from SEQ ID NO:33.

As used herein, the phrase “effective amount” or a “therapeutically effective amount”, which are used interchangeably herein, when used in connection with an active agent (such as a drug) is meant a nontoxic but sufficient amount of the active agent to provide the desired effect. The amount of active agent (such as a drug) that is “effective” will vary from subject to subject, depending on the age and general condition of the individual, the particular active agent or agents, and the like. Thus, it is not always possible to specify an exact “effective amount.” However, an appropriate “effective amount” in any individual case may be determined by one of ordinary skill in the art using routine experimentation.

As used herein, the phrase “encoded by” refers to a nucleic acid sequence which codes for a polypeptide sequence, wherein the polypeptide sequence or a portion thereof contains an amino acid sequence of at least 3 amino acids, more preferably at least 8 amino acids, and even more preferably at least 15 amino acids from a polypeptide encoded by the nucleic acid sequence.

As used herein, the term “exon” refers to a portion of the gene sequence that is transcribed and is found in the mature messenger RNA derived from the gene, but is not necessarily a part of the sequence that encodes the final gene product.

The term “expression”, as used herein, refers to the production of a functional end-product. Expression of a gene involves transcription of the gene and translation of the mRNA into a precursor or mature protein. “Antisense inhibition” refers to the production of antisense RNA transcripts capable of suppressing the expression of the target transcript. “Co-suppression” refers to the production of sense RNA transcripts capable of suppressing the expression of identical or substantially similar foreign or endogenous genes (U.S. Pat. No. 5,231,020).

As used herein, the term “fragment” of a nucleic acid sequence refers to a contiguous sequence of approximately at least 6 nucleotides, preferably at least about 8 nucleotides, more preferably at least about 10 nucleotides, and even more preferably at least about 15 nucleotides, and most preferable at least about 20 nucleotides identical or complementary to a region of the specified nucleotide sequence.) Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, and “N” for any nucleotide.

As used herein, the term “gene” refers to a nucleic acid sequence that undergoes transcription as a result of the activity of at least one promoter. A gene may encode for a particular polypeptide, or alternatively, code for a RNA molecule. A gene includes one or more exons and one or more regulatory or control sequences and may include one or more introns. The phrase “target gene” as used herein, refers to a nucleic acid sequence, such as, but not limited to, a nucleic acid sequence of interest that encodes a polypeptide of interest or alternatively, a RNA molecule of interest. The term “target gene” can also refer to a gene to be identified or knocked-out according to the methods described herein.

As used herein, the term “genotype” refers to the identity of alleles present in a subject or in a test sample.

As used herein, the term “genotyping” refers to the process of determining the genotype of a subject.

As used herein, the terms “homologous”, “substantially similar” and “corresponding substantially” are used interchangeably. They refer to nucleic acid or protein fragments wherein changes in one or more nucleotide bases or amino acids does not affect the ability of the nucleic acid or protein fragment to mediate gene expression or produce a certain phenotype. These terms also refer to modifications of the nucleic acid or protein fragments of the instant invention such as deletion or insertion of one or more nucleotides or amino acids that do not substantially alter the functional properties of the resulting nucleic acid or protein fragment relative to the initial, unmodified fragment. It is therefore understood, as those skilled in the art will appreciate, that the invention encompasses more than the sequences exemplified herein.

As used herein, the term “identity” refers to the relatedness of two sequences on a nucleotide-by-nucleotide basis over a particular comparison window or segment. Thus, identity is defined as the degree of sameness, correspondence or equivalence between the same strands (either sense or antisense) of two DNA segments (or two amino acid sequences).

“Percentage of sequence identity” is calculated by comparing two optimally aligned sequences over a particular region, determining the number of positions at which the identical base or amino acid occurs in both sequences in order to yield the number of matched positions, dividing the number of such positions by the total number of positions in the segment being compared and multiplying the result by 100. Optimal alignment of sequences may be conducted by the algorithm of Smith & Waterman, Appl. Math., 2:482 (1981), by the algorithm of Needleman & Wunsch, J. Mol. Biol., 48:443 (1970), by the method of Pearson & Lipman, Proc. Natl. Acad. Sci., (USA) 85:2444 (1988) and by computer programs which implement the relevant algorithms (for example, Clustal Macaw Pileup (which is publicly available on the Internet; Higgins et al., CABIOS. 5L151-153 (1989)), FASTDB (Intelligenetics), BLAST (National Center for Biomedical Information; Altschul et al., Nucleic Acids Research, 25:3389-3402 (1997)), PILEUP (Genetics Computer Group, Madison, Wis.) or GAP, BESTFIT, FASTA and TFASTA (Wisconsin Genetics Software Package Release 7.0, Genetics Computer Group, Madison, Wis.). (See U.S. Pat. No. 5,912,120.)

As used herein, the term “isoform” refers to a particular form of a protein, wherein different isoforms of a protein differ in sequence, by either change or insertion/deletion, or covalent modification at one or more amino acids.

As used herein, the terms “isolated” or “purified”, used interchangably, when used in connection with biological molecules such as nucleic acids or proteins means that the molecule is substantially free of other biological molecules such as nucleic acids, proteins, lipids, carbohydrates or other material such as cellular debris and growth media. Generally, the term “isolated” or “purified” are not intended to refer to a complete absence of such material or to absence of water, buffers, or salts, unless they are present in amounts that substantially interfere with the methods of the present invention.

As used herein, an “isolated nucleic acid fragment or sequence” is a polymer of nucleic acid (RNA or DNA) that is single- or double-stranded, optionally containing synthetic, non-natural or altered nucleotide bases. An isolated nucleic acid fragment in the form of a polymer of DNA may be comprised of one or more segments of cDNA, genomic DNA or synthetic DNA.

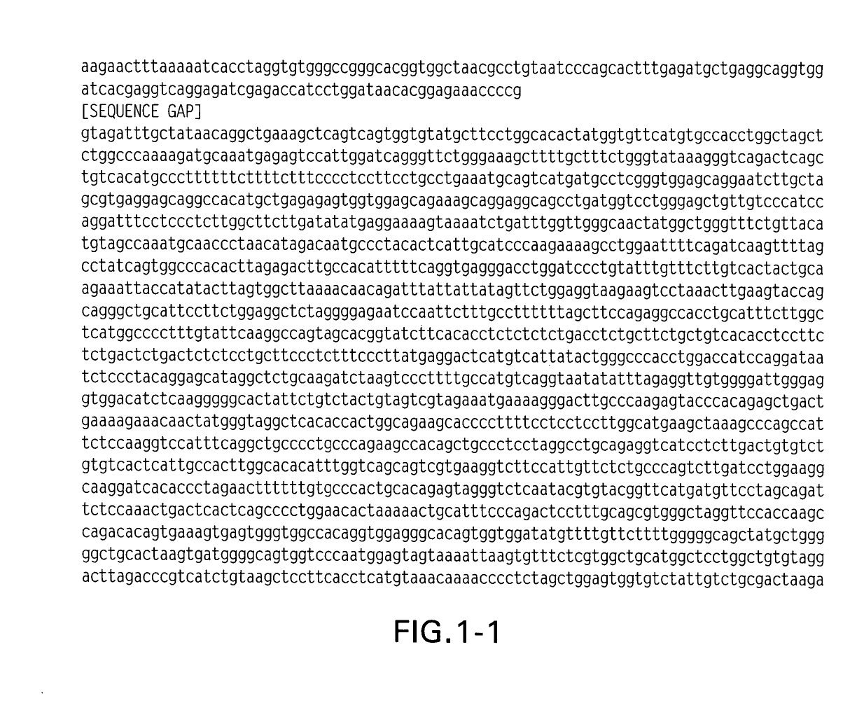

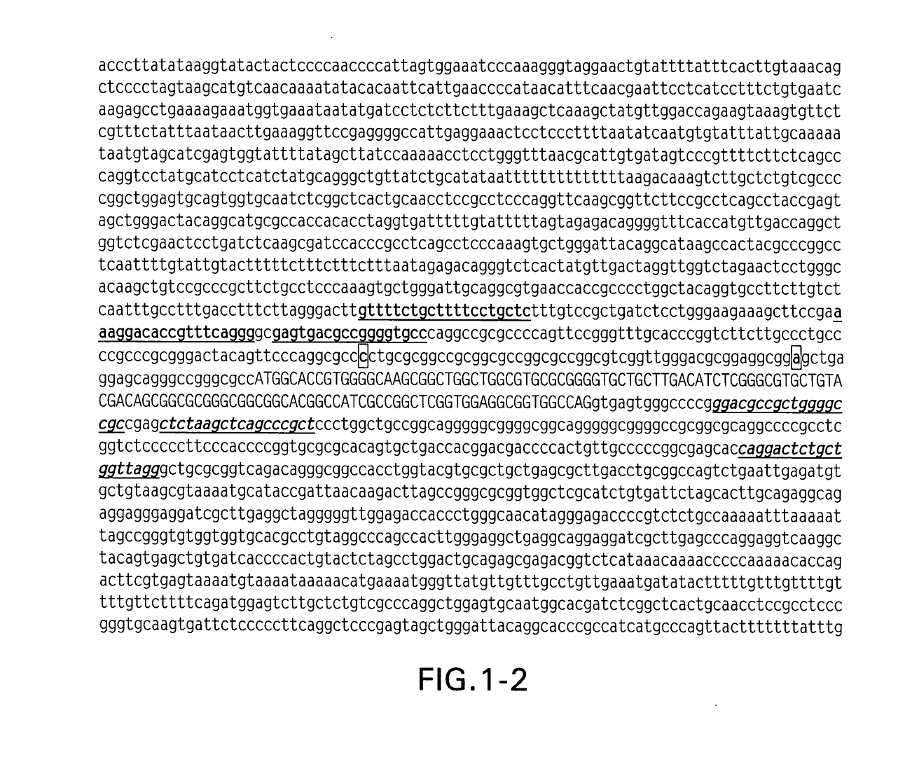

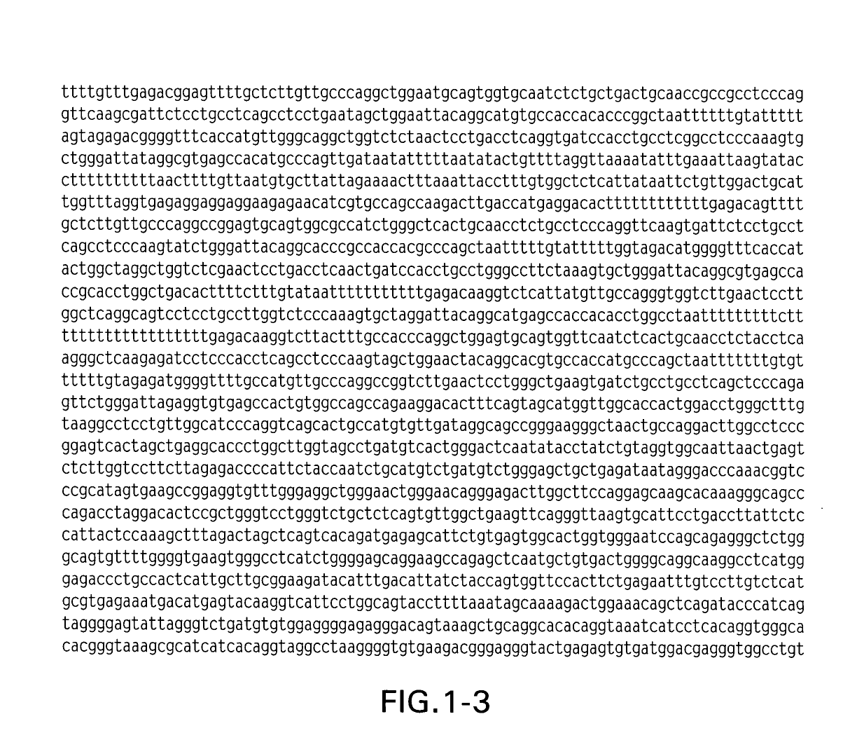

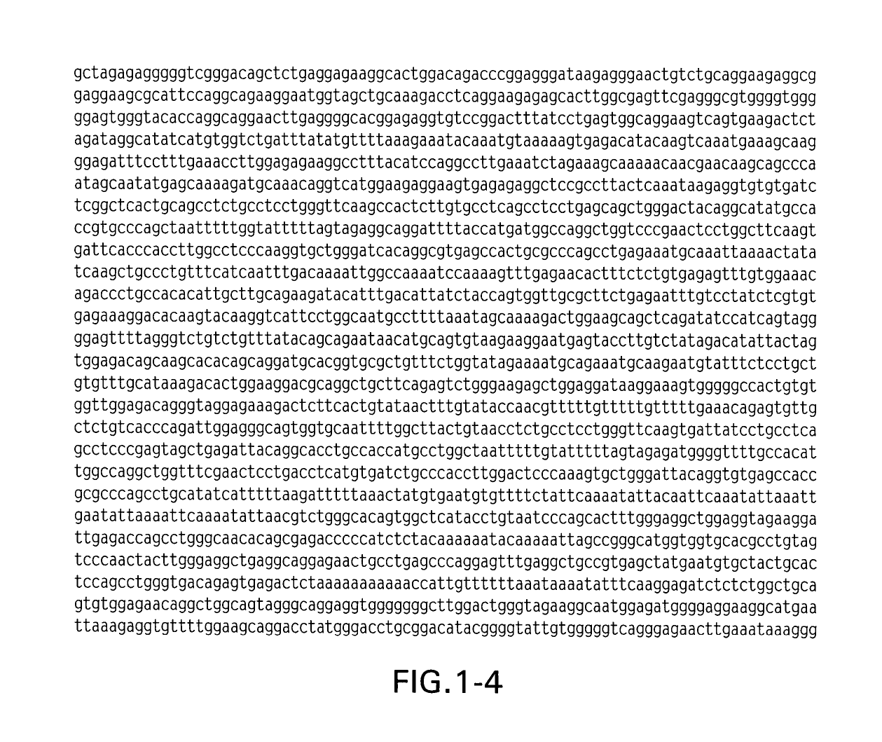

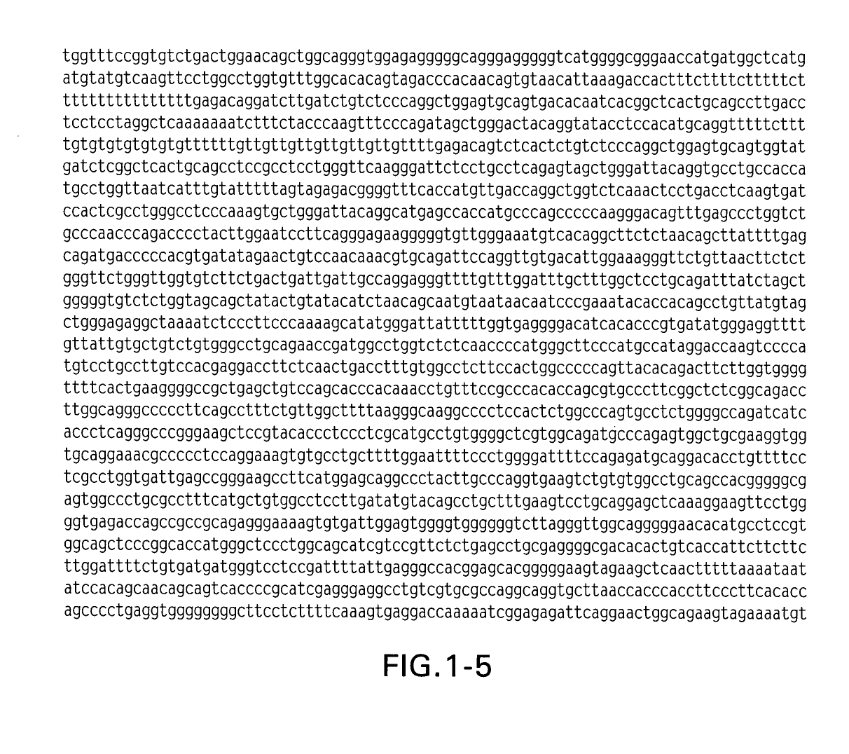

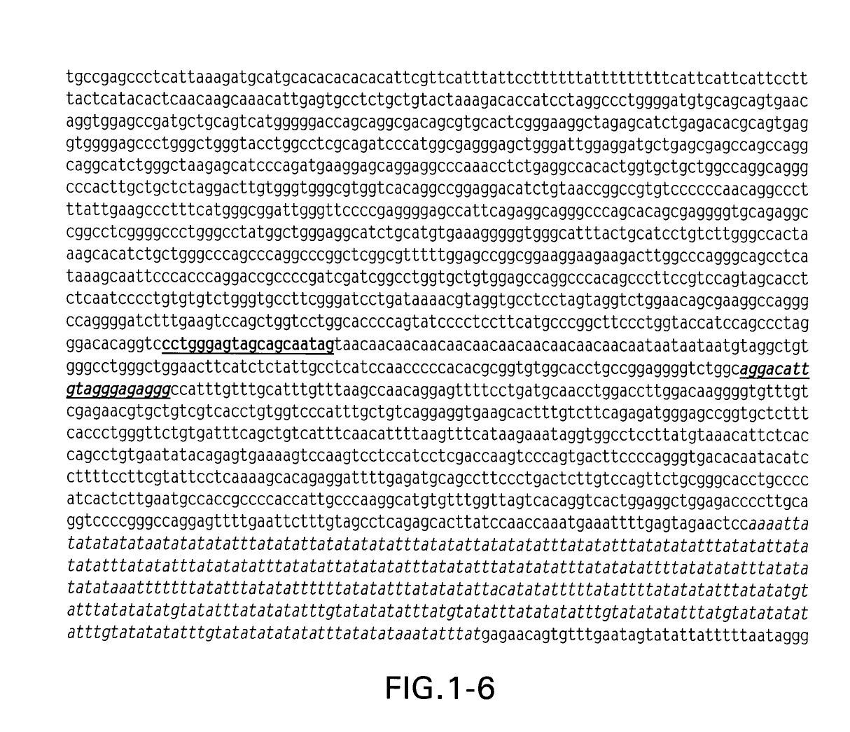

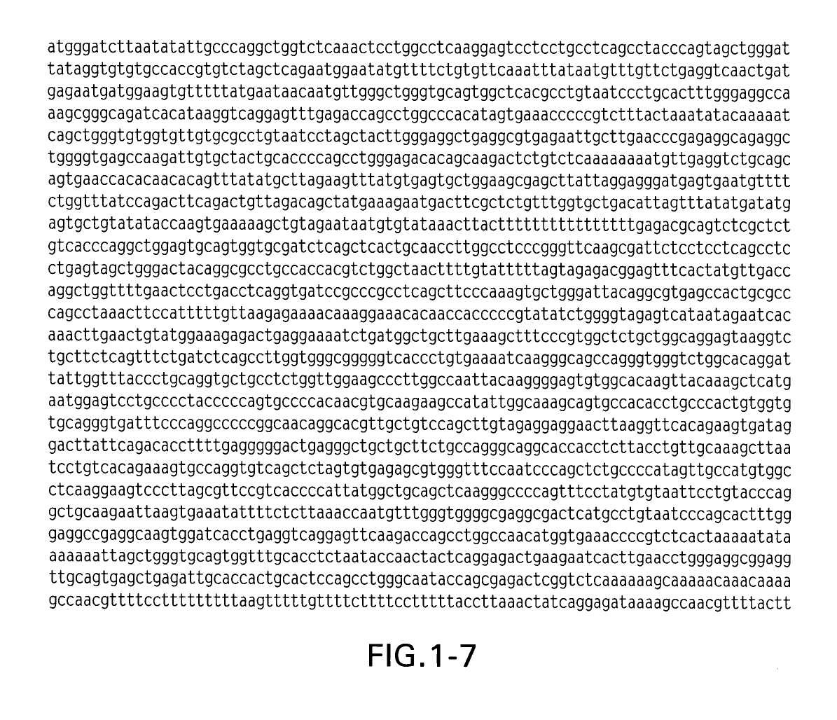

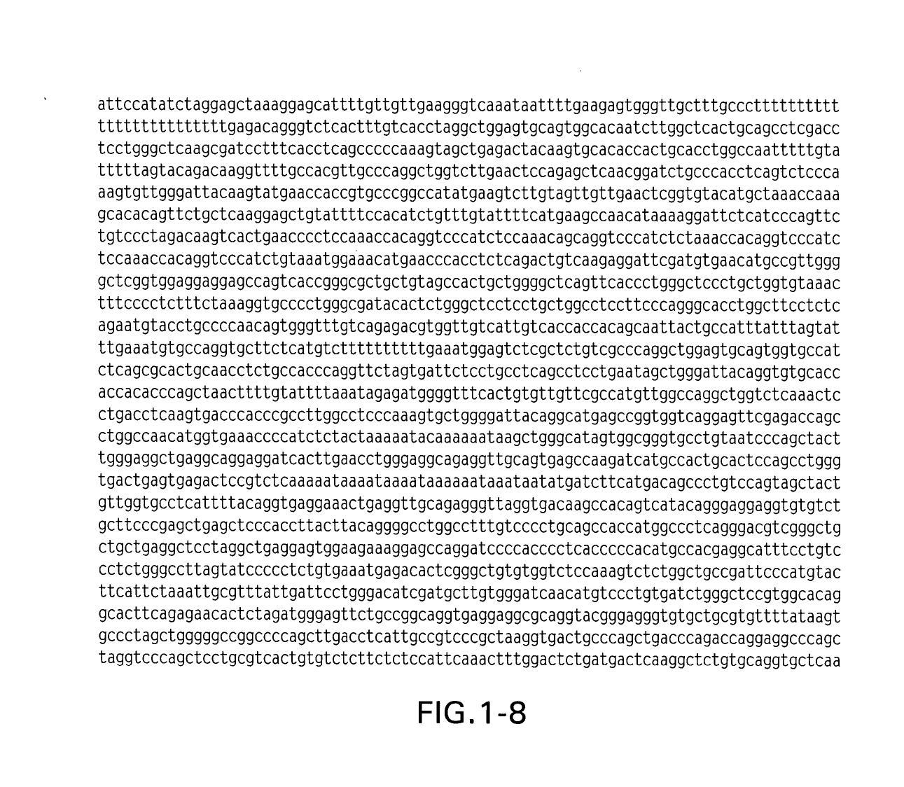

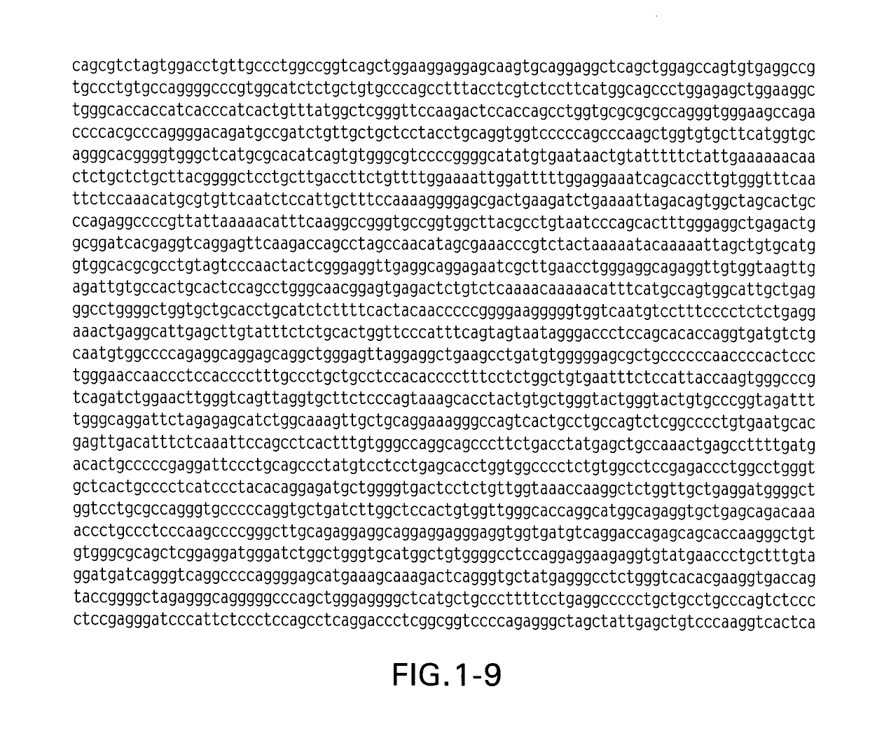

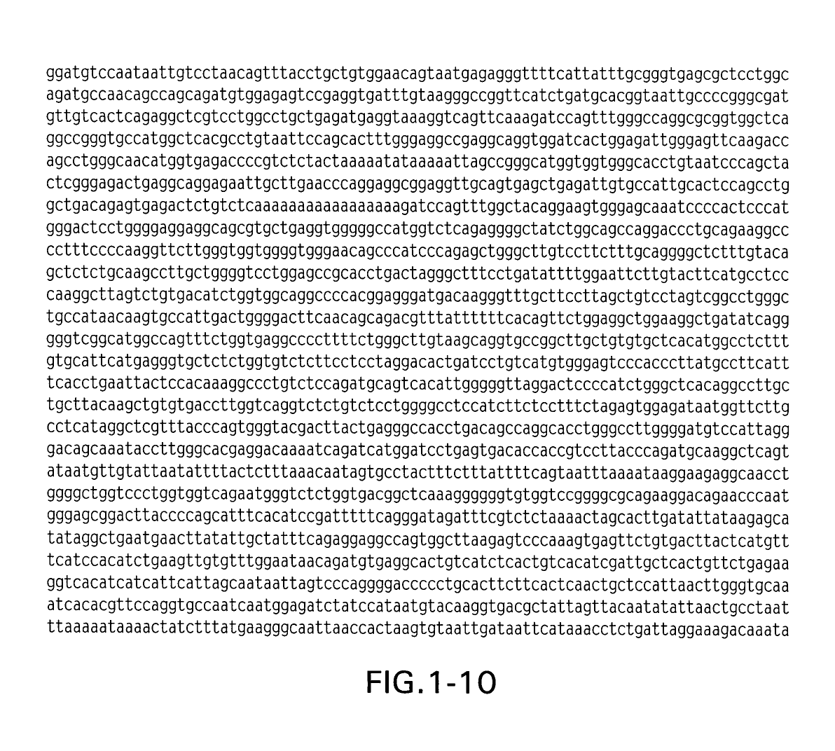

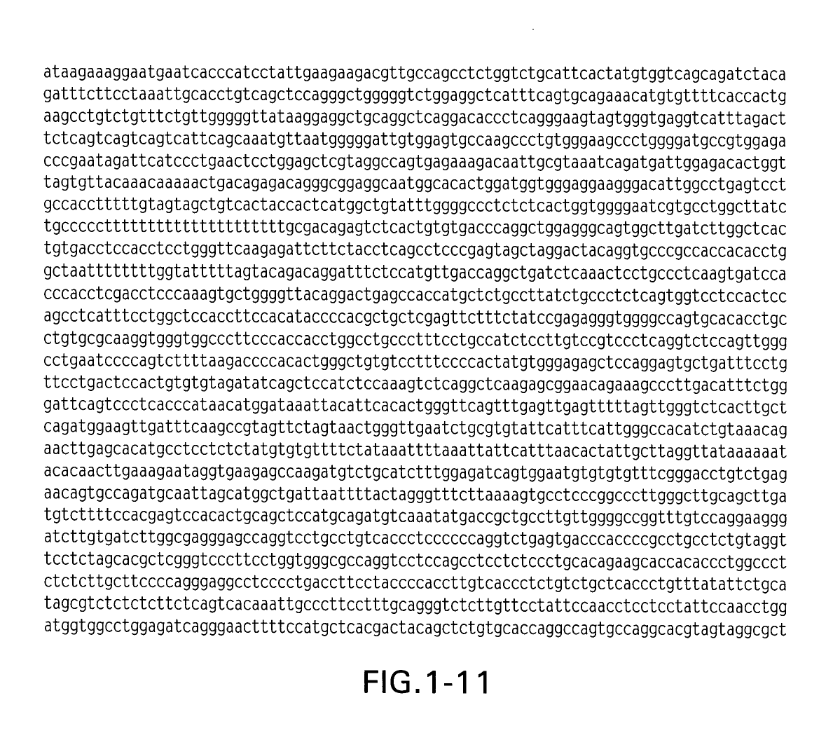

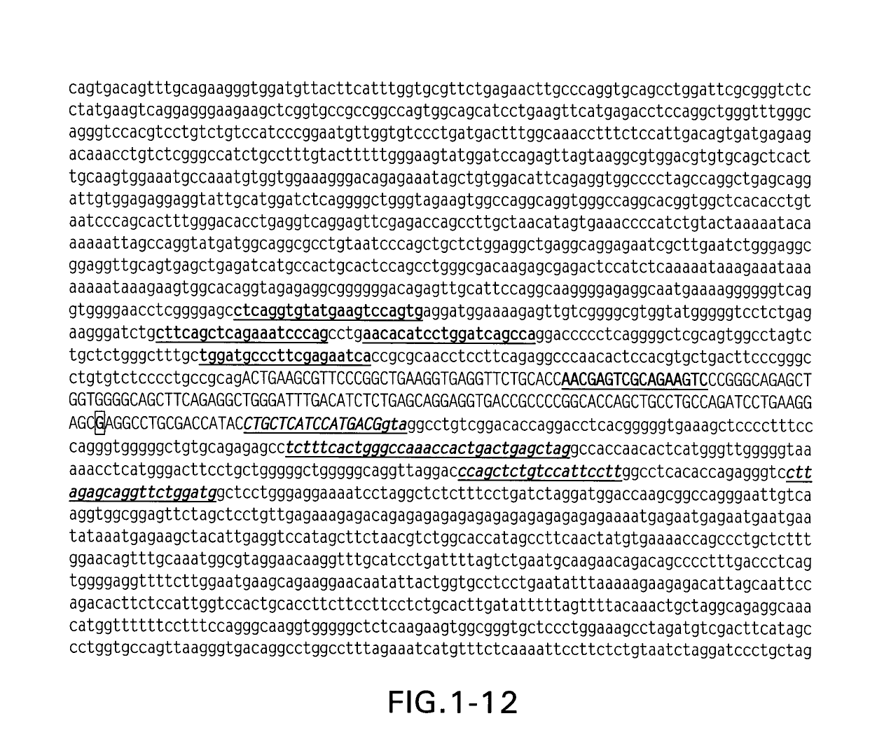

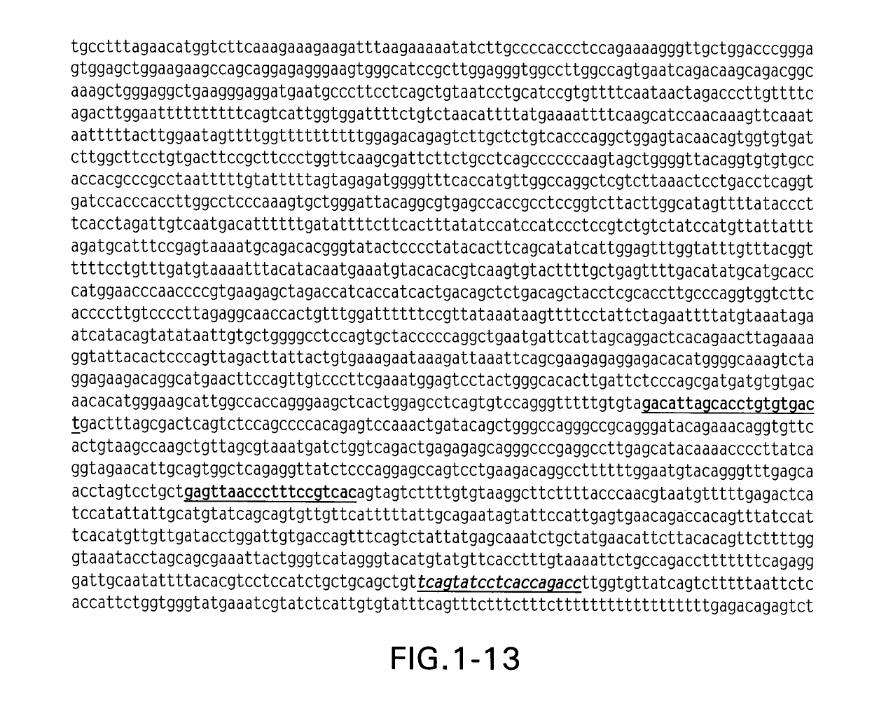

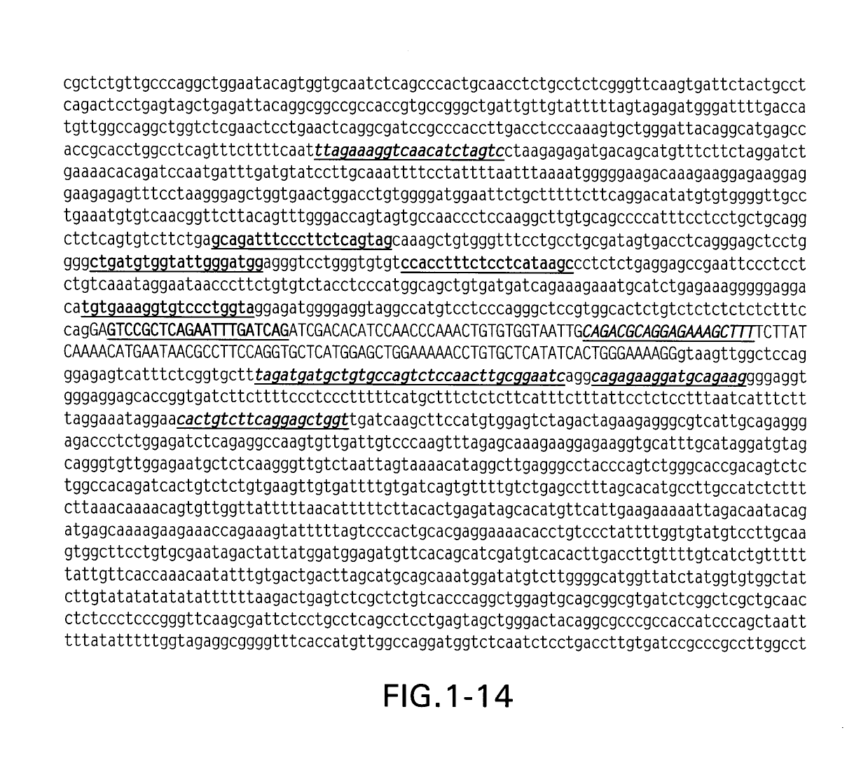

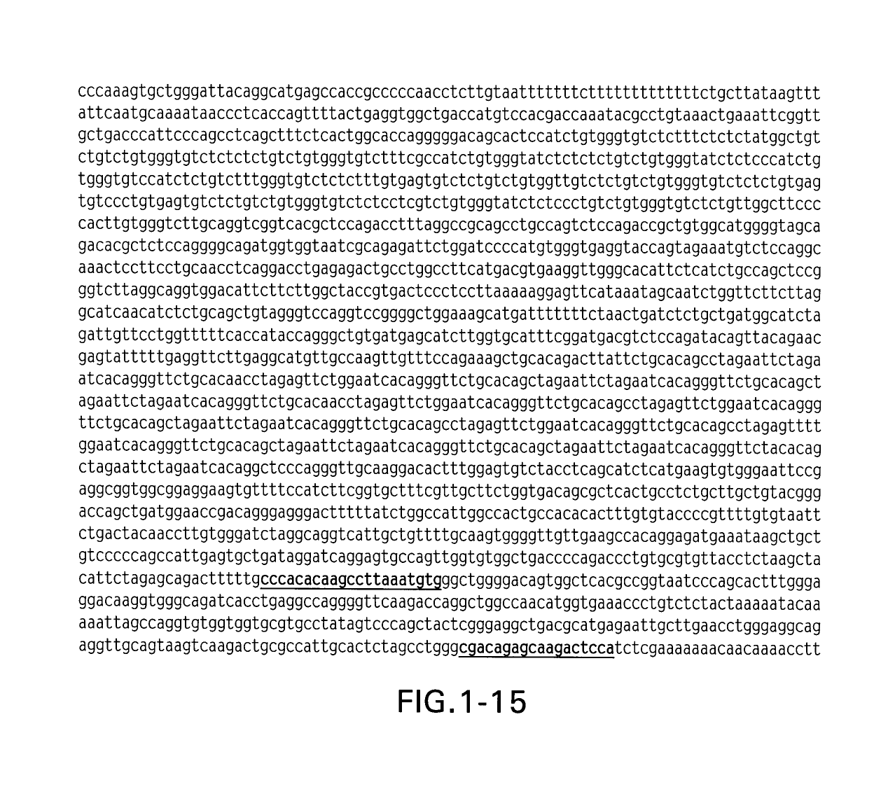

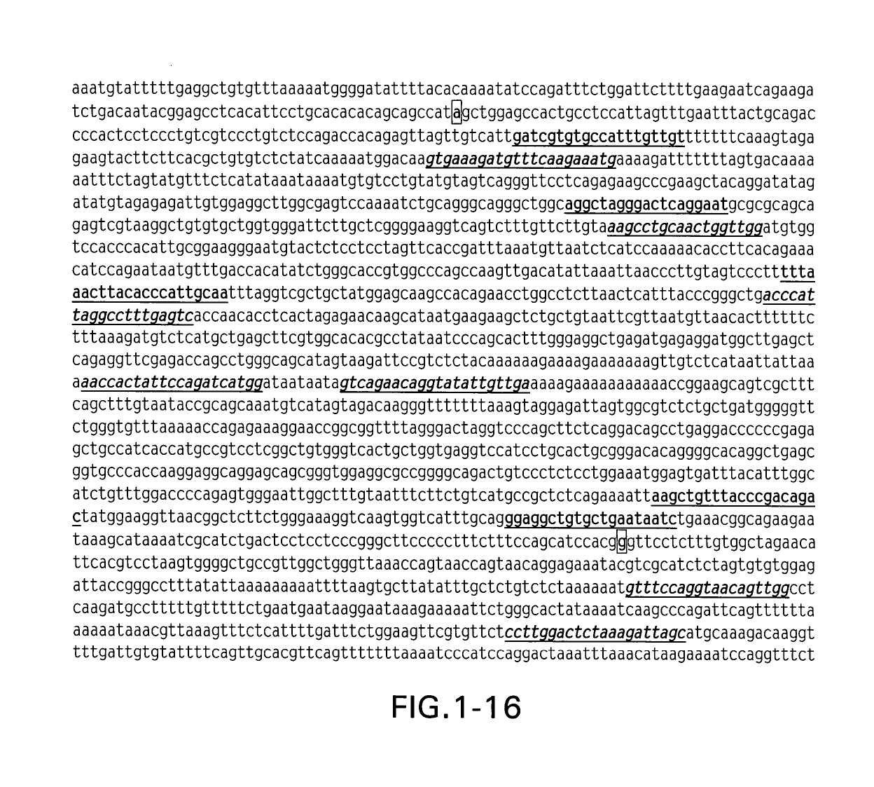

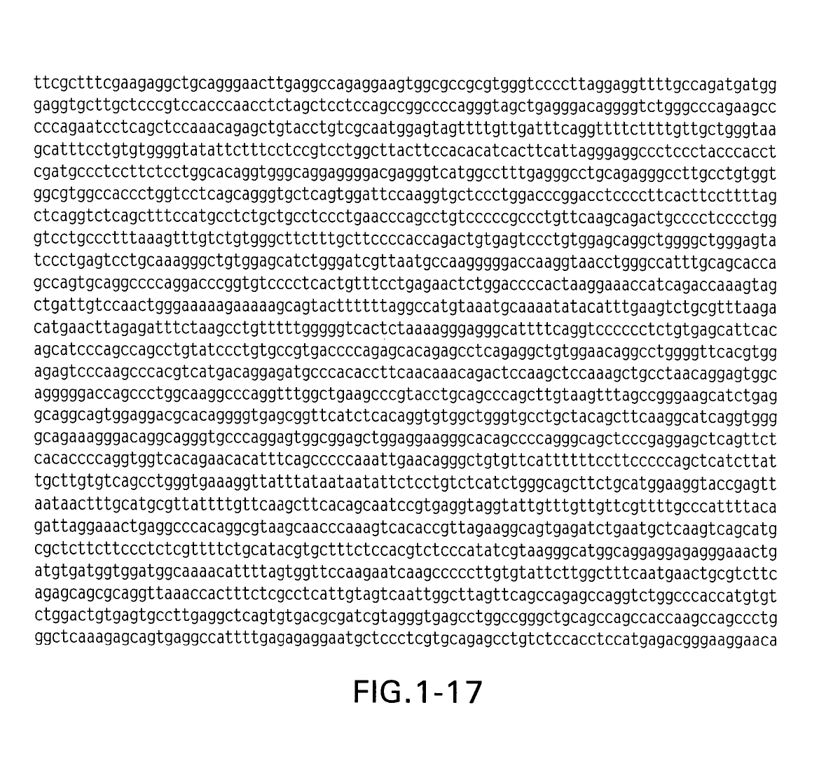

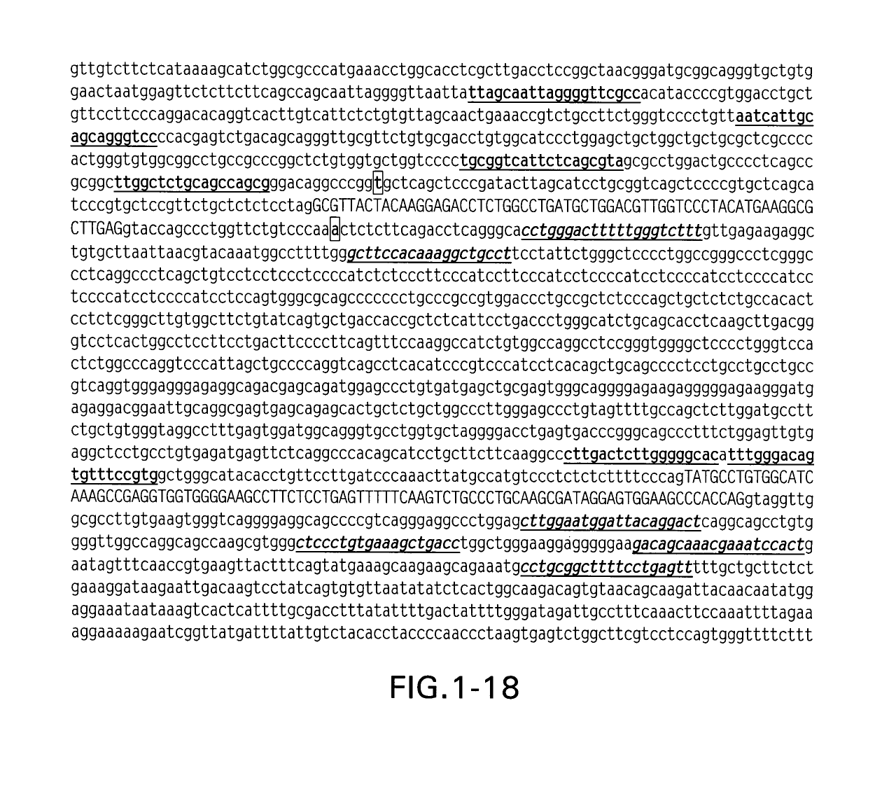

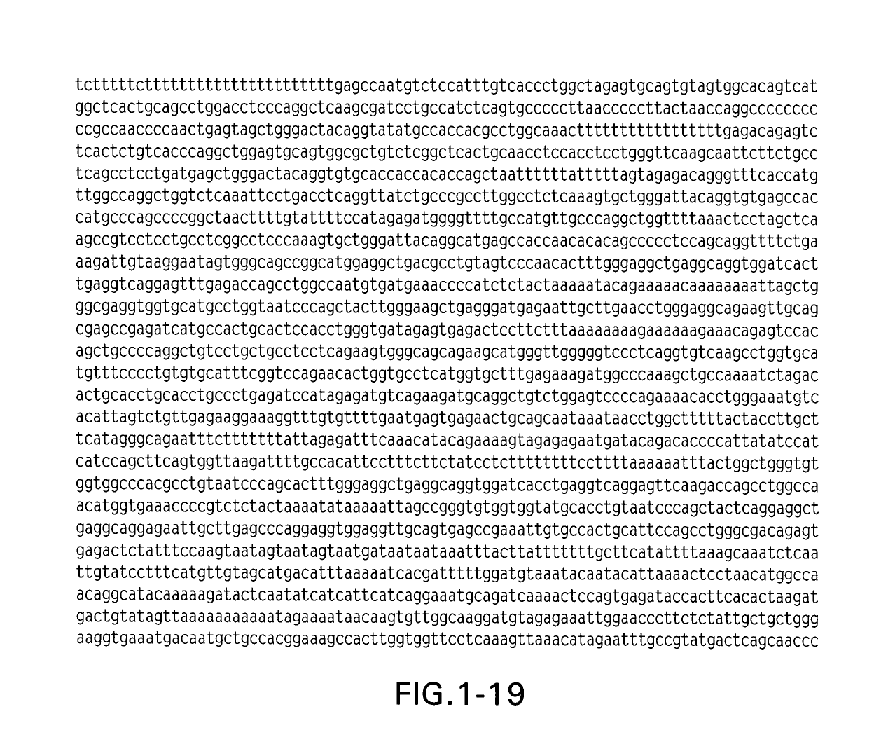

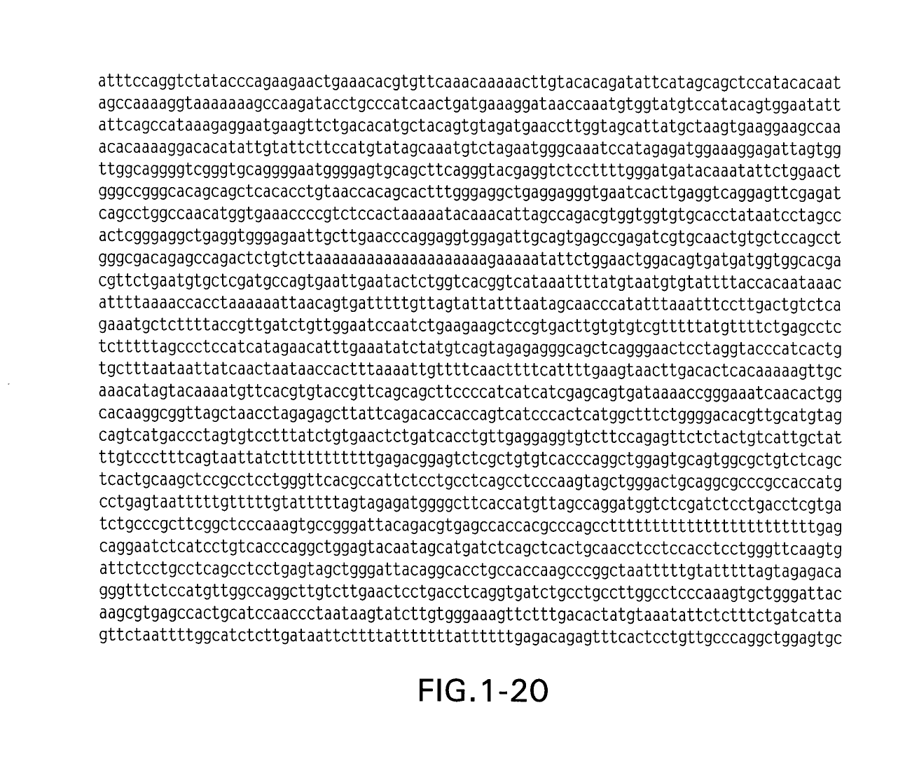

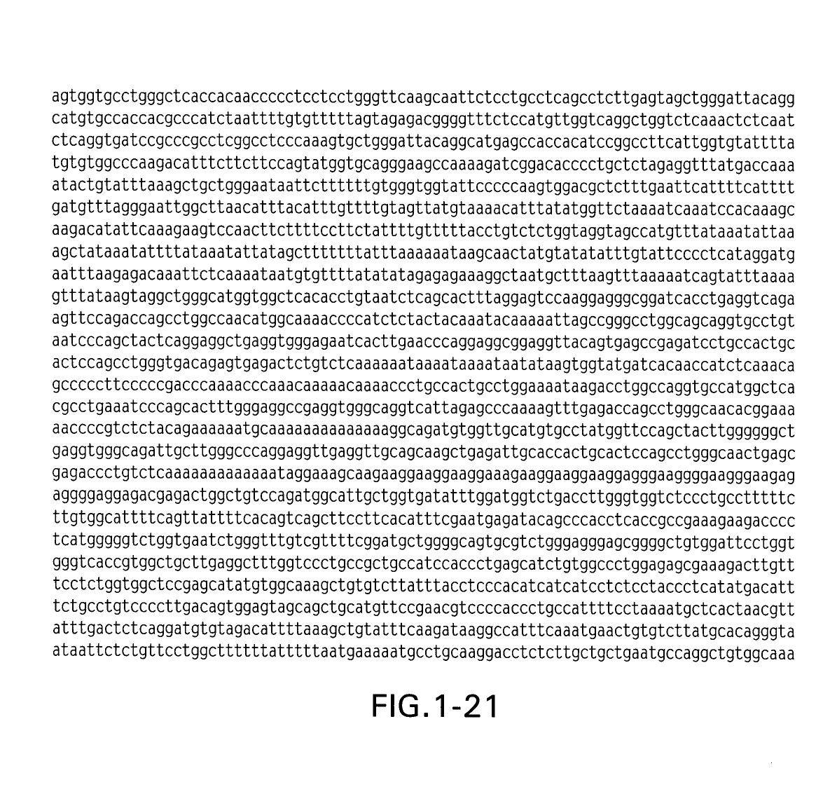

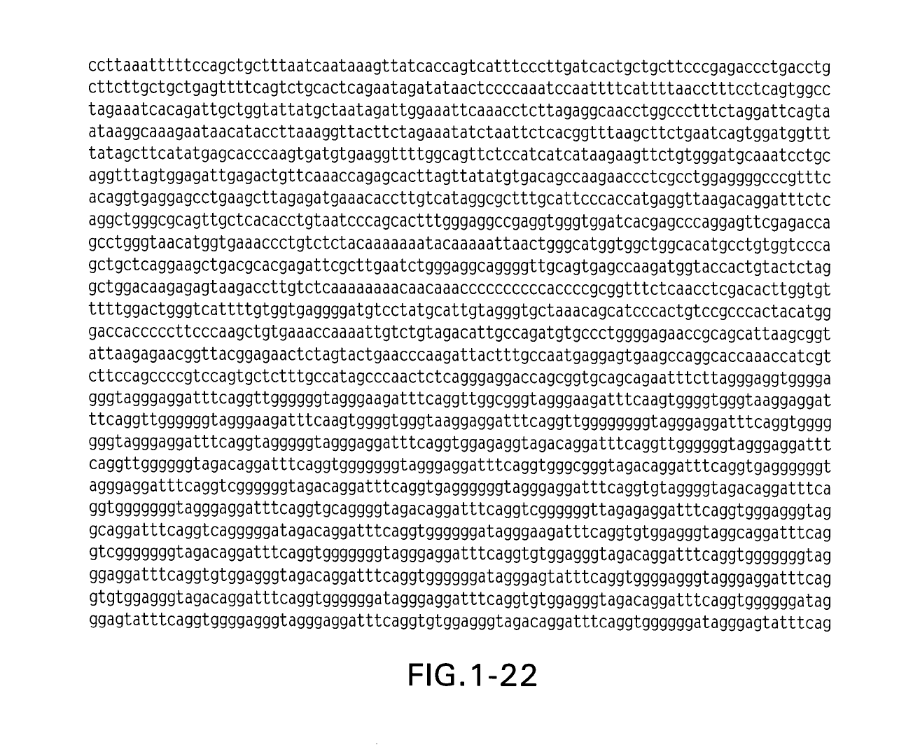

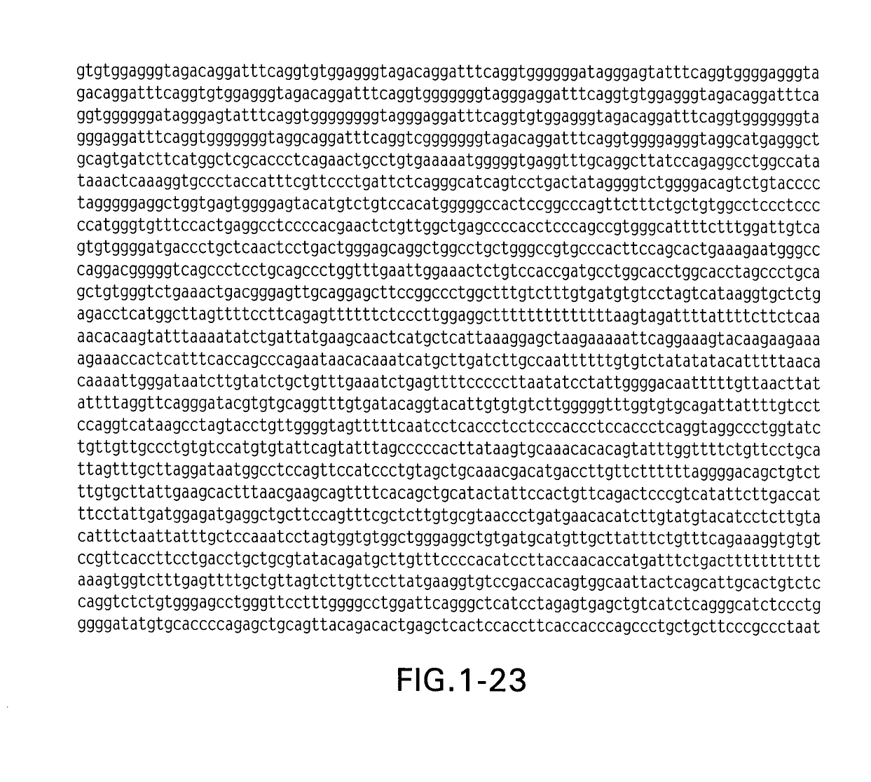

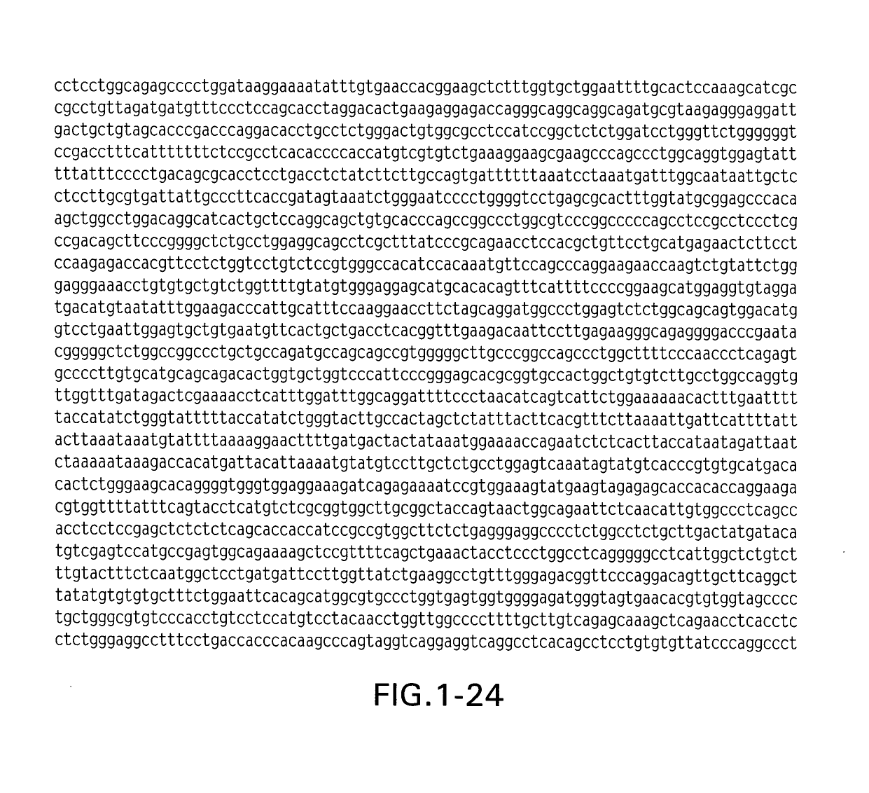

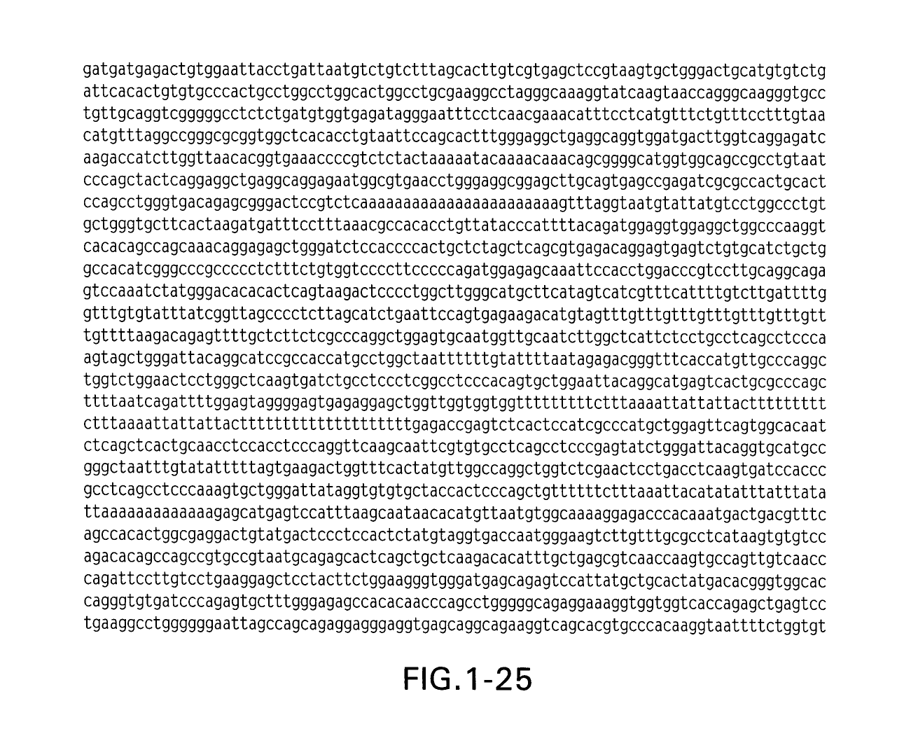

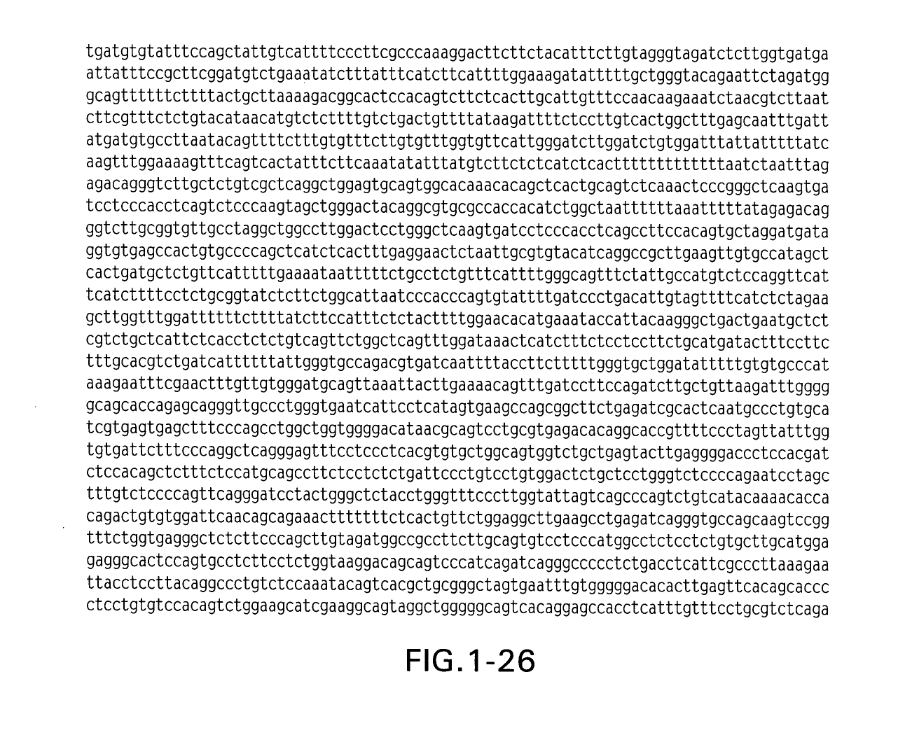

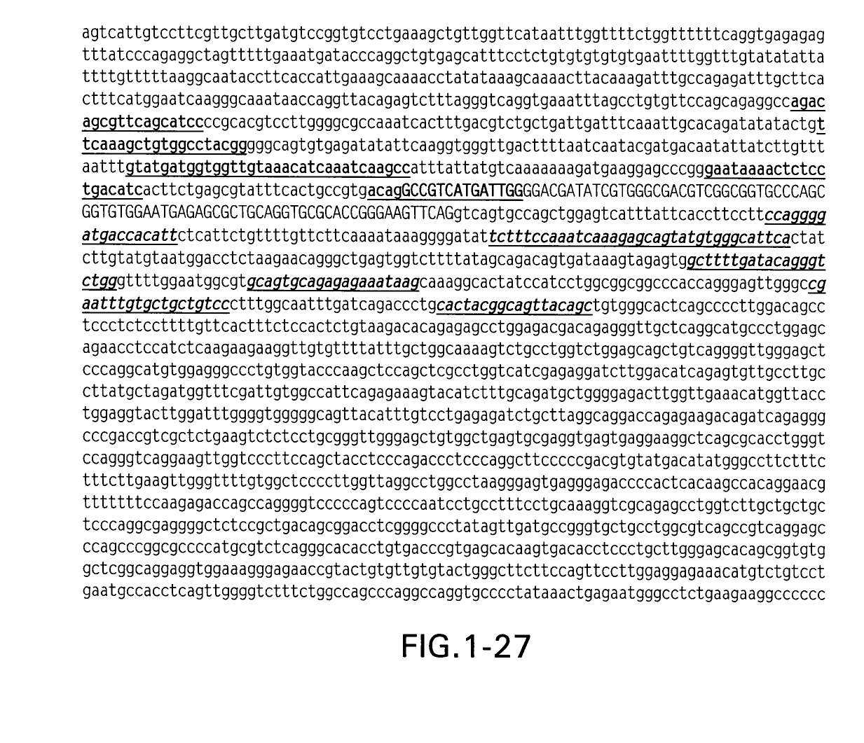

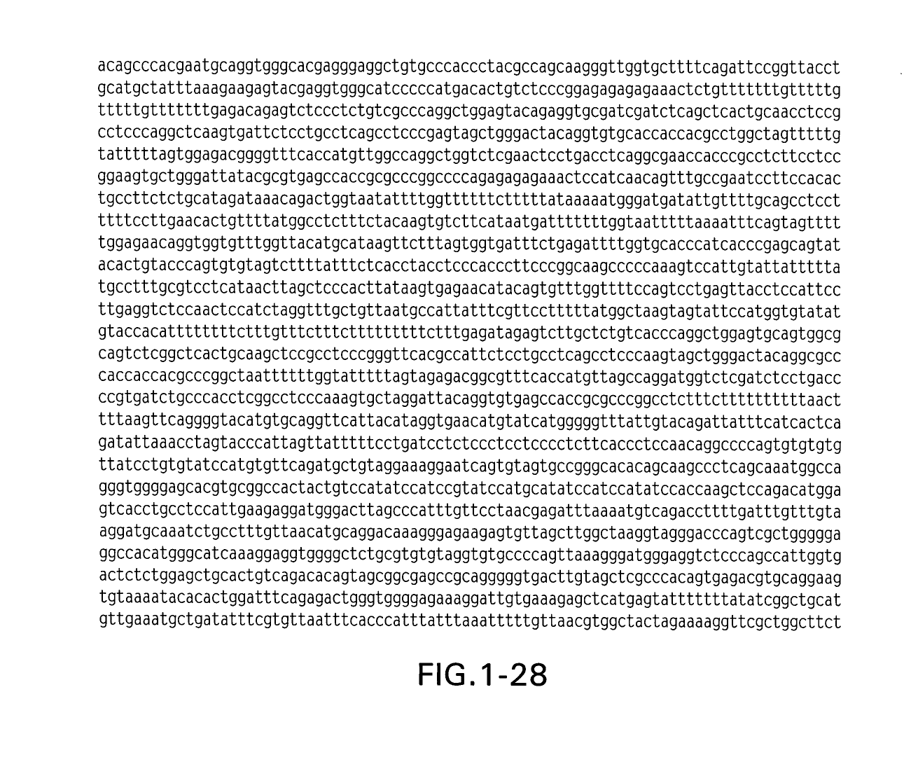

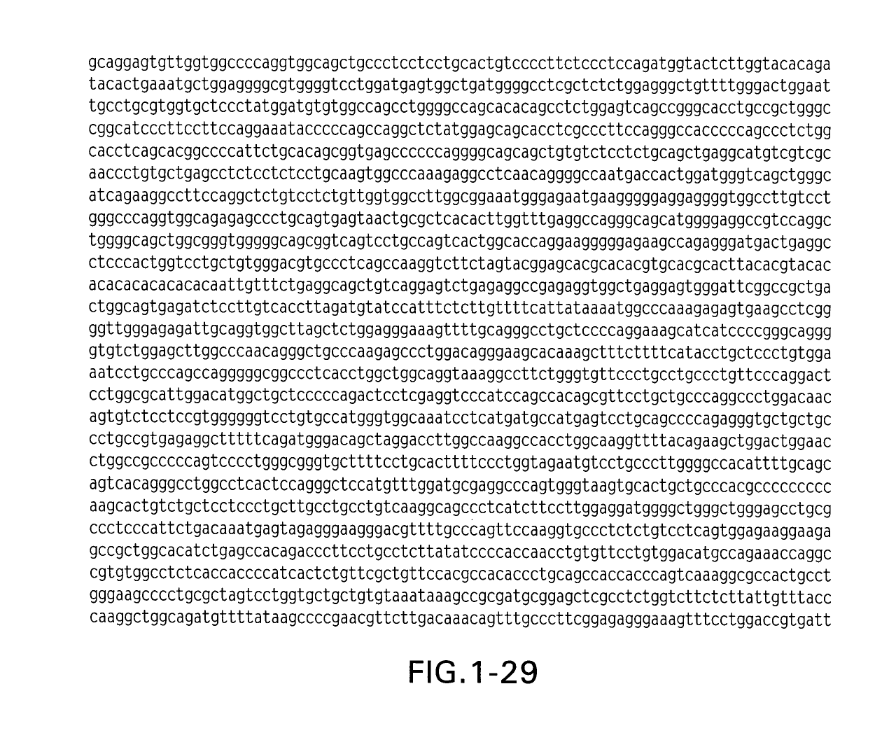

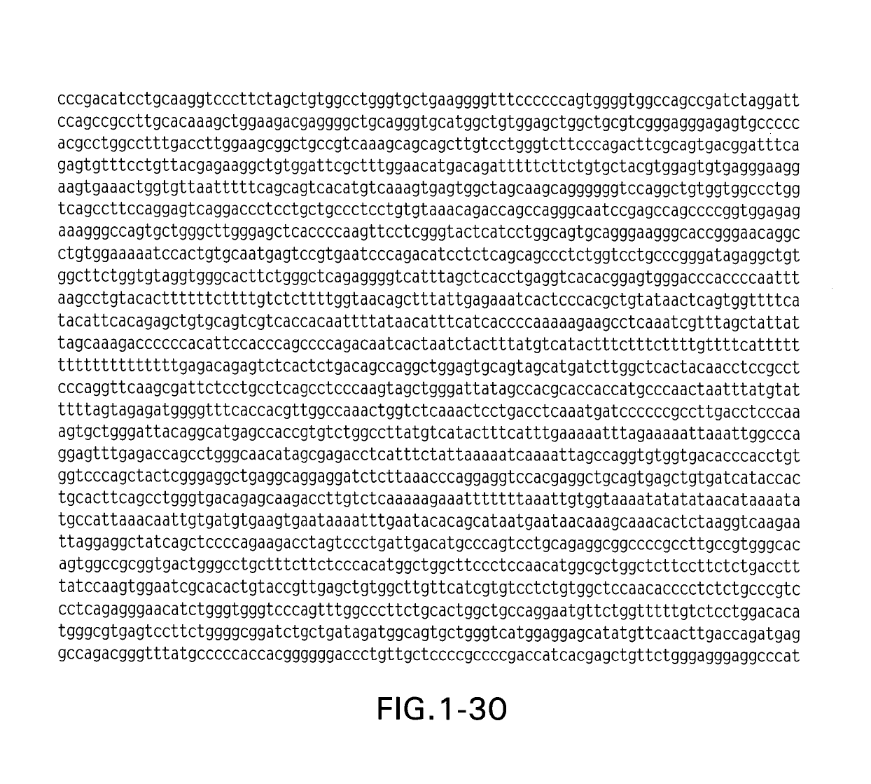

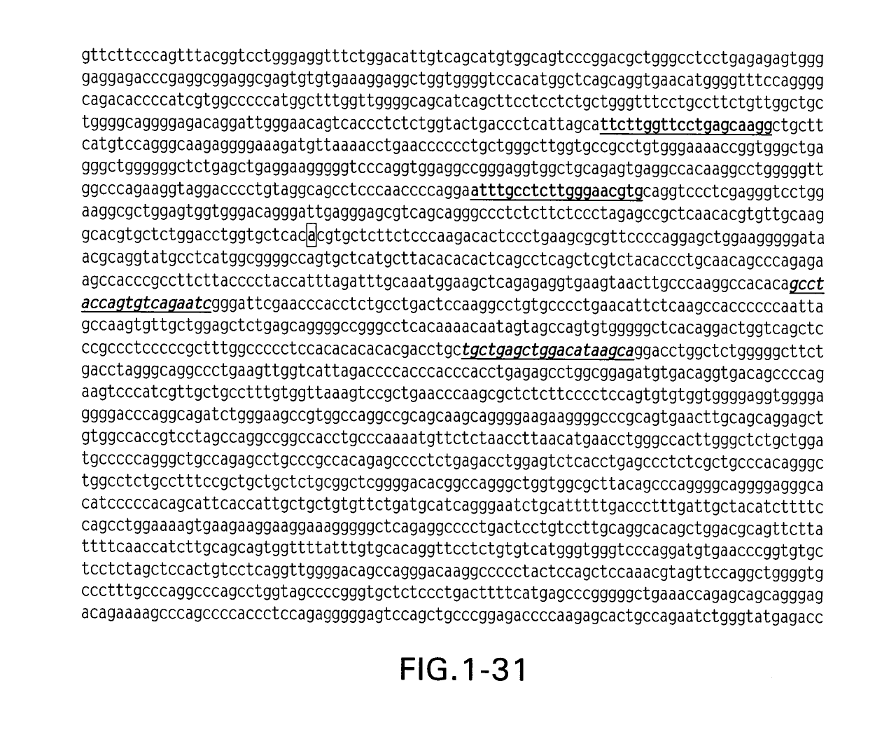

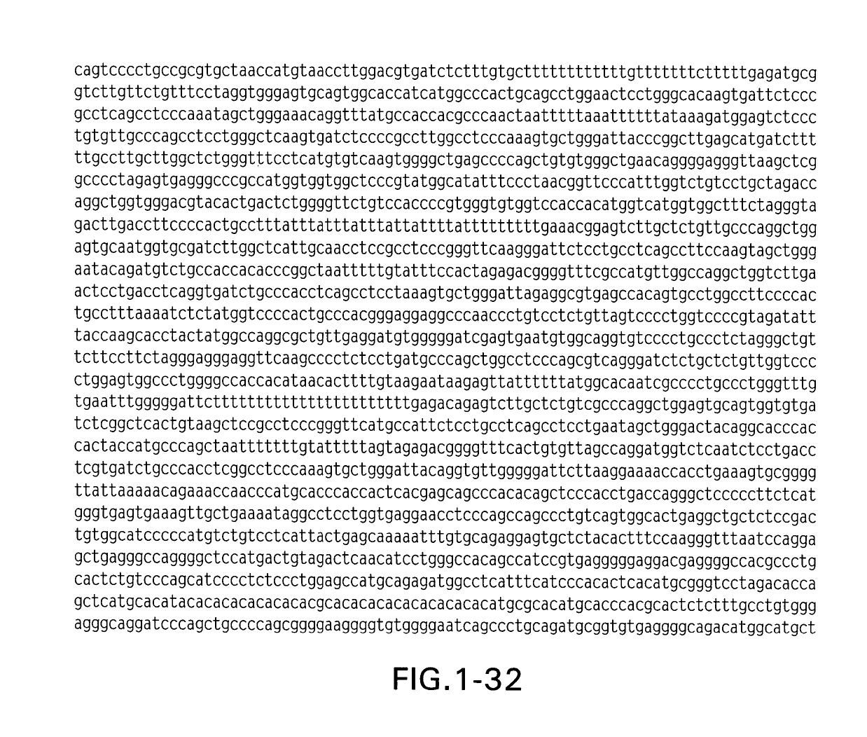

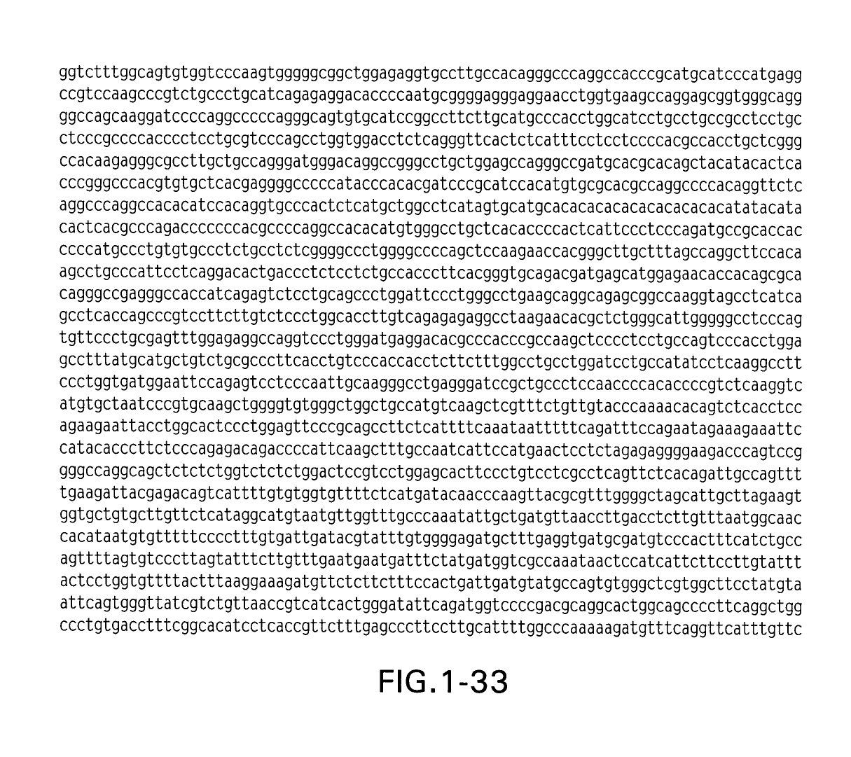

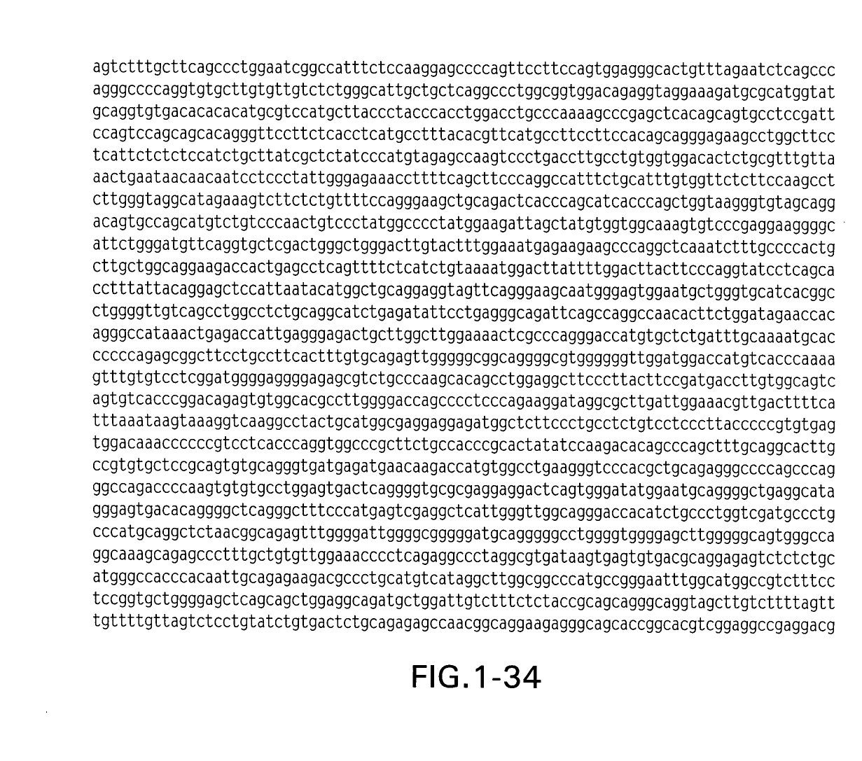

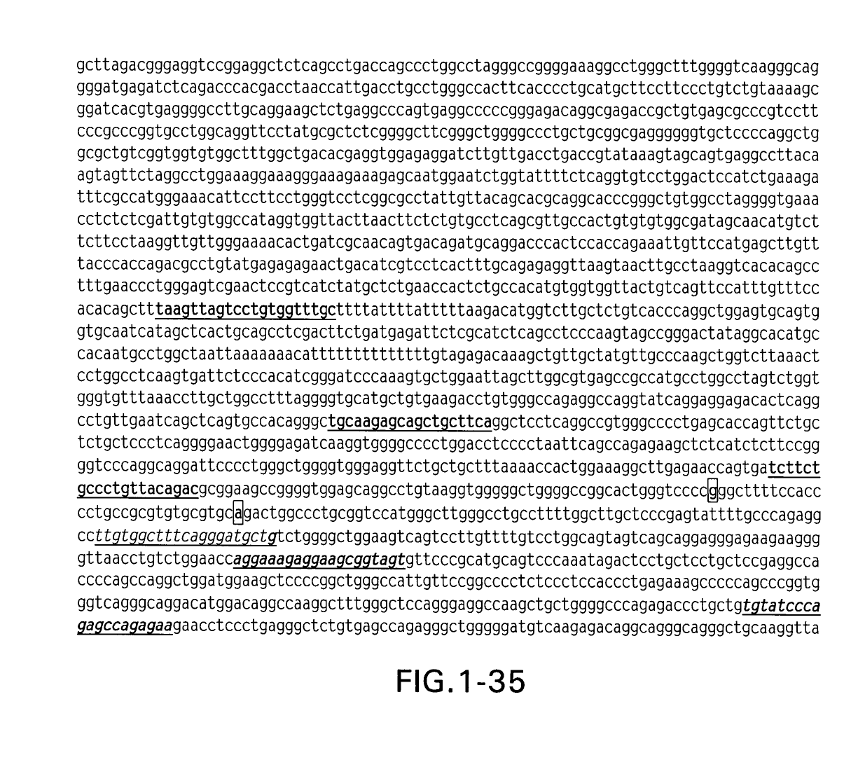

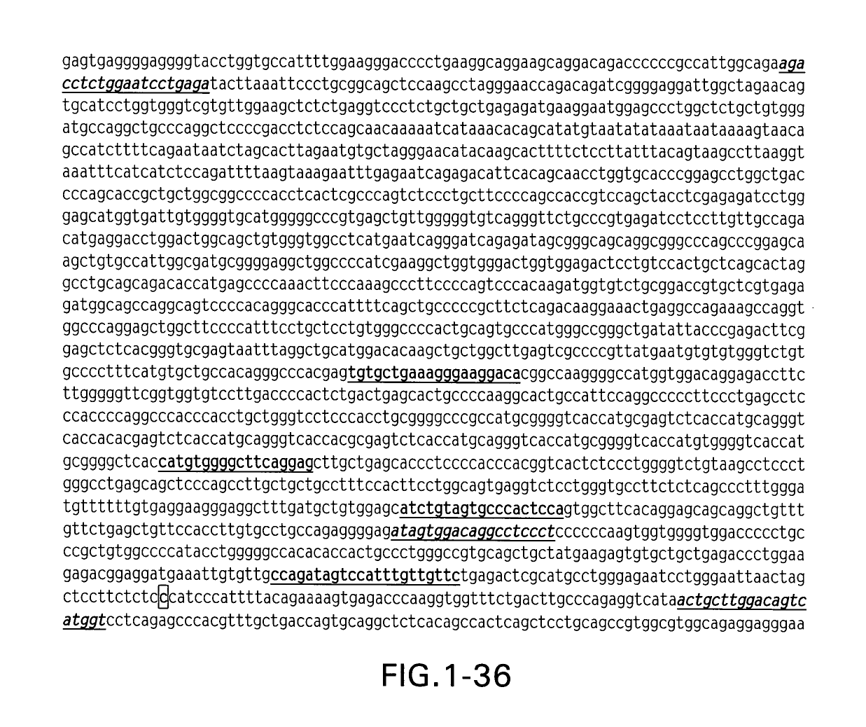

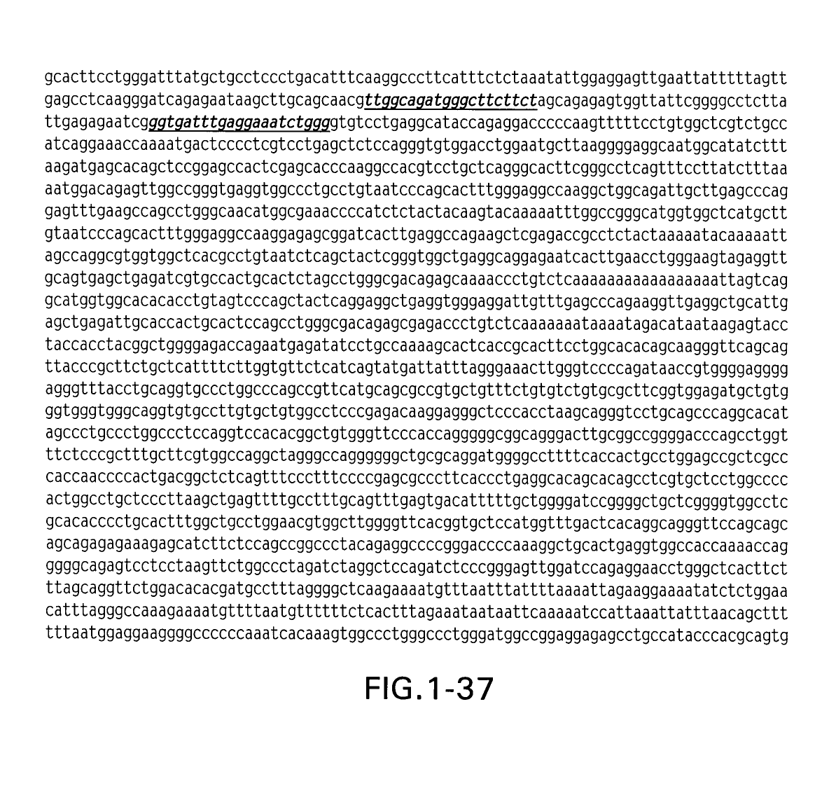

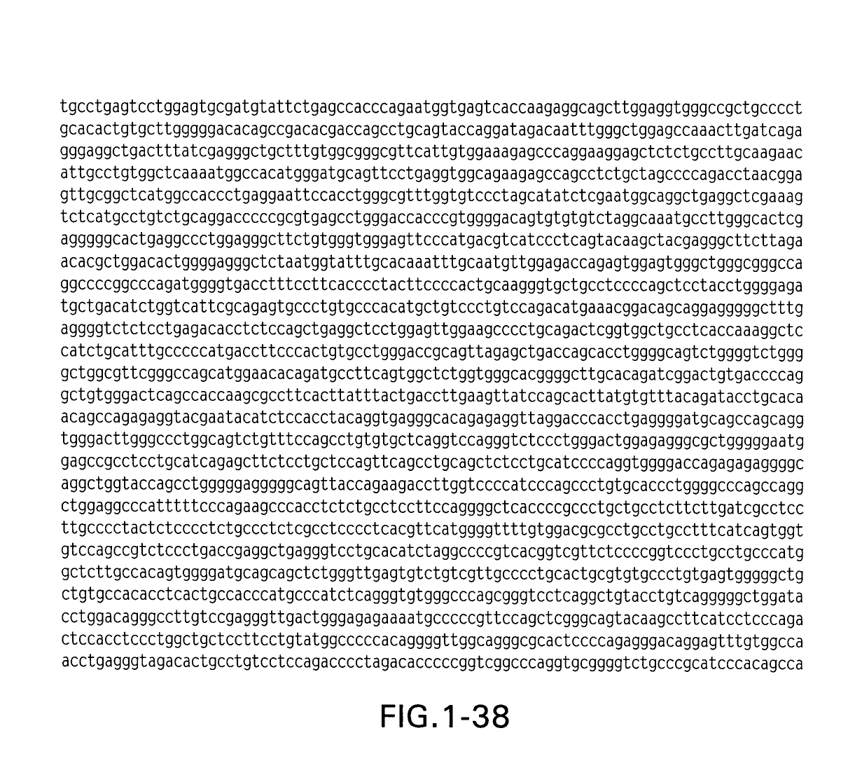

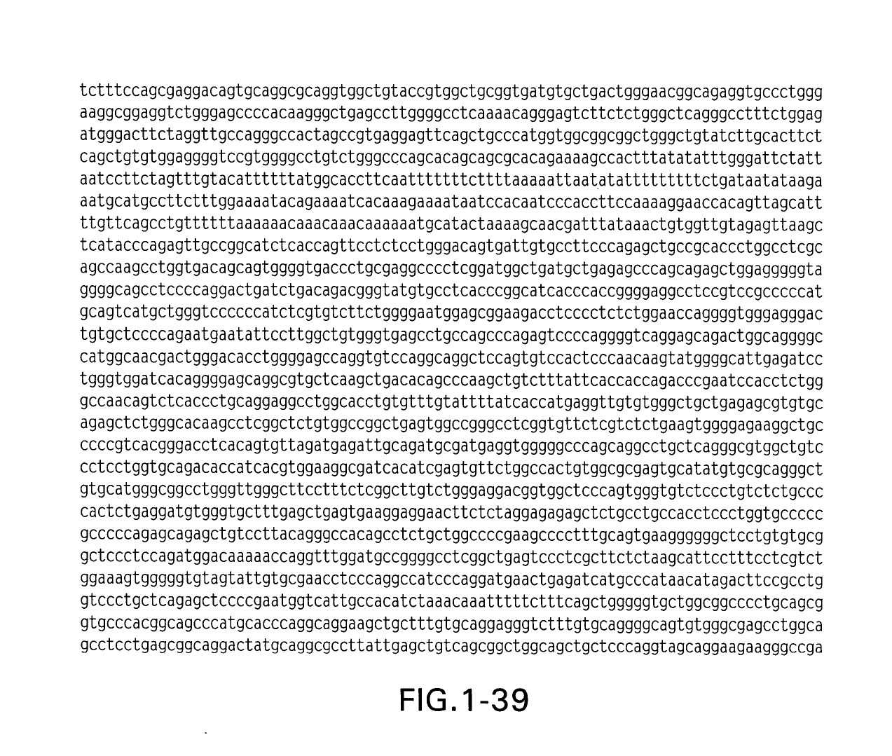

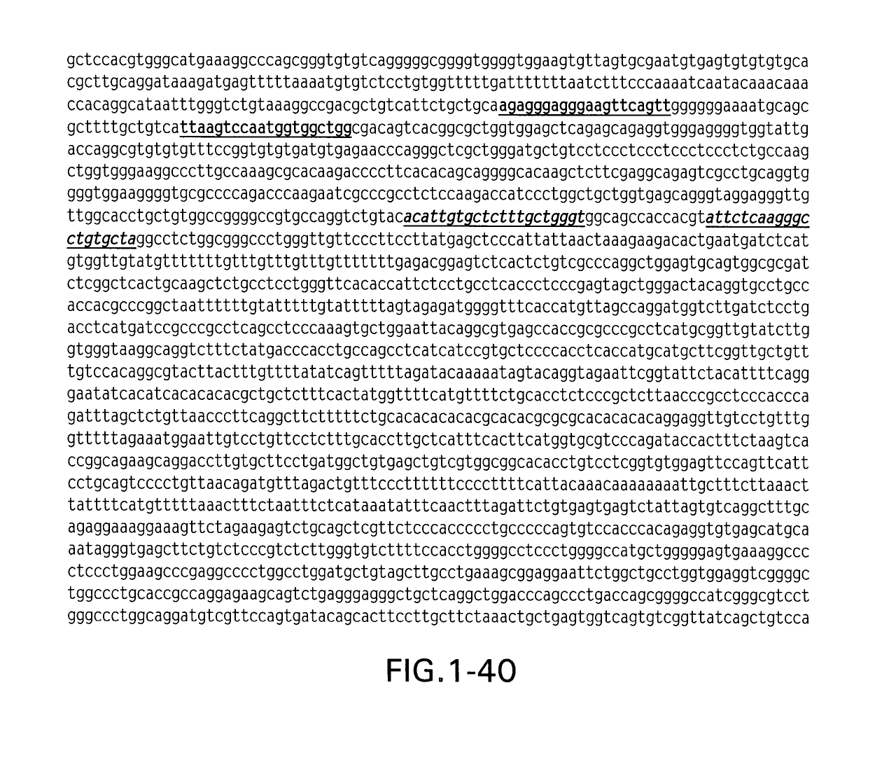

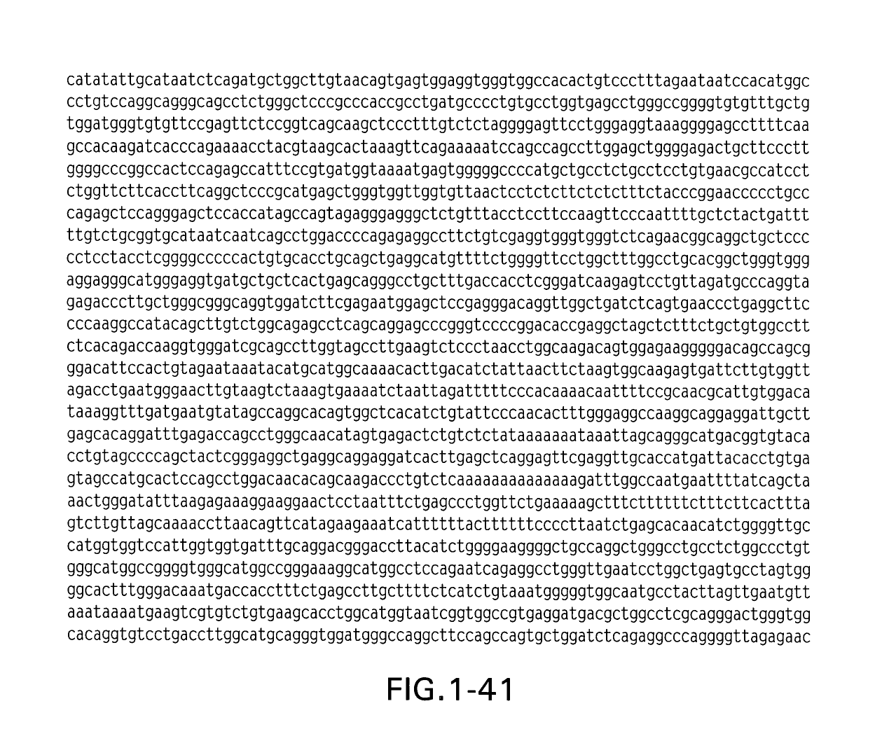

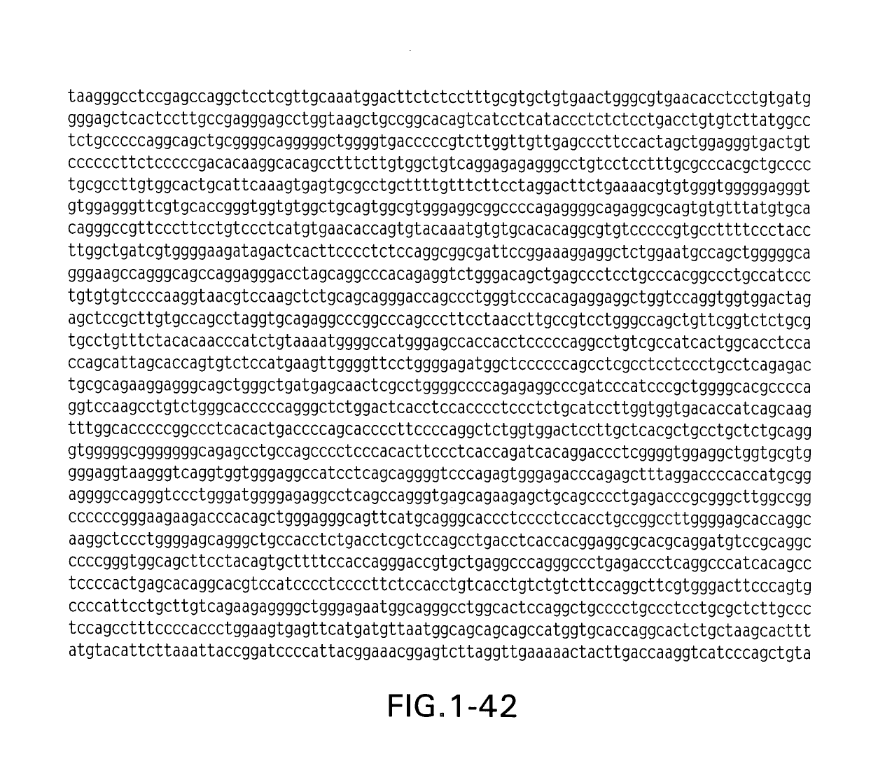

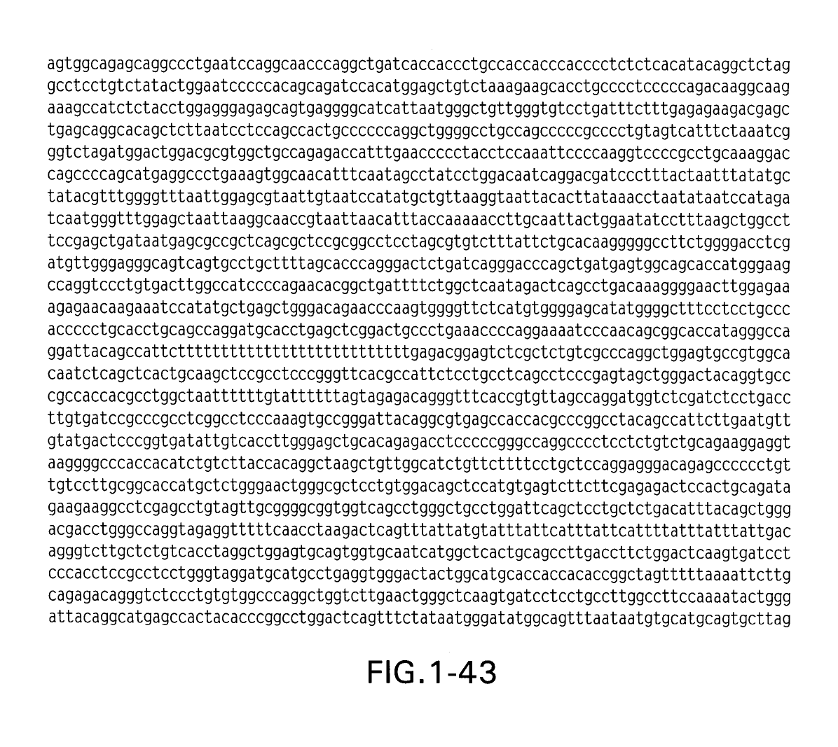

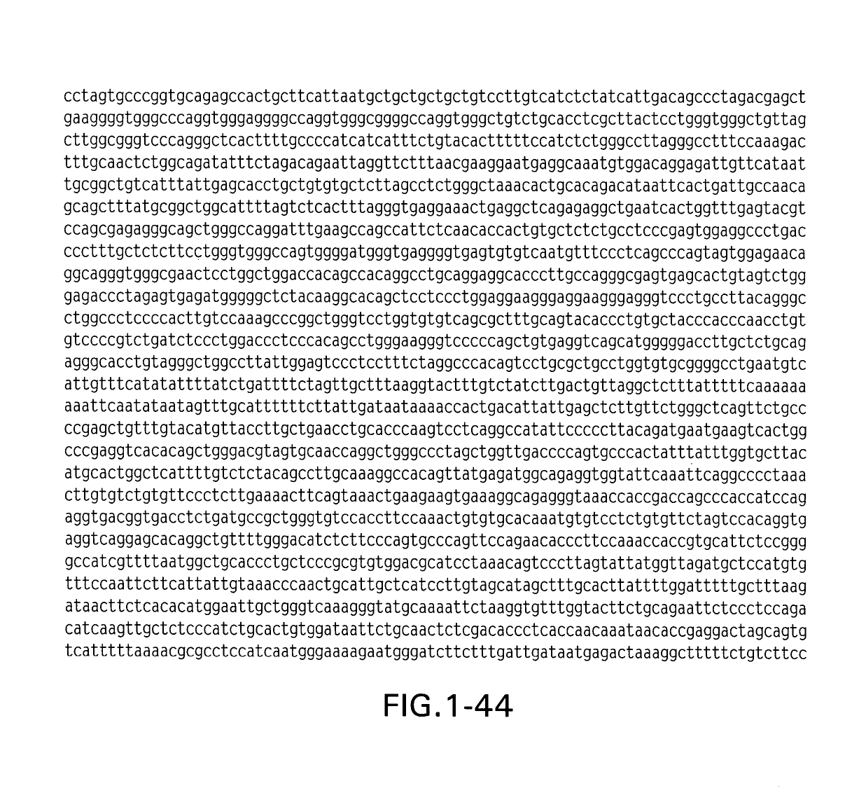

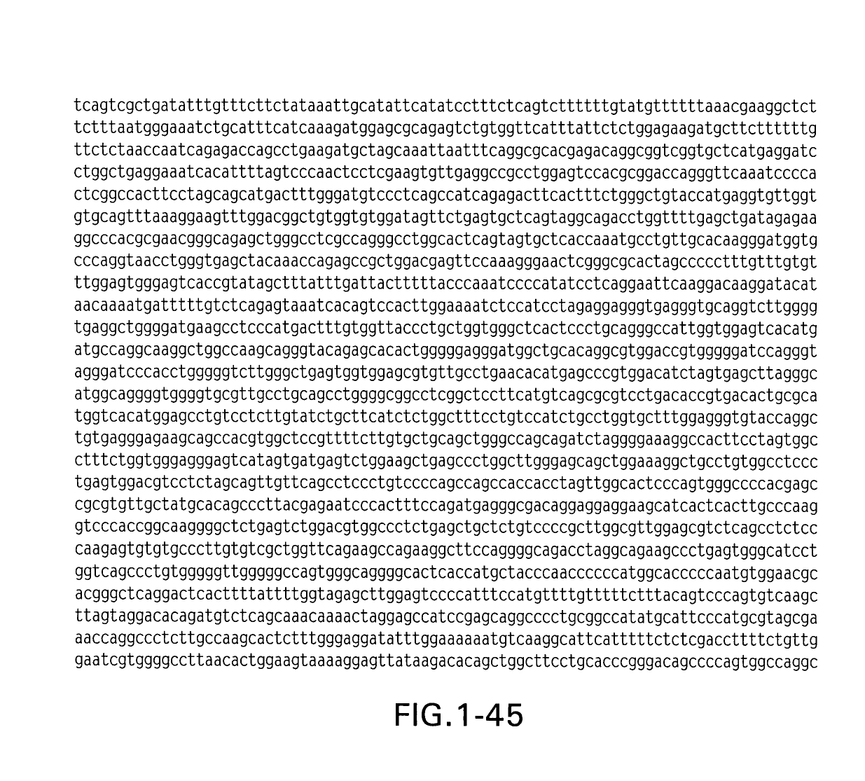

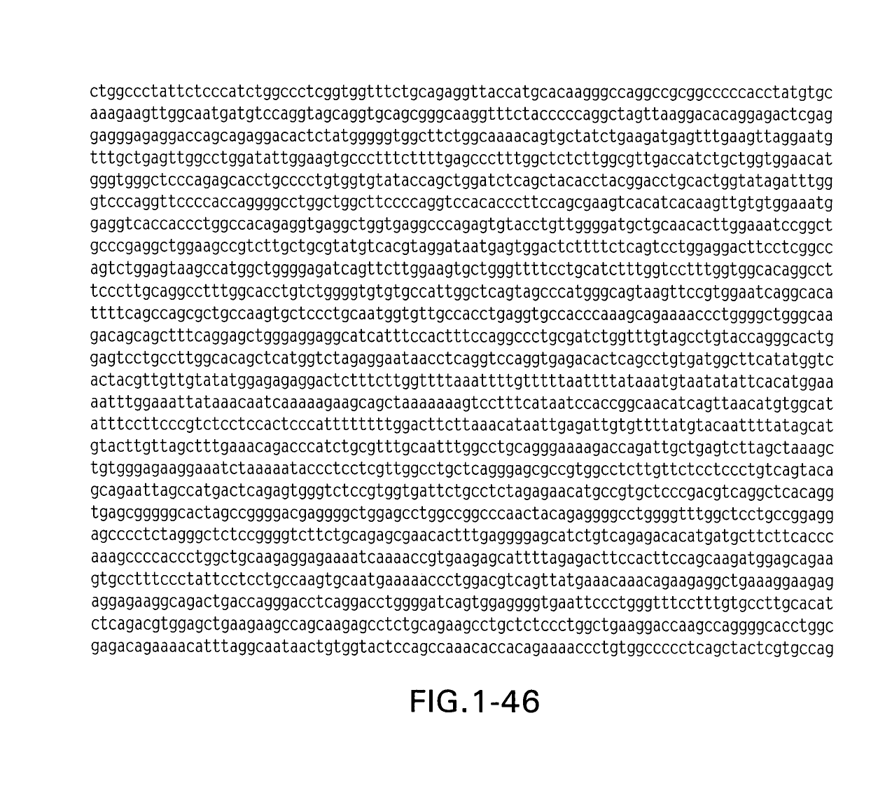

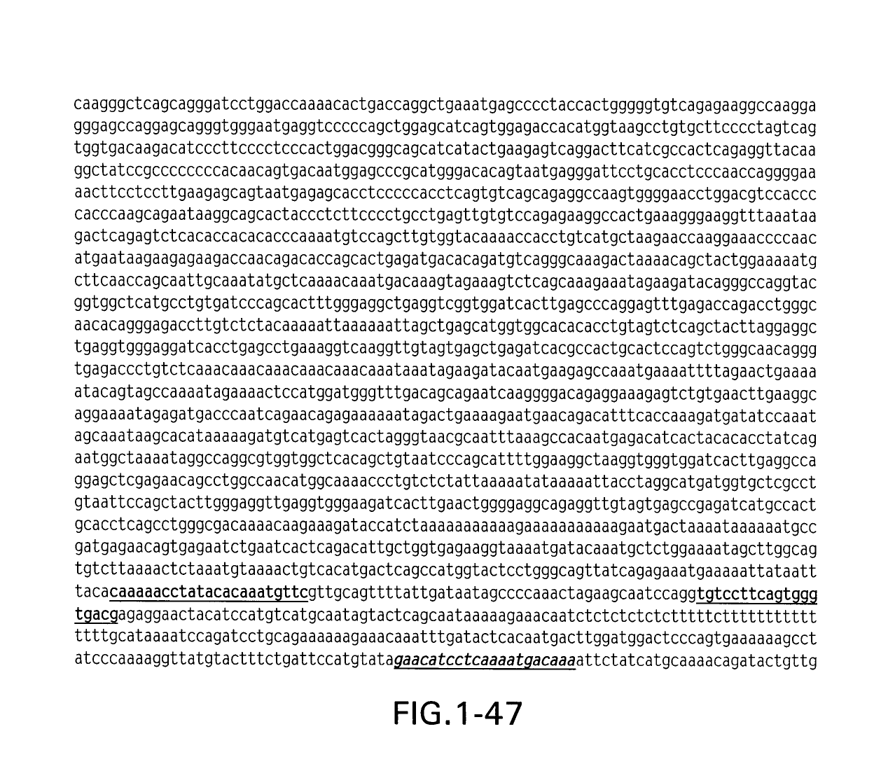

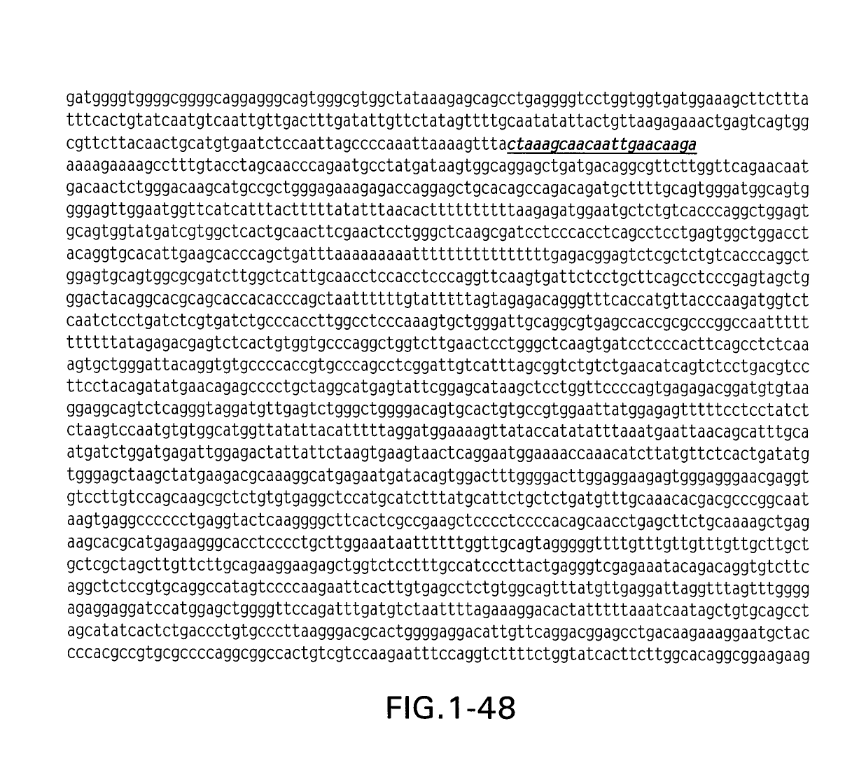

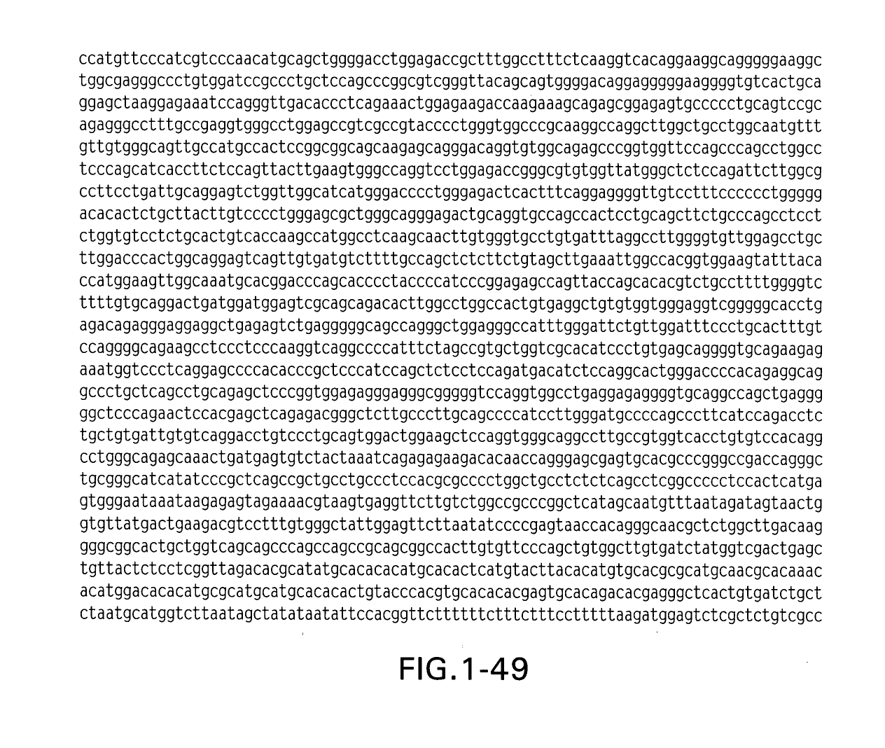

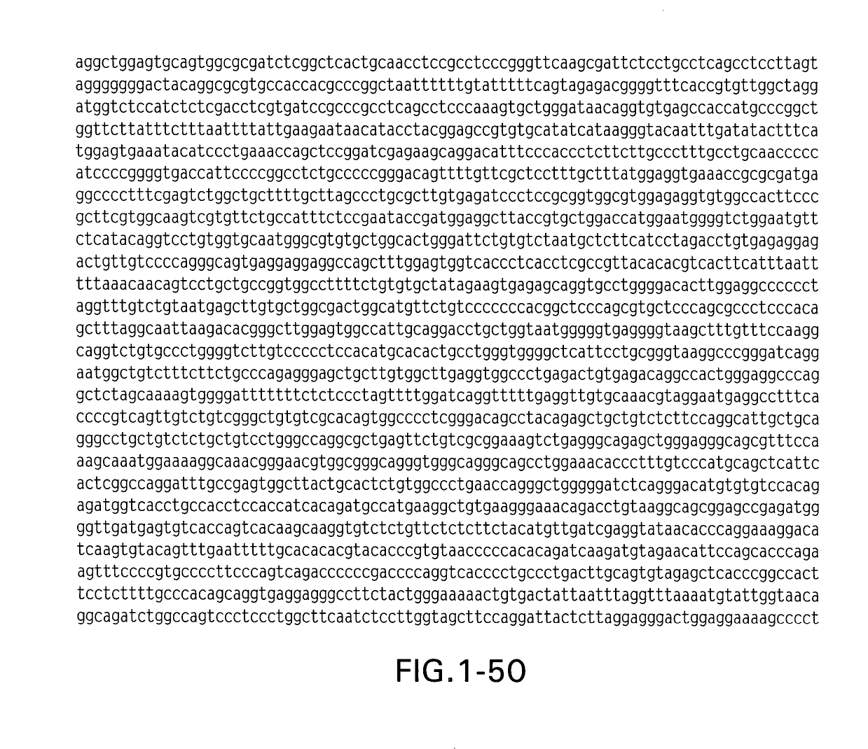

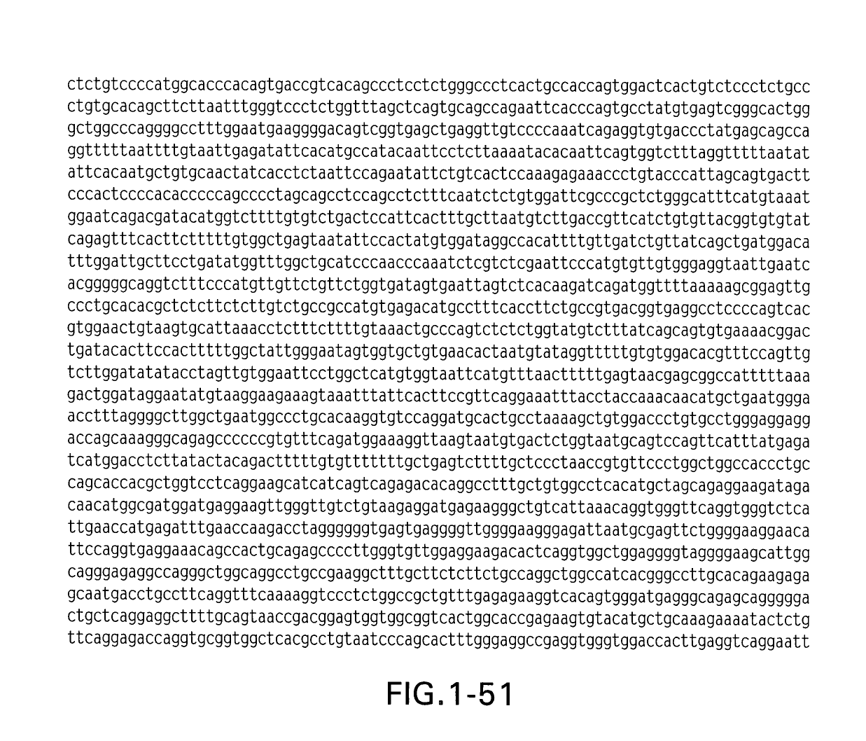

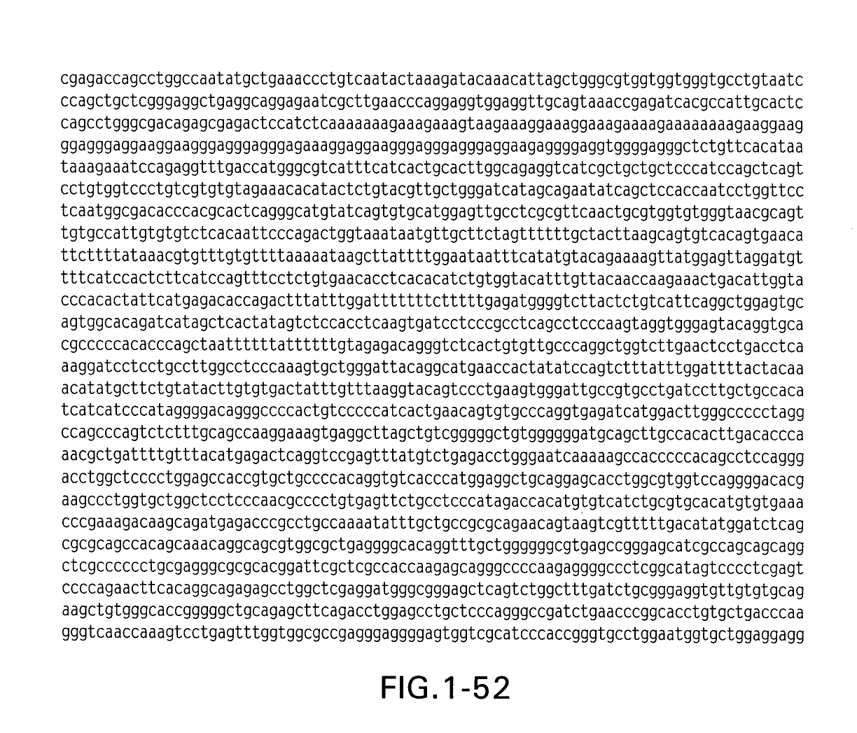

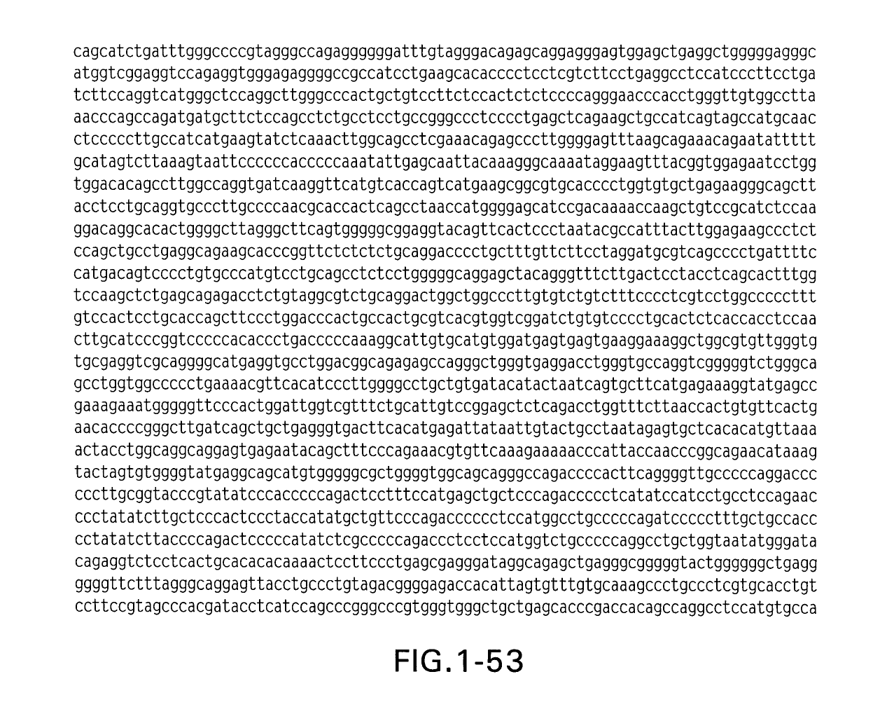

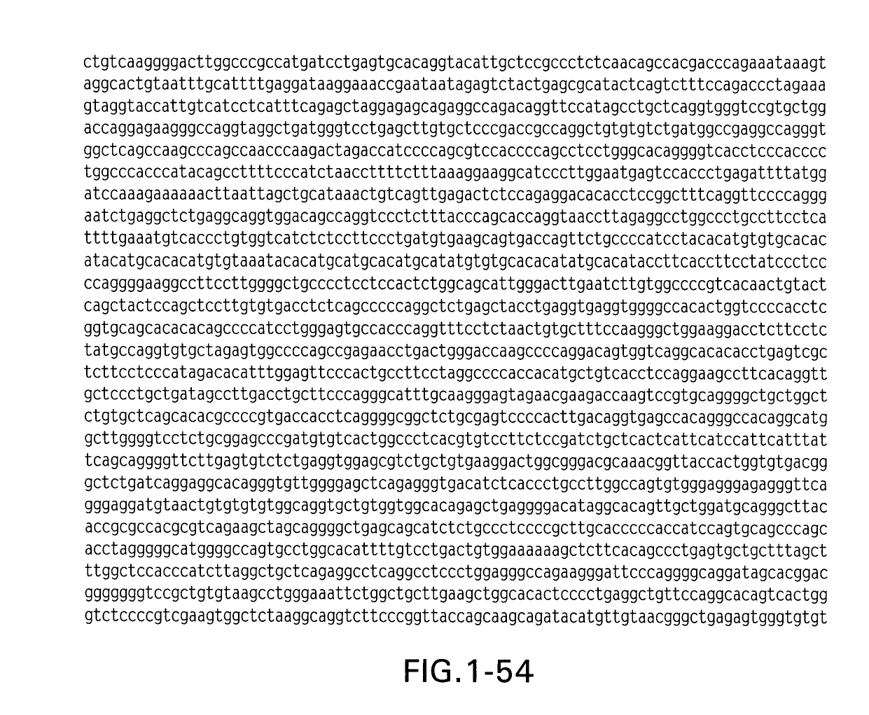

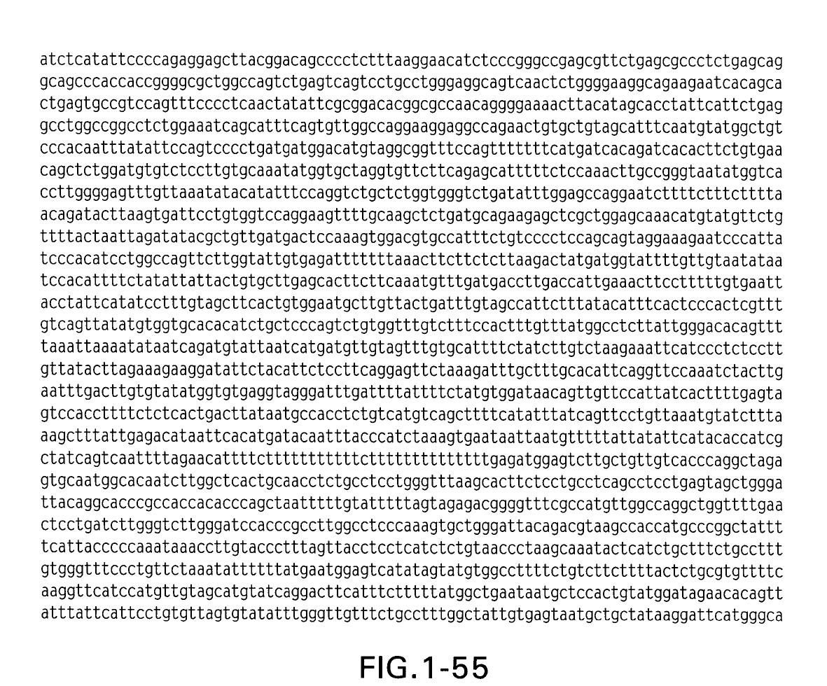

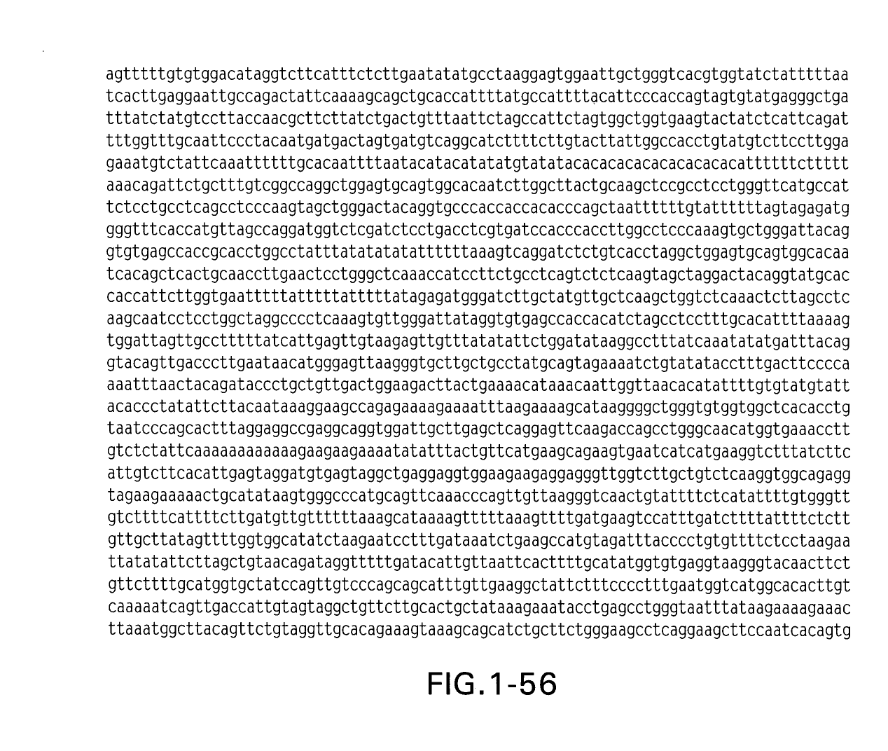

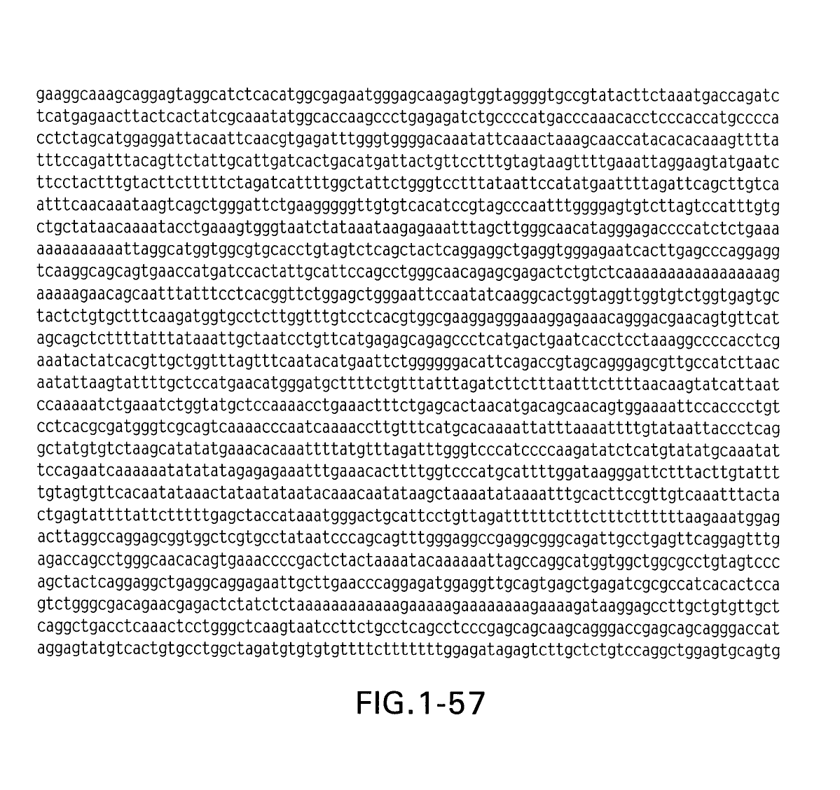

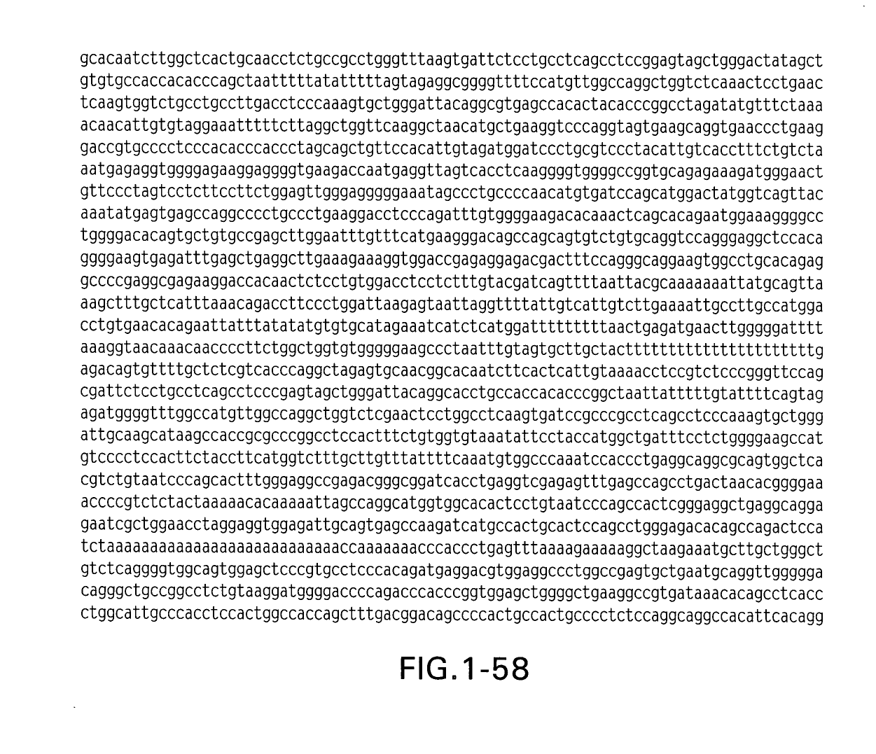

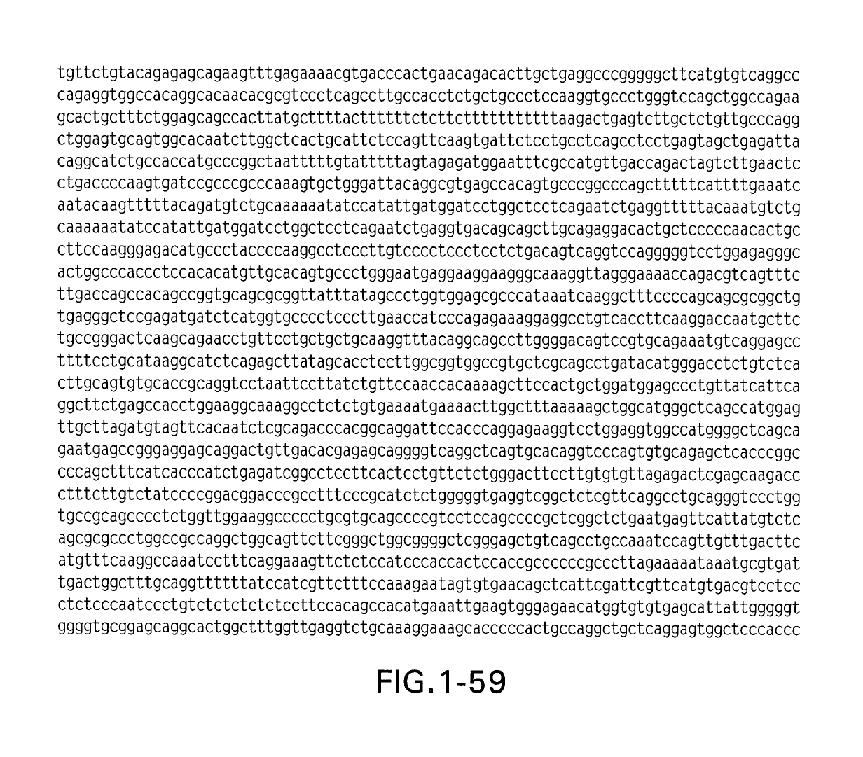

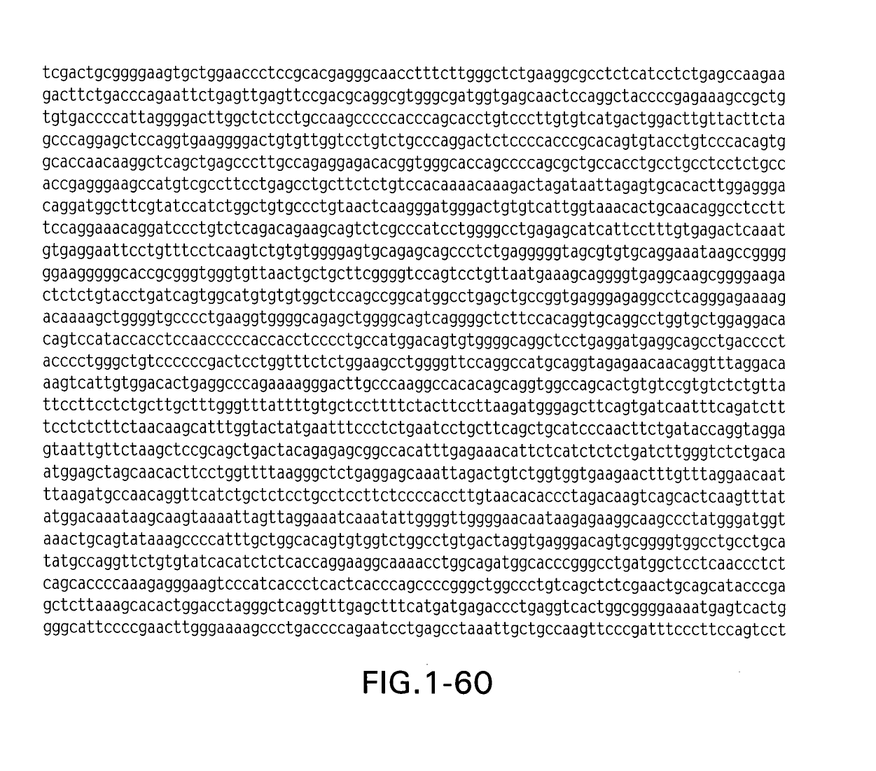

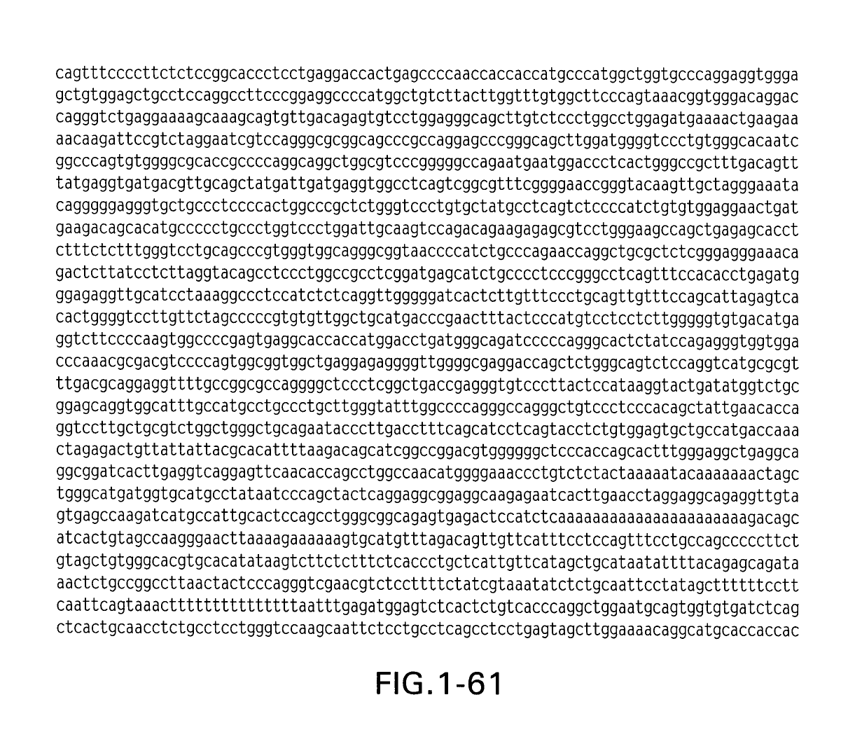

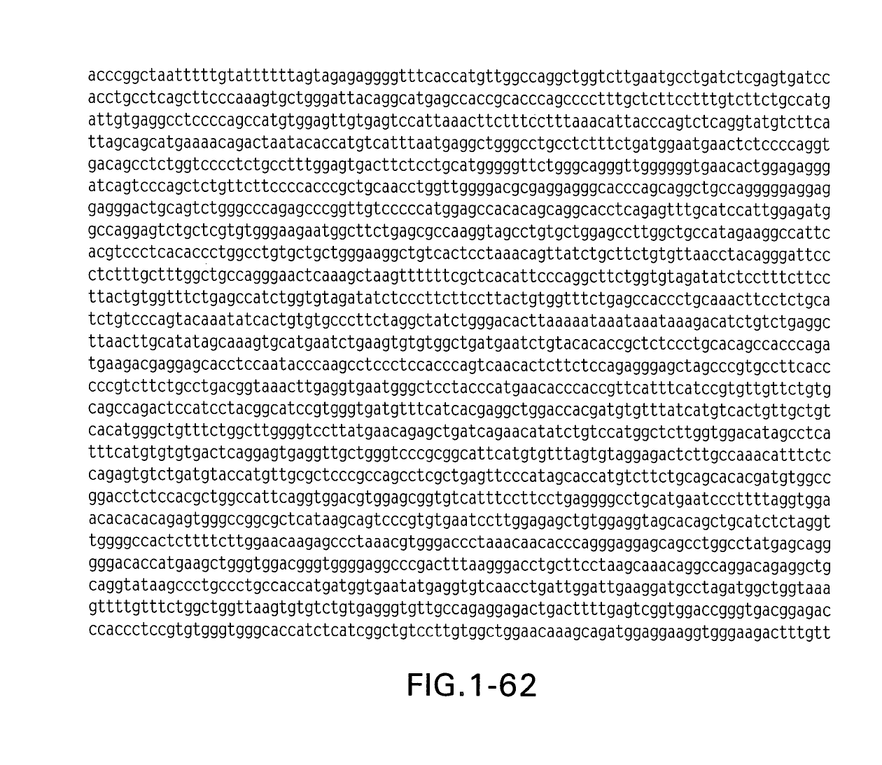

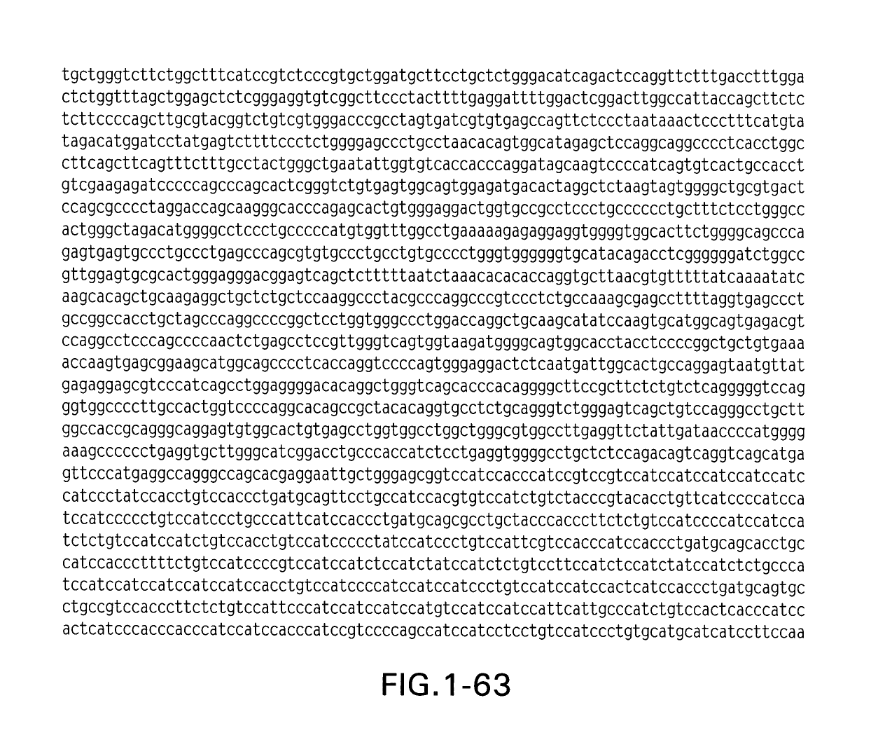

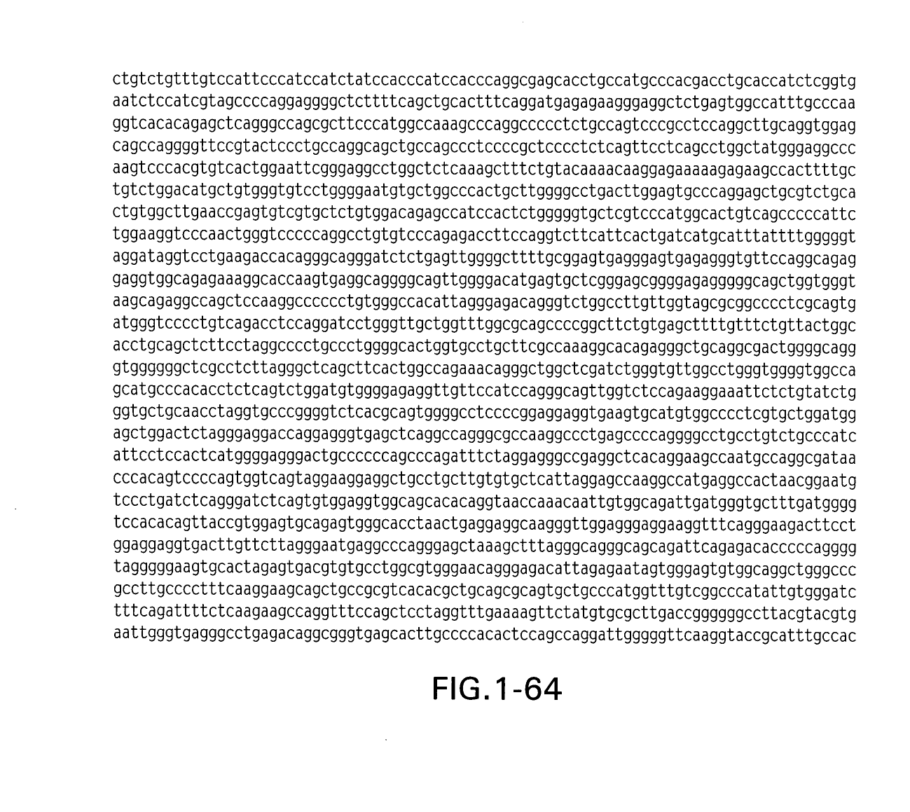

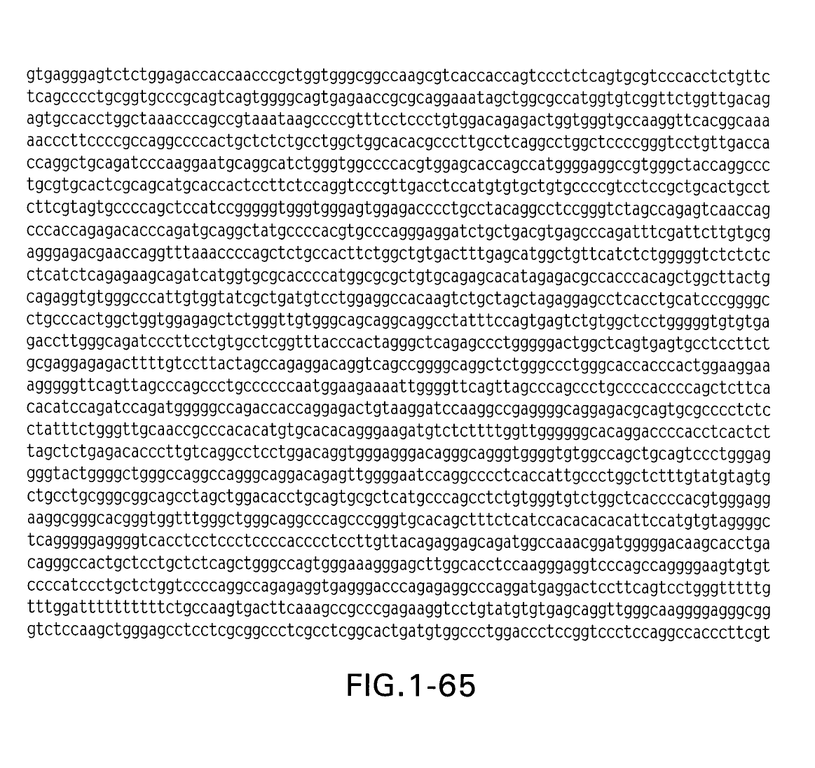

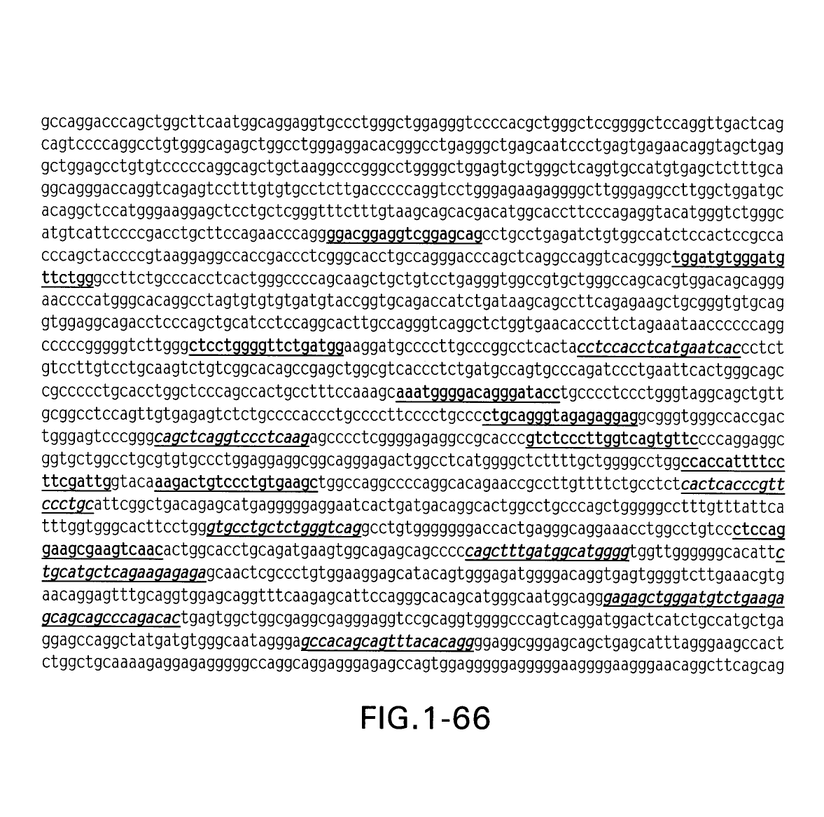

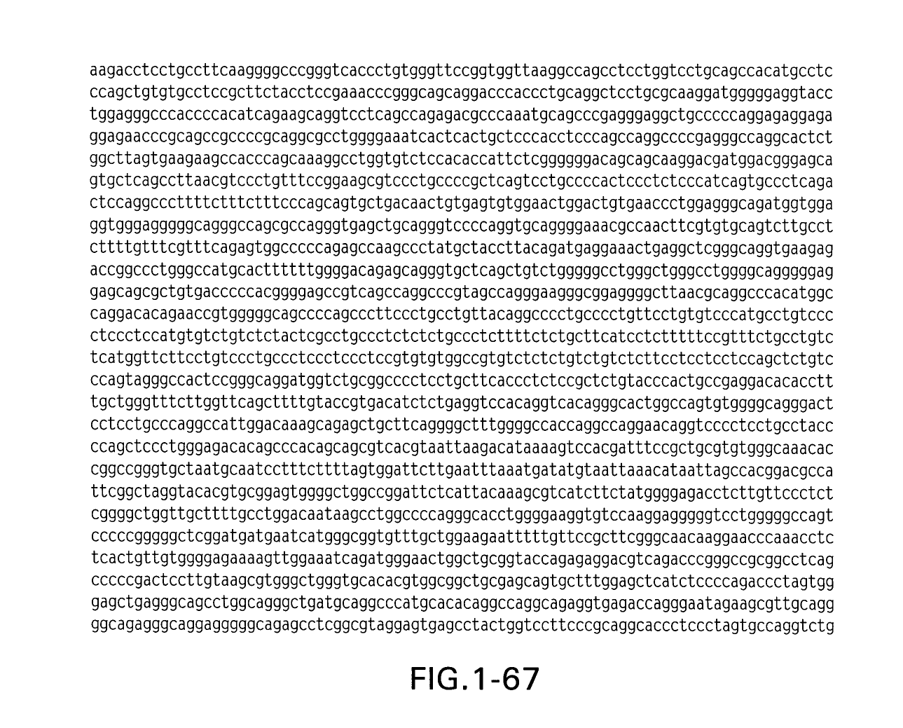

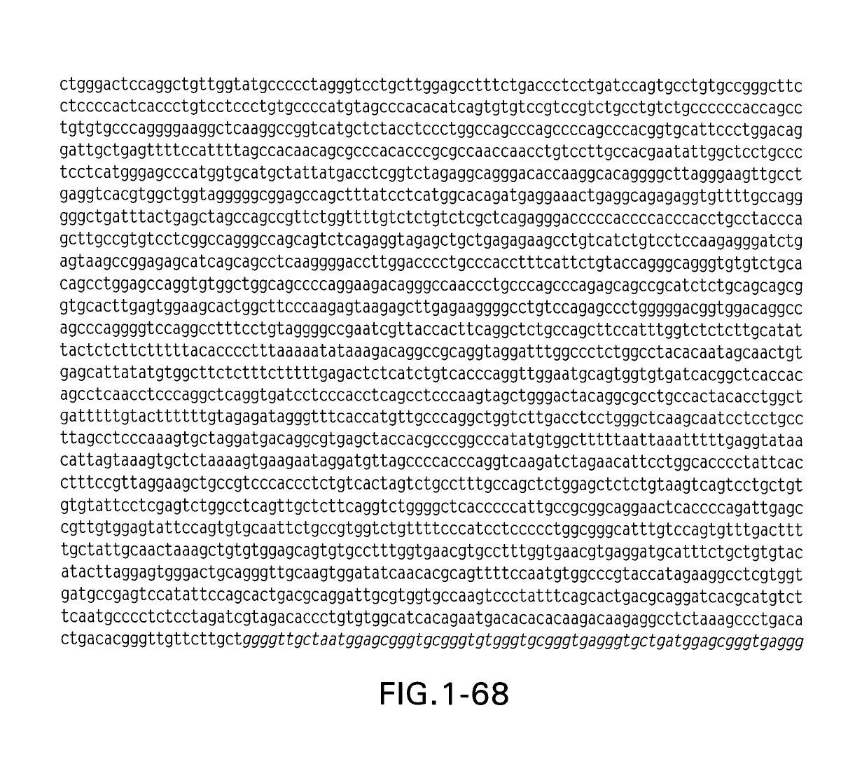

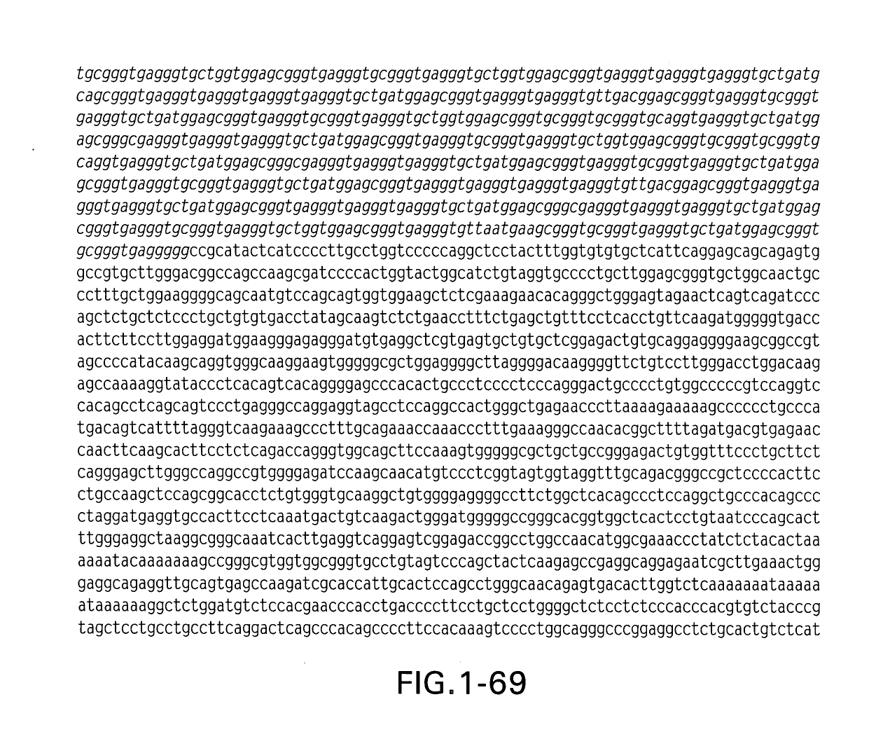

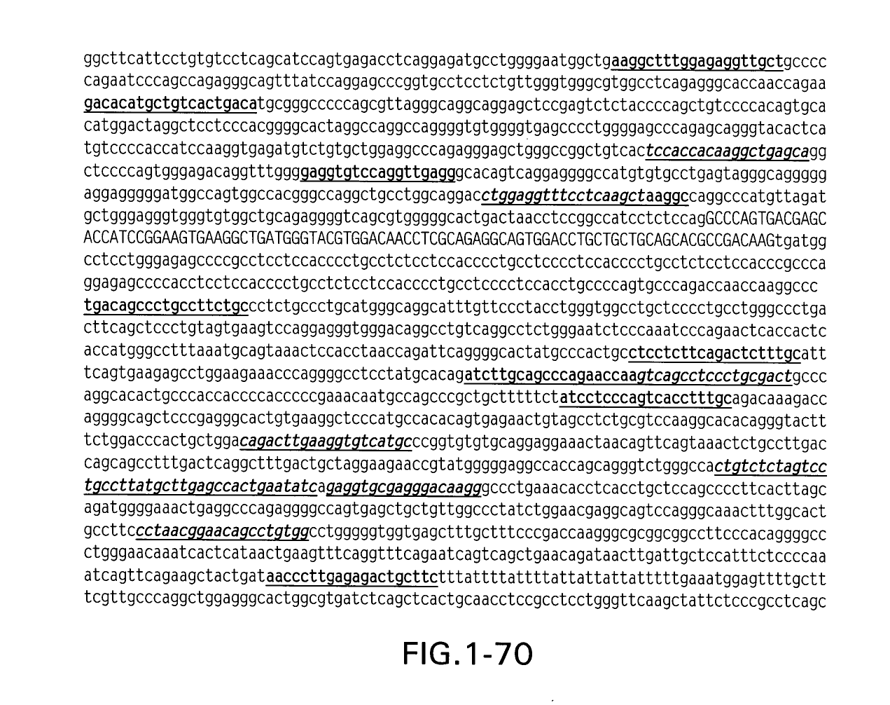

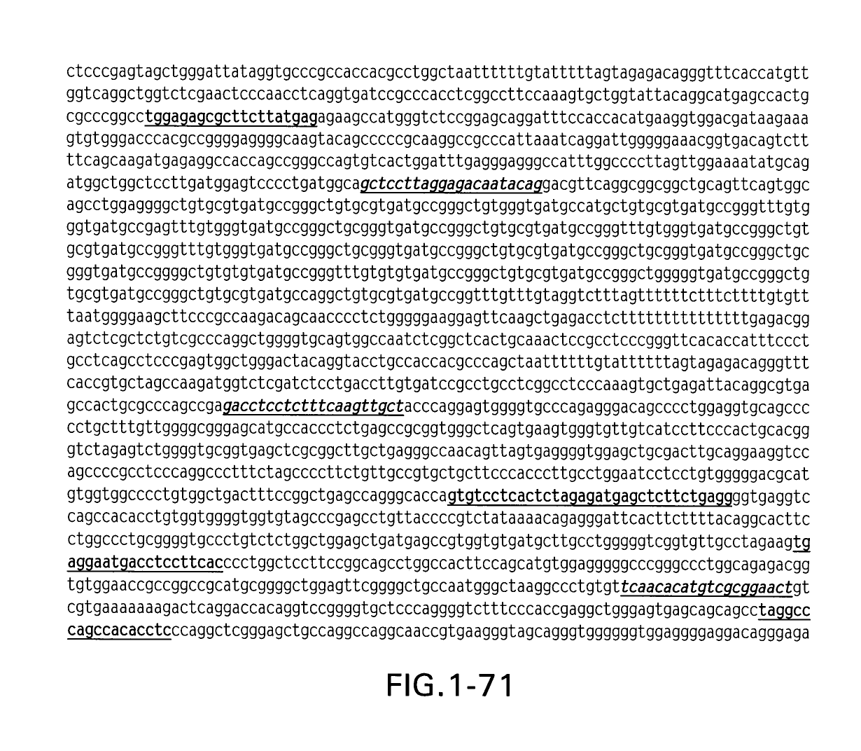

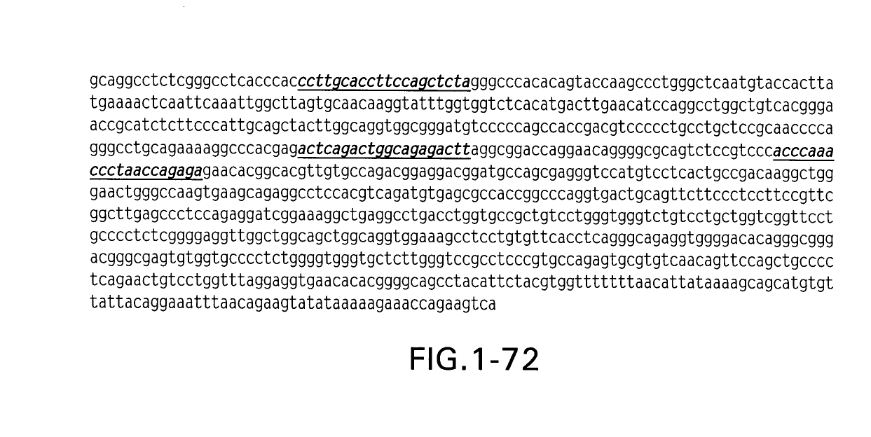

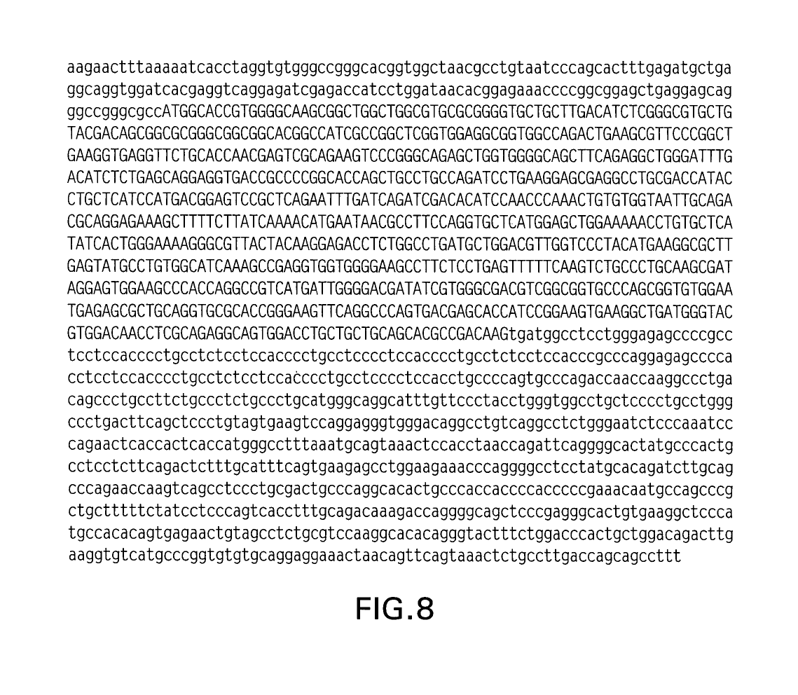

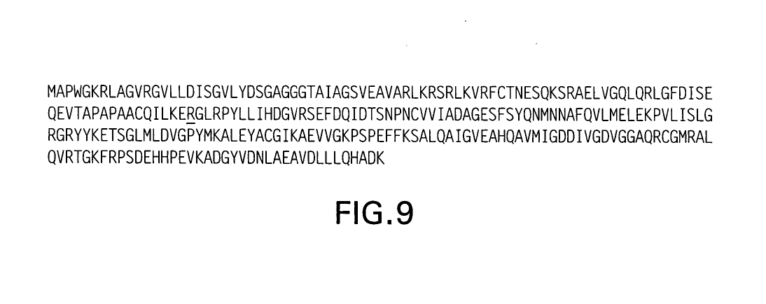

As used herein, the term “Lhpp” refers to an enzyme known as phospholysine phosphohistidine inorganic pyrophosphate phosphatase, and the term “LHPP” refers to the corresponding messenger RNA, and DNA sequences that functionally regulate expression thereof. Lhpp was originally purified from swine brain in 1957 (See, Seal et al., J Biol Chem 228:193-9 (1957)), and subsequently has been purified from several additional mammalian sources (See, Felix et al., J Biochem 147:111-8 (1975); Yoshida et al., Cancer Research 42:3256-31 (1982); Hachimori et al., J Biochem 93:257-64 (1983); Smimova et al., Arch Biochem Biophys 287:135-40 (1991); Hiraishi et al., Arch Biochem Biophys 341:153-9 (1997)). The enzyme has been characterized in vitro as efficiently catalyzing the hydrolysis of P—N bonds in phosphohistidine and phospholysine, and less efficiently catalyzing the hydrolysis of P—N or P—O bonds in imidodiphosphate and pyrophosphate, respectively. Lhpp may be a protein histidine or lysine phosphoamidase, i.e., an enzyme that modifies the N-linked phosphorylation state of other proteins. The human LHPP has been cloned. Functional human Lhpp enzyme has been purified following heterologous expression in E. coli (See, Yokoi et al., J Biochem 133:607-14 (2003)). The nucleic acid sequence of LHPP messenger RNA is shown in SEQ ID NO:9 and in FIG. 8. The amino acid sequence of Lhpp is shown in SEQ ID NO:10 and in FIG. 9. As shown in FIG. 1, LHPP messenger RNA has 7 exons. The locations of these exons are provided below in Table A.

TABLE A

| Exon | Start in SEQ ID NO: 1 | End in SEQ ID NO: 1 |
|---|---|---|
| 1 | 3001 | 3163 |
| 2 | 25305 | 25492 |
| 3 | 29588 | 29741 |
| 4 | 38127 | 38190 |
| 5 | 39202 | 39294 |
| 6 | 58346 | 58437 |
| 7 | 154430 | 155313 |

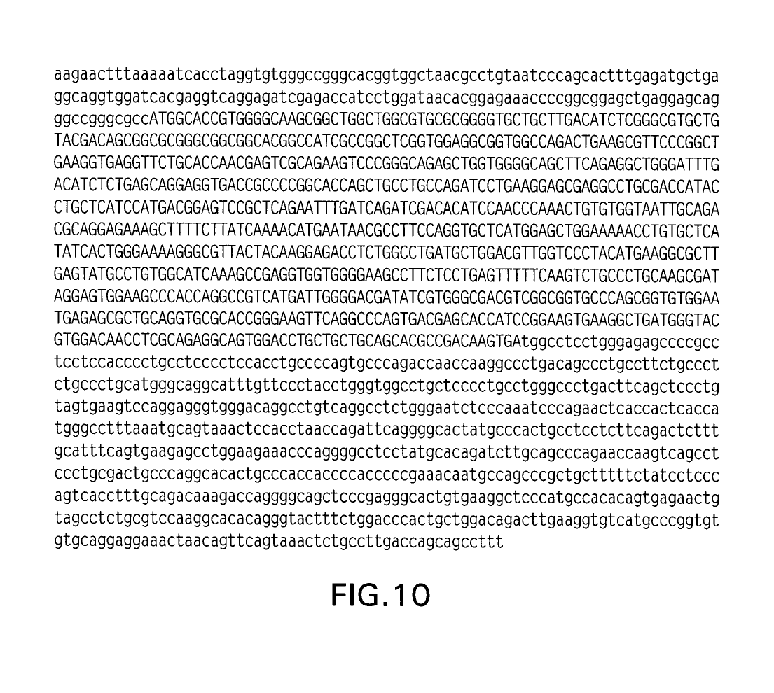

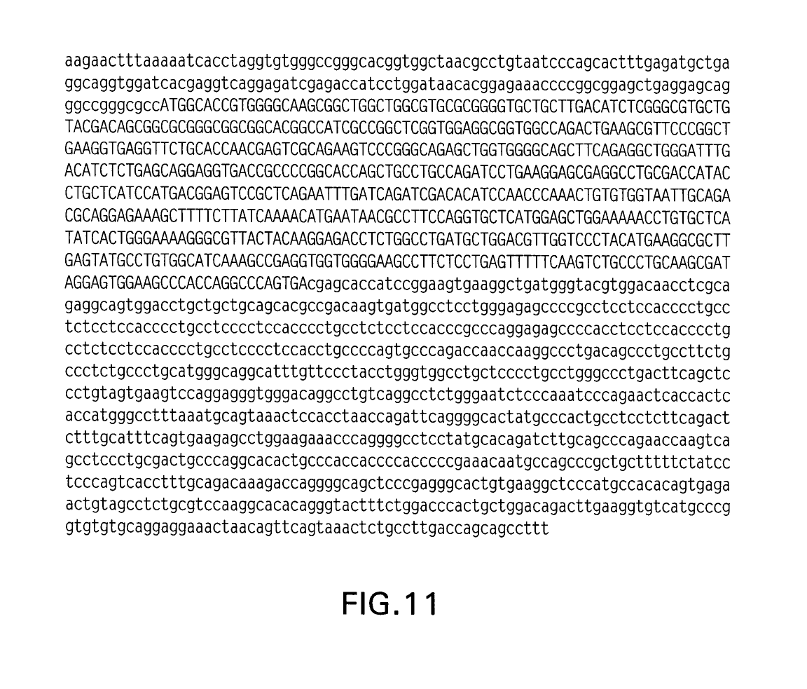

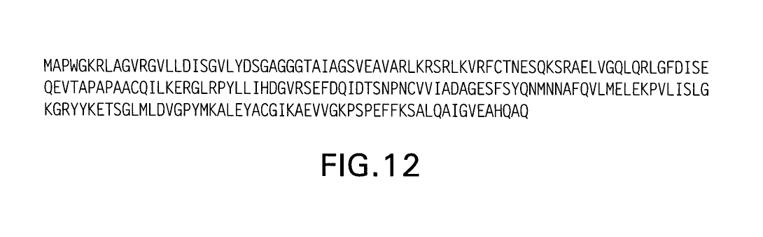

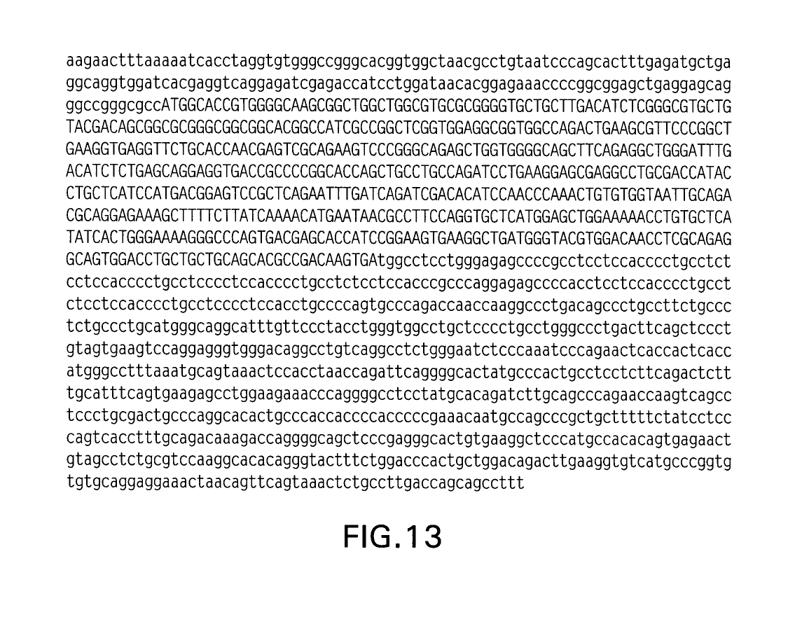

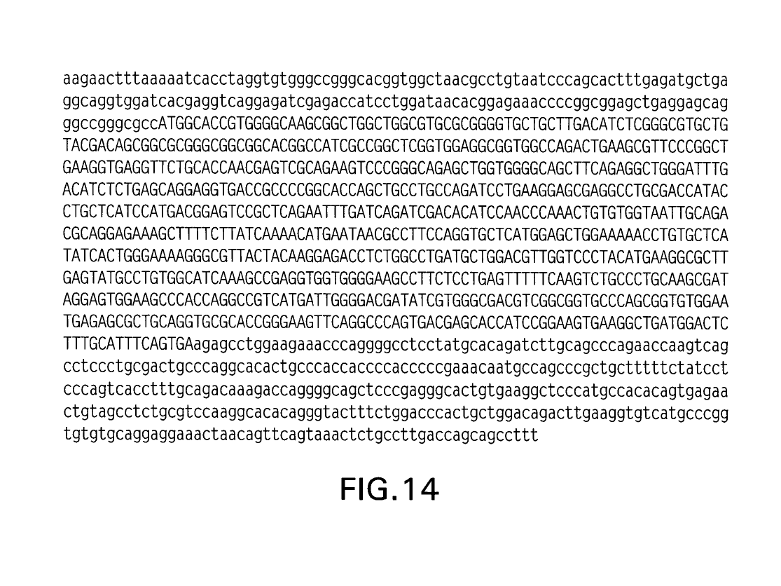

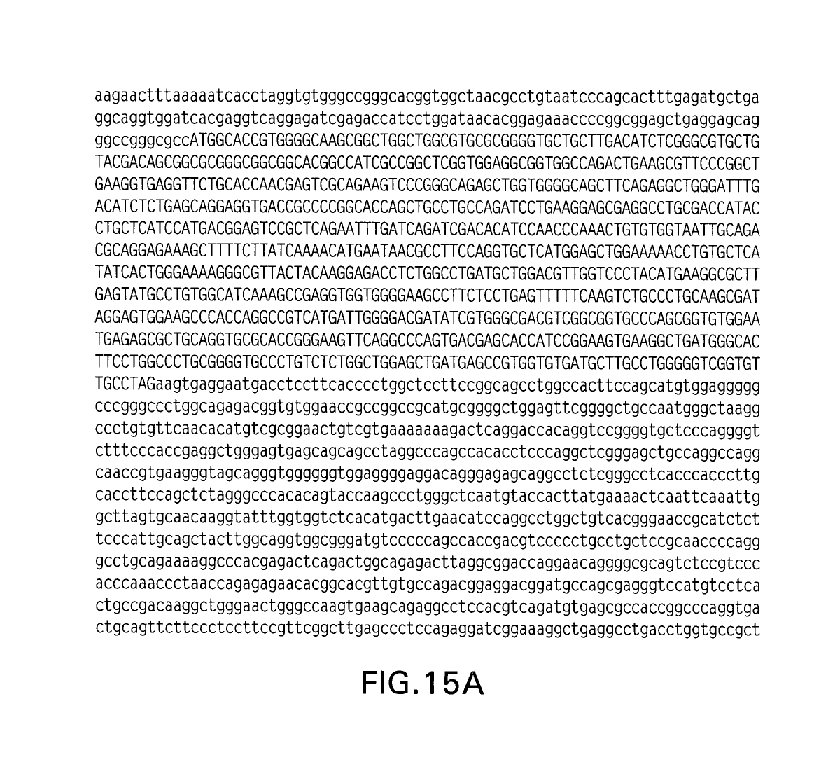

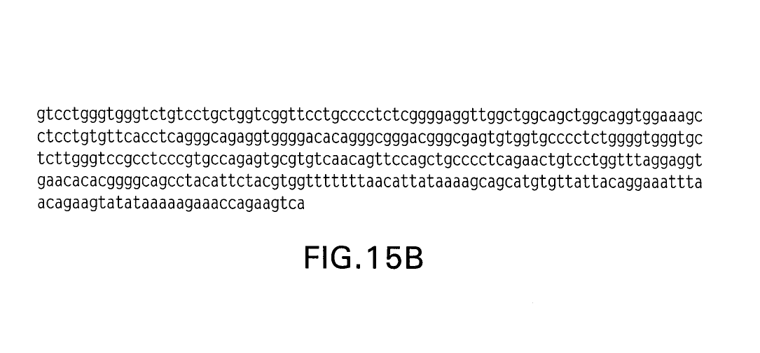

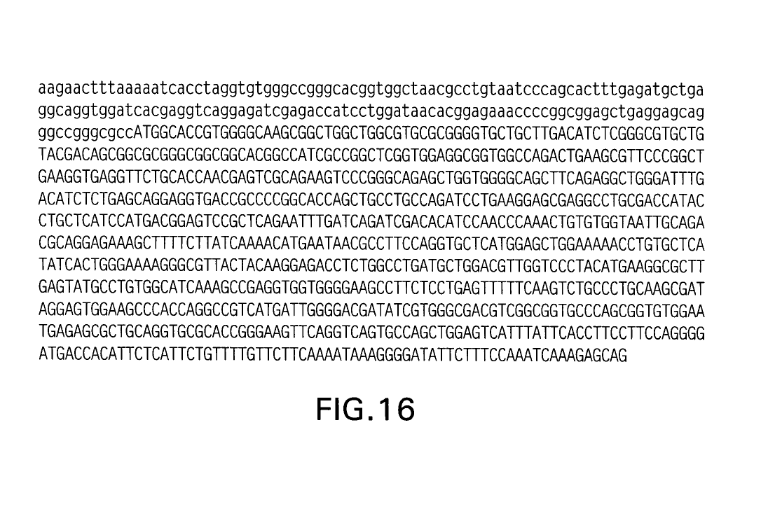

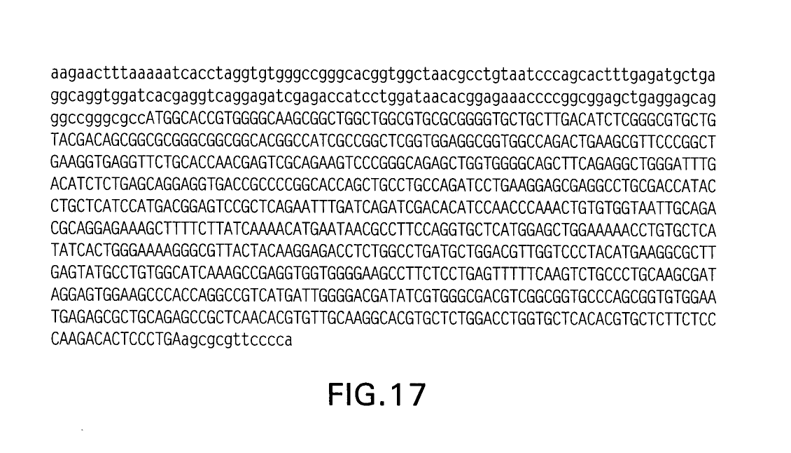

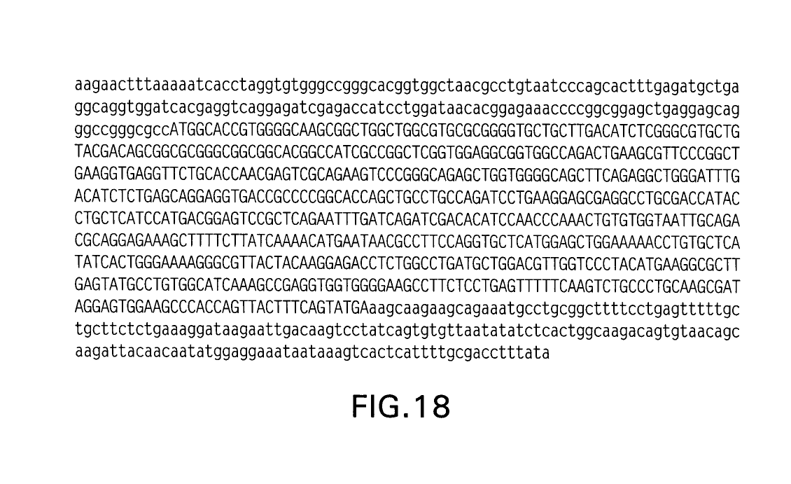

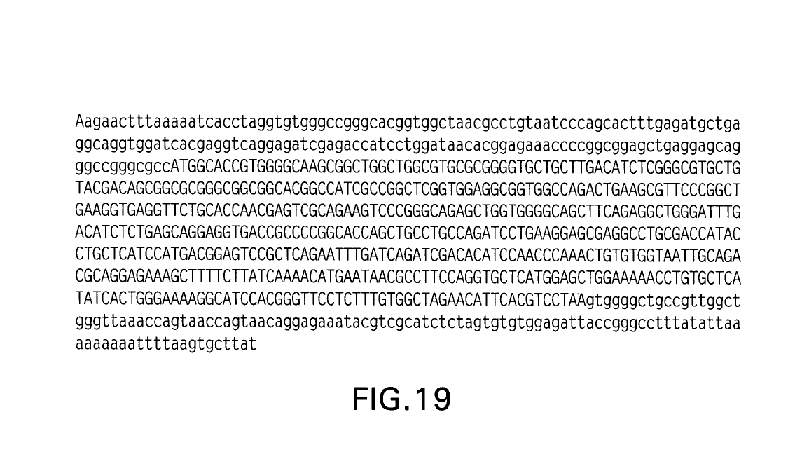

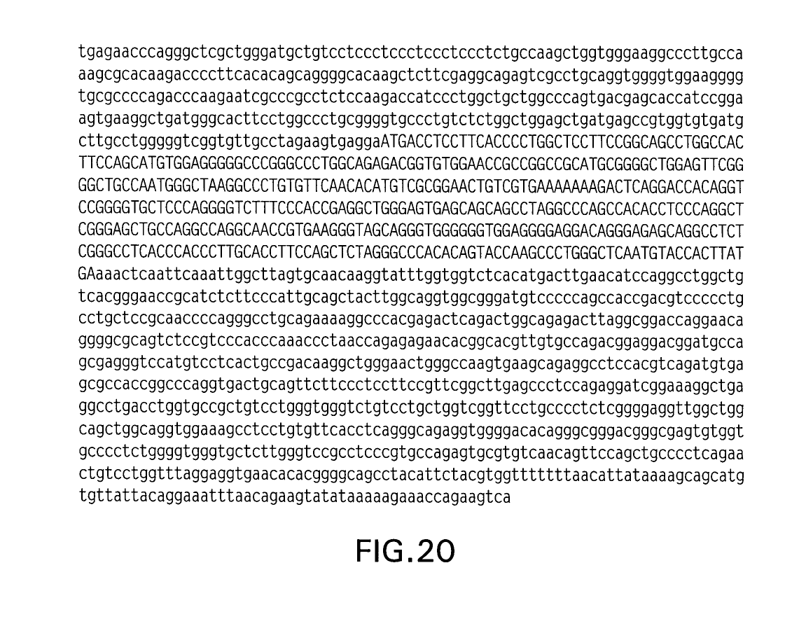

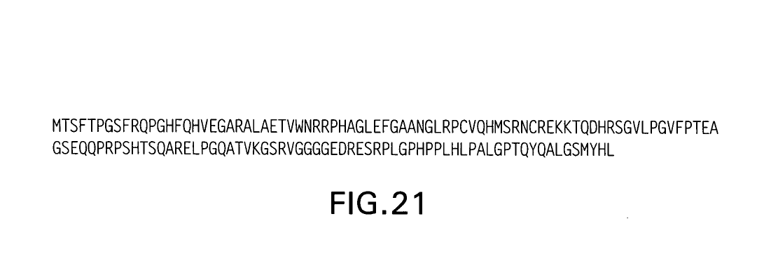

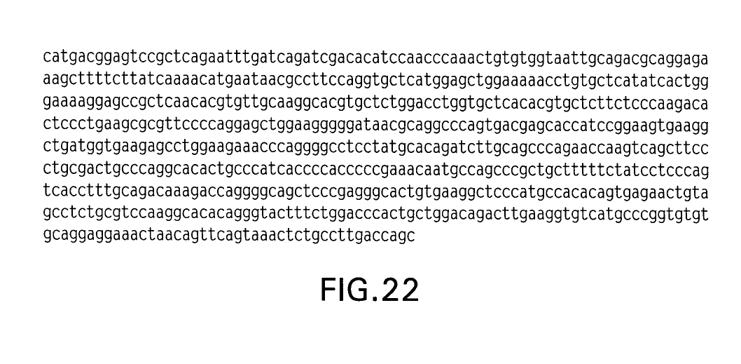

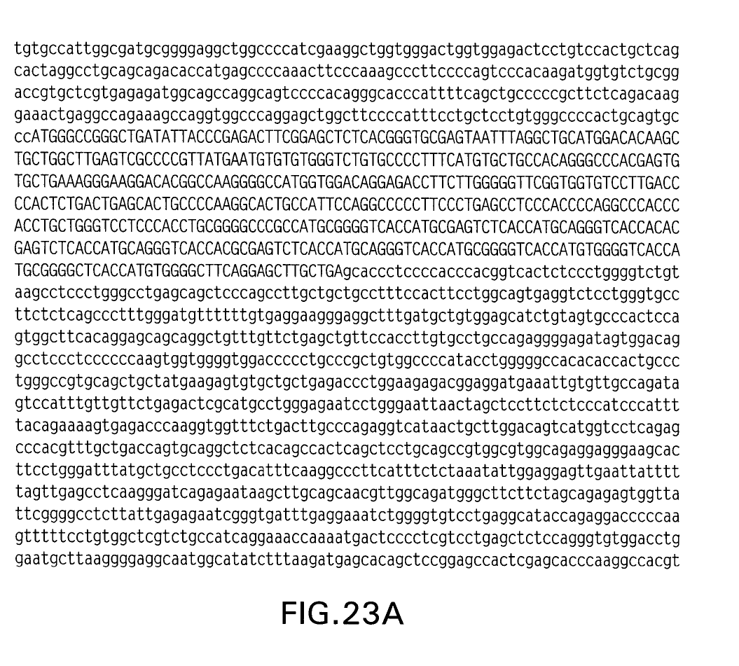

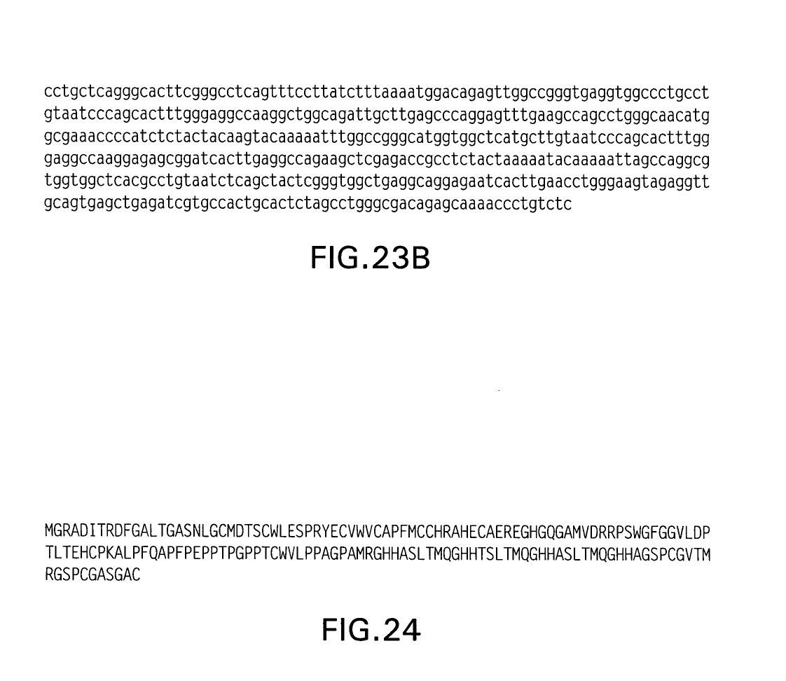

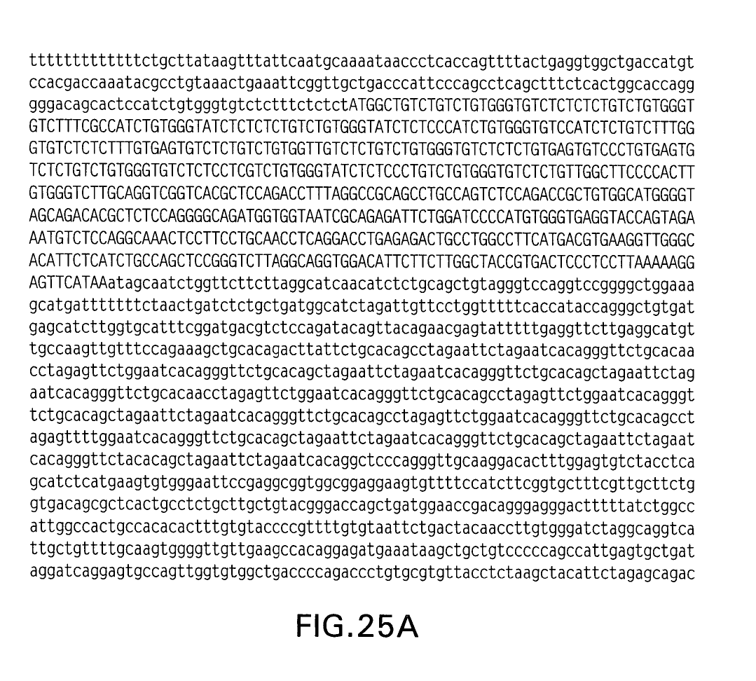

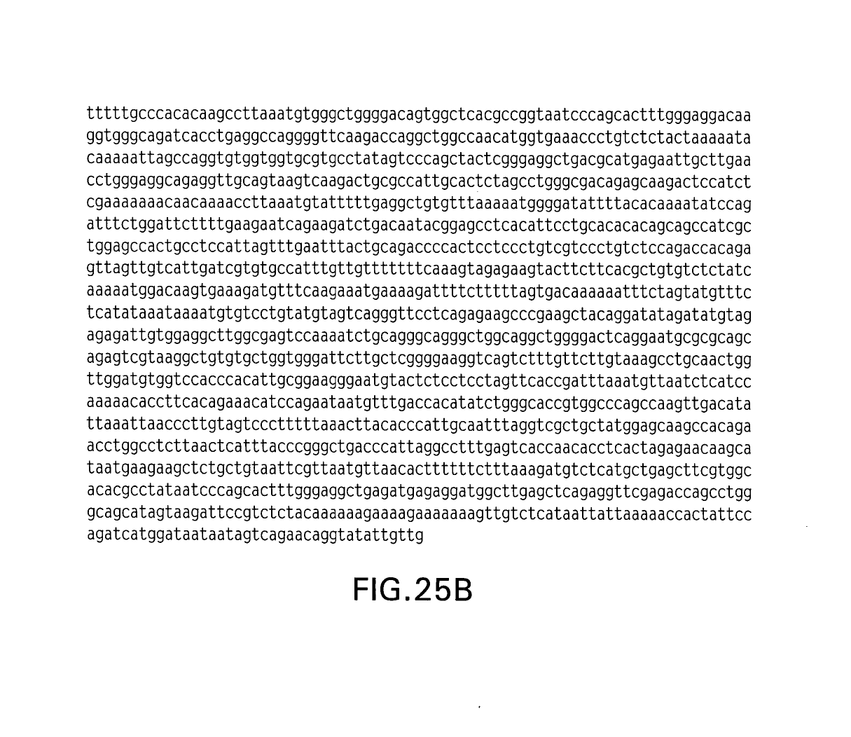

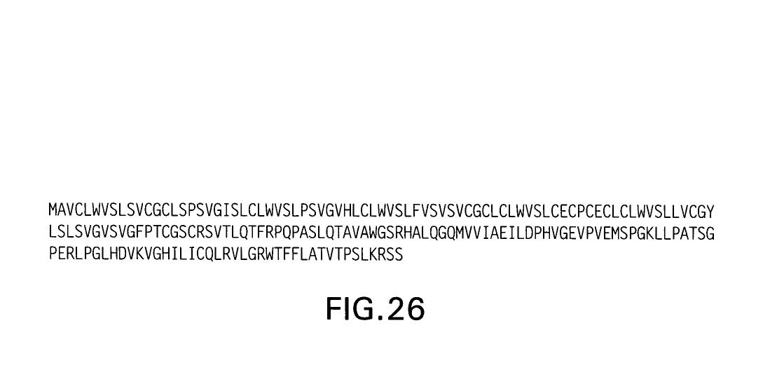

In addition, there is a naturally occurring polymorphic site in Lhpp (R94Q) in which amino acid 94 is either arginine or glutamine in the two naturally occurring isoforms. In the corresponding naturally occurring polymorphic site in LHPP messenger RNA (281G>A), base 281 of the open reading frame is either guanine or adenine in the two naturally occurring alleles. Further, Lhpp is encoded from a naturally occurring splice variant of LHPP that is shown in SEQ ID NO:11 (See FIG. 10).

As used herein, the term “locus” refers to a location on a chromosome of a nucleic acid molecule corresponding to a gene or a physical or phenotypic feature, where physical features include polymorphic sites.

As used herein, the term “major depression or a related disorder” refers to any Mood Disorder or Anxiety Disorder described in the Diagnostic and Statistical Manual (DSM-IV-TR, American Psychiatric Association, 2000). Mood Disorders include, but are not limited to, Depressive Disorders (DSM-IV-TR 296.2x, 296.3x, 300.4, 311), Bipolar Disorders (DSM-IV-TR 296.0x, 296.40, 296.4x, 296.5x, 296.6x, 296.7, 296.89, 301.13, 296.80) and Mood Disorder Not Otherwise Specified (DSM-IV-TR 296.90). Anxiety Disorders include, but are not limited to, Panic Disorders (DSM-IV-TR 300.01, 300.21), Phobic Disorders (DSM-IV-TR 300.29, 300.22, 300.23), Obsessive-Compulsive Disorder (DSM-IV-TR 300.3), Post-Traumatic Stress Disorder (DSM-IV-TR 309.81), Acute Stress Disorder (DSM-IV-TR 308.3), Generalized Anxiety Disorder (DSM-IV-TR 300.02) and Anxiety Disorder Not Otherwise Specified (DSM-IV-TR 300.00). Extensive lists of symptoms and diagnostic criteria for each of these disorders are found in the DSM-IV-TR sections cited above.

As used herein, the terms “modulates” “modulation” or “modulating” as used interchangeably herein, refer to both upregulation (for example, activation or stimulation (for example, by agonizing or potentiating)) and downregulation (for example, inhibition or suppression (for example, by antagonizing, reducing, decreasing or inhibiting)).

As used herein, the term “naturally occurring” refers to a DNA molecule, a messenger RNA, a protein, an allele, an isoform, a polymorphic site, a splice variant or a protein variant, wherein the existence in nature of said DNA molecule, messenger RNA, protein, allele, isoform, polymorphic site, splice variant or protein variant is supported by either (a) direct experimental evidence or (b) algorithmic assembly from a database of nucleic acid or protein sequences. Alleles, isoforms, polymorphic sites, splice variants and protein variants might also be created by experimental manipulation.

As used herein, the term “naturally occurring splice variant of DEP2-1” includes but is not limited to the sequences shown in SEQ ID NO:5, SEQ ID NO:6, SEQ ID NO:7 and SEQ ID NO:8.

As used herein, the term “naturally occurring splice variant of LHPP” includes but is not limited to the sequences shown in SEQ ID NO:12, SEQ ID NO:14, SEQ ID NO:16, SEQ ID NO:18, SEQ ID NO:20, SEQ ID NO:22, SEQ ID NO:24 and SEQ ID NO:26. As used herein, the term “naturally occurring protein variant of Lhpp” includes but is not limited to the sequences shown in SEQ ID NO:13, SEQ ID NO:15, SEQ ID NO:17, SEQ ID NO:19, SEQ ID NO:21, SEQ ID NO:23, SEQ ID NO:25 and SEQ ID NO:27.

As used herein, the phrase “3′ non-coding sequences” refer to mRNA sequences located downstream of a coding sequence.

As used herein, the term “non-human animal” includes all vertebrate animals, except humans. It also includes an individual animal in all stages of development, including embryonic and fetal stages. A “transgenic animal” is any animal containing one or more cells bearing genetic information altered or received, directly or indirectly, by deliberate genetic manipulation at a subcellular level, such as by targeted recombination or microinjection or infection with recombinant virus.

Mice are often used for transgenic animal models because they are easy to house, relatively inexpensive, and easy to breed. However, other non-human transgenic animals may also be made in accordance with the present invention such as, but not limited to, primates, mice, goat, sheep, rabbits, dogs, cows, cats, guinea pigs, rats, zebrafish and nematodes. Transgenic animals are those which carry a transgene, that is, a cloned gene introduced and stably incorporated which is passed on to successive generations.

As used herein, the term “nucleic acid” refers to a polymeric form of nucleotides of any length, either ribonucleotides or deoxyribonucleotides. This term includes double- and single-stranded DNA, as well as double- and single-stranded RNA. It also includes modifications, such as methylation or capping and unmodified forms of the polynucleotide. The terms “polynucleotide,” “oligomer,” “oligonucleotide,” and “oligo” are used interchangeably herein.

As used herein, the phrase “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation. In another example, the complementary RNA regions of the invention can be operably linked, either directly or indirectly, 5′ to the target mRNA, or 3′ to the target mRNA, or within the target mRNA, or a first complementary region is 5′ and its complement is 3′ to the target mRNA.

Polymerase chain reaction (“PCR”) is a technique used to amplify DNA millions of fold, by repeated replication of a template, in a short period of time. (Mullis et al., Cold Spring Harbor Symp. Quant. Biol. 51:263-273 (1986); Erlich et al., European Patent Application No. 50,424; European Patent Application No. 84,796; European Patent Application No. 258,017; European Patent Application No. 237,362; Mullis, European Patent Application No. 201,184; Mullis et al., U.S. Pat. No. 4,683,202; Erlich, U.S. Pat. No. 4,582,788; and Saiki et al., U.S. Pat. No. 4,683,194). The process utilizes sets of specific in vitro synthesized oligonucleotides to prime DNA synthesis. The design of the primers is dependent upon the sequences of DNA that are desired to be analyzed. The technique is carried out through many cycles (usually 20-50) of melting the template at high temperature, allowing the primers to anneal to complementary sequences within the template and then replicating the template with DNA polymerase.

As used herein, the term “polymorphic site” refers to a nucleic acid sequence comprising one or more consecutive nucleotides that differ between alleles, or to a protein sequence comprising one or more consecutive amino acids that differ between isoforms.

As used herein, the term “polymorphism” refers to a sequence variation observed in a subject at a polymorphic site. Polymorphisms include nucleotide or amino acid substitutions, insertions and deletions and may, but need not, result in detectable differences in gene expression or protein function.

The terms “polypeptide” and “protein” are used interchangeably herein and indicate at least one molecular chain of amino acids linked through covalent and/or non-covalent bonds. The terms do not refer to a specific length of the product. Thus peptides, oligopeptides and proteins are included within the definition of polypeptide. In addition, protein fragments, analogs, mutated or variant proteins, fusion proteins and the like are included within the meaning of polypeptide.

As used herein, the term “primer” refers to an oligonucleotide, whether naturally occurring, such as in a purified restriction digest, or produced synthetically, which is capable of acting as a point of initiation of synthesis when placed under conditions in which synthesis of a primer extension product that is complementary to a nucleic acid strand is induced (such as in the presence of nucleotides and an inducing agent such as DNA polymerase and at a suitable temperature and pH). Primers can be single or double stranded. If double stranded, the primer is first treated to separate its strands before being used to prepare extension products. The exact length of the primers will depend on many factors, including temperature, source of primer and the use of the method. Primers preferably have a length of at least 10 contiguous nucleotides. For example, primers can have a length of 10 contiguous nucleotides, 15 contiguous nucleotides, 20 contiguous nucleotides, 25 contiguous nucleotides, etc.

As used herein, the term “probe” refers to an oligonucleotide, whether naturally occurring, such as in a purified restriction digest, produced synthetically, recombinantly or by polymerase chain reaction amplification which is capable of hybridizing to another oligonucleotide or nucleic acid of interest. A probe may be single-stranded or double-stranded. Probes can be labeled with a detectable label so as to make said probe detectable in a detection system. The detectable label used is not critical.

As used herein, the term “promoter” refers to a DNA sequence capable of controlling the transcription of a RNA. Promoters may be derived in their entirety from a native gene, or be composed of different elements derived from different promoters found in nature, or even comprise synthetic DNA segments.

As used herein, the term “protein variant” refers to a polypeptide that is encoded from a splice variant, wherein two protein variants differ in the inclusion/exclusion of one or more blocks of consecutive amino acids.

The terms “recombinant construct”, “construct”, “expression construct” and “recombinant expression construct” are used interchangeably herein. These terms refer to a functional unit of genetic material that can be inserted into the genome of a cell or expressed in vitro using standard methodology well known to one skilled in the art. Such construct may be itself or may be used in conjunction with a vector. If a vector is used then the choice of vector is dependent upon the method that will be used to transform host plants as is well known to those skilled in the art. For example, a plasmid vector can be used. The skilled artisan is well aware of the genetic elements that must be present on the vector in order to successfully transform, select and propagate host cells comprising any of the isolated nucleic acid fragments of the invention. The skilled artisan will also recognize that different independent transformation events will result in different levels and patterns of expression (Jones et al., EMBO J. 4:2411-2418 (1985); De Almeida et al., Mol. Gen. Genetics 218:78-86 (1989)), and thus that multiple events must be screened in order to obtain lines displaying the desired expression level and pattern. Such screening may be accomplished by Southern analysis of DNA, Northern analysis of mRNA expression, Western analysis of protein expression, or phenotypic analysis.

As used herein, the term “regulatory sequences” refers to a DNA or RNA sequence capable of controlling the expression of a RNA or protein. Regulatory sequences may include, but are not limited to, promoters, translation leader sequences, introns, and polyadenylation recognition sequences.

As used herein, the phrase “RNA transcript” or “RNA molecule” as used interchangeable herein, refers to the product resulting from RNA polymerase-catalyzed transcription of a DNA sequence. “Messenger RNA (mRNA)” refers to the RNA that is without introns and that can be translated into protein by the cell.

As used herein, the phrase “sense RNA” refers to RNA molecule that includes the mRNA and can be translated into protein within a cell or in vitro. As used herein, the phrase, “antisense RNA” refers to an RNA molecule that is complementary to all or part of a target primary transcript or mRNA and that blocks the expression of a target gene (U.S. Pat. No. 5,107,065). The complementarity of an antisense RNA may be with any part of the specific gene transcript, i.e., at the 5′ non-coding sequence, 3′ non-coding sequence, introns, or the coding sequence. As used herein, the phrase, “functional RNA” refers to antisense RNA, ribozyme RNA, or other RNA that may not be translated but yet has an effect on cellular processes. The terms “complement” and “reverse complement” are used interchangeably herein with respect to mRNA transcripts, and are meant to define the antisense RNA of the message.

As used herein, the term “single nucleotide polymorphism” or “SNP” refers typically, to a specific pair of nucleotides observed at a single polymorphic site. In some cases, which are rare, three or four nucleotides may be found.

As used herein, the term “splice variant” refers to a particular form of a messenger RNA, wherein two splice variants share either (a) a transcriptional start site or (b) an open reading frame, but differ in the inclusion/exclusion of one or more exons.

As used herein, the term “subject” refers to an animal, preferably a mammal, including a human or non-human. The animal can be a domesticated or non-domesticated animal.

As used herein, the term “treating” refers to reversing, alleviating, inhibiting the progress of, or preventing at least one overt symptomatic manifestation of the disorder or condition to which such term applies, or one or more symptoms of such disorder or condition. The term “treatment” as used herein, refers to the act of treating, as “treating” is defined herein. For the present invention, the term “treat” means to alleviate or eliminate one or more symptoms, behavior or events associated with a depressive disorder.

DEP2-1 Transcripts and Proteins Encoded Thereby

In one embodiment, the present invention relates to the discovery of a novel transcript of DEP2, named DEP2-1. FIG. 2 illustrates the isolated nucleic acid sequence for DEP2-1 (SEQ ID NO:2), which is 1197 base pairs in length. Nucleotides 1 to 315 of SEQ ID NO:2 comprise an exon that was not previously known to be in any naturally occurring transcript of DEP2. The remaining portion of SEQ ID NO:2 (from nucleotides 316 to 1197) are shared with LHPP and naturally occurring splice variants thereof, and correspond to nucleotides 882 to 1760 of SEQ ID NO:9. DEP2-1 was initially assembled using Genecarta software (Compugen, Tel Aviv, Israel) from publicly available expressed sequence tags (“ESTs”). Specifically, the proprietary algorithms identified that certain ESTs (GenBank accession numbers BI669229, BI489679, BI756098, Z44231, R15274, R11923, BX952014, BI754006, H51555, H51378 and BG397886) each comprised of sequences from within both nucleotides 1-315 and nucleotides 316-1197 of SEQ ID NO:2. These two sequence blocks are not contiguous in the human genome, implying that SEQ ID NO:2 is a spliced transcript. To confirm that DEP2-1 is a naturally occurring transcript, four IMAGE clones (h5173309, h5194531, h5197955 and h4565014, corresponding to B1489679, B1756098, B1754006 and BG397886, respectively) were completely sequenced in both directions (see Example 5). The contiguous sequence of nucleotides 18-1197 of SEQ ID NO:2 was thereby confirmed. IMAGE clones h5173309, h5194531 and h5197955 are from brain, and include only sequences shown in SEQ ID NO:2. Among the ESTs that assembled to form DEP2-1, all were from brain except for BG397886, which was from a tonsillar primary B cell line. IMAGE clone h4565014 corresponds to BG397886. The sequence of this clone included nucleotides 298-1197 followed by a polyadenine tail and a further 77 nucleotide sequence that did not match DEP2. Further, a single nucleotide polymorphism (1141G>A) was discovered at nucleotide 1141 of SEQ ID NO:2. Rapid amplification of cDNA ends was performed to determine the 5′ end(s) of DEP2-1 (see Example 6). These experiments identified two 5′ ends in human spinal cord RNA, at nucleotides 1 and 74 of SEQ ID NO:2. Two series of PCR experiments were also performed to determine whether the first exon (nucleotides 1-315) of DEP2-1 is included in additional transcripts with upstream exons (see Example 7). The first experiments used forward primers in an upstream exon of LHPP and reverse primers in the first exon of DEP2-1. The second experiments used forward primers in an upstream exon of LHPP and reverse primers in a downstream exon of LHPP. No sequence from the first exon of DEP2-1 was amplified in either set of experiments. In total, these experimental results demonstrate that DEP2-1 is a naturally occurring transcript, that it is not an alternative splice variant of LHPP, and thus does not encode a naturally occurring protein variant of Lhpp.

The isolated nucleic acid sequence of DEP2-1 has two coding regions, which are each illustrated in capital letters in FIG. 2. The first coding region (nucleotides 351 to 770 in SEQ ID NO:2) may encode for the protein, referred to herein as Dep2-1a, shown in FIG. 3A (SEQ ID NO:3). Dep2-1a is 140 amino acids in length. The second coding region (nucleotides 811 to 1161 in SEQ ID NO:2) may encode for the protein, referred to herein as, Dep2-1b, shown in FIG. 3B (SEQ ID NO:4). Dep2-1b is 117 amino acids in length.

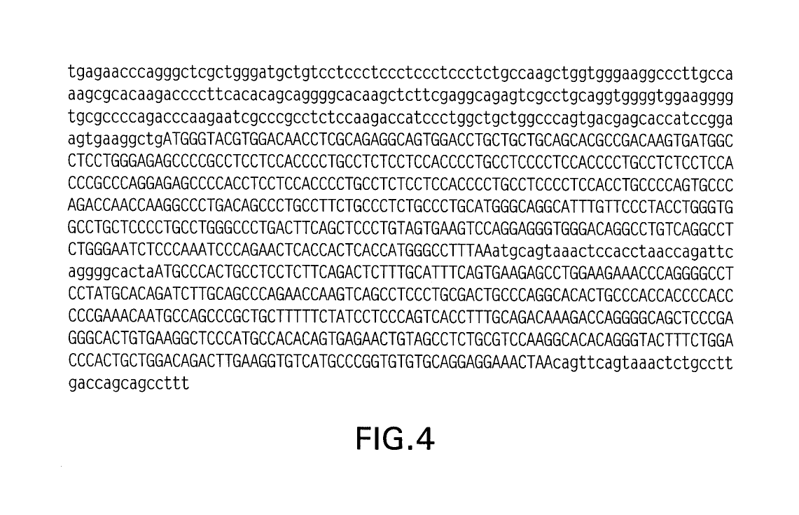

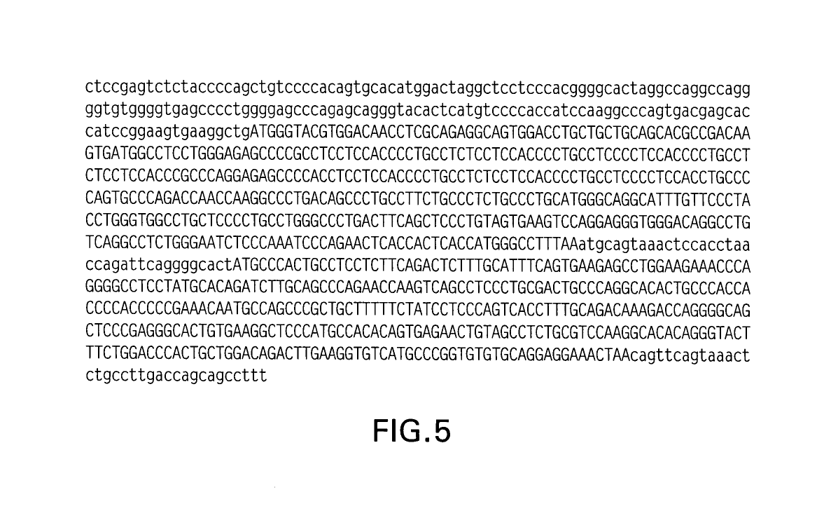

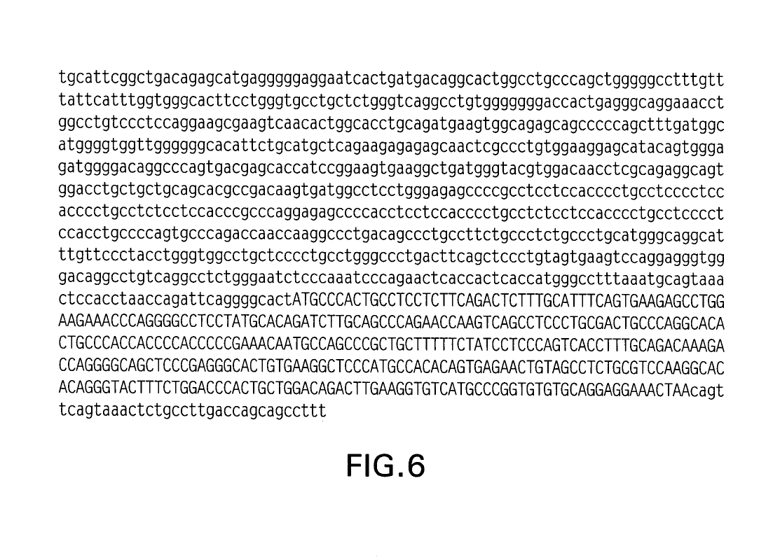

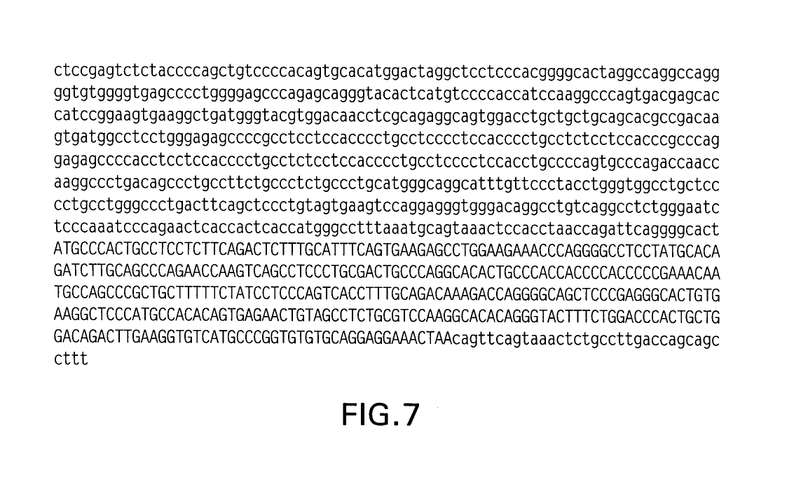

In addition, naturally occurring splice variants of DEP2-1 have been identified by the inventors of the present invention. These transcripts were assembled using Genecarta software (Compugen, Tel Aviv, Israel) from publicly available expressed sequence tags (“ESTs”). These splice variants of the DEP2-1 are shown in SEQ ID NO:5, SEQ ID NO:6, SEQ ID NO:7 and SEQ ID NO:8. SEQ ID NO:5 is also shown in FIG. 4 and is 1092 base pairs in length. The coding regions are shown in capital letters in this figure. The first and second coding regions encode for Dep2-1a (See, FIG. 3A (SEQ ID NO:3)) and Dep2-1b (FIG. 3B (See, SEQ ID NO:4)). SEQ ID NO:5 was assembled on the basis of three ESTs (BG759116, BF976531 and BQ706325), all of B cell origin. SEQ ID NO:6 is shown in FIG. 5 and is 1022 base pairs in length. The coding regions are shown in capital letters in this figure. The first and second coding regions encode for Dep2-1a (See, FIG. 3A (SEQ ID NO:3)) and Dep2-1b (FIG. 3B (See, SEQ ID NO:4)). SEQ ID NO:6 was assembled on the basis of two ESTs (BI226948 and BE396637), both from a Burkitt's lymphoma cell line. SEQ ID NO:7 is shown in FIG. 6 and is 1186 base pairs in length. The coding region is shown in capital letters in this figure. The coding region encodes for Dep2-1b (See, FIG. 3A (SEQ ID NO:4)). SEQ ID NO:7 was assembled on the basis of a single EST (CF454636), from the peripheral nervous system. SEQ ID NO:8 is shown in FIG. 7 and is 1005 base pairs in length.

The coding region is shown in capital letters in this figure. The coding region encodes for Dep2-1b (See, FIG. 3B (SEQ ID NO:4)). SEQ ID NO:8 was assembled on the basis of a single EST (BE560698), from a Burkitt's lymphoma cell line.

The ESTs described in the preceding paragraph were used to assemble the 5′ ends of the variant DEP2-1 transcripts. None of these ESTs contain the entire transcript sequence. In all cases, the 3′ end of each of these transcripts is common to all these sequences as well as to LHPP as well as to some of the splice variants thereof and can be found in multiple ESTs. These ESTs are listed below in Table B.

TABLE B

| GenBank Accession | Tissue Source (If Specified) |
|---|---|
| AA292585 | Ovarian tumor |
| AA308083 | Colon L KM12C (HCC) metastasis into mouse (liver) |
| AA379353 | Skin |
| AA379626 | Skin |
| AA635531 | Normal prostate |
| AA669836 | Bone Marrow stroma |
| AA677990 | Liver and spleen |
| AA725685 | |
| AA912377 | Dorsal root ganglion |
| AI086359 | Pooled human melanocyte, fetal heart, and pregnant uterus |
| AI139589 | Placenta |
| AI97875 | Anaplastic oligodendroglioma |
| AI221142 | Pooled |
| AI272203 | Oligodendroglioma |
| AI361781 | B-cell, chronic lymphotic leukemia |
| AI378120 | B-cell, chronic lymphotic leukemia |
| AI420571 | Prostate |
| AI439876 | Lymphoma, follicular mixed small and large cell |
| AI475774 | B-cell, chronic lymphotic leukemia |
| AI492345 | Kidney |
| AI565021 | Well-defferentiated endometrial adenocarcinoma, 7 pooled tumors |
| AI582637 | Kidney |
| AI598057 | Adenocarcinoma |
| AI805094 | Prostate |
| AI972592 | Prostate |
| AW139347 | |
| AW301045 | Moderately differentiated adenocarcinoma |
| AW511836 | Moderately-differentiated endometrial adenocarcinoma, 3 pooled |
| | tumors |
| AW512618 | Lymphoma, follicular mixed small and large cell |
| AW954516 | |
| BE675320 | B-cell, chronic lymphotic leukemia |
| BF740030 | Kidney |
| BF949274 | Nervous_normal |
| BF986056 | Placenta_normal |
| BF986061 | Placenta_normal |
| BG027502 | Osteosarcoma, cell line |
| BG150734 | Normal epithelium |
| BI819728 | Pooled brain, lung, testis |
| BQ644034 | Hepatocellular carcinoma, cell line |
| BU539737 | Adenocarcinoma, cell line |
| BU683755 | Primary lung cystic fibrosis epithelial cells |
| BU753421 | Placenta |
| BX096697 | Well-differentiated endometrial adenocarcinoma, 7 pooled tumors |
| BX330376 | Placenta |
| BX361464* | Placenta |
| BX423161 | Adult brain |
| CD631211 | |
| CD631212 | |
| CK823134*\hat{ } | Islets of Langerhans |
| CN265311 | Embryonic stem cells, embryoid bodies derived from H1, H7 and H9 |
| | cells |
| N38886 | Multiple sclerosis lesions |

*Reverse direction EST sequence also present in the public domain
\hat{ }Two additional sequences from the same clone are also present in the public domain.

It should be noted that the present invention also encompasses isolated nucleotide sequences (and the corresponding encoded proteins) having sequences comprising, corresponding to, identical to, or complementary to at least about 90% identity to SEQ ID NO:2. (All integers (and portions thereof) between 90% and 100% are also considered to be within the scope of the present invention with respect to percent identity.) For example, the present invention encompasses an isolated nucleic acid or fragment thereof comprising (a) a nucleotide sequence having at least 90% identity to SEQ ID NO:2; or (b) a complement comprising a nucleotide sequence having at least 90% identity to SEQ ID NO:2. Such sequences may be derived from any source, either isolated from a natural source, or produced via a semi-synthetic route, or synthesized de novo.

The invention also includes a purified polypeptide that has at least about 90% amino acid similarity or identity to the amino acid sequences of SEQ ID NO:3 or SEQ ID NO:4 of the above-noted proteins which are, in turn, encoded by the above-described nucleic acid sequences.

The present invention also encompasses an isolated nucleic acid sequence which encodes a polypeptide having the amino acid sequence of SEQ ID NO: 3 or SEQ ID NO:4.

Once DEP2-1 or any naturally occurring variants thereof have been isolated, they may then be introduced into either a prokaryotic or eukaryotic host cell through the use of a vector or construct. The vector, for example, a bacteriophage, cosmid or plasmid, may comprise a nucleic acid sequence having a nucleotide sequence of SEQ ID NO:2, or nucleotides 351-770 or 811-1161 thereof, as well as any regulatory sequence (such as, but not limited to a promoter) which is functional in the host cell and is able to elicit expression of the protein encoded by the nucleotide sequence. Alternatively, the vector may comprise a complement comprising a nucleotide sequence of SEQ ID NO:2 or nucleotides 351-770 or 811-1161 thereof, as well as any regulatory sequence. The regulatory sequence (for example, a promoter) is in operable association with, or operably linked to, the sequence of SEQ ID NO:2, or nucleotides 351-770 or 811-1161 thereof. Examples of promoters that can be used include LTR or the SV40 promoter, the E. coli lac or trp, the phage lambda P sub L promoter and other promoters known to those of skill in the art. Additionally, nucleic acid sequences which encode other proteins, oligosaccharides, lipids, etc. may also be included within the vector as well as other regulatory sequences such as a polyadenylation signal (for example, the poly-A signal of SV-40T-antigen, ovalalbumin or bovine growth hormone). The choice of sequences present in the construct is dependent upon the desired expression products as well as the nature of the host cell.

Once the vector has been constructed, it can be introduced (namely, transformed or transfected) into host cells, such as mammalian (such as, but not limited to, simian, canine, feline, bovine, equine, rodent, murine, etc.) or non-mammalian (such as, but not limited to, insect, reptile, fish, avian, etc.) cells, using any method known to those of skill in the art including, but not limited to, electroporation, calcium phosphate precipitation, DEAE dextran, lipofection, and receptor mediated endocytosis, polybrene, particle bombardment, and microinjection. Alternatively, the vector can be delivered to the cell as a viral particle (either replication competent or deficient). Examples of viruses useful for the delivery of nucleic acid include, but are not limited to, lentivirus, adenoviruses, adeno-associated viruses, retroviruses, herpesviruses, and vaccinia viruses. Other viruses suitable for delivery of nucleic acid sequences into cells that are known to those of skill in the art may be equivalently used in the present invention.

The engineered host cells can be cultured in conventional nutrient media modified as appropriate for activating the promoter sequences, selecting transfected cells, etc. The culture conditions, such as temperature, pH and the like, are those previously used with the host cell selected for expression, and will be apparent to those of skill in the art.

The engineered host cells containing the incorporated vector(s) can be identified using hybridization techniques that are well known to those of skill in the art or by using the polymerase chain reaction to amplify specific polynucleotide sequences. If the nucleic acid sequence transferred to the cells produces a protein that can be detected, for example, by means of an immunological or enzymatic assay, then the presence of recombinant protein can be confirmed by performing the assays either on the medium surrounding the cells or on cellular lysates.

Non-Human Transgenic Animals

In another embodiment, the present invention relates to non-human transgenic animals that contain the transcripts that arise from DEP2 as well as methods of making said animals. Specifically, the nucleic acid sequences that can be used in said non-human transgenic animals include: (a) LHPP (SEQ ID NO:9); (b) naturally occurring splice variants of LHPP (SEQ ID NO:11, SEQ ID NO:12, SEQ ID NO:14, SEQ ID NO:16, SEQ ID NO:18, SEQ ID NO:20, SEQ ID NO:22, SEQ ID NO:24 and SEQ ID NO:26; (c) DEP2-1 (SEQ ID NO:2); (d) naturally occurring splice variants of DEP2-1 (SEQ ID NO:5, SEQ ID NO:6, SEQ ID NO:7 or SEQ ID NO:8); (e) DEP2-2 (SEQ ID NO:28); (f) DEP2-3 (SEQ ID NO:30); (g) GenBank sequence AK127935 (SEQ ID NO:31); and (h) GenBank sequence AW867792 (SEQ ID NO:33).

A variety of methods can be used to create the non-human transgenic animals. For example, the generation of a specific alteration of a nucleic acid sequence of a target gene is one approach that can be used. Alterations can be accomplished by a variety of enzymatic and chemical methods used in vitro. One of the most common methods uses a specific oligonucleotide as a mutagen to generate precisely designed deletions, insertions and point mutations in a target gene. Secondly, a wildtype human gene or humanized non-human animal gene could be inserted by homologous recombination. It is also possible to insert an altered or mutated (singly or multiply) human gene as genomic or minigene constructs.

Additionally, non-human transgenic animals can also be made wherein at least one endogenous target gene is “knocked-out”. The creation of knock-out animals allows those of skill in the art to assess in vivo function of the gene that has been “knocked-out”. The knock-out of at least one target gene may be accomplished in a variety of ways. One strategy that can be used to “knock-out” a target gene is by the insertion of artificially modified fragments of the endogenous gene by homologous recombination. In this technique, mutant alleles are introduced by homologous recombination into embryonic stem (“ES”) cells. The embryonic stem cells containing a knock out mutation in one allele of the gene being studied are introduced into a blastocyst. The resultant animals are chimeras containing tissues derived from both the transplanted ES cells and host cells. The chimeric animals are mated to assess whether the mutation is incorporated into the germ line. Those chimeric animals each heterozygous for the knock-out mutation are mated to produce homozygous knock-out mice. A second strategy that can be used to “knock-out” at least one gene involves using siRNA and shRNA and oocyte microinjection or transfection or microinjection into embryonic stem cells as described further herein.

The present invention contemplates that the somatic and germ cells of said non-human transgenic animal comprise an exogenous and stably transmitted nucleic acid sequence of SEQ ID NO:2 (DEP2-1). Additionally, the present invention further contemplates that the somatic and germ cells of the transgenic animals comprise an exogenous and stably transmitted nucleic acid sequence having a nucleotide sequence selected from the group consisting of: SEQ ID NO:9, SEQ ID NO:11, SEQ ID NO:12, SEQ ID NO:14, SEQ ID NO:16, SEQ ID NO:18, SEQ ID NO:20, SEQ ID NO: 22, SEQ ID NO:24, SEQ ID NO:26, SEQ ID NO:28, SEQ ID NO:30, SEQ ID NO:31 and SEQ ID NO:33, with the proviso that its somatic and germ cells do not comprise an exogenous and stably transmitted nucleic acid having a nucleotide sequence of SEQ ID NO:2. The methods for creating such transgenic animals will be discussed in more detail below.

The present invention further contemplates non-human transgenic animals wherein a nucleic acid comprising a nucleotide sequence of SEQ ID NO:2 (DEP2-1) is knocked out in said animal. Additionally, the present invention contemplates a non-human transgenic animal wherein a nucleic acid having a nucleotide sequence selected from the group consisting of: SEQ ID NO:9, SEQ ID NO:11, SEQ ID NO:12, SEQ ID NO:14, SEQ ID NO:16, SEQ ID NO:18, SEQ ID NO:20, SEQ ID NO: 22, SEQ ID NO:24, SEQ ID NO:26, SEQ ID NO:28, SEQ ID NO:30, SEQ ID NO:31 and SEQ ID NO:33 is knocked out, with the proviso that a nucleic acid sequence of SEQ ID NO:2 is not modified or altered. The methods for creating such “knock-out” animals will be described in more detail below.

To create a non-human transgenic animal containing an exogenous and stably transmitted nucleic acid of a target gene or other nucleic acid sequence, a nucleic sequence of interest can be inserted into a non-human animal germ line using standard techniques of oocyte microinjection or transfection or microinjection into embryonic stem cells. Alternatively, if it is desired to knock-out or replace an endogenous gene, homologous recombination using embryonic stem cells or siRNA or shRNA using oocyte microinjection or transfection or microinjection of embryonic stem cells can be used.

For oocyte injection, at least one nucleic acid sequence of interest that is operably linked to the promoter can be inserted into the pronucleus of a just-fertilized non-human animal oocyte. This oocyte is then reimplanted into a pseudopregnant foster mother. The liveborn non-human animal can then be screened for integrants by analyzing the animal's DNA (using polymerase chain reaction for example) such as from the tail, for the presence of the polynucleotide sequence of interest. Chimeric non-human animals are then identified. The nucleic acid can be a complete genomic sequence injected as a YAC or chromosome fragment, a cDNA, or a minigene containing the entire coding region and other elements found to be necessary for optimum expression.

Retroviral or lentiviral infection (See, Lois C, et al., Science, 295:868-872 (2002) (which teaches methods for transgenics using lentiviral transgenesis)) of early embryos can also be done to insert an altered gene. In this method, the altered gene is inserted into a retroviral vector which is used to directly infect mouse embryos during the early stages of development to generate a chimera, some of which will lead to germline transmission (Jaenisch, R., Proc. Natl. Acad. Sci. USA, 73: 1260-1264 (1976)).

Homologous recombination using embryonic stem cells allows for the screening of gene transfer cells to identify the rare homologous recombination events. Once identified, these can be used to generate chimeras by injection of at least one non-human animal blastocyst and a proportion of the resulting animals will show germline transmission from the recombinant line. This gene targeting methodology is especially useful if inactivation of the gene is desired. For example, inactivation of the gene can be done by designing a polynucleotide fragment which contains sequences from an exon flanking a selectable marker. Homologous recombination leads to the insertion of the marker sequences in the middle of an exon, inactivating the gene. DNA analysis of individual clones can then be used to recognize the homologous recombination events.

Alternatively, “knock-out” of a target gene can be accomplished using siRNA or shRNA. In one strategy, oocyte microinjection can be used as described herein. Specifically, at least one nucleic acid sequence of interest that expresses at least one RNA molecule that is siRNA or shRNA, and that is operably linked to at least one promoter (such as a RNA pol III dependent promoter), is prepared using the methods described herein. This nucleic acid is introduced into a non-human animal fertilized oocyte, preferably by injection. The fertilized oocyte is then allowed to develop into an embryo. The resulting embryo is then transferred into a pseudopregnant female non-human animal and then allowed to give birth. Liveborn non-human animals are then screened for chimeric animals that contain the nucleic acid by obtaining a sample and analyzing the animal's DNA (using techniques such as polymerase chain reaction) and such chimeric non-human animals are identified. When these non-human animals are treated with an inducing agent, transcription is induced, the siRNA or shRNA expressed, and the target gene is repressed or “knocked-out”. In the absence of the inducing agent, the gene is not repressed or “knocked-out”.

In a second strategy, microinjection of embryonic stem cells can be used as described herein. Specifically, at least one nucleic acid sequence of interest that expresses at least one RNA molecule that is siRNA or shRNA, and is operably linked to at least one RNA pol III dependent promoter sequence of the present invention, is prepared using the methods described herein. This nucleic acid is introduced into non-human animal embryonic stem cells which can be used to generate chimeras by introducing these embryonic stem cells, preferably by injection, into at least one non-human animal blastocyst. The resulting blastocyst is then implanted into a pseudopregnant female non-human animal and then allowed to give birth to a chimeric non-human animal. PCR can be used to identify the animals of interest. Liveborn non-human animals are then screened for chimeric animals that contain the nucleic acid by obtaining and analyzing a sample of said animal's DNA (using techniques such as polymerase chain reaction) and such chimeric non-human animals are identified. This chimeric non-human animal can then be used in breeding to produce a transgenic non-human animal that stably contain this nucleic acid within their genome. As with the previous method, when these non-human animals are treated with an inducing agent, transcription is induced, the siRNA or shRNA expressed, and the target gene is repressed or “knocked-out”. In the absence of the inducing agent, the gene is not repressed or “knocked-out”.

Methods of making transgenic animals are described, for example, in Wall et al., J. Cell Biochem., 49(2):113-20 (1992); Hogan, et al., “Manipulating the mouse embryo”, A Laboratory Manual. Cold Spring Harbor Laboratory Press, Cold Spring Harbor, N.Y. (1992); in WO 91/08216 or U.S. Pat. No. 4,736,866 the disclosures of which are hereby incorporated by reference in their entirety.

Method of Modifying or Altering Expression of Nucleic Acid Molecules

In another embodiment, the present invention relates to methods of modifying or altering the expression of nucleic acid sequences. The present invention contemplates that the nucleic acid sequence whose expression is modified or altered is SEQ ID NO:2. The present invention further contemplates that the nucleic acid sequence whose expression is modified or altered is a nucleic acid having a nucleotide sequence of at least one of SEQ ID NO:9, SEQ ID NO:11, SEQ ID NO:12, SEQ ID NO:14, SEQ ID NO:16, SEQ ID NO:18, SEQ ID NO:20, SEQ ID NO: 22, SEQ ID NO:24, SEQ ID NO:26, SEQ ID NO:28, SEQ ID NO:30, SEQ ID NO:31 or SEQ ID NO:33.

Methods for modifying or altering the expression of a nucleic acid sequence are well known to those skilled in the art. Specifically, said methods involve exposing a cell or administering to a subject (such as a transgenic non-human animal (for example, a transgenic non-human animal having at least one nucleic acid molecule knocked-out)) containing a nucleic acid whose expression is to be modified or altered at least one nucleic acid molecule. The methods described herein could be useful, such as in transgenic non-human animals (such as in transgenic non-human animals having at least one nucleic acid molecule knocked-out), as animal models for major depression or a related disorder. Nucleic acid molecules such as antisense molecules, aptamers, triplexing agents, ribozymes, siRNA, or co-suppression (co-suppressor) RNA can be used in said methods.

An antisense molecule, aptamer, triplexing agent, ribozyme or siRNA are DNA, RNA or chemically modified or hybrid sequences thereof of varying length that are single or double stranded. These nucleic acid molecules are complementary to a target nucleic acid sequence, such as a mRNA of a nucleic acid (a) having a nucleotide sequence of SEQ ID NO:2; or (b) of at least one of SEQ ID NO:9, SEQ ID NO:11, SEQ ID NO:12, SEQ ID NO:14, SEQ ID NO:16, SEQ ID NO:18, SEQ ID NO:20, SEQ ID NO: 22, SEQ ID NO:24, SEQ ID NO:26, SEQ ID NO:28, SEQ ID NO:30, SEQ ID NO:31 or SEQ ID NO:33, and can be a coding sequence, a polynucleotide sequence comprising an intron-exon junction, a regulatory sequence, such as a promoter sequence, or the like. The degree of complementarity is such that the nucleic acid molecule can interact specifically with the target nucleic acid sequence in a cell. Depending on the total length of the nucleic acid molecule, one or a few mismatches with respect to the target nucleic acid sequence can be tolerated without losing the specificity of the nucleic acid molecule for the target sequence. Thus, a few mismatches, if any, would be tolerated, for example, in an antisense molecule containing, for example, 20 consecutive nucleotides, whereas several mismatches will not affect the hybridization efficiency of an antisense molecule that is complementary to a full length of a target mRNA encoding a protein (such as, Dep2-1a, Dep2-1b, Dep2-2, Dep2-4 or Dep2-5). The number of mismatches that can be tolerated can be estimated, using well known formulas for determining hybridization kinetics (See, Sambrook et al., Molecular Cloning: A Laboratory Manual, 2^{nd} Edition (1989)) or can be determined empirically using methods known in the art, particularly by determining that the presence of the antisense molecule, aptamer, triplexing agent, ribozyme or siRNA in a cell modifies or alters (such as by decreasing) the level of expression of the target sequence in a cell.

A nucleic acid molecule useful as an antisense molecule, aptamer, triplexing agent, ribozyme or siRNA can reduce or inhibit translation or cleave a target nucleic acid, thereby reducing or inhibiting the amount of the protein encoded by said target nucleic acid in a cell. For example, an antisense molecule can bind to an mRNA to form a double stranded molecule that cannot be translated in a cell. Antisense oligonucleotides of about 15 to 50 consecutive nucleotides are preferred since they are easily synthesized and can hybridize specifically with a target nucleic acid, although longer antisense molecules can be used. When the antisense molecule is contacted directly with a target cell, it can be operatively associated with a chemically reactive group such as, but not limited to, iron-linked EDTA, which cleaves a target RNA at the site of hybridization. A triplexing agent, in comparison, can stall transcription (Maher et al., Antisense Res. Devel., 1:227 (1991); Helene, Anticancer Drug Design, 6:569 (1991)). Aptamers adopt highly specific three-dimensional conformations that enable them to bind to a specific location on a molecule whose activity is being affected. Methods for making antisense molecules, aptamers and triplexing agents are well known in the art.

A ribozyme is a catalytic RNA molecule that cleaves RNA in a sequence-specific manner. Ribozymes that cleave themselves are called cis-acting ribozymes, while ribozymes that cleave other RNA molecules are called trans-acting ribozymes. Nucleic acids molecules can encode ribozymes designed to cleave particular mRNA transcripts, thus preventing expression of a polypeptide. A ribozyme sequence can have a sequence from a hammerhead, axhead, or hairpin ribozyme, and may be modified to have either slow cleavage activity or enhanced cleavage activity. For example, nucleotide substitutions can be made to modify cleavage activity (see, e.g., Doudna and Cech, Nature, 418:222-228 (2002)). Hammerhead ribozymes are useful for destroying particular mRNAs, although various ribozymes that cleave mRNA at site-specific recognition sequences can be used. Hammerhead ribozymes cleave mRNAs at locations dictated by flanking regions that form complementary base pairs with the target mRNA. The sole requirement is that the target RNA contain a 5′-UG-3′ nucleotide sequence. The construction and production of hammerhead ribozymes is known in the art. See, for example, U.S. Pat. No. 5,254,678. Hammerhead ribozyme sequences can be embedded in a stable RNA such as a transfer RNA (tRNA) to increase cleavage efficiency in vivo (Perriman, R. et al., Proc. Natl. Acad. Sci. USA, 92(13):6175-6179 (1995); de Feyter, R. and Gaudron, J., Methods in Molecular Biology, Vol. 74, Chapter 43, “Expressing Ribozymes in Plants”, Edited by Turner, P. C, Humana Press Inc., Totowa, N.J.).

siRNA useful in the present invention can be obtained, for example, using an in vitro transcription system or can be synthesized chemically, and can be contacted with cells (or administered to a subject) as RNA molecules. siRNA also can be expressed from an encoding nucleic acid, which can be contacted with cells (or administered to a subject). siRNAs can be designed using techniques well known to those skilled in the art.

Another nucleic acid molecule that is useful in the present methods also can be a co-suppression RNA that reduces or inhibits transcription of a target nucleic acid, such as a nucleic acid (a) having a nucleotide sequence of SEQ ID NO:2; or (b) of at least one of SEQ ID NO:9, SEQ ID NO:11, SEQ ID NO:12, SEQ ID NO:14, SEQ ID NO:16, SEQ ID NO:18, SEQ ID NO:20, SEQ ID NO: 22, SEQ ID NO:24, SEQ ID NO:26, SEQ ID NO:28, SEQ ID NO:30, SEQ ID NO:31 or SEQ ID NO:33. A co-suppressor RNA, like an siRNA, comprises (or encodes) an RNA comprising an inverted repeat, which includes a first oligonucleotide that selectively hybridizes to the target nucleic acid or gene and, in operative linkage, a second oligonucleotide that is complementary and in a reverse orientation to the first oligonucleotide. In comparison to an siRNA, which comprises a functional portion of a transcribed region of the target nucleic acid or target gene and reduces or inhibits translation of RNA transcribed from the nucleic acid or gene, a co-suppressor RNA comprises a functional portion of a transcriptional regulatory region of the target nucleic acid or gene (namely, a promoter region) and reduces or inhibits transcription of the nucleic acid or gene. Methods for making co-suppression RNA are well known in the art.

Polymorphism Detection/Genotyping

In another embodiment, the present invention relates methods of genotyping one or more subjects. The information obtained from the genotyping of subjects can be used in a variety of different ways. For example, the genotyping of subjects can be used to diagnose those subjects suffering from major depression or a related disorder or at risk of developing major depression or a related disorder, provide a prognosis for or predict or diagnose a response to treatment for a subject suffering from major depression or a related disorder, or identify subjects for selection or inclusion in a clinical trial for treating major depression or a related disorder. Additionally, genotypes can be used to analyze the results of a clinical trial for subjects being treated for major depression or a related disorder. Specifically, the relationship the genotypes of subjects and the clinical outcome of said subjects can be determined.

Genotyping involves obtaining a test sample from said subject(s). The subject may or may not be experiencing any symptoms of major depression or a related disorder at the time the test sample is obtained. In this embodiment, a test sample is any biological sample which contains the DNA of the subject. Test samples can be prepared using techniques well known to those skilled in the art such as by obtaining a specimen from an individual and, if necessary, disrupting any cells contained therein to release DNA. Examples of test samples include, but are not limited to, whole blood, serum, plasma, cerebrospinal fluid, sputum, bronchial washing, bronchial aspires, urine, lymph fluids, and various external secretions of the respiratory, intestinal and genitourinary tracts, tears, saliva, milk, white blood cells, myelomas and the like, etc.

Once the test sample(s) is obtained, it is analyzed, using routine techniques known in the art, in order to determine the presence or absence of specific sequences (alleles) for: (a) at least one polymorphic site in nucleotides 1 to 315 of SEQ ID NO:2; (b) a T-C polymorphism at position (nucleotide) 135 of SEQ ID NO:2; (c) a A-G polymorphism at position 209 of SEQ ID NO:2; (d) a G-A polymorphism at position 241 of SEQ ID NO:2;

(e) at least one polymorphic site selected from nucleotides 77402 and 79906 of SEQ ID NO:1; (f) at least one polymorphic site in SEQ ID NO:1; or (g) any combinations of (a)-(f). Additionally, the test sample may optionally be further analyzed for a C-G polymorphism at position −1019 of a human serotonin receptor 1A gene (“HTR1A”). For example, the identification of at least one polymorphic site at nucleotide 38048, 77402 or 79906 in SEQ ID NO:1 in combination with the identification of a C-G polymorphism at position −1019 of a human serotonin receptor 1A gene in a test sample obtained from a subject may indicate that the subject is at risk of developing major depression or a related disorder.

The genotype of the subject can be determined based on the combination of sequences present at one of more polymorphic sites. Once the genotype of the subject has been determined, then further determinations can be made, such as, diagnosing whether the subject has major depression or a related disorder or is at risk of developing major depression or a related disorder, providing a prognosis for or predicting the response to treatment for a subject having major depression or a related disorder, determining whether the subject should be selected for inclusion in a clinical trial for treatment of major depression or a related disorder, or analyzing the relationship between genotypes of subjects and their clinical outcome. Additionally, if the test sample is also analyzed for the presence of sequences at a C-G polymorphism at position −1019 in the HTR1A gene, then the genotype(s) at one or more polymorphic sites in DEP2 may be used in combination with the genotype at HTR1A to make further determinations, as elaborated above.

As briefly discussed above, techniques for identifying the presence or absence of at least one sequence (allele) at a polymorphic site in a test sample are well known in the art and include, but are not limited to direct sequencing, amplification, fragment length polymorphism assays, mobility based assays, hybridization assays and mass spectroscopy. These techniques will be discussed briefly below.

Direct Sequencing

The presence or absence of a sequence at a polymorphic site may be determined by direct nucleotide sequencing. Methods for direct sequencing are known in the art. For example, following amplification of the DNA from the test sample, the DNA can be sequenced using manual sequencing techniques, such as those that employ radioactive marker nucleotides, or by automated sequencing. The results of the sequencing can be displayed using any suitable method known in the art. The sequence is examined and the presence or absence of a given sequence at a polymorphic site is determined.

Amplification

The presence or absence of a sequence at a polymorphic site may be determined using amplification techniques, such as PCR. PCR involves the use of primers to amplify a region of a DNA sequence from the test sample containing the polymorphic site of interest. The design of primers is well known to those skilled in the art. For example, primers can be designed that hybridize only to a portion of SEQ ID NO:1 or a portion of SEQ ID NO:2 (hereinafter “the wildtype”). If these wildtype primers result in a PCR product, then the subject has the wildtype allele (namely, SEQ ID NO:1 or SEQ ID NO:2). Similarly, primers can be designed that hybridize only to a portion of SEQ ID NO:1 or a portion of SEQ ID NO:2 containing a variant sequence at one or more polymorphic sites (hereinafter “the variant”). If these variant primers result in a PCR product, then the subject has the variant allele (namely, SEQ ID NO:1 or SEQ ID NO:2). The presence of an amplification product only when wildtype primers are used, or only when variant primers are used, indicate a homozygous wildtype or variant genotype, respectively. The presence of an amplification product when either wildtype or variant primers are used indicates a heterozygous genotype. Amplification methods other than PCR can be used. Such methods include strand displacement, the QB replicase system, the repair chain reaction, ligase chain reaction, rolling circle amplification and ligation activated transcription.

Fragment Length Polymorphism Assays

The presence or absence of sequences at a polymorphic site may be determined using a fragment length polymorphism assay. In a fragment length polymorphism assay, a unique DNA banding pattern based on cleaving the DNA at a series of positions is generated using an enzyme (such as, but not limited to, a restriction endonuclease). DNA fragments from the test sample containing a variant sequence will have a different banding pattern than DNA fragments generated from the wildtype.

For example, sequences at a polymorphic site can be detected using a restriction fragment length polymorphism assay (“RFLP”). The region of interest in the DNA is first isolated using PCR. The PCR products are then cleaved with restriction enzymes known to give a unique length fragment for a given variant sequence. The restriction-enzyme digested PCR products are separated and detected (such by gel electrophoresis) and visualized (such as, but not limited to, by ethidium bromide staining). The length of the fragments is compared to molecular weight markers or fragments generated from wildtype and variant controls (for example, vectors containing the wildtype and variant sequences, respectively).

Sequences (alleles) at a polymorphic site can also be detected using a CLEAVASE fragment length polymorphism assay (“CFLP”; Third Wave Technologies, Madison, Wis.; See, U.S. Pat. No. 5,888,780). This assay is based on the observation that when single strands of DNA fold on themselves, they assume higher order structures that are highly individual to the precise sequence of the DNA molecule. These secondary structures involve partially duplexed regions of DNA such that single stranded regions are juxtaposed with double stranded DNA hairpins. The CLEAVASE I enzyme, is a structure-specific, thermostable nuclease that recognizes and cleaves the junctions between these single-stranded and double-stranded regions.

The region of interest is first isolated using routine techniques known in the art, such as by PCR. Next, DNA strands are separated by heating. The reactions are cooled to allow intrastrand secondary structure to form. The PCR products are then treated with the CLEAVASE I enzyme to generate a series of fragments that are unique to a given wildtype or variant sequence. The CLEAVASE enzyme treated PCR products are separated and detected (such by gel electrophoresis) and visualized (such as, but not limited to, by ethidium bromide staining). The length of the fragments is compared to molecular weight markers or fragments generated from wild-type and variant controls.

Mobility Based Assays

The presence or absence of a sequence (allele) at a polymorphic site may be determined by a single strand conformation polymorphism assay (“SSCP”). In this technique, PCR products from the region to be tested are heat denatured and rapidly cooled to avoid the reassociation of complementary strands. The single strands then form sequence dependent conformations that influence electrophoretic mobility. The different mobilities can then be analyzed by electrophoresis.

Alternatively, the assessment of a polymorphism may be by a heteroduplex assay. In this analysis, the DNA sequence to be tested is amplified, denatured and renatured to itself or to known wildtype DNA (namely, from SEQ ID NO:1 or SEQ ID NO:2). Heteroduplexes between different alleles contain DNA “bubbles” at mismatched basepairs that can affect electrophoretic mobility. Therefore, electrophoresis can be used to indicate the presence or absence of wildtype and variant sequences.

Hybridization Assays

The presence or absence of a sequence (allele) at a polymorphic site can be detected in a hybridization assay. In a hybridization assay, the presence or absence of a given sequence (allele) is determined based on the ability of the DNA from the test sample to hybridize to a complementary DNA molecule (such as, but not limited to, a probe). The hybridization of a probe to DNA from the test sample is subsequently detected. Detection of hybridization only to a wildtype probe, or only to a variant probe, indicate a homozygous wildtype or variant genotype, respectively. Detection of hybridization to both wildtype and variant probes indicates a heterozygous genotype. A number of hybridization assays using a variety of technologies for hybridization and detection are available. Examples of some of these assays are provided below.

Solution Based Detection

The presence or absence of polymorphisms can be determined using any solution based detection techniques known in the art. An example of such a technique that can be used is TaqMan® (Applied Biosystems, Forest City, Calif.; see, Holland et al; Proc. Natl. Acad. Sci. USA 88:7276-7280 (1991); and Gelmini et al. Clin. Chem. 43:752-758 (1997)). TaqMan® allows for the real-time quantification of PCR. TaqMan® probes are widely commercially available, and the TaqMan® system (Applied Biosystems) is well known in the art. TaqMan® probes anneal between the upstream and downstream primer in a PCR reaction. They contain a 5′-fluorophore and a 3′-quencher. During amplification the 5′-3′ exonuclease activity of the Taq polymerase cleaves the fluorophore off the probe. Since the fluorophore is no longer in close proximity to the quencher, the fluorophore will be allowed to fluoresce. The resulting fluorescence may be measured, and is in direct proportion to the amount of target sequence that is being amplified.

Another technique that can be used is a Molecular Beacon (See, Tyagi et al., Nat. Biotechnol. 14:303-308 (1996); and Tyagi et al., Nat. Biotechnol. 16:49-53 (1998)), the beacons are hairpin-shaped probes with an internally quenched fluorophore whose fluorescence is restored when bound to its target. The loop portion acts as the probe while the stem is formed by complimentary “arm” sequences at the ends of the beacon. A fluorophore and quenching moiety are attached at opposite ends, the stem keeping each of the moieties in close proximity, causing the fluorophore to be quenched by energy transfer. When the beacon detects its target, it undergoes a conformational change forcing the stem apart, thus separating the fluorophore and quencher. This causes the energy transfer to be disrupted to restore fluorescence. Any suitable fluorophore known in the art can be used. For example, fluorophores that can be used include, but are not limited to, FAM, HEX®, NED®, ROX®, Texas Red®. Quenchers that can be used include, but are not limited to, Dabcyl and TAMRA.

Another technique that can be used is Pyrosequencing™ (Pyrosequencing, Inc. Westborough, Mass.). This technique is based on the hybridization of a primer to a single stranded, PCR-amplified, DNA template in the presence of DNA polymerase, ATP sulfurylase, luciferase and apyrase enzymes and the adenosine 5′ phosphosulfate (“APS”) and luciferin substrates. In the second step, the first of four deoxynucleotide triphosphates (“DCNTP”) is added to the reaction and the DNA polymerase catalyzes the incorporation of the deoxynucleotide triphosphate into the DNA strand, if it is complementary to the base in the template strand. Each incorporation event is accompanied by release of pyrophosphate (“PPi”) in a quantity equimolar to the amount of incorporated nucleotide. In the last step, the ATP sulfurylase quantitatively converts PPi to ATP in the presence of adenosine 5′-phosphosulfate. This ATP drives the luciferase-mediated conversion of luciferin to oxyluciferin that generates visible light in amounts that are proportional to the amount of ATP. The light produced in the luciferase-catalyzed reaction is detected by a charge coupled device (“CCD”) camera and seen as a peak in a pyrogram™. Each light signal is proportional to the number of nucleotides incorporated.

Detection of Hybridization Using Reverse Solid Phase Detection

The presence or absence of polymorphisms can also be determined using reverse solid phase detection, such as, but not limited to, a microarray, such as a DNA chip assay. In a DNA chip assay, a series of probes are affixed to a solid support. The probes are designed to be unique to a given polymorphism. The DNA obtained from the test sample is contacted with the DNA “chip” and hybridization is detected. Any DNA “chip” assay known in the art can be used in the methods of the present invention. For example, the DNA chip assay can be a GeneChip assay (Affymetrix, Santa Clara, Calif.; See, U.S. Pat. No. 6,045,996). The GeneChip technology uses miniaturized, high-density arrays of probes affixed to a “chip.” Alternatively, a DNA microchip containing electronically captured probes (Nanogen, San Diego, Calif.; See, U.S. Pat. No. 6,068,818) can be used. Also, a “bead array” can also be used (Illumina, San Diego, Calif.; See WO 99/67641 and WO 00/39587). Illumina uses a BEAD ARRAY technology that combines fiber optic bundles and beads that self-assemble into an array.

Solid Phase Detection

In solid phase detection, hybridization of a probe to the sequence of interest, such as a polymorphism, is detected directly by visualizing a bound probe by using Southern blotting. In this technique, genomic DNA is isolated from a subject. The DNA is then cleaved with a series of restriction enzymes that cleave infrequently in the genome and not near any of the markers being assayed. The DNA is then separated (such as, but not limited to, by agarose gel electrophoresis) and transferred to a membrane. At least one probe which has been labeled with, for example, a radioactive, fluorescent or enzymatic label, specific for the polymorphism being detected is allowed to contact the membrane under a condition of low, medium, or high stringency conditions. Unbound probe is removed and the presence of binding is detected by visualizing the labeled probe.

Enzymatic Detection of Hybridization

The presence or absence of polymorphisms can be detected using an assay that detects hybridization by enzymatic cleavage of specific structures (“INVADER assay”, Third Wave Molecular Diagnostics, Madison, Wis.; See, U.S. Pat. No. 6,001,567). The INVADER assay detects specific DNA and RNA sequences by using structure-specific enzymes to cleave a complex formed by the hybridization of overlapping probes. Elevated temperature and an excess of one of the probes enable multiple probes to be cleaved for each target sequence present without temperature cycling. These cleaved probes then direct cleavage of a second labeled probe. The secondary probe can be 5′-end labeled (such as, but not limited to, with fluorescein) that is quenched by an internal dye. Upon cleavage, the dequenched fluorescein labeled product may be detected using a standard fluorescence plate reader.

The INVADER assay detects specific mutations and SNPs in unamplified genomic DNA. The isolated DNA sample is contacted with the first probe specific either for a SNP or wild type sequence and allowed to hybridize. Then a secondary probe, specific to the first probe, and containing the fluorescein label, is hybridized and the enzyme is added. Binding is detected using a fluorescent plate reader and comparing the signal of the test sample to known positive and negative controls.

Hybridization of a bound probe can be detected using a TaqMan® assay using the techniques described previously herein.

In still further embodiments, polymorphisms are detected using any single base extension (“SBE”) methods known in the art (See U.S. Pat. Nos. 5,888,819 and 6,004,744). For example, a shifted termination assay (“STA”) can be performed. The STA method involves designing a detection primer that is complementary to a target DNA. The detection primer is labeled with any detectable label known in the art. The 3′-terminal of detection primer ends at the base just before the target base. The detection primer hybridizes to the target nucleic acid sequence. When performing a primer extension reaction, if the first base is the target base, a primer extension reaction will be terminated at the target base position without incorporating any of the labeled nucleotides. No color reaction will be detected. If the target base is changed by any type of mutation, including point mutation (SNP), deletion, insertion, and translocation, a primer extension reaction will continue through the target base position, and multiple labeled nucleotides will be incorporated into the extended detection primer. A strong color reaction will be observed. A STA can be performed on a DNA sequence or using fluorescence polarization.

Another SBE that can be performed is a SNP-IT primer extension assay (Orchid Biosciences, Princeton, N.J.; See, U.S. Pat. No. 5,952,174). In this assay, SNPs are identified using a specially synthesized DNA primer and a DNA polymerase to selectively extend the DNA chain by one base at the suspected SNP location. DNA in the region of interest is amplified and denatured. PCR is then performed using miniaturized systems called microfluidics. Detection is accomplished by adding a label to the nucleotide suspected of being at the polymorphic site. Incorporation of the label into the DNA can be detected by any method known in the art.

Mass Spectroscopy

The presence or absence of polymorphisms can be detected using a MassARRAY system (Sequenom, San Diego, Calif.; See, U.S. Pat. No. 6,043,031). DNA is isolated from test samples using routine procedures known to those skilled in the art. Next, specific DNA regions containing the polymorphism of interest, about 200 base pairs in length, are amplified by PCR. The amplified fragments are then attached by one strand to a solid surface and the non-immobilized strands are removed by standard denaturation and washing. The remaining immobilized single strand then serves as a template for automated enzymatic reactions that produce genotype specific diagnostic products.

Very small quantities of the enzymatic products, typically five to ten nanoliters, are then transferred to a SpectroCHIP array for subsequent automated analysis with the SpectroREADER mass spectrometer. Each spot is preloaded with light absorbing crystals that form a matrix with the dispensed diagnostic product. The MassARRAY system uses MALDI-TOF (Matrix Assisted Laser Desorption Ionization-Time of Flight) mass spectrometry. In a process known as desorption, the matrix is hit with a pulse from a laser beam. Energy from the laser beam is transferred to the matrix and it is vaporized resulting in a small amount of the diagnostic product being expelled into a flight tube. As the diagnostic product is charged when an electrical field pulse is subsequently applied to the tube they are launched down the flight tube towards a detector. The time between application of the electrical field pulse and collision of the diagnostic product with the detector is referred to as the time of flight. This is a very precise measure of the product's molecular weight, as a molecule's mass correlates directly with time of flight with smaller molecules flying faster than larger molecules. The entire assay is completed in less than 0.0001 second, enabling samples to be analyzed in a total of 3-5 second including repetitive data collection. The SpectroTYPER software then calculates, records, compares and reports, the genotypes at the rate of three seconds per sample.

Kits

The present invention also provides kits that enable or allow for the detection of a genotype of one or more subjects. These kits are useful for diagnosing those subjects suffering from major depression or a related disorder or at risk of developing major depression or a related disorder, providing a prognosis for or predicting a response to treatment for a subject suffering from major depression or a related disorder, identifying subjects for selection or inclusion in a clinical trial for treating major depression or a related disorder, or for analyzing the relationship between genotypes of subjects being treated for major depression or a related disorder and their clinical outcome.

The kits can be produced in a variety of ways. For example, the kits contain at least one reagent useful for detecting (a) at least one polymorphic site in SEQ ID NO:1; (b) at least one polymorphic site in nucleotides 1 to 315 of SEQ ID NO:2; (c) a T-C polymorphism at position 135 of SEQ ID NO:2; (d) a A-G polymorphism at position 209 of SEQ ID NO:2; (e) a G-A polymorphism at position 241 of SEQ ID NO:2; (f) at least one polymorphic site in SEQ ID NO:1; (g) a polymorphic site in nucleotide 77402 of SEQ ID NO:1; (h) a polymorphic site in nucleotide 79906 in SEQ ID NO:1; or (i) any combinations of (a)-(h). Additionally, any of the kits described above in (a)-(i) can further contain at least one reagent useful for detecting a C-G polymorphism at position −1019 in a human serotonin receptor 1A gene. Examples of the at least one reagent that can be included in the kits described herein are one or more primers for amplifying the region of DNA containing the polymorphic site or one or more probes that bind to or near the polymorphic site. In addition, the kits can further contain (a) instructions for determining the genotype of a subject; (b) ancillary reagents such as buffering agents, nucleic acid stabilizing reagents, protein stabilizing reagents, and signal producing systems (such as, but not limited to, fluorescence generating systems); or (c) positive and/or negative control(s). The kit may be packaged in any suitable manner, typically with the elements in a single container or various containers as necessary.

RNA and Protein Detection and Quantification Assays

In another embodiment, the present invention relates methods for detecting or quantifying mRNA or protein in a test sample obtained from one or more subjects. The information obtained by detecting or quantifying mRNA or protein in a test sample obtained from a subject can be used in a variety of different ways. For example, the presence, absence or amount of mRNA or protein detected or quantified in subjects can be used to diagnose those subjects suffering from major depression or a related disorder or at risk of developing major depression or a related disorder, provide a prognosis for or predict or diagnose a response to treatment for a subject suffering from major depression or a related disorder or identifying subjects for selection or inclusion in a clinical trial for treating major depression or a related disorder. Additionally, the presence, absence or amount of mRNA or protein can be used to analyze the results of a clinical trial for subjects being treated for major depression or a related disorder. Specifically, the relationship between the presence, absence or amount of the mRNA or protein detected or quantified in the test samples and the clinical outcome of said subjects can be determined.

The methods described herein involve obtaining a test sample from said subject(s). The subject may or may not be experiencing any symptoms of major depression or a related disorder at the time the test sample is obtained. In this embodiment, a test sample is any biological sample which contains the RNA or protein of the subject. Test samples can be prepared using techniques well known to those skilled in the art such as by obtaining a specimen from an individual and, if necessary, disrupting any cells contained therein to release RNA or protein. Examples of test samples include, but are not limited to, whole blood, serum, plasma, cerebrospinal fluid, sputum, bronchial washing, bronchial aspires, urine, lymph fluids, and various external secretions of the respiratory, intestinal and genitourinary tracts, tears, saliva, milk, white blood cells, myelomas and the like, etc.

Once the test sample(s) is obtained, it is analyzed, using routine techniques known in the art, in order to determine or quantify the presence, absence or amount of: (a) at least one mRNA which comprises nucleotides 1 to 315 of SEQ ID NO:2; (b) at least one mRNA transcribed from SEQ ID NO:1; (c) at least one protein having an amino acid sequence of SEQ ID NO:3 or SEQ ID NO:4; (d) at least one polypeptide translated from SEQ ID NO:1; or (e) any combinations of (a)-(d). Additionally, the test sample may optionally be further analyzed for the presence, absence or amount of mRNA transcribed from the HTR1A gene or a polypeptide translated from the HTR1A gene.

As discussed above, once a test sample is obtained, it can be analyzed, using routine techniques known in the art for the presence, absence or amount of at least one mRNA transcribed from SEQ ID NO:1. Examples of mRNAs transcribed from SEQ ID NO:1 include, but are not limited to, SEQ ID NO:2, SEQ ID NO:5, SEQ ID NO:6, SEQ ID NO:7, SEQ ID NO:8, SEQ ID NO:9, SEQ ID NO:11, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:31 or SEQ ID NO:33. Alternatively, the test sample can be analyzed for the presence, absence or amount of at least one polypeptide translated from SEQ ID NO:1. Examples of polypeptides translated from SEQ ID NO:1 include, but are not limited to, SEQ ID NO:3, SEQ ID NO:4, SEQ ID NO:10, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:32 and SEQ ID NO:34.

Once the presence, absence or amount of a mRNA or a protein as specified in (a)-(e) above has been determined or quantified in a test sample, then further determinations can be made, such as, diagnosing whether the subject has major depression or a related disorder or is at risk of developing major depression or a related disorder, providing a prognosis for or predicting the response to treatment for a subject having major depression or a related disorder, determining whether the subject should be selected for inclusion in a clinical trial for treatment of major depression or a related disorder, or analyzing the relationship between the frequency of presence or relative amounts of at least one mRNA or polypeptide in subjects, and their clinical outcome. Additionally, if the test sample is further analyzed for the presence, absence or amount of mRNA transcribed from the HTR1A gene or a polypeptide translated from the HTR1A gene, the information pertaining to mRNA(s) or polypeptide(s) transcribed or translated from DEP2 may be used in combination with the information pertaining to mRNA(s) or polypeptide(s) transcribed or translated from HTR1A to make further determinations, as elaborated above.

For example, a test sample can be obtained from a subject. The test sample can then be analyzed using routine techniques known in the art, in order to determine or quantify the presence, absence or amount of (a) at least one mRNA which comprises nucleotides 1 to 315 of SEQ ID NO:2; (b) at least one mRNA transcribed from SEQ ID NO:1; (c) at least one protein having an amino acid sequence of SEQ ID NO:3 or SEQ ID NO:4; (d) at least one polypeptide translated from SEQ ID NO:1; or (e) any combinations of (a)-(d). If, for example, the presence of (a) at least one mRNA which comprises nucleotides 1 to 315 of SEQ ID NO:2; (b) at least one mRNA transcribed from SEQ ID NO:1; (c) at least one protein having an amino acid sequence of SEQ ID NO:3 or SEQ ID NO:4; (d) at least one polypeptide translated from SEQ ID NO:1; or (e) any combinations of (a)-(d) is detected, then a diagnosis can be made for said subject related to major depression or a related disorder or related to risk of developing major depression or a related disorder. This information can also be useful for providing a prognosis for or predicting the response to treatment for a subject already diagnosed as suffering from major depression or a related disorder. Moreover, this information can be used to determine whether or not the subject should or could be selected for inclusion in a clinical trial for treatment of major depression or a related disorder. Further, the frequency of presence of: (a) at least one mRNA which comprises nucleotides 1 to 315 of SEQ ID NO:2; (b) at least one mRNA transcribed from SEQ ID NO:1; (c) at least one protein having an amino acid sequence of SEQ ID NO:3 or SEQ ID NO:4; (d) at least one polypeptide translated from SEQ ID NO:1; or (e) any combinations of (a)-(d) can be used to analyze the results of a clinical trial for subjects being treated for major depression or a related disorder. Specifically, the relationship between the presence of said mRNA, protein, polypeptide or combinations thereof in the test samples and the clinical outcome of said subjects can be determined. Similarly, any of the above further determinations might be made on the basis of absence of: (a) at least one mRNA which comprises nucleotides 1 to 315 of SEQ ID NO:2; (b) at least one mRNA transcribed from SEQ ID NO:1; (c) at least one protein having an amino acid sequence of SEQ ID NO:3 or SEQ ID NO:4; (d) at least one polypeptide translated from SEQ ID NO:1; or (e) any combinations of (a)-(d), or on the basis of detection or quantification that an amount of: (a) at least one mRNA which comprises nucleotides 1 to 315 of SEQ ID NO:2; (b) at least one mRNA transcribed from SEQ ID NO:1; (c) at least one protein having an amino acid sequence of SEQ ID NO:3 or SEQ ID NO:4; (d) at least one polypeptide translated from SEQ ID NO:1; or (e) any combinations of (a)-(d), is within a certain range.

Techniques for identifying the presence, absence or amount of mRNAs or proteins in a test sample are well known in the art. For example, techniques for identifying the presence, absence or amount of mRNAs include, but are not limited to, reverse transcriptase, cDNA microarrays, quantitative PCR and Northern blotting. Techniques for identifying the presence, absence or amount of proteins include, but are not limited to, ELISA, RIA, Western blotting, fluorescence activated cell sorting and immunohistochemical analysis. These techniques will be discussed briefly below.

RNA Techniques

Reverse transcriptase can be used to prepare a cDNA by used of an oligo dT primer which is annealed to the poly A sequence of the RNA. Examples of reverse transcriptases that can be used include, but are not limited to, ImProm-II Reverse Transcriptase (Promega, Madison, Wis.) and BD Powerscript Reverse Transcriptase (BD Biosciences, Franklin Lakes, N.J.). Methods for using reverse transcriptases to prepare and obtain cDNA molecules are well known in the art and are described in Sambrook, J. et al., Molecular Cloning: A Laboratory Manual, 2nd edition, Cold Spring Harbor Press, Cold Spring Harbor, N.Y. (1989).

A cDNA microarray is an array of multiple cDNA molecules, fixed in addressable locations (such as on a chip assay), to which complementary nucleic acids in applied samples may hybridize (see Hegde et al., Biotechniques 29(3):548-562 (2000)). cDNA microarrays provide for qualitative and quantitative analysis of mRNA expression of the molecules contained in the array.

Quantitative PCR allows for the direct monitoring of the progress of a PCR amplification as it is occurring, without the need for repeated sampling of the reaction products. In quantitative PCR, the reaction products may be monitored as they are generated and are tracked after they rise above background but before the reaction reaches a plateau. The number of cycles required to achieve a chosen level of fluorescence varies directly with the concentration of amplifiable targets at the beginning of the PCR process, enabling a measure of signal intensity to provide a measure of the amount of target DNA in a sample in real time. Quantitative PCR according to the present invention may be performed on any suitable instrument, including, but not limited to, Mx4000 or Mx3000P (Stratagene, La Jolla, Calif.), ABI7700 or ABI7000 (Applied BioSystems Inc., Foster City, Calif.), MJ Opticon (MJ Research, Watertown, Mass.), iCycler (Bio-Rad, Hercules, Calif.), RotorGene 3000 (Corbett Life Sciences, Mortlake, NSW, Australia), and the SmartCycler (Cepheid, Sunnyvale, Calif.).

In solid phase detection, hybridization of a probe to the sequence of interest, such as an RNA, is detected directly by visualizing a bound probe by using Northern blotting. In this technique, RNA is isolated from a subject. The RNA is then cleaved with a series of restriction enzymes that cleave infrequently in the genome and not near any of the markers being assayed. The RNA is then separated (such as, but not limited to, by agarose gel electrophoresis) and transferred to a membrane. At least one probe which has been labeled with, for example, a radioactive, fluorescent or enzymatic label, specific for the polymorphism being detected is allowed to contact the membrane under a condition of low, medium, or high stringency conditions. Unbound probe is removed and the presence of binding is detected by visualizing the labeled probe.

Protein Techniques

ELISA involves the fixation of a test sample containing a protein substrate of interest to a surface such as a well of a microliter plate. A substrate specific antibody coupled to an enzyme is applied and allowed to bind to the substrate. Presence of the antibody is then detected and quantitated by a colormetric reaction employing the enzyme coupled to the antibody. Enzymes commonly in ELISAs include, but are not limited to, horseradish peroxidase and alkaline phosphatase. If well calibrated and within the linear range of response, the amount of substrate present in the sample is proportional to the amount of color produced. A substrate standard is generally employed to improve quantitative accuracy.

Another technique that can be used is a radioimmunoassay (“RIA”). One version of RIA involves the precipitation of a desired substrate (such as a protein of interest) with a specific antibody and detectably labeled antibody binding protein (the antibody binding protein can be labeled with any detectable isotope known in the art) immobilized on a precipitable carrier, such as, but not limited to, agarose beads. The number of counts in the precipitated pellet is proportional to the amount of substrate present in the test sample. In an alternate version of RIA, a labeled substrate (such as a protein of interest) and an unlabelled antibody binding protein are employed. A test sample containing an unknown amount of substrate is added in varying amounts. The decrease in precipitated counts from the labeled substrate is proportional to the amount of substrate in the added sample.

Western blot involves separation of a substrate (such as a protein of interest) from another protein by means of an acrylamide gel followed by transfer of the substrate to a membrane (such as, but not limited to, nylon or PVDF). The presence of the substrate is then detected by antibodies specific to the substrate. The antibodies are then detected by antibody binding reagents. Antibody binding reagents may include, but are not limited to, protein A or other antibodies. The Antibody binding reagents may labeled with a detectable label as described previously herein. Detection may be by autoradiography, calorimetric reaction or chemiluminescence. Western blotting allows for both the quantitation of an amount of substrate and a determination of the substrate's identity by a relative position on the membrane which is indicative of a migration distance in the acrylamide gel during electrophoresis.

Fluorescence activated cell sorting (“FACS”) involves detection of a substrate (such as a protein of interest) in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

Immunohistochemical analysis involves detection of a substrate (such as a protein of interest) in situ in fixed cells by substrate specific antibodies. The substrate specific antibodies may be enzyme linked or linked to fluorophores. Detection is by microscopy and subjective evaluation. If enzyme linked antibodies are employed, a calorimetric reaction may be required.

Kits

The present invention also provides kits that enable or allow for the detection or quantification of (a) at least one mRNA which comprises nucleotides 1 to 315 of SEQ ID NO:2; (b) at least one mRNA transcribed from SEQ ID NO:1; (c) at least one protein having an amino acid sequence of SEQ ID NO:3 or SEQ ID NO:4; (d) at least one polypeptide translated from SEQ ID NO:1; or (e) any combinations of (a)-(d) in one more subjects. These kits are useful for diagnosing those subjects suffering from major depression or a related disorder or at risk of developing major depression or a related disorder, providing a prognosis for or predicting a response to treatment for a subject suffering from major depression or a related disorder, identifying subjects for selection or inclusion in a clinical trial for treating major depression or a related disorder, or for analyzing the results of a clinical trial for treating major depression or a related disorder relationship.

The kits can be produced in a variety of ways. For example, the kits contain at least one reagent useful for detecting or quantifying the presence, absence or amount of: (a) at least one mRNA which comprises nucleotides 1 to 315 of SEQ ID NO:2; (b) at least one mRNA transcribed from SEQ ID NO:1; (c) at least one protein having an amino acid sequence of SEQ ID NO:3 or SEQ ID NO:4; (d) at least one polypeptide translated from SEQ ID NO:1; or (e) any combinations of (a)-(d). Additionally, any of the kits described above in (a)-(e) can further contain at least one reagent useful for detecting or quantifying the presence, absence or amount of the presence, absence or amount of mRNA transcribed from the HTR1A gene or a polypeptide translated from the HTR1A gene. Examples of the at least one reagent that can be included in the kits described herein are a reverse transcriptase, one or more primers for amplifying cDNA or at least one antibody. In addition, the kits can further contain (a) instructions describing how to detect or quantify the presence, absence or amount of at least one mRNA or at least one protein in a test sample; (b) ancillary reagents such as buffering agents, nucleic acid stabilizing reagents, protein stabilizing reagents, and signal producing systems (such as, but not limited to, fluorescence generating systems); or (c) positive and/or negative control(s). The kit may be packaged in any suitable manner, typically with the elements in a single container or various containers as necessary.

Screening Assays and Methods of Treatment

In another embodiment, the present invention relates to methods (also referred to herein as “screening assays” or “screening methods”) for identifying compositions, namely candidate or test compounds or agents (such as, but not limited to, small molecules, antibodies, nucleic acids, peptides, peptidomimetics, or other drugs), which: (a) bind to a protein translated from SEQ ID NO:1; (b) modulate the activity or expression of a protein translated from SEQ ID NO:1 (such as by inhibiting, reducing or decreasing the activity, reducing or decreasing the expression, or by stimulating or increasing the activity, or stimulating or increasing the expression, of the protein); or (c) modulate the expression of an mRNA molecule transcribed from SEQ ID NO:1 (such as by reducing or decreasing the expression or by stimulating or increasing the expression. Since genetic linkage between DEP2 and major depressive disorder has been established, it is thought that compositions identified pursuant to the screening methods described herein may be useful in treating major depression or a related disorder.

Examples of proteins translated from SEQ ID NO:1 include, but are not limited to, (i) Lhpp (SEQ ID NO:10), (ii) naturally occurring protein variants of Lhpp (SEQ ID NO:13, SEQ ID NO:15, SEQ ID NO:17, SEQ ID NO:19, SEQ ID NO:21, SEQ ID NO:23, SEQ ID NO:25 and SEQ ID NO:29); (iii) Dep2-1a (SEQ ID NO:3); (iv) Dep2-1b (SEQ ID NO:4); Dep2-2 (SEQ ID NO:27); (v) Dep2-4 (SEQ ID NO:32); and (vi) Dep2-5 (SEQ ID NO:34). Examples of RNA molecules transcribed from SEQ ID NO:1 include, but are not limited to, SEQ ID NO:2, SEQ ID NO:5, SEQ ID NO:6, SEQ ID NO:7, SEQ ID NO:8, SEQ ID NO:9, SEQ ID NO:11, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:31 or SEQ ID NO:33.

As will be discussed in more detail herein, the present invention includes a method of determining whether a composition, identified in accordance with the methods described herein, is a potential therapy for major depression or a related disorder by initially administering the composition to a mammal (for example, an animal model). One may then monitor for major depression-related symptoms of the animal or the level or activity of a protein translated from SEQ ID NO:1 in the test subject. A decrease in the appearance of such symptoms indicates the potential suitability of the composition of interest in the treatment of major depression or related disorders. Such a finding in an animal model would then lead to use of the composition in human clinical trials.

Suitable animal models for such experiments include, but are not limited to, behavioral despair or mouse forced swim test (Arch. Int. Pharmacodyn. 229:327-336 (1977), Psychopharmacology 94:147-160 (1988)); tail suspension test (Psychopharmacology 85:367-370 (1985)); elevated plus maze test (Psychopharmacology 92:180-185 (1987)); open field test (Behav. Brain Res. 134:49-57 (2002)); dark-light transitions test (Pharmacol. Biochem. Behav. 15:695-699 (1981)); Irwin test (Brain Res. Vol. 835:18-26 (1999); Psychopharmacology 147:2-4 (1999)); inescapable stress test (learned helplessness) (Seligman and Maier, J. Exp. Psychol 74:1-9, (1967)); chronic mild stress (Ducottet et al., Prog. Neuro-Psychopharmacol. Biol. Psychiatry 27:625-631 (2003); Kopp et al., Behav. Pharmacol. 10:73-83 (1989)); and novelty-suppressed feeding model (Bodnoff et al., Psychopharamcology 97:277-279 (1989)).

The present invention additionally relates to the compositions identified by use of the above screening methods as well as to methods of using these compositions in the treatment of major depression or a related disorder. More specifically, once a composition of interest has been identified, the composition may be used in clinical trials to determine whether it actually alleviates the symptoms of major depression or a related disorder or at least decreases the severity thereof.

Also, it is submitted that the proteins described herein may be used to characterize the physical properties of compositions which may be used to ultimately treat major depression or a related disorder and thus in the “design” of such compositions. Thus, based upon such properties, one may design a composition or compound that has the ability to have a significant degree of binding affinity to a protein translated from SEQ ID NO:1, thereby modulating the activity of the protein. Such a composition or compound could then be used in the treatment of major depression or a related disorder.

Furthermore, one may detect binding of a test composition to a protein translated from SEQ ID NO:1 by subjecting the protein to, for example, nuclear magnetic resonance (“NMR”) alone and in the presence of the composition. Characteristic changes in the NMR spectrum of the protein may then allow one to determine whether and how the composition has bound to the protein. This procedure may be repeated for a series of compounds, enabling discovery of relationships between compound structure and binding to the target protein. This iterative process is known as “structure-activity relationships by NMR” or “SAR by NMR” (Shuker et al., Science 274:1531-1534 (1996); SAR by NMR is described in U.S. Pat. Nos. 5,891,643, 5,989,827, 5,804,390, 6,043,024 and 6,897,337).

Similarly, one may identify the structure of a composition bound to the protein by x-ray diffraction techniques. By iterative operation of this technique, one may optimize lead compositions or compounds so as to develop the most efficacious therapeutic compositions or compounds for the treatment of major depression or a related disorder.

One method of identifying compositions that modulate the amount or activity of a protein translated from SEQ ID NO:1 or that modulate the expression of an mRNA molecule that is transcribed from SEQ ID NO:1 is a reporter gene assay. It is well known to those skilled in the art that a reporter gene assay may be carried out in an intact cell transfected with the reporter gene construct, in extracts from a cell transfected with the reporter gene construct, or in a cellular extract (for example, reticulocyte lysate) to which the reporter gene construct is added. It is further recognized that reporter gene assays may be carried out using cells or extracts that naturally contain a protein translated from SEQ ID NO:1 or an mRNA molecule transcribed from SEQ ID NO:1, cells into which a vector for the expression of a protein translated from SEQ ID NO:1 or an mRNA molecule transcribed from SEQ ID NO:1 that has been transfected (transiently or stably), or extracts to which a purified or partially purified amino acid translated from SEQ ID NO:1 or an mRNA molecule transcribed from SEQ ID NO:1 is added. In the present invention, it is preferred that a protein translated from SEQ ID NO:1 or an mRNA molecule transcribed from SEQ ID NO:1 be purified from a human cell or tissue, or from expression in an heterologous system. Further, it is also well known in the field that reporter gene assays may be conducted in cells or extracts that are of human origin, or that come from a different mammal or organism. It is additionally recognized that there are many regulatory sequences (such as promoters) that can be used to initiate transcription in a reporter gene construct, and that the choice of a regulatory sequence may be determined more by the particular cell or extract in which the assay will be conducted. It is still further well known that there are a variety of reporter genes that are amenable to screening assays, including high throughput screening assays. Examples of reporter genes include those which are themselves fluorescent, luminescent or have easily detected spectral characteristics (for example, a green fluorescent protein), as well as those having well-characterized fluorescent, luminescent or colorimetric substrates (for example, beta-galactosidase, luciferases). It is finally recognized that certain cofactors may be added as purified or partially purified components to a reporter gene assay. A discussion of reporter systems can be found in Current Protocols in Pharmacology (2003), Units 6.2.1-6.2.11, Wiley & Sons, Inc.

An additional embodiment of reporter gene assays involve the use of at least one substrate for a protein translated from SEQ ID NO:1. The substrate can be added before the protein is exposed to the test composition or simultaneously with the test composition, provided that the protein is exposed to the substrate for a time and under conditions sufficient to allow the protein to react with the substrate in order to produce a reaction product. Any substrate wherein the phosphorylation of said substrate is capable of being modified by a protein translated from SEQ ID NO:1 can be used in the reporter gene assays described herein. Proteins translated from SEQ ID NO:1 that can be used to modify the phosphorylation of said substrate, include, for example, proteins having an amino acid sequence selected from the group consisting of: SEQ ID NO:10, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:32 and SEQ ID NO:34. Examples of substrates that can be used include, but are not limited to, phosphohistidine, phospholysine, phosphodiimide, pyrophosphate or any peptide or protein that is phosphorylated on a histidine or a lysine. For example, a reporter gene assay for screening a composition for the ability to inhibit activity of SEQ ID NO:10, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:32 or SEQ ID NO:34 can be performed. The method involves exposing a protein to a test composition and then measuring the presence or absence of a reaction product or complex. The lack of a reaction product or complex indicates that the composition has the ability to inhibit the activity of the protein. Prior to exposing the protein to the test composition, a substrate can be added to the protein. Alternatively, the substrate can be added when the protein is exposed to the test composition.

An additional substrate that can be used is a radioactive enzyme substrate. In such an embodiment, the reporter gene encodes an enzyme (for example, chloramphenicol acetyltransferase) having a substrate that is readily separated from the corresponding reaction product. This type of radioactive detection assay may be utilized in order to identify a compound that binds to or modulates a protein translated from SEQ ID NO:1 or that modulates the expression of an mRNA molecule transcribed from SEQ ID NO:1. It is well known to those skilled in the art that the separation and detection of radioactive compounds may be accomplished by a variety of chromatographic and other methods. A discussion of radioactive reporter gene assays can be found in Current Protocols in Pharmacology (2003), Units 6.4.1-6.4.11, Wiley & Sons.

An additional assay that may be used to detect a composition or compound having the ability to modulate the activity or expression of a protein translated from SEQ ID NO:1 or modulate the expression of an mRNA molecule transcribed from SEQ ID NO:1 is the scintillation proximity assay. This assay is based upon the binding of a radiolabeled tracer to a protein translated from SEQ ID NO:1 or an mRNA molecule transcribed from SEQ ID NO:1 that has been exposed to the composition or compound of interest. The scintillant is incorporated into small fluoromicrospheres to which target macromolecules (for example, proteins or mRNAs) attach. If a radioactive molecule (for example, ³H) binds to the target, it is brought close enough to the bead to stimulate the scintillant to produce light. On the other hand, unbound radioactivity is not detected if the bead is outside the distance subatomic particles produced by the decay are likely to travel. Thus compositions or compounds that bind to a protein translated from SEQ ID NO:1 or an mRNA molecule transcribed from SEQ ID NO:1 may be detected by changes in the amount of scintillant-emitted light. A discussion of scintillation proximity assays can be found in Current Protocols in Pharmacology (2003), Unit 9.4.9-9.4.10, Wiley & Sons, Inc.

Another assay which may be utilized in the identification of compositions that affect the binding or that modulate a protein translated from SEQ ID NO:1 to mRNA is a filter binding assay. An example of the filter binding assay that may be utilized for a protein translated from SEQ ID NO:1 involves immobilization of an RNA molecule (for example, all or part of an mRNA transcribed from SEQ ID NO:1) on a solid support, exposure of the immobilized RNA to a protein translated from SEQ ID NO:1 in the absence or presence of compositions thought to bind or inhibit protein translated from SEQ ID NO:1, and quantitation of a protein translated from SEQ ID NO:1 on the solid support. It is well known to those skilled in the art that the solid support may be a nitrocellulose or other filter, or any of a variety of beads or microparticles. It is further recognized that a protein translated from SEQ ID NO:1 used in the assay may be purified from an heterologous expression system, and will advantageously be tagged such that it can be detected using commonly available reagents. For example, a protein translated from SEQ ID NO:1 may be a fusion to a ‘tag’ sequence expressed in E. coli (Tateiwa et al., Journal of Neuroimmunology 120:161-69 (2001)). Compositions that bind to or inhibit a protein translated from SEQ ID NO:1 may be identified by a increase or reduction in the amount of a protein translated from SEQ ID NO:1 on the solid support, relative to a reaction in which no test compound was added.

Another type of assay that may be useful to screen for compositions that bind to protein translated from SEQ ID NO:1 is a fluorescence polarization assay. This method detects molecular interactions and is based on the concept that fluorescent molecules excited by light polarized in one plane will emit a fluorescent signal again in a polarized manner. The rotational relaxation time is proportional to the molecular volume if other physical variables are unchanged. Thus, when binding to a larger molecule restricting rotation and tumbling, the emission remains polarized, such polarization can be calculated and is directly proportional to the fraction of bound ligand. Change in fluorescence polarization thus accounts for the ratio of bound versus total ligand. For a protein translated from SEQ ID NO:1, one embodiment of a fluorescent polarization assay would involve a fluorescently labeled polynucleotide comprising all or part of a nucleic acid of SEQ ID NO:1, SEQ ID NO:2, SEQ ID NO:5, SEQ ID NO:6, SEQ ID NO:7, SEQ ID NO:8, SEQ ID NO:9, SEQ ID NO:11, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:31 or SEQ ID NO:33. Compositions that bind to a protein translated from SEQ ID NO:1 may be detected by a reduction of fluorescent polarization attributable to the labeled polynucleotide. A discussion of fluorescent polarization assays can be found in Current Protocols in Pharmacology, (2003), Units 9.4.12-9.4.13 Wiley & Sons, Inc.

Another type of assay that may be used to screen for compositions that bind to a protein translated from SEQ ID NO:1 is the spin-screening assay. This method detects molecular interactions, and is based on the concept that the sedimentation rate of molecules or molecular complexes in solution depends on mass and shape. In particular, the sedimentation rate of a small molecule alone is expected to be substantially different from that of the same small molecule bound to a macromolecule. Thus, when a directional force is applied (for example, by spinning a solution at high speed in a centrifuge), small molecules that bind to a protein translated from SEQ ID NO:1 can be readily separated from other small molecules in a mixture that do not. Separation of bound from unbound small molecules can also be accomplished by including a size exclusion filter within the centrifuge tube, such that unbound small molecules pass through the filter but bound small molecules do not. It is recognized by those skilled in the art that molecules separated in this fashion can be identified by a variety of spectroscopic and other methods. In one embodiment, a spin-screening assay includes detection by mass spectrometry.

In another embodiment, the present invention relates to methods of determining the in vivo activity of a composition identified as a potential therapy for the treatment of major depression or a related disorder. These methods involve obtaining at least two (2) test samples from a subject, preferably a human, being treated for major depression or a related disorder. A first test sample can be considered to be a test sample that is obtained at a period in time before the subject has begun a course of treatment with the test composition. Alternatively, a first test sample can also be considered to be a test sample obtained at a period in time during which a subject has been receiving a course of treatment with the test composition. A second test sample can be considered to be a test sample that is obtained at a period in time that is subsequent to the obtaining of the first test sample. For example, the second test sample can be obtained after a period of time has elapsed after the subject has begun an initial course of treatment with the test composition (meaning that the subject had not previously received the test composition prior obtaining the first test sample). Alternatively, if a subject has been receiving treatment with a test composition, a first test sample can be obtained from said subject. After a period of time has elapsed (for example, three (3) months) during which said subject is still being treated with the test composition, a second test sample can be obtained from said subject. A discussion of what constitutes a test sample and examples of test samples has already been provided previously herein and is incorporated herein by reference.

Once the test samples are obtained, they are analyzed, using routine techniques known in the art (which have been discussed previously herein), to determine or quantify the: (a) amount or activity of a protein translated from SEQ ID NO:1; or (b) amount of mRNA transcribed from SEQ ID NO:1, in each of the test samples. The amount or activity of a protein translated from SEQ ID NO:1 or the amount of mRNA transcribed from SEQ ID NO:1 that was detected or quantified in each of the test samples is compared. If, for example, the amount or activity of protein or the amount of mRNA determined or quantified in the second test sample (which was the test sample obtained after the subject began a course of treatment with the composition) is the same (namely, equal) as the amount or activity of protein or the amount or activity of mRNA determined or quantified in the first test sample (which was the test sample obtained from the subject prior to undergoing said course of treatment with the composition), this indicates that the composition lacks therapeutic activity. In contrast, if the amount or activity of the protein or the amount of mRNA determined or quantified in the second test sample has changed, namely, has increased or decreased, when compared to the amount or activity of the protein or the amount of mRNA determined or quantified in the first test sample, this indicates that the composition possesses some type or degree of therapeutic activity.

In addition, in yet another embodiment, the present invention relates to methods for determining the presence or absence of activity of a composition identified pursuant to the screening methods described herein that is being used to treat a subject suffering from major depression or a related disorder. The method involves observing the phenotype of said subject prior to the subject being administered the test composition (“first visit”). For example, observation of the subject's phenotype should be based on a method that has been validated as a measure of major depression or a related disorder. Such validated methods include, but are not limited to: the Hamilton depression rating scale (Hamilton, J. Neurol. Neurolsurg. Psychiatry 23:56-62 (1960), Schedule for affective disorders and schizophrenia (Spitzer and Endicott, Schedule for affective disorders and schizophrenia, lifetime version. New York, N.Y.: New York State

Psychiatric Institute, Biometrics Research. 1975), Montgomery-Åsberg depression rating score (Montgomery, Br. J. Psychiatry 134:382-389 (1979)) and the Structured clinical interview for DSM-IV (First et al., Structured Clinical Interview for DSM-IV. Washington, D.C.: American Psychiatric Press 1997). After observation, the subject is administered the test composition for a time and under conditions that are sufficient for the composition to either: (a) bind to, inhibit, increase, decrease or reduce the amount of a protein translated from SEQ ID NO:1; or (b) increase or reduce the amount of a mRNA molecule transcribed from SEQ ID NO:1. After the subject has been administered the test composition for a time and under the conditions described above, the phenotype of the subject is again observed, preferably, using the same validated method as was used to establish the initial phenotype. Observable improvement in the phenotype of the subject at the second observation compared to the first observation indicates that the composition has some type or degree of therapeutic activity. A lack of observable differences in the phenotype of the subject at the first observation compared to the second observation indicates that the composition does not possess therapeutic activity. The steps of observing the phenotype of the subject and administering the composition to said subject can be repeated for as long as the treating physician deems necessary. The physician may then compare the phenotype of the subject between any pair of observations to judge whether the composition has some type or degree of therapeutic activity. In one aspect of this embodiment, commonly used in clinical research, the phenotype observed at the time of the last administration of the test composition is referred to as the “last visit”. Eventually, the phenotype of the subject prior to initiation of treatment is compared with the phenotype of the subject at the last visit. Observable differences in the phenotype of the subject prior to initiation of treatment compared to the last visit indicates that the composition possesses some type or degree of therapeutic activity. A lack of observable differences in phenotype of the subject prior to initiation of treatment compared to the last visit indicates that the composition does not possess any type of therapeutic activity.

In another embodiment, the present invention relates to the compositions identified by methods described herein in the prevention of major depression or a related disorder or the treatment of major depression or a related disorder. More specifically, the present invention contemplates a method for at least substantially preventing in a subject major depression or a related disorder by administering to a subject in need of treatment thereof, a therapeutically effective amount of at least one composition that has been identified by the hereinbefore described methods that: (a) modulates the activity of a protein translated from SEQ ID NO:1; (b) reduces the amount of a protein translated from SEQ ID NO:1; (c) increases the amount of a protein translated from SEQ ID NO:1; or (d) modulates the level of expression of an mRNA molecule transcribed from SEQ ID NO:1. Administration of a prophylactic composition can occur prior to the manifestation of symptoms characteristic of major depression or a related disorder.

Additionally, the present invention further contemplates a method of treating a subject suffering from major depression or a related disorder by administering to a subject in need of treatment thereof, a therapeutically effective amount of at least one composition that has been identified by the hereinbefore described methods that: (a) modulates the activity of a protein translated from SEQ ID NO:1; (b) reduces the amount of a protein translated from SEQ ID NO:1; (c) increases the amount of a protein translated from SEQ ID NO:1; or (d) modulates the level of expression of an mRNA molecule transcribed from SEQ ID NO:1.

By way of example, and not of limitation, examples of the present invention shall now be given.

### EXAMPLE 1

### Identification of Genetic Linkage and Association between DEP2 and Major Depressive Disorder

Genetic linkage between DEP2 and major depressive disorder was established in a pedigree-based study in the Mormon population of Utah. The ascertainment and characteristics of a majority of these pedigrees has been described (Abkevich et al., Am. J. Hum. Genet. 73:1271-1281 (2003)). In the study described herein, a total of 93 pedigrees that contain a minimum of four females affected with major depressive disorder (DSM-IV-TR sections 296.2x or 296.3x) were selected for genetic analysis. These pedigrees comprised 744 affected females.

Affected individuals were genotyped and genome-wide linkage analysis was performed as described (Abkevich et al., op. cit.). Two meaningful differences between the present study and our previously published work are: first, that additional pedigrees were ascertained; second, that the definition of affected status was different as it did not include bipolar disorder in this study.

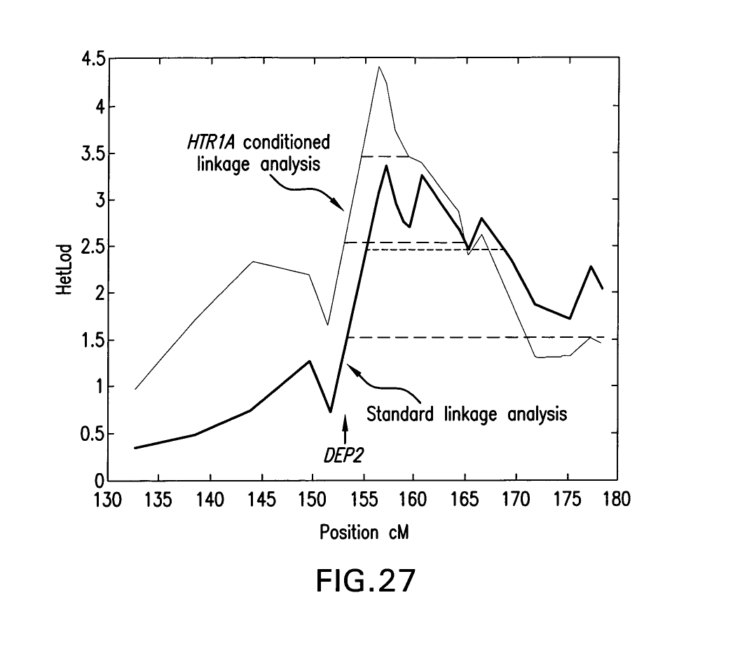

Using a dominant genetic model and considering only females with major depressive disorder as affected, evidence of linkage on chromosome 10 at marker D10S1676 was observed (heterogeneity LOD score (HLOD) 2.4). Upon genotyping of additional markers in the 26 centimorgan (“cM”) interval between DIOS2322 and D10S1700, the linkage evidence increased to a peak HLOD of 3.4 at D10S214 (FIG. 27 and Table 1).

The serotonin receptor 1A (Htr1a) is a therapeutic target in the management of depressive and anxiety disorders (Barnes and Sharp, Neuropharmacology 38:1083-1152 (1999)). A common polymorphic site in the corresponding gene (HTR1A) has been described, such that the 1019^{th} nucleotide upstream of the transcriptional start site naturally occurs as either cytosine or guanosine (Wu and Comings, Psych. Genet. 9:105-106 (1999)). Results of in vitro experiments suggest that the variant allele (−1019G) prevents binding of a transcriptional repressor, resulting in enhanced Htr1a expression (Lemonde et al., J. Neurosci., 23:8788-8799 (2003)). Either the −1019G allele or homozygous −1019GG genotype has been associated with depression, suicide, bipolar disorder, panic disorder with agoraphobia, neuroticism and decreased anti-depressant response (Arias et al., Mol. Psych. 7:930-932 (2002); Strobel et al., J. Neural Transm., 110-1445-1453 (2003); Lemonde et al., J. Neurosci., 23:8788-8799 (2003); Rothe et al., Int. J. Neuropsychopharmacol. 7:189-192 (2004); Huang et al., Int. J. Neuropsychopharmacol. 7:441-451 (2004); Serretti et al., Int. J. Neuropsychopharmacol 7:453-460 (2004); Lemonde et al., Int. J. Neuropsychopharmacol. 7:501-506 (2004); Arias et al., J. Psychopharmacol. 19:166-172 (2005)).

In the Utah population, HTR1A genotype −1019GG was 1.3-fold over-represented among individuals affected with major depressive disorder compared to unaffected individuals (p=0.044). Hence, linkage analysis was stratified according to HTR1A −1019 alleles. That is, only individuals with major depressive disorder, and also carrying one or two copies of the HTR1A-1019G risk allele, were considered affected. In a genome-wide HTR1A-conditional linkage analysis using a dominant genetic model and also restricted to female sex, the observed evidence of linkage on chromosome 10 strengthened to a peak HLOD of 3.1 at D10S1222. Upon inclusion of additional marker data in the 26 cM interval between DIOS2322 and DIOS1700, the linkage evidence increased to a peak HLOD of 4.4 at DIOS575 (FIG. 27 and Table 1).

The conditional linkage method improved upon the previously performed traditional linkage analysis in three ways. First, as noted above, it revealed stronger evidence supporting linkage of a dominant gene to major depressive disorder in females on chromosome 10 in the vicinity of D10S575. Second, it narrowed the linkage region (as defined by a drop of HLOD of either 1 or 2 from the peak value), such that the location of the linked gene was better defined. Third, and most importantly, it revealed linkage evidence in a distinct subset of pedigrees. Further investigation of those pedigrees was crucial to the discovery of DEP2 as a gene linked to major depressive disorder.

As a next step to identify a gene linked to major depressive disorder, each gene in the conditional linkage region was resequenced in representative affected females from each of sixteen pedigrees. These pedigrees were selected on the basis of having a familial HLOD of at least 0.4. Among these pedigrees, six had not shown linkage evidence without stratification on the basis of HTR1A alleles. The frequencies of variant alleles among the 22 chromosomes that segregated with major depressive disorder within these pedigrees was compared to the frequencies among 60 control chromosomes. For six single nucleotide polymorphisms (“SNPs”) within SEQ ID NO:1, statistically significant frequency differences were observed. Additionally, a statistical trend was observed for seventh SNP in SEQ ID NO:1 (Tables 2 and 3). For six of these seven SNPs, statistically significant frequency differences were also observed between the 22 chromosomes that segregated with major depressive disorder and a much larger set of control chromosomes (Table 4). Two of these SNPs (DEP2.0003 and DEP2.0004) were in complete linkage disequilibrium with each other. Between these markers, only DEP2.0003 was analyzed further.

To confirm the relationship between DEP2 genotypes and major depressive disorder, genetic association studies comparing genotype frequencies between individuals affected with major depressive disorder (not ascertained on the basis of familial history of disease) and healthy controls were performed in two populations. Consistent with the dominant linkage model, DEP2 genotypes were grouped into dichotomous variables such that carriers of a DEP2 risk allele (heterozygous or homozygous) were compared to non-carriers. Following the conditionality of DEP2 linkage on carriage of the HTR1A −1019G allele, this genotype was similarly included in statistical models as a dichotomous variable. Sex and all first-order interaction terms between genotypes or between genotype and sex were also included in statistical models. Non-significant terms (p>0.05) were sequentially dropped from statistical models using a backward elimination process.

In the Mormon population, DEP2.0003 (odds ratio for the T allele 1.40, 95% confidence interval 1.00-1.94) and DEP2.0006 (odds ratio for the A allele 2.03, 95% confidence interval 0.99-4.48) were associated with major depressive disorder (Tables 5 and 6). For each marker, the frequency of DEP2 allele carriage was highest among −1019G-positive cases, and approximately equal among all other groups. Additionally, the same DEP2 alleles were both linked to and associated with major depression in the Mormon population. There was also a significant DEP2.0003 genotype-by-sex interaction. In an Ashkenazi Jewish population, DEP2.0003 (odds ratio for the T allele 0.59, 95% confidence interval 0.35-0.99) and DEP2.0005 (odds ratio for the A allele 0.43, 95% confidence interval 0.24-0.75) were associated with major depressive disorder (Tables 7 and 8). For each marker, the frequency of DEP2 allele carriage was lowest among −1019G-positive cases, and approximately equal among all other groups. There was no association of DEP2.0005 in the Mormon population (Table 9), or of DEP2.0006 in the Jewish population (Table 10), with major depressive disorder.

The DEP2 polymorphisms associated with major depressive disorder differ between Mormon and Jewish populations, and opposite alleles at DEP2.0003 were associated with major depressive order between the two populations. This sort of situation is not unusual in psychiatric genetics, in fact it has been observed for most of the genes that have been linked to schizophrenia (Harrison and Weinberger, Mol. Psych. 10:40-68 (2005)). The most parsimonious explanation for these results is that functional alleles of DEP2 arose on different haplotypes in the Mormon and Jewish populations.

TABLE 1

| Microsatellite Marker | HLOD | Conditional HLOD |
|---|---|---|
| D10S1656 | 0.8 | 1.9 |
| D10S2322 | 0.7 | 1.9 |
| D10S575 | 3.1 | 4.4 |
| D10S214 | 3.4 | 4.2 |
| D10S1703 | 2.9 | 3.8 |
| D10S1782 | 2.7 | 3.6 |
| D10S1222 | 2.7 | 3.5 |
| D10S1727 | 2.7 | 3.5 |
| D10S1676 | 3.3 | 3.6 |
| D10S1439 | 2.7 | 3.1 |
| D10S1134 | 2.5 | 3.0 |
| D10S1248 | 2.8 | 2.3 |
| D10S505 | 2.4 | 1.6 |
| D10S1770 | 1.9 | 1.6 |
| D10S1651 | 1.8 | 1.7 |
| D10S590 | 1.7 | 1.7 |
| D10S212 | 2.3 | 1.9 |
| D10S1711 | 2.0 | 1.8 |
| D10S1700 | 2.0 | 1.8 |

TABLE 2

| Marker Name | Alleles | Location in SEQ ID NO: 1 |
|---|---|---|
| DEP2.0001 | A, C | 3005 |
| DEP2.0002 | C, A | 33241 |
| DEP2.0003 | C, T | 38048 |
| DEP2.0004 | G, A | 38215 |
| DEP2.0005 | G, A | 77402 |
| DEP2.0006 | G, A | 77333 |
| DEP2.0007 | C, T | 79906 |

TABLE 3

| Marker Name | Linked Chromosomes | Control Chromosomes | P |
|---|---|---|---|
| DEP2.0001 | 10/22 | 5/60 | 0.0004 |
| DEP2.0002 | 10/22 | 12/58 | 0.02 |
| DEP2.0003 | 13/22 | 17/60 | 0.02 |
| DEP2.0004 | 13/22 | 17/60 | 0.02 |
| DEP2.0005 | 14/22 | 25/60 | 0.09 |
| DEP2.0006 | 4/22 | 1/60 | 0.02 |
| DEP2.0007 | 3/22 | 0/60 | 0.02 |

TABLE 4

| Marker Name | Linked Chromosomes | Control Chromosomes | P |
|---|---|---|---|
| DEP2.0001 | 10/22 | 105/356 | 0.15 |
| DEP2.0002 | 10/22 | 121/684 | 0.003 |
| DEP2.0003 | 13/22 | 184/686 | 0.002 |
| DEP2.0004 | 13/22 | 184/686 | 0.002 |
| DEP2.0005 | 14/22 | 251/696 | 0.01 |
| DEP2.0006 | 4/22 | 17/696 | 0.003 |
| DEP2.0007 | 3/22 | 20/708 | 0.03 |

TABLE 5

| HTR1A | | DEP2.0003 T+ | | Males | | Females | |
|---|---|---|---|---|---|---|---|
| Genotype | Status | Number | % | Number | % | Number | % |
| G+ | Case | 197/342 | 58 | 69/113 | 61 | 128/229 | 56 |
| | Control | 85/182 | 47 | 42/101 | 42 | 43/81 | 53 |
| CC | Case | 33/77 | 43 | 20/37 | 54 | 13/40 | 32 |
| | Control | 28/62 | 45 | 11/31 | 35 | 17/31 | 55 |

TABLE 6

| | |
|---|---|
| HTR1A | DEP2.0006 A+ |

| Genotype | Status | Number | % |
|---|---|---|---|
| G+ | Case | 29/347 | 8 |
| | Control | 7/183 | 4 |
| CC | Case | 3/77 | 4 |
| | Control | 3/63 | 5 |

TABLE 7

| | |
|---|---|
| HTR1A | DEP2.0003 T+ |

| Genotype | Status | Number | % |
|---|---|---|---|
| G+ | Case | 51/132 | 39 |
| | Control | 35/64 | 55 |
| CC | Case | 29/50 | 58 |
| | Control | 13/21 | 62 |

TABLE 8

| | |
|---|---|
| HTR1A | DEP2.0005 A+ |

| Genotype | Status | Number | % |
|---|---|---|---|
| G+ | Case | 71/132 | 54 |
| | Control | 49/65 | 75 |
| CC | Case | 34/50 | 68 |
| | Control | 17/22 | 77 |

TABLE 9

| | |
|---|---|
| HTR1A | DEP2.0005 A+ |

| Genotype | Status | Number | % |
|---|---|---|---|
| G+ | Case | 122/347 | 35 |
| | Control | 75/183 | 41 |
| CC | Case | 21/11 | 35 |
| | Control | 27/63 | 43 |

TABLE 10

| | |
|---|---|
| HTR1A | DEP2.0006 A+ |

| Genotype | Status | Number | % |
|---|---|---|---|
| G+ | Case | 10/132 | 8 |
| | Control | 4/65 | 6 |
| CC | Case | 1/50 | 2 |
| | Control | 3/22 | 14 |

### EXAMPLE 2

### Detection of A Brain-Specific DEP2 Transcript by Northern Blotting

Northern blotting was performed using a probe from the 3′ UTR (nt. 1295 to 1713) of LHPP on a multi-tissue blot containing poly(A) RNA from the following human tissues: brain, placenta, skeletal muscle, heart, kidney, pancreas, liver, lung, spleen, and colon.

Methods

The pre-made poly(A) RNA Northern blot was product #3140 from Ambion (Austin Tex.). PCR was conducted to amplify a product that lies entirely in the 3′ UTR of SEQ ID NO:9 (nucleotides 1295 to 1713).

| | |
|---|---|
| Forward primer: | |
| GAATCTCCCAAATCCCAGAACTCA | (SEQ ID NO:68) |
| Reverse primer: | |
| ACACCGGGCATGACACCTTCAAGT | (SEQ ID NO:69) |

The DNA product was labeled using an AmbionStrip-EZ DNA kit (Ambion, Austin Tex.) and [α-32P] dATP. The blot was hybridized overnight at 42 degrees Celsius in ULTRAhyb Ultrasensitive Hybridization Buffer (Ambion, Austin Tex.). The blot was washed 2×15 minutes at low stringency (2× SSPE, 0.1% SDS) and 2×15 minutes at high stringency (0.1× SSPE, 0.1% SDS). All procedures were carried out per the manufacturer's instructions.

Results

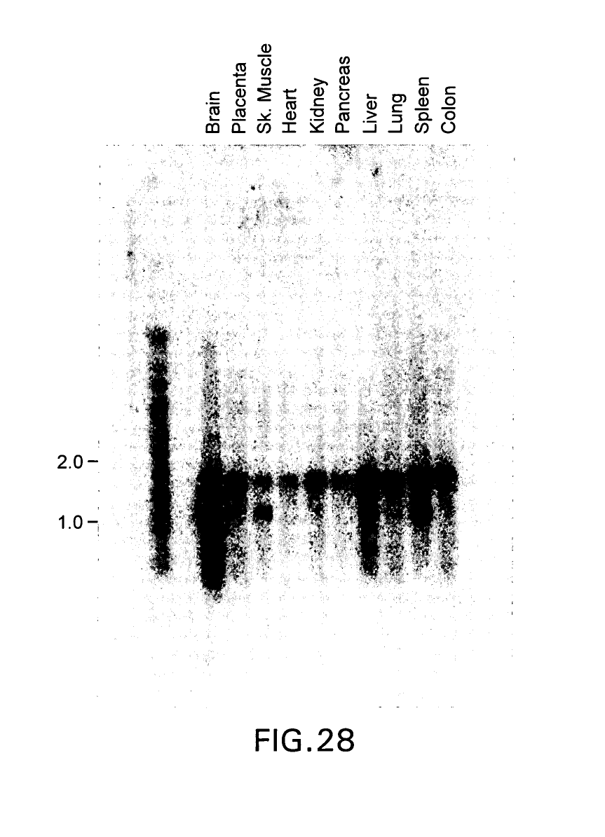

FIG. 28 shows the existence of at least two DEP2 transcripts. An approximately 1.7 kb transcript was present in approximately equal abundance across all tissues tested. An approximately 1.1 kb transcript was very abundant in brain and observed at low levels in skeletal muscle and lung.

Conclusion

The 1.7 kb transcript is consistent with LHPP (SEQ ID NO:9) in the literature (Yokoi et al, J Biochem 133:607-613 (2003)). The existence of a novel DEP2 transcript of approximately 1.1 kb was established. Furthermore, this transcript appears to be most abundant in brain.

### EXAMPLE 3

### Observation of Enhanced DEP2 Transcript Expression on Microarrays

Probe sets within DEP2 are present on Affymetrix U133 Plus and U133Av2 microarrays. These probe sets are annotated as recognizing LHPP, however they are within sequences that are common to SEQ ID NO:2, SEQ ID NO:5, SEQ ID NO:6, SEQ ID NO:7, SEQ ID NO:8, SEQ ID NO:9, SEQ ID NO:11, and SEQ ID NO:12.

Methods

Datasets from microarray experiments that had been conducted for unrelated purposes were mined to learn additional information regarding the expression level of DEP2 transcripts in normal human tissues. An alpha value of 1E-12 was used for statistical significance.

Results

Data from Affymetrix U133 Plus and U133Av2 microarrays are shown in Table 11 and Table 12, respectively. Results considered statistically significant are in boldface type.

TABLE 11

| Tissue type | Intensity | p-value |
|---|---|---|
| spinal cord (BD) | 1192.5 | 0 |
| univ ref (BD) | 658.0 | 4.10E−30 |
| brain (AM) | 616.6 | 1.00E−27 |
| brain (AM) | 604.8 | 5.88E−39 |
| caudate nucleus (AM) | 592.9 | 5.61E−45 |
| basal ganglia (AM) | 508.4 | 1.74E−39 |
| hippocampus (AM) | 388.4 | 8.36E−25 |
| brain (AM) | 323.4 | 1.61E−16 |
| brain (BD) | 230.5 | 7.06E−20 |
| hypothalamus (AM) | 228.9 | 8.77E−17 |
| cerebellum (BD) | 198.0 | 4.54E−16 |
| adrenal gland (BD) | 172.3 | 6.39E−09 |
| univ ref (S) | 153.5 | 1.94E−07 |
| salivary gland (BD) | 148.9 | 0.00013 |
| salivary gland (BD) | 128.9 | 7.24E−08 |
| prostate (BD) | 107.1 | 4.30E−05 |
| testis (AM) | 105.1 | 0.000276 |
| retina (BD) | 103.0 | 0.00229 |
| ileum (AM) | 100.8 | 0.000296 |
| pericardium (AM) | 93.5 | 0.000186 |
| lymph node (AM) | 90.6 | 2.71E−05 |
| thyroid gland (BD) | 87.8 | 2.28E−06 |
| kidney (AM) | 87.4 | 0.00573 |
| trachea (BD) | 86.2 | 4.03E−06 |
| aorta (AM) | 84.3 | 0.000571 |
| colon proximal (AM) | 81.8 | 1.23E−05 |
| liver (BD) | 77.5 | 2.72E−06 |
| prostate (BD) | 73.7 | 0.02 |
| thyroid (AM) | 71.0 | 0.00019 |
| fetal liver (BD) | 68.6 | 0.000435 |
| kidney (BD) | 67.5 | 0.000257 |
| right atrium (AM) | 66.2 | 0.03 |
| colon distal (AM) | 65.3 | 0.000397 |
| testis (BD) | 59.5 | 0.000539 |
| thymus (BD) | 55.1 | 0.00131 |
| spleen (AM) | 54.5 | 0.00491 |
| Vena cava (AM) | 53.2 | 0.05 |
| jejunum (AM) | 47.5 | 0.02 |
| uterus (BD) | 46.9 | 0.00417 |
| pancreas (BD) | 46.2 | 0.03 |
| bone marrow (BD) | 45.9 | 0.05 |
| bladder (AM) | 41.2 | 0.00923 |
| Left ventricle (AM) | 39.2 | 0.15 |
| left atrium (AM) | 37.8 | 0.13 |
| duodenum (AM) | 35.0 | 0.04 |
| thymus (BD) | 35.0 | 0.12 |
| placenta (BD) | 34.3 | 0.01 |
| prostate (BD) | 34.1 | 0.03 |
| right ventricle (AM) | 31.3 | 0.14 |
| heart (BD) | 30.8 | 0.08 |
| lung (BD) | 29.4 | 0.04 |
| bone marrow (BD) | 25.6 | 0.04 |
| breast (AM) | 24.8 | 0.09 |
| skeletal muscle (BD) | 15.5 | 0.16 |
| stomach (AM) | 14.8 | 0.25 |
| ovary (AM) | 14.4 | 0.21 |
| pancreas (AM) | 8.7 | 0.37 |
| fetal brain (BD) | 7.4 | 0.32 |

(AM) = purchased from Ambion
(BD) = purchased from BD Biosciences
(S) = purchased from Sigma

TABLE 12

| Tissue type | Intensity | p-value |
|---|---|---|
| Frontal cortex | 897.9 | 0 |
| Thalamus | 806.1 | 0 |
| Basal ganglia | 634.7 | 0 |
| Temporal cortex | 348.3 | 0 |
| Occipital cortex | 294.6 | 0 |
| Parietal cortex | 274.8 | 0 |
| Medulla | 257.9 | 0 |
| Cerebellum | 253.1 | 0 |
| Universal 1 | 83.2 | 0.02 |
| Heart | 47.5 | 0.22 |
| Stomach | 42.2 | 0.05 |
| Prostate | 34.2 | 0.08 |
| Universal 2 | 0.7 | 0.49 |
| Pancreas | −20.7 | 0.7 |

All RNAs were purchased from Ambion.

Conclusion

Based on observation of statistically significant intensity data for every central nervous system sample examined (except a single fetal brain sample), and lack of statistically significant intensity data for any other sample examined, it appears that DEP2 transcripts are preferentially expressed in the central nervous system. Because the microarray probe sets are complementary to sequences common to several naturally occurring DEP2 transcripts, attributing intensity data from these probe sets specifically to LHPP may be misleading.

### EXAMPLE 4

### Tissue Distribution of DEP2 Transcripts

The tissue distributions of human DEP2 transcripts were determined using quantitative reverse transcription polymerase chain reaction (QPCR). Assays were conducted for each DEP2 transcript for which there was more supportive evidence (bioinformatic or experimental) than a single expressed sequence tag. Because of the linkage and association of DEP2 to major depressive disorder, there was particular focus on the distributions of these transcripts in the brain.

Methods

Human total RNAs were purchased from either Ambion, Inc. (Austin, Tex.) or BD Biosciences (Franklin Lakes, N.J.).

Reverse transcription and PCR were conducted using the Invitrogen Platinum Thermoscript One Step System qRTPCR kit following the manufacturer's instructions. 50 ng DNAse-treated total RNA was used as a template for each reaction. All Ct readings were normalized to 28S rRNA. A dilution series of Universal Human Reference (BD Biosciences) was used to generate a standard curve for these analyses. Relative expression levels were determined by the Relative Standard Curve Method described in the ABI Prism User Bulletin Number 2 with 28s rRNA assayed as an endogenous control for each sample. Equivalent reverse-transcription efficiency was assumed for gene-to-gene comparison in the absence of quantitative standards such as purified RNA transcripts.

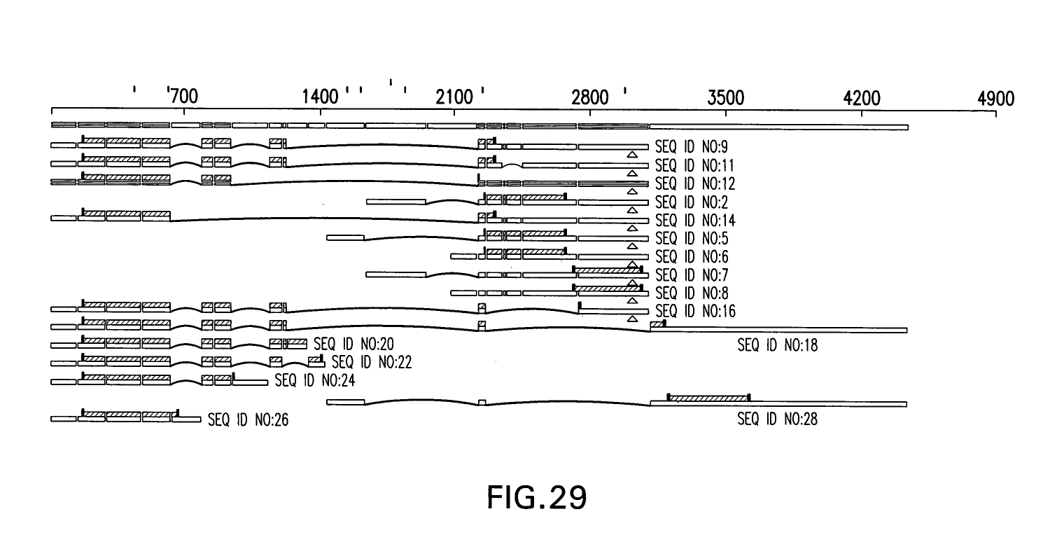

A schematic of DEP2 transcripts is shown in FIG. 29.

The following primers and probe were used for amplification and detection of DEP2-1 mRNA (SEQ ID NO:2). These primers and probe do not discriminate against a naturally occurring splice variant of DEP2-1 (SEQ ID NO:7).

Set 1 (inter-exon)

(SEQ ID NO:35)

5′-CACGTACCCATCAGCCTTCAC-3′

(SEQ ID NO:36)

5′-CCTGTGGAAGGAGCATACAGT-3′

(SEQ ID NO:37)

5′-\56-FAM\CCCAGTGACGAGCACCATCCGG\36-TAMSp\-3′
(probe)
Set 2 (intra-exon)

(SEQ ID NO:38)

5′-CAACACTGGCACCTGCAGAT-3′

(SEQ ID NO:39)

5′-CCACCCCATGCCATCAA-3′

(SEQ ID NO:40)

5′-\56-FAM\AAGTGGCAGAGCAGCCCCCAGC\36-TAMSp\-3′
(probe)

The following primers and probe were used for amplification and detection of a splice variant of DEP2-1 (SEQ ID NO:5).

(SEQ ID NO:35)

5′-CACGTACCCATCAGCCTTCAC-3′

(SEQ ID NO:41)

5′-CCCGCCTCTCCAAGACCAT-3′

(SEQ ID NO:37)

5′-\56-FAM\CCCAGTGACGAGCACCATCCGG\36-TAMSp\-3′
(probe)

The following primers and probe were used for amplification and detection of a splice variant of DEP2-1 (SEQ ID NO:6). These primers and probe do not discriminate against another naturally occurring splice variant of DEP2-1 (SEQ ID NO:8).

(SEQ ID NO:35)

5′-CACGTACCCATCAGCCTTCAC-3′

(SEQ ID NO:42)

5′-GGTACACTCATGTCCCCACCAT-3′

(SEQ ID NO:37)

5′-\56-FAM\CCCAGTGACGAGCACCATCCGG\36-TAMSp\-3′
(probe)

The following primers and probe were used for amplification and detection of LHPP mRNA (SEQ ID NO:9).

(SEQ ID NO:35)

5′-CACGTACCCATCAGCCTTCAC-3′

(SEQ ID NO:43)

5′-GCGCACCGGGAAGTTCAG-3′

(SEQ ID NO:37)

5′-\56-FAM\CCCAGTGACGAGCACCATCCGG\36-TAMSp\-3′
(probe)

The following primers and probe were used for amplification and detection of a splice variant of LHPP (SEQ ID NO:12).

(SEQ ID NO:44)

5′-TGCAAGCGATAGGAGTGGAA-3′

(SEQ ID NO:45)

5′-GGTTGTCCACGTACCCATCAG-3′

(SEQ ID NO:46)

5′-\56-FAM\CCCACCAGGCCCAGTGACGAGC\36-TAMSp\-3′
(probe)

The following primers and probe were used for amplification and detection of a splice variant of LHPP (SEQ ID NO:20).

(SEQ ID NO:47)

5′-GCGCACCGGGAAGTTCAG-3′

(SEQ ID NO:48)

5′-TGAAGAACAAAACAGAATGAGAATGTG-3′

(SEQ ID NO:49)

5′-\56-FAM\CCAGCTGGAGTCATTTATTCACCTTCCTTCC\36-
TAMSp\-3′
(probe)

The following primers and probe were used for amplification and detection of a splice variant of LHPP (SEQ ID NO:24).

(SEQ ID NO:50)

5′-CCACCAGTTACTTTCAGTATGAAAGCA-3′

(SEQ ID NO:51)

5′-TATCCTTTCAGAGAAGCAGCAAAAAC-3′

(SEQ ID NO:52)

5′-\56-FAM\CAGAAATGCCTGCGGCTTTTCCTG\36-TAMSp\-3′
(probe)

The following primers and probe were used for amplification and detection of DEP2-2 (SEQ ID NO:28).

(SEQ ID NO:53)

5′-CGCCCCAGACCCAAGAATC-3′

(SEQ ID NO:54)

5′-CAGGAAGTGCCCATCAGCCT-3′

(SEQ ID NO:55)

5′-\56-FAM\CCCGCCTCTCCAAGACCATCCCT\36-TAMSp\-3′
(probe)

The following primers and probe were used for amplification and detection of DEP2-3 (SEQ ID NO:30).

(SEQ ID NO:56)

5′-AGCACCATCCGGAAGTGAAG-3′

(SEQ ID NO:57)

5′-GCTGCAAGATCTGTGCATAGGA-3′

(SEQ ID NO:58)

5′-\56-FAM\CTGATGGTGAAGAGCCTGGAAGAAACCCA\36-
TAMSp\-3′
(probe)

The following primers and probe were used for amplification and detection of AK127935 (SEQ ID NO:31).

(SEQ ID NO:59)

5-CAATTTAGGTCGCTGCTATGGA-3′

(SEQ ID NO:60)

5′-TGGTGACTCAAAGGCCTAATGG-3′

(SEQ ID NO:61)

5′-\56-FAM\CCTGGCCTCTTAACTCATTTACCCGGG\36-
TAMSp\-3′

The following primers and probe were used for amplification and detection of AW867792 (SEQ ID NO:33).

(SEQ ID NO:62)

5′-TGGAGGCAGCCTCGCTTTA-3′

(SEQ ID NO:63)

5′-TTGGAGGAAGAGTTCTCATGCA-3′\

(SEQ ID NO:64)

5′-\56-FAM\CCCGCAGAACCTCCACGCTGTT\36-TAMSp\-3′

Cycle threshold (Ct) values were qualitatively interpreted as follows:

| | |
|---|---|
| Ct > 35 | probably noise |
| Ct = 30-35 | possibly low abundance transcript, reliability |
| | assessed by shape of Ct curve |
| Ct = 25-30 | moderate abundance transcript |
| Ct = 20-25 | high abundance transcript |
| Ct < 20 | very high abundance transcript |

Results

Observed Ct values are shown in Table 13. For SEQ ID NO:5, only inter-exon results are shown.

TABLE 13

| | SEQ ID NO: | | | | | | | | | |
|---|---|---|---|---|---|---|---|---|---|---|
| | 2 | 5 | 9 | 12 | 20 | 24 | 28 | 30 | 31 | 33 |
| Adrenal gland (MP) | 27.41 | 38.66 | 23.27 | 22.54 | 25.68 | 30.16 | 38.01 | 27.7 | 26.68 | 26.64 |
| Amygdala (AM) | 20.48 | 32 | 22.91 | 21.83 | 26.78 | 30.47 | 38.93 | 27.12 | 27.93 | 27.15 |
| Basal ganglia (AM) | 19.8 | 36.52 | 22.84 | 21.02 | 26.54 | 30.86 | 37.54 | 26.82 | 27.87 | 26.67 |
| Brain (AM) | 19.82 | 31.78 | 22.86 | 21.34 | 26.37 | 30.34 | 36.91 | 26.36 | 27.31 | 26.75 |
| Brain (MP) | 24.5 | 37.02 | 22.91 | 21.99 | 26.01 | 29.9 | 40 | 26.22 | 26.21 | 25.7 |
| Caudate nucleus (AM) | 20.36 | 33.16 | 22.58 | 21.05 | 25.52 | 29.79 | 36.34 | 26.69 | 25.92 | 25.63 |
| Fetal brain (AM) | 26.24 | 31.91 | 23.97 | 22.83 | ND | ND | ND | ND | ND | ND |
| Fetal liver (MP) | 31.03 | 40 | 40 | 23.53 | 33.18 | 31.48 | 39.34 | 29.88 | 30.27 | 29.93 |
| Globus pallidus (AM) | 17.54 | 32.81 | 22.59 | 21 | 26.23 | 30.74 | 40 | 25.71 | 26.99 | 26.52 |
| Heart (AM) | 30.96 | 40 | 26.49 | 25.12 | 40 | 31.59 | 39.7 | 32.78 | 32.72 | 29.93 |
| Heart (MP) | 29.59 | 34.83 | 23.87 | 24.38 | 31.43 | 30.17 | 38.33 | 30.14 | 28.71 | 28.33 |
| Hippocampus (AM) | 21.09 | 32.7 | 22.58 | 21.22 | 26.2 | 30.17 | 40 | 26.47 | 27.03 | 26.21 |
| Hypothalamus (AM) | 19.83 | 28.99 | 22.43 | 20.23 | 26.66 | 30.43 | 33.54 | 25.85 | 27.42 | 26.86 |
| Kidney (AM) | 31.94 | 32.1 | 23.38 | 21.81 | 31.6 | 30.06 | 35.26 | 29.37 | 30.24 | 29.38 |
| Kidney (MP) | 27.93 | 38.31 | 22.51 | 21.31 | 26.64 | 30.96 | 39.4 | 26.67 | 27.54 | 27.62 |
| Liver (AM) | 30.01 | 40 | 23.8 | 22.48 | 32.56 | 32.36 | 40 | 29.74 | 30.65 | 29.51 |
| Liver (MP) | 30.12 | 40 | 23.67 | 22.41 | 29.47 | 31.65 | 40 | 27.96 | 28.59 | 28.12 |
| Lung (AM) | 40 | 32.29 | 25.44 | 25.11 | 33.74 | 32.6 | 39.22 | 32.82 | 31.34 | 29.92 |
| Lung (MP) | 28.01 | 36.39 | 24.55 | 23.66 | 28.05 | 31.7 | 37.84 | 28.46 | 28.39 | 28.21 |
| Medulla (AM) | 18.77 | 40 | 22.5 | 21.2 | 26.17 | 30.87 | 40 | 25.52 | 27.09 | 26.5 |
| Orbital frontal cortex (AM) | 18.84 | 30.1 | 22.47 | 20.92 | 25.86 | 30.12 | 35.83 | 25.65 | 26.96 | 25.94 |
| Peripheral leukocytes (BD) | 25.15 | 29.52 | 24.18 | 23.35 | 27.4 | 31.12 | 31.81 | 27.7 | 27.09 | 27.52 |
| Placenta (MP) | 29.52 | 40 | 25.02 | 24.75 | 33.54 | 31.93 | 38.76 | 35.4 | 29.48 | 28.93 |
| Pons (AM) | 19.05 | 38.91 | 22.25 | 20.7 | 26.34 | 30.59 | 40 | 25.05 | 27.54 | 26.94 |
| Prefrontal cortex (AM) | 20.25 | 37.69 | 22.59 | 21.34 | 26.06 | 30.06 | 40 | 26.66 | 27.15 | 26.04 |
| Prostate (MP) | 28.26 | 33.94 | 24.84 | 23.54 | 28.4 | 31.48 | 34.97 | 29.31 | 28.3 | 28.09 |
| Salivary gland (MP) | 29.25 | 31.94 | 25.1 | 24.14 | 31.38 | 31.47 | 34.7 | 29.37 | 29.39 | 29.02 |
| Skeletal muscle (MP) | 30.18 | 40 | 26.01 | 25.27 | 40 | 33.16 | 40 | 30.85 | 30.33 | 29.51 |
| Small intestine (AM) | 28.42 | 36.4 | 24.03 | 23.19 | 30.37 | 31.34 | 39.58 | 29.81 | 29.4 | 29.17 |
| Spinal cord (AM) | 18.12 | 31.91 | 21.89 | 20.59 | 27.21 | 31.32 | 37.64 | 24.45 | 27.67 | 26.99 |
| Spinal cord (MP) | 22.88 | 37.62 | 22.34 | 21.29 | 27.4 | 31.32 | 39.49 | 24.74 | 27.56 | 27.32 |
| Spleen (MP) | 28.97 | 27.06 | 25.32 | 24.34 | 30.36 | 32.28 | 29.81 | 29.17 | 28.68 | 28.91 |
| Testis (MP) | 25.85 | 40 | 22.65 | 21.96 | 24.48 | 27.75 | 40 | 26.53 | 24 | 25.04 |
| Thalamus (AM) | 19.81 | 40 | 21.81 | 20.5 | 26.29 | 30.09 | 40 | 26.28 | 27.33 | 26.48 |
| Thymus (MP) | 26.57 | 28.63 | 24.44 | 23.52 | 27.08 | 30.48 | 31.79 | 29.56 | 27.04 | 27.26 |
| Thyroid gland (MP) | 27.56 | 32.25 | 22.87 | 21.72 | 26.23 | 30.05 | 35.03 | 26.86 | 26.96 | 27.28 |
| Trachea (MP) | 27.85 | 31.73 | 24.44 | 23.53 | 28.36 | 31.18 | 34.36 | 28.83 | 28.2 | 28.12 |
| Uterus (MP) | 28.04 | 40 | 24.54 | 23.79 | 28.84 | 32.16 | 40 | 29.17 | 28.3 | 28.31 |

(AM) = purchased from Ambion
(BD) or (MP) = purchased from BD Biosciences
ND = not done
Data for SEQ ID NO: 2 are from primer/probe set 1.

DEP2-1 (SEQ ID NO:2) was detected as a very high or high abundance transcript in all central nervous system samples tested (Ct range 17.5-24.5) except for fetal brain (Ct 26.2), and as a moderate or low abundance transcript in other tissues (Ct range 25.2-31.9) except for lung (Ct 40). Similar results were obtained using a second set of primers and probe within the first exon of DEP2-1 (nucleotides 1-315 of SEQ ID NO:2).

A splice variant of DEP2-1 (SEQ ID NO:5) was reliably detected in spleen, thymus, hypothalamus and peripheral leukocytes (Ct range 27.1-29.5).

A splice variant of DEP2-1 (SEQ ID NO:6) was not reliably detected (not shown).

LHPP (SEQ ID NO:9) was detected as a high or moderate abundance transcript in all samples tested (Ct range 21.8-26.5) except fetal liver (Ct 40). Expression in central nervous system was in general slightly higher than in other tissues.

A splice variant of LHPP (SEQ ID NO:12) was detected as a high abundance transcript in all samples tested (Ct range 20.2-25.2). Expression in central nervous system was in general slightly higher than in other tissues.

A splice variant of LHPP (SEQ ID NO:20) was detected as a moderate abundance transcript in all central nervous system samples tested (Ct range 25.5-27.4), as well as in several other tissues.

A splice variant of LHPP (SEQ ID NO:24) was detected as a moderate abundance transcript in a few samples, and as a low abundance transcript in all others.

DEP2-2 (SEQ ID NO:28) was detected as a low abundance transcript (smallest Ct 29.8).

DEP2-3 (SEQ ID NO:30) was detected as a moderate abundance transcript in all central nervous system samples tested (Ct range 24.4-27.1), as well as in several other tissues.

AK127935 (SEQ ID NO:31) was detected as a moderate abundance transcript in all central nervous system samples tested (Ct range 25.9-27.9), as well as in several other tissues.

AW867792 (SEQ ID NO:33) was detected as a moderate abundance transcript in all samples tested (Ct range 25.0-30.0). Expression in central nervous system was in general slightly higher than in other tissues.

Conclusions

SEQ ID NO:2, SEQ ID NO:5, SEQ ID NO:9, SEQ ID NO:12, SEQ ID NO:20, SEQ ID NO:24, SEQ ID NO:28, SEQ ID NO:30, SEQ ID NO:31 and SEQ ID NO:33 are naturally occurring transcripts arising from DEP2 (SEQ ID NO:1). Some of the signal observed for SEQ ID NO:5 may be attributable to SEQ ID NO:7, see Examples 5-7 below for independent experimental evidence that SEQ ID NO:5 is a naturally occurring transcript arising from DEP2. Failure to detect SEQ ID NO:6 (or SEQ ID NO:8, which would be amplified and detected by the same primer/probe set) cannot be taken as evidence that it is not a naturally occurring transcript arising from DEP2, without use of a positive control to ensure that the assay worked. Expression of transcripts arising from DEP2, relative to 28S rRNA, was generally higher in the central nervous system than in other tissues. The difference between central nervous system and other tissues was strongest for DEP2-1 (SEQ ID NO:2).

### EXAMPLE 5

### Establishing the Sequence of DEP2-1

DEP2-1 (SEQ ID NO:2) which is a novel sequence that has never been described previously, comprises distinct protein coding capacity for Dep2-1a (SEQ ID NO:3) and Dep2-1b (SEQ ID NO:4), and is highly and preferentially expressed in the central nervous system. These characteristics make it of particular interest as a candidate to explain the linkage and association of DEP2 to major depressive disorder. DEP2-1 clones were sequenced to establish whether the sequence predicted by mining EST sequence databases was correct.

Methods

IMAGE clones h3175509, h5194531, h5197955 and h4565014 were obtained from the American Type Culture Collection (“ATCC”), P.O. Box 1549, Manassas, Va. 20108. DNA sequencing was performed using standard methods well known to those practiced in the art.

Results

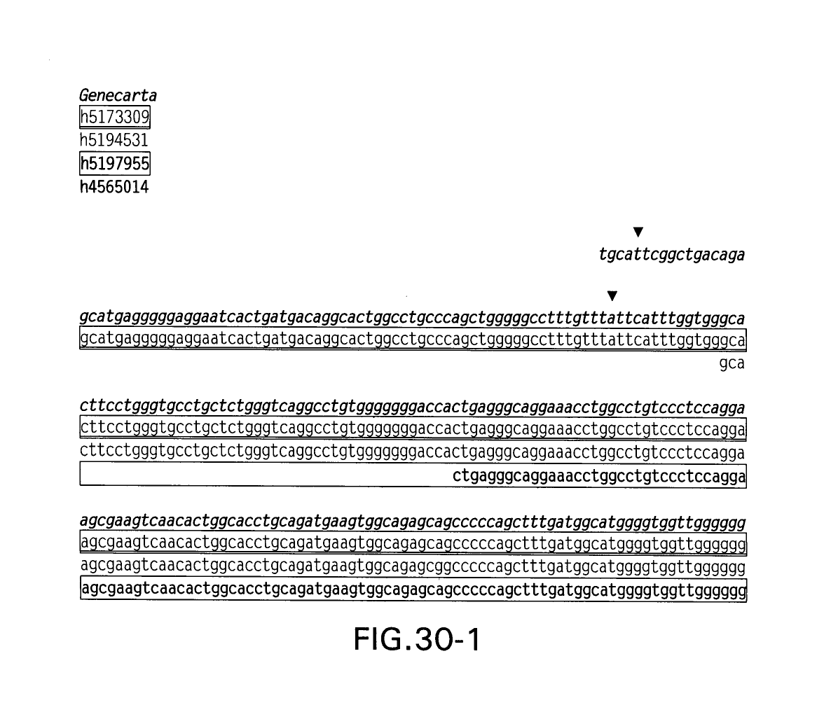

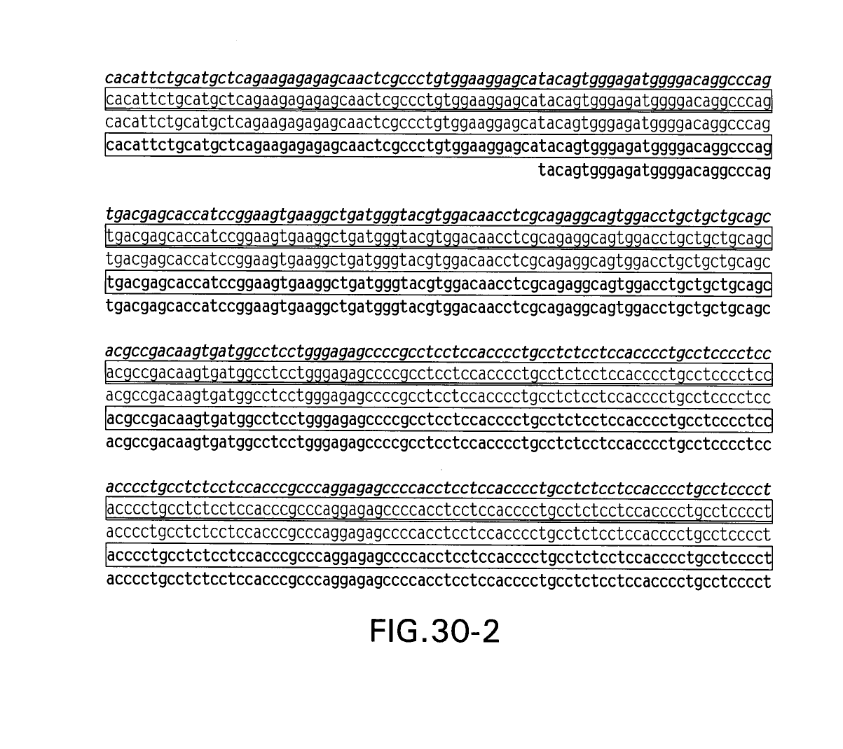

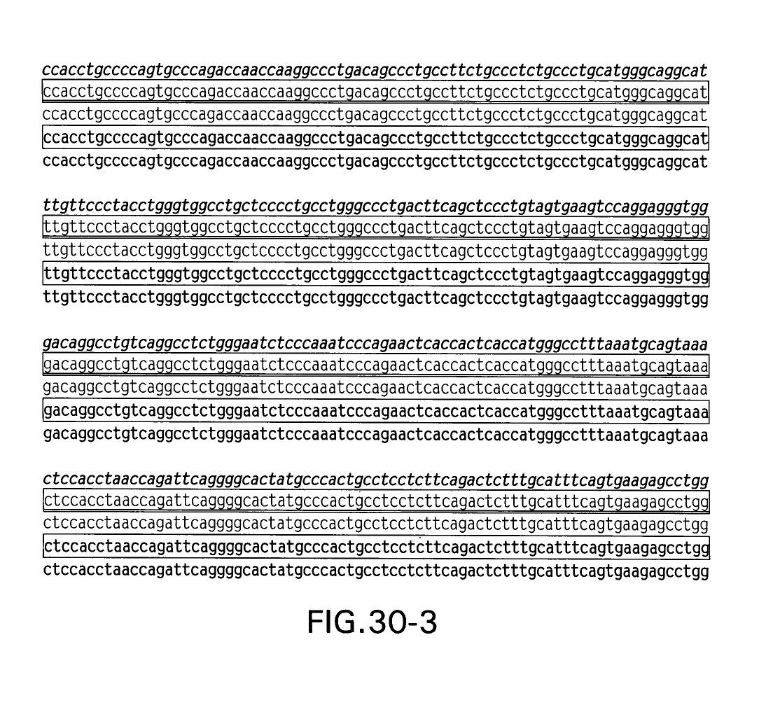

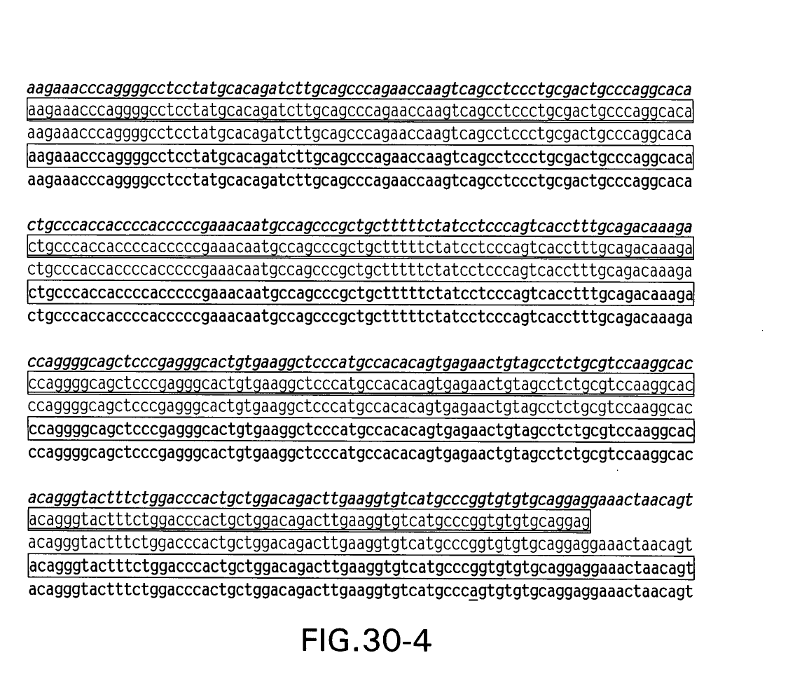

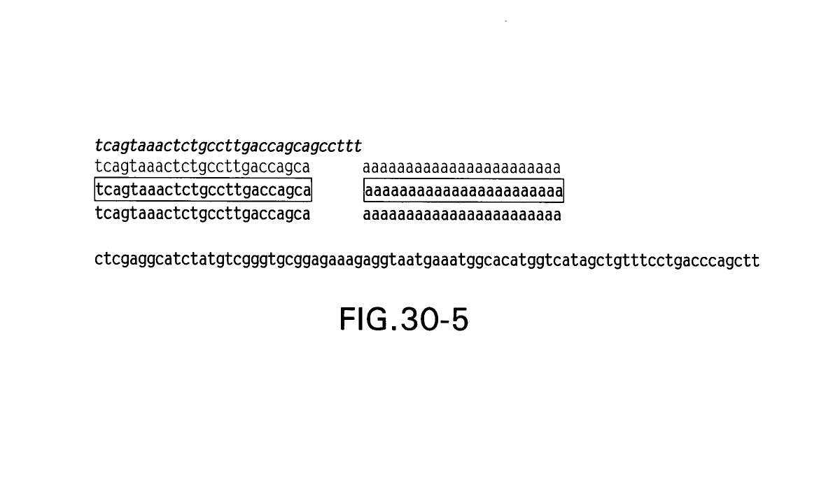

Aligned sequences of the four IMAGE clones and the sequence predicted by Genecarta software from EST sequences are shown in FIG. 30. Clone h4565014 contains sequence, downstream of a polyadenylate tract, that does not match DEP2.

Conclusion

The full-length sequence of DEP2-1 has been established. A novel single nucleotide polymorphism has been identified.

### EXAMPLE 6

### Characterization of the 5′ ends of DEP2-1 by RLM-RACE

RNA ligase-mediated rapid amplification of cDNA ends (RLM-RACE) was performed on a pool of human spinal cord RNA to determine the 5′ ends of DEP2-1 (SEQ ID NO:2).

Methods

Human spinal cord total RNA (# 636554 (also called 64113-1)) was obtained from BD Biosciences Clontech (Palo Alto, Calif.). The FirstChoice RLM-RACE kit and was purchased from Ambion (Austin, Tex.). The following gene-specific RACE primers were used.

| | |
|---|---|
| 5′RACE gene specific outer primer: | |
| TCTCCCACTGTATGCTCCTTCCA | (SEQ ID NO:65) |
| 5′RACE gene specific inner primer: | |
| CTCTGCCACTTCATCTGCAGGT | (SEQ ID NO:66) |

RLM-RACE was performed using 10 μg human spinal cord total RNA according to manufacturer's instructions, except as noted below. Total RNA was treated with calf intestine alkaline phosphatase to remove free 5′phosphates from molecules such as rRNA, fragmented mRNA, tRNA and contaminating DNA (this step entailed treatment with 3 μL calf intestine alkaline phosphatase (CIP) for 1.5 h at 37° C.). Full length mRNA molecules that contain a 5′ methylated guanosine (CAP) should not have been affected by this treatment. The RNA was then treated with tobacco acid pyrophosphatase to remove the CAP structure from full length mRNA, leaving a 5′phosphate. An RNA adapter oligonucleotide was ligated to full-length mRNA using T4 RNA ligase. The adapter could not ligate to dephosphorylated RNA molecules since they lacked a 5′phosphate. A random-primed reverse transcription reaction and nested PCR (using gene-specific inner primers) was then used to amplify the 5′ ends of a specific transcript. As a negative control, RNA was treated with calf intestinal alkaline phosphatase but not with tobacco acid pyrophosphatease, such that T4 RNA ligase should have had no substrate to ligate to the RNA adapter oligonucleotide. RLM-RACE products were separated by electrophoresis, extracted from the gel, purified, and sequenced using standard methods well known to those practiced in the art.

Results

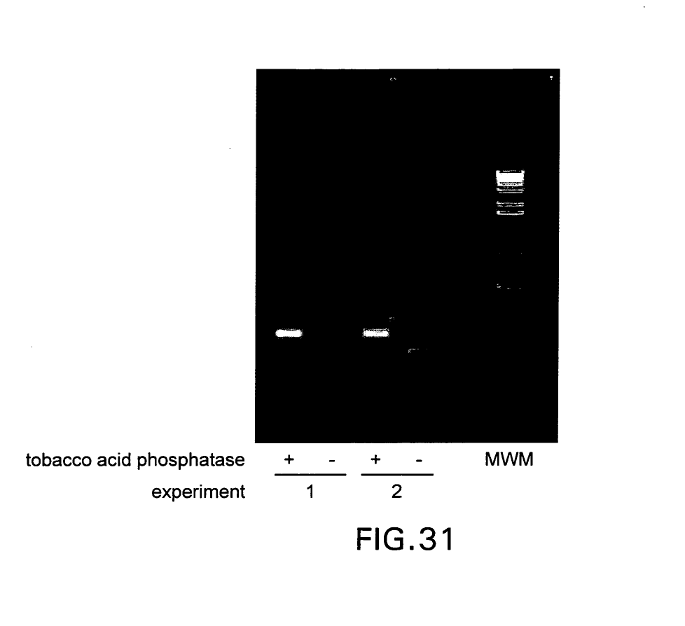

RLM-RACE was performed on 2 different lots of human spinal cord mRNA, with identical results (FIG. 31). Two bands (approximately 168 and 243 nucleotides) were observed using tobacco acid pyrophosphatase-treated RNA. These were not observed in negative control reactions.

Sequencing revealed 5′cDNA ends at nucleotides 1 and 75 of SEQ ID NO:2 (FIG. 30). Clean splices at the junction between the 5′RACE Adapter and the mRNA were not observed. This suggests that there was a mixed population of DNA within a single band on the gel, and that transcription initiation may also occur within a few bases upstream or downstream of the major transcription start sites.

Conclusion

Two major transcription start sites of DEP2-1 have been identified.

### EXAMPLE 7

### Characterization of the 5′ ends of DEP2-1 by exon-bridging PCR

Two exon-bridging RT-PCR experiments were conducted to learn whether DEP2-1 was a naturally occurring splice variant of LHPP, or only originates from one or more distinct transcriptional start sites. In a first experiment, PCR was conducted using a reverse primer within the first exon (nucleotides 1-315) of DEP2-1 (SEQ ID NO:2) and a forward primer within an upstream exon of LHPP (SEQ ID NO:9). This PCR was designed to amplify any transcript originating from the LHPP transcription start and comprising exon 1 of DEP2-1. In a second experiment, PCR was conducted using a forward primer within an upstream exon of LHPP and a reverse primer in the exon common to LHPP and DEP2-1. This PCR was designed to amplify any transcript containing both exons of DEP2-1 as well as upstream sequences from LHPP.

Methods

cDNA was prepared from 3 different lots of human spinal cord mRNA (BD Biosciences Clontech) using the SuperScript III First Strand Synthesis System (Invitrogen, Carlsbad, Calif.). The following primers were used.

Experiment 1

| | | |
|---|---|---|
| Forward: | TGCAAGCGATAGGAGTGGAA | (SEQ ID NO:44) |
| Reverse: | CCACCCCATGCCATCAA | (SEQ ID NO:39) |

Experiment 2

| | | |
|---|---|---|
| Forward: | TGCAAGCGATAGGAGTGGAA | (SEQ ID NO:44) |
| Reverse: | CACGTACCCATCAGCCTTCAC | (SEQ ID NO:67) |

PCR, electrophoresis and sequencing were performed using standard methods well known to those practiced in the art.

Results

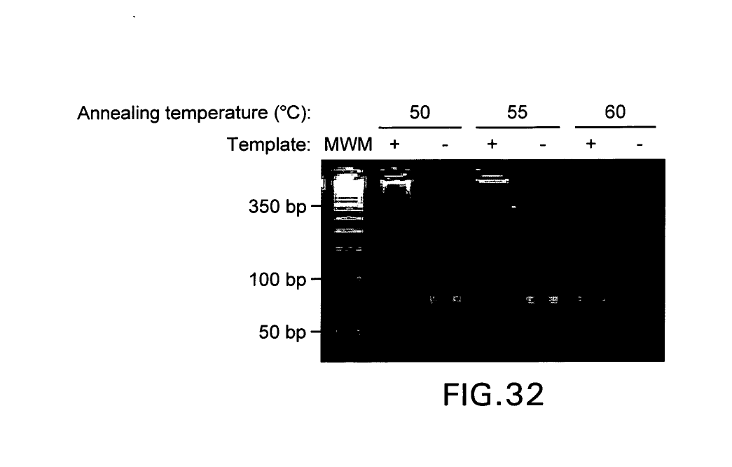

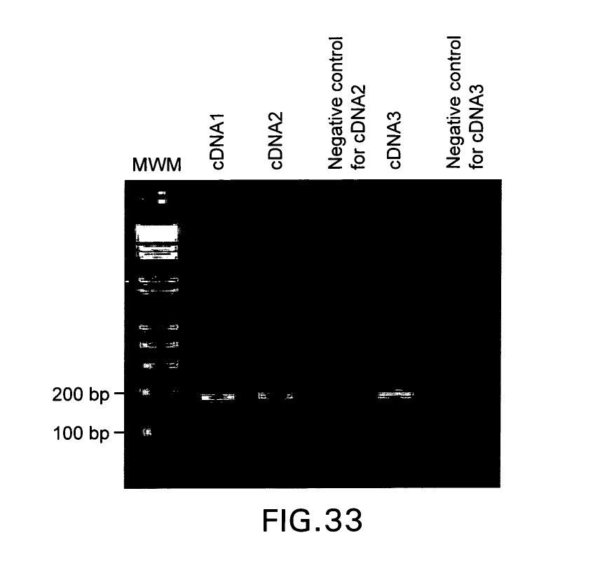

In experiment 1, products were observed following PCR and agarose gel electrophoresis (FIG. 32). These were weak and not consistently observed. Sequencing revealed that these were PCR artifacts not containing any sequence from SEQ ID NO:2. In experiment 2, products were observed following PCR and agarose gel electrophoresis (FIG. 33). This was expected, as the primer pair used amplifies LHPP (SEQ ID NO:9). No sequence from exon 1 (nucleotides 1-315) of SEQ ID NO:2 was detected in these products.

Conclusion

DEP2-1 and LHPP do not share a transcriptional start site.

### EXAMPLE 8

### Expression of LHPP, a Naturally Occurring Splice Variant Thereof, and DEP2-1, in Human Neuronal and Glial Cell Lines

To establish feasibility of cell-based assays to screen for compositions that modulate the activity or expression of DEP2 products, quantitative PCR (“QPCR”) experiments were conducted to detect expression of DEP2-1 (SEQ ID NO:2), LHPP (SEQ ID NO:9) and a naturally occurring splice variant thereof (SEQ ID NO:12).

Methods

Cells of six ATCC cell lines (SH-SY5H, SK-N_SH, LN18, H4, Ntera2 and U87MG) were suspended in RNALater (Ambion, Austin Tex.). Total RNA was isolated from each cell line using TRIZOL reagent (Invitrogen, Carlsbad Calif.) and purified using RNeasy columns (Qiagen, Valencia Calif.). Reverse transcription and PCR conditions were done as described in Example 4.

Results

Observed Ct values are shown in Table 14.

TABLE 14

| | SEQ ID NO: | | |
|---|---|---|---|
| | 2 | 9 | 12 |
| SH-SY5H | 40 | 40 | 40 |
| SK-N_SH | 31.71 | 24.82 | 23.65 |
| LN18 | 31.02 | 25.16 | 24.01 |
| H4 | 30.04 | 25.79 | 24.72 |
| Ntera2 | 29.35 | 24.91 | 23.76 |
| U87MG | 31.57 | 27.19 | 26.17 |

Conclusions

Cell-based assays to screen for compositions that modulate expression of SEQ ID NO:2, SEQ ID NO:9 or SEQ ID NO:12, or that modulate expression or activity of the corresponding proteins Dep2-1a (SEQ ID NO:3), Dep2-1b (SEQ ID NO:4) or Lhpp (SEQ ID NO:10), may be feasible using five of the six tested cell lines.

One skilled in the art would readily appreciate that the present invention is well adapted to carry out the objects and obtain the ends and advantages mentioned, as well as those inherent therein. The molecular complexes and the methods, procedures, treatments, molecules, specific compounds described herein are presently representative of preferred embodiments, are exemplary, and are not intended as limitations on the scope of the invention. It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising,” “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. An isolated nucleic acid or fragment thereof comprising (a) a nucleotide sequence having at least 90% identity to SEQ ID NO:2; or (b) a complement comprising a nucleotide sequence having at least 90% identity to SEQ ID NO:2.

2. A purified polypeptide or fragment thereof comprising an amino acid sequence having at least 90% identity to: SEQ ID NO:3 or SEQ ID NO:4.

3. A vector comprising:
a) an isolated nucleic acid sequence comprising a nucleotide sequence having at least 90% identity to: (i) SEQ ID NO:2, (ii) nucleotides 351 to 770 of SEQ ID NO:2; or (iii) nucleotides 811 to 1161 of SEQ ID NO:2 or a complement comprising a nucleotide sequence having at least 90% identity to (i) SEQ ID NO:2, (ii) nucleotides 351 to 770 of SEQ ID NO:2; operably linked to
b) a regulatory sequence.

4. A host cell comprising the vector of claim 3.

5. A non-human transgenic animal having
a) an exogenous and stably transmitted nucleic acid comprising a nucleotide sequence of SEQ ID NO:2; or
b) a knock-out of a nucleic acid comprising a nucleotide sequence of SEQ ID NO:2.

6. A non-human transgenic animal having:
a) an exogenous and stably transmitted nucleic acid having a nucleotide sequence selected from the group consisting of: SEQ ID NO:9, SEQ ID NO:11, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:31 and SEQ ID NO:33, with the proviso that said animal does not comprise an exogenous and stably transmitted nucleic acid having a nucleotide sequence of SEQ ID NO:2; or
b) a knock-out of a nucleic acid comprising a nucleotide sequence selected from the group consisting of: SEQ ID NO:9, SEQ ID NO:11, SEQ ID NO:12, SEQ ID NO:14, SEQ ID NO:16, SEQ ID NO:18, SEQ ID NO:20, SEQ ID NO: 22, SEQ ID NO:24, SEQ ID NO:26, SEQ ID NO:28, SEQ ID NO:30, SEQ ID NO:31 and SEQ ID NO:33, with the proviso that a nucleic acid having a nucleotide sequence of SEQ ID NO:2 is not knocked out.

7. A method of modifying or altering the expression of SEQ ID NO:2 in a cell or animal, the method comprising the steps of:
a) exposing said cell or administering to said subject a nucleic acid molecule, wherein said nucleic acid molecule modifies or alters the expression of SEQ ID NO: 2; and
b) modifying or altering the expression of SEQ ID NO:2.

8. A method of modifying or altering the expression of a nucleic acid sequence having a nucleotide sequence selected from the group consisting of: SEQ ID NO:9, SEQ ID NO:11, SEQ ID NO:12, SEQ ID NO:14, SEQ ID NO:16, SEQ ID NO:18, SEQ ID NO:20, SEQ ID NO: 22, SEQ ID NO:24, SEQ ID NO:26, SEQ ID NO:28, SEQ ID NO:30, SEQ ID NO:31 and SEQ ID NO:33 in a cell or animal, with the proviso that the expression of a nucleic acid having the sequence of SEQ ID NO: 2 is not modified or altered, the method comprising the steps of:
a) exposing said cell or administering to said subject a nucleic acid molecule, wherein said nucleic acid molecule modifies or alters the expression of a nucleotide sequence selected from the group consisting of: SEQ ID NO:9, SEQ ID NO:11, SEQ ID NO:12, SEQ ID NO:14, SEQ ID NO:16, SEQ ID NO:18, SEQ ID NO:20, SEQ ID NO: 22, SEQ ID NO:24, SEQ ID NO:26, SEQ ID NO:28, SEQ ID NO:30, SEQ ID NO:31 and SEQ ID NO:33; and
b) modifying or altering the expression of SEQ ID NO:9, SEQ ID NO:11, SEQ ID NO:12, SEQ ID NO:14, SEQ ID NO:16, SEQ ID NO:18, SEQ ID NO:20, SEQ ID NO: 22, SEQ ID NO:24, SEQ ID NO:26, SEQ ID NO:28, SEQ ID NO:30, SEQ ID NO:31 or SEQ ID NO:33.

9. The method of claims 7 or 8, wherein the nucleic acid molecule is an antisense molecule, a small interfering RNA, a co-suppression RNA, an aptamer, a ribozyme or a triplexing agent.

10. A method of determining a genotype of a subject at a polymorphic site in nucleotides 1 to 315 of SEQ ID NO: 2 in a test sample, the method comprising the steps of:
a) obtaining a test sample comprising DNA of a subject;
b) analyzing the test sample for at least one polymorphic site in nucleotides 1 to 315 of SEQ ID NO:2;
c) identifying the allele(s) present at said polymorphic site in said test sample; and
d) determining the genotype of a subject based on the identification of the allele(s) at said polymorphic site in said test sample.

11. A method of determining a genotype of a subject at nucleotides 77402 or 79906 of SEQ ID NO:1 in a test sample, the method comprising the steps of:
a) obtaining a test sample comprising DNA of a subject;
b) analyzing the test sample for at least one polymorphic site selected from the group consisting of nucleotides 77402 and 79906 of SEQ ID NO:1;
c) determining the allele(s) present at said polymorphic site in said test sample; and
d) determining the genotype of a subject based on the identification of the allele(s) at said polymorphic site in said test sample.

12. A method of identifying a subject having major depression or a related disorder, or at risk of developing major depression or a related disorder, the method comprising the steps of:
a) obtaining a test sample subject comprising DNA of a subject;
b) analyzing the test sample for at least one polymorphic site in SEQ ID NO: 1;
c) identifying at least one allele at said polymorphic site; and
d) identifying whether said subject has major depression or a related disorder or is at risk of developing major depression or a related disorder based on the allele(s) identified at said polymorphic site(s) in said test sample.

13. A method of providing a prognosis for or predicting a response to treatment for a subject having major depression or a related disorder, the method comprising the steps of:
a) obtaining a test sample comprising DNA of a subject;
b) analyzing the test sample for at least one polymorphic site in SEQ ID NO:1;
c) identifying at least one allele(s) at said polymorphic site; and
d) providing a prognosis for and predicting the response to treatment for a subject having major depression or a related disorder based on the allele(s) identified at said polymorphic site(s) in said test sample.

14. The method of claims 10, 11, 12 or 13, wherein the method further comprises the step of analyzing the test sample(s) for a C-G polymorphism at position −1019 in a human serotonin receptor 1A (HTR1A) gene.

15. The method of claims 10, 11, 12, 13, or 14, wherein the analyzing is accomplished using direct sequencing, polymerase chain reaction, ligase chain reaction, a fragment length polymorphism assay, a single strand conformation polymorphism analysis, a heteroduplex assay, hybridization, Taqman®, Molecular Beacon, Pyrosequencing, a microarray, Southern blotting, an Invader assay, a single base extension assay, or mass spectrometry.

16. A kit comprising:
a) at least one reagent for determining a genotype of a subject at a polymorphic site in SEQ ID NO:1 in a test sample; and
b) instructions for determining the genotype of the subject.

17. A kit comprising:
a) at least one reagent for determining a genotype of a subject at nucleotide 77402 in SEQ ID NO:1 in a test sample; and
b) instructions for determining the genotype of the subject.

18. A kit comprising:
a) at least one reagent for determining a genotype of a subject at nucleotide 79906 in SEQ ID NO:1 in a test sample; and
b) instructions for determining the genotype of the subject.

19. A kit comprising:
a) at least one reagent for determining a genotype of a subject for a C-G polymorphism at position −1019 in a human serotonin receptor 1A (HTR1A) gene;
b) at least one reagent for determining a genotype of a subject for a polymorphic site in SEQ ID NO:1, in a test sample; and
c) instructions for determining the genotype of the subject.

20. A method of detecting or quantifying an mRNA which comprises nucleotides 1 to 315 of SEQ ID NO:2 in a test sample, the method comprising the steps of:
c) obtaining a test sample subject comprising mRNA of a subject;
b) analyzing the test sample for a mRNA comprising at least 15 contiguous nucleotides of nucleotides 1 to 315 of SEQ ID NO:2; and
c) detecting or quantifying said mRNA in said test sample.

21. A method of identifying a subject having major depression or a related disorder, or at risk of developing major depression or a related disorder, the method comprising the steps of:
a) obtaining a test sample subject comprising subject mRNA;
b) analyzing the test sample for at least one mRNA transcribed from SEQ ID NO: 1; and
c) identifying whether said subject has major depression or a related disorder or is at risk of developing major depression or a related disorder based on the presence, absence or amount of at least one of the mRNAs recited in step b) in said test sample.

22. A method of providing a prognosis for or predicting a response to treatment for a subject having major depression or a related disorder, the method comprising the steps of:
a) obtaining a test sample comprising mRNA of a subject;
b) analyzing the test sample for at least one mRNA transcribed from SEQ ID NO: 1; and
c) providing a prognosis for and predicting the response to treatment for a subject having major depression or a related disorder based on the presence, absence or amount of at least one of the mRNAs recited in step b) in said test sample.

23. The method of claims 20, 21 or 22, wherein the method further comprises the step of analyzing the test sample(s) for an mRNA transcribed from a human serotonin receptor 1A (HTR1A) gene.

24. The method of claims 20, 21, 22 or 23, wherein the analyzing is accomplished using reverse transcription, quantitative polymerase chain reaction, cDNA microarrays, or Northern blotting.

25. The method of claims 20, 21, 22, 23 or 24, wherein the mRNA transcribed from SEQ ID NO: 1 has the nucleotide sequence of: SEQ ID NO:2, SEQ ID NO:5, SEQ ID NO:6, SEQ ID NO:7, SEQ ID NO:8, SEQ ID NO:9, SEQ ID NO:11, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:31 or SEQ ID NO:33.

26. A kit comprising:
a) at least one reagent for detecting or quantifying an mRNA transcribed from SEQ ID NO:1 in a test sample; and
b) instructions for detecting or quantifying the mRNA transcribed from SEQ ID NO:1 in the test sample.

27. A kit comprising:
a) at least one reagent for detecting or quantifying an mRNA from a serotonin receptor 1A (HTR1A) gene in a test sample;
b) at least one reagent for detecting or quantifying an mRNA transcribed from SEQ ID NO:1, in a test sample; and
c) instructions for detecting or quantifying the mRNA from a serotonin receptor 1A (HTR1A) gene and the mRNA transcribed from SEQ ID NO:1 in the test sample.

28. The kit of claims 26 or 27, wherein the mRNA transcribed from SEQ ID NO:1 has the nucleotide sequence of: SEQ ID NO:2, SEQ ID NO:5, SEQ ID NO:6, SEQ ID NO:7, SEQ ID NO:8, SEQ ID NO:9, SEQ ID NO:11, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:31 or SEQ ID NO:33.

29. A method of detecting or quantifying the amount of a protein having an amino acid sequence selected from the group consisting of: SEQ ID NO:3 and SEQ ID NO:4 in a test sample, the method comprising the steps of:
a) obtaining a test sample subject comprising at least one polypeptide of a subject; and
b) detecting or quantifying the amount of a protein having an amino acid sequence selected from the group consisting of SEQ ID NO: 3 and SEQ ID NO:4 in said test sample.

30. A method of identifying a subject having major depression or a related disorder, or at risk of developing major depression or a related disorder, the method comprising the steps of:
a) obtaining a test sample comprising at least one polypeptide of a subject;
b) analyzing the test sample for at least one polypeptide translated from SEQ ID NO:1; and
c) identifying whether said subject has major depression or a related disorder or is at risk of developing major depression or a related disorder based on the presence, absence or amount of at least of the polypeptides recited in step b) in said test sample.

31. A method of providing a prognosis for or predicting a response to treatment for a subject having major depression or a related disorder, the method comprising the steps of:
a) obtaining a test sample comprising at least one polypeptide of a subject;
b) analyzing the test sample for at least one polypeptide translated from SEQ ID NO:1; and
c) providing a prognosis for and predicting the response to treatment for a subject having major depression or a related disorder based on the presence, absence or amount of at least one of the polypeptides recited in step b) in said test sample.

32. The method of claims 30 or 31, wherein step b) further comprises the step of analyzing the test sample for a polypeptide translated from a human serotonin receptor 1A (HTR1A) gene of said subject.

33. The method of claims 29, 30, 31 or 32, wherein the detection is accomplished using ELISA, RIA, Western blotting, fluorescence activated cell sorting or immunohistochemical analysis.

34. The method of claims 30, 31, 32 or 33, wherein the polypeptide translated from SEQ ID NO:1 has an amino acid sequence selected from the group consisting of SEQ ID NO:3, SEQ ID NO:4, SEQ ID NO:10, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:32 and SEQ ID NO:34.

35. A kit comprising:
a) at least one reagent for detecting or quantifying a polypeptide translated from SEQ ID NO:1 in a test sample; and
b) instructions for detecting or quantifying the polypeptide translated from SEQ ID NO:1 in the test sample.

36. A kit comprising:
a) at least one reagent for detecting or quantifying a polypeptide encoded by a serotonin receptor 1A (HTR1A) gene in a test sample;
b) at least one reagent for detecting or quantifying a polypeptide translated from SEQ ID NO:1, in a test sample; and
c) instructions for detecting or quantifying the polypeptide encoded by a serotonin receptor 1A (HTR1A) gene and the polypeptide translated from SEQ ID NO:1 in the test sample.

37. The kit of claims 35 or 36, wherein the polypeptide translated from SEQ ID NO:1 has an amino acid sequence selected from the group consisting of SEQ ID NO:3, SEQ ID NO:4, SEQ ID NO:10, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:32 and SEQ ID NO:34.

38. A method of screening a composition for the ability to bind to a protein translated from SEQ ID NO:1, the method comprising the steps of:
a) exposing said protein to a composition for a time and under conditions sufficient for said test composition to bind to said protein to form protein/composition complexes; and
b) detecting presence or absence of said complexes, wherein the presence of said complexes indicates a composition having the ability to bind to said protein.

39. The method of claim 38, wherein a composition having the ability to bind to said protein can be used to treat major depression or a related disorder in a subject.

40. The method of claim 38, wherein the presence or absence of the complexes is detected by mass spectrometry.

41. A method of detecting binding of a composition to a protein translated from SEQ ID NO:1, the method comprising the steps of:
a) subjecting said protein to nuclear magnetic resonance and recording the resulting spectrum;
b) subjecting said protein to nuclear magnetic resonance in the presence of said composition and recording the resulting spectrum; and
c) detecting the difference between said spectrum of step a) and said spectrum of step b) and comparing said difference to a control, said comparison indicating whether said composition binds to said protein.

42. A method of identifying the structure of a composition bound to a protein translated from SEQ ID NO:1, the method comprising the steps of:
a) exposing said protein to a composition known to bind to said protein; and
b) observing the resulting X-ray diffraction pattern of said resulting bound composition of step a), said diffraction pattern indicating the structure of said composition.

43. The method of claims 41 or 42, wherein a composition detected as binding to said protein can be used to treat major depression or a related disorder in a subject.

44. A method of screening a composition for the ability to modulate the activity of a protein translated from SEQ ID NO:1, the method comprising the steps of:
a) providing a composition;
b) exposing the protein to a substrate for sufficient time and conditions to allow the substrate to react with the protein in order to produce a reaction product or complex;
c) exposing the protein to the composition; and
d) measuring said reaction product or complex, wherein a decreased or increased amount of said reaction product or complex, as compared to the amount of reaction product or complex produced in the absence of said composition, indicates a composition having the ability to modulate the activity of said protein.

45. A method of screening a composition for the ability to modulate the activity of a protein translated from SEQ ID NO:1, the method comprising the steps of:
a) providing a composition;
b) simultaneously exposing the protein to the composition and to a substrate, wherein the protein is exposed to the substrate for sufficient time and conditions to allow the substrate to react with the protein in order to produce a reaction product or complex; and
c) measuring presence or absence of said reaction product or complex, wherein a lack of said reaction product or complex indicating a composition having the ability to modulate the activity of said protein.

46. The method of claims 44 or 45, wherein the protein modifies the phosphorylation of the substrate.

47. The method of claim 46, wherein the protein translated from SEQ ID NO:1 has an amino acid sequence selected from the group consisting of SEQ ID NO:10, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:32 and SEQ ID NO:34.

48. The method of claims 44, 45, 46 or 47, wherein the substrate is selected from the group consisting of: phosphohistidine, phospholysine, phosphodiimide, pyrophosphate and a peptide or protein phosphorylated on histidine or lysine.

49. A method of screening a composition for the ability to modulate activity of a protein translated from SEQ ID NO:1, in a cell, the method comprising the steps of:
a) exposing said cell to said composition; and
b) measuring the amount of activity of said protein in said cell, wherein a decreased or increased amount of activity of said protein, as compared to a cell which has not been exposed to said composition, indicates a composition having the ability to modulate the activity of said protein.

50. The method of claims 44, 45 or 49, wherein a composition having an ability to modulate the activity of a protein can be used to treat major depression or a related disorder in a subject.

51. A method of screening a composition for the ability to modulate expression of a protein translated from SEQ ID NO:1, in a cell, the method comprising the steps of:
a) exposing said cell to said composition; and
b) measuring the amount of said protein in said cell, wherein a decreased or increased amount of said protein, as compared to a cell which has not been exposed to said composition, indicates a composition having the ability to modulate the expression of said protein.

52. A method of screening a composition for the ability to modulate the level of expression of a protein translated from SEQ ID NO:1, the method comprising the steps of:
a) exposing an in vitro transcription and translation system comprising a regulatory sequence from SEQ ID NO:1 functionally connected to the open reading frame for a detectable protein, to a composition for a time and under conditions sufficient for said test whether said composition modulates the level of expression of the detectable protein; and
b) detecting the level of expression of the detectable protein, wherein a reduction or an increase in the level of expression of the detectable protein indicates that said composition has the ability to modulate the level of expression of a protein translated from SEQ ID NO:1.

53. The method of claims 51 or 52, wherein the composition having an ability to modulate the expression of the protein or the level of expression of the protein can be used to treat major depression or a related disorder in a subject.

54. The method of claims 49, 51 or 52, wherein the protein translated from SEQ ID NO:1 has an amino acid sequence selected from the group consisting of SEQ ID NO:3, SEQ ID NO:4, SEQ ID NO:10, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:32 and SEQ ID NO:34.

55. A method of treating major depression or a related disorder in a subject in need of said treatment comprising the step of administering a composition identified as modulating the activity of a protein translated from SEQ ID NO:1, to said subject, in an amount sufficient to effect said treatment.

56. The method of claim 55, wherein said composition inhibits or reduces the activity of the protein.

57. The method of claim 55, wherein said composition increases the activity of the protein.

58. The method of claim 55, wherein said composition decreases the activity of the protein.

59. A method of treating major depression or a related disorder in a subject in need of said treatment comprising reducing the amount of a protein translated from SEQ ID NO:1 in said subject, to a level sufficient to effect said treatment.

60. The method of claim 59, wherein said reduction results from complete binding or partial binding of a composition to said protein.

61. A method of treating major depression or a related disorder in a subject in need of said treatment comprising increasing the amount of a protein translated from SEQ ID NO:1, to a level sufficient to effect said treatment.

62. The methods of claims 59 or 61, wherein said method involves administering to said subject a therapeutically effective amount of a protein translated from SEQ ID NO:1.

63. The method of claims 55 or 62, wherein the protein translated from SEQ ID NO:1 has an amino acid sequence selected from the group consisting of SEQ ID NO:3, SEQ ID NO:4, SEQ ID NO:10, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:32 and SEQ ID NO:34.

64. A method of treating major depression or a related disorder in a subject in need of said treatment comprising the step of administering a composition identified as modulating the level of expression of an mRNA molecule transcribed from SEQ ID NO:1 to said subject, in an amount sufficient to effect said treatment.

65. The method of claim 64, wherein the mRNA molecule transcribed from SEQ ID NO:1 has the nucleotide sequence of SEQ ID NO:2, SEQ ID NO:5, SEQ ID NO:6, SEQ ID NO:7, SEQ ID NO:8, SEQ ID NO:9, SEQ ID NO:11, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:31 or SEQ ID NO:33.

66. A method of determining the therapeutic activity of a composition used to treat major depression or a related disorder comprising the steps of:
a) determining the amount of a protein translated from SEQ ID NO:1, in a test sample from a subject treated with said composition; and
b) comparing the amount of said protein in said test sample with the amount of protein present in a test sample from said subject prior to treatment, an equal amount of said protein in said test sample of said treated subject indicating lack of therapeutic activity of said composition and a changed amount of said protein in said test sample of said treated subject indicating therapeutic activity of said composition.

67. A method of determining the level of therapeutic activity of a composition used to treat major depression or a related disorder comprising the steps of:
a) determining the activity of a protein translated from SEQ ID NO:1, in a test sample from a subject treated with said composition; and
b) comparing the amount of activity of said protein in said test sample with the amount of activity of protein present in a test sample from said subject prior to treatment, an equal amount of activity of said protein in said test sample of said treated subject indicating lack of therapeutic activity of said composition and a changed amount of activity of said protein in said test sample of said treated subject indicating therapeutic activity of said composition.

68. The method of claims 66 or 67, wherein the protein translated from SEQ ID NO:1 has an amino acid sequence selected from the group consisting of SEQ ID NO:3, SEQ ID NO:4, SEQ ID NO:10, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:32 and SEQ ID NO:34.

69. A method of determining the level of in vivo activity of a composition used to treat major depression or a related disorder comprising the steps of:
a) determining the amount of a mRNA molecule transcribed from SEQ ID NO:1, in a test sample from a subject treated with said composition; and
b) comparing the amount of said mRNA molecule in said test sample with the amount of mRNA molecule present in a test sample from said subject prior to treatment, an equal amount of said mRNA molecule in said test sample of said treated subject indicating lack of therapeutic activity of said composition and a changed amount of said mRNA molecule in said test sample of said treated subject indicating therapeutic activity of said composition.

70. The method of claim 69, wherein the mRNA molecule transcribed from SEQ ID NO:1 has the nucleotide sequence of SEQ ID NO:2, SEQ ID NO:5, SEQ ID NO:6, SEQ ID NO:7, SEQ ID NO:8, SEQ ID NO:9, SEQ ID NO:11, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:31 or SEQ ID NO:33.

71. A method of determining presence or absence of activity of a composition used to treat major depression or a related disorder comprising the steps of:
a) observing phenotype of a subject according to a method validated as a measure of major depression or a related disorder;
b) administering said composition to said subject for a time and under conditions sufficient for said composition to bind to, inhibit, increase or reduce the activity of, or increase or reduce the amount of a protein translated from SEQ ID NO:1;
c) repeating step a) with said subject of step b); and
d) comparing said phenotype of step a) and said phenotype of step c), a difference in step c) as compared to step a) indicating presence of activity of said composition and the lack of a difference indicating absence of activity of said composition.

72. The method of claim 71, wherein the protein translated from SEQ ID NO:1 has an amino acid sequence selected from the group consisting of SEQ ID NO:3, SEQ ID NO:4, SEQ ID NO:10, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:32 and SEQ ID NO:34.

73. A method of determining presence or absence of activity of a composition used to treat major depression or a related disorder comprising the steps of:
a) observing phenotype of a subject according to a method validated as a measure of major depression or a related disorder;
b) administering said composition to said subject for a time and under conditions sufficient for said composition to increase or reduce the amount of a mRNA molecule transcribed from SEQ ID NO:1;
c) repeating step a) with said subject of step b); and
d) comparing said phenotype of step a) and said phenotype of step c), a difference in step c) as compared to step a) indicating presence of activity of said composition and the lack of a difference indicating absence of activity of said composition.

74. The method of claim 73, wherein the mRNA molecule transcribed from SEQ ID NO:1 has the nucleotide sequence of SEQ ID NO:2, SEQ ID NO:5, SEQ ID NO:6, SEQ ID NO:7, SEQ ID NO:8, SEQ ID NO:9, SEQ ID NO:11, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:31 or SEQ ID NO:33.
